(19) United States
(12) Patent Application Publication
(10) Pub. No.: US 2022/0399506 A1
(43) Pub. Date: Dec. 15, 2022

(54) ELECTROLUMINESCENT DEVICE

(71) Applicant: BEIJING SUMMER SPROUT TECHNOLOGY CO., LTD., Beijing (CN)

(72) Inventors: Chi Yuen Raymond Kwong, Beijing (CN); Le Wang, Beijing (CN); Cuifang Zhang, Beijing (CN); Qiang Wang, Beijing (CN); Han Zhang, Beijing (CN); Junfei Wang, Beijing (CN); Qi Zhang, Beijing (CN); Nannan Lu, Beijing (CN); Zhihong Dai, Beijing (CN); Menglan Xie, Beijing (CN)

(21) Appl. No.: 17/732,591

(22) Filed: Apr. 29, 2022

(30) Foreign Application Priority Data
Apr. 30, 2021 (CN) 202110464325.5
Mar. 10, 2022 (CN) 202210231301.X

(51) Int. Cl. H01L 51/00; C07D 487/16; C09K 11/06; C07F 15/00

(52) U.S. Cl. CPC H01L 51/0072; C07D 487/16; C09K 11/06; H01L 51/0067; C07F 15/0033; H01L 51/0085; H01L 51/5012

(57) ABSTRACT

Provided is an electroluminescent device. The organic electroluminescent device comprises an anode, a cathode and an organic layer disposed between the anode and the cathode, where the organic layer comprises a first compound having a structure of H-L-E and a second compound comprising a ligand Lₐ having a structure of Formula C. Such a new material combination consisting of the first compound and the second compound can obtain higher efficiency in the device, significantly extend a lifetime, and provide better device performance. Further provided are a display assembly comprising the electroluminescent device and a compound combination.

### CROSS-REFERENCE TO RELATED APPLICATION(S)

This application claims priority to Chinese Patent Application No. CN202110464325.5 filed on Apr. 30, 2021 and Chinese Patent Application No. CN202210231301.X filed on Mar. 10, 2022, the disclosure of which are incorporated herein by reference in their entireties.

### TECHNICAL FIELD

The present disclosure relates to electronic devices, for example, electroluminescent devices. More particularly, the present disclosure relates to an electroluminescent device comprising a new material combination of a first compound having a structure of H-L-E and a second compound comprising a ligand Lₐ having a structure of Formula C in an organic layer.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

US20180337340A1 has disclosed an organic electroluminescent compound and an organic electroluminescent device comprising the same. The organic electroluminescent device comprises an organic layer comprising one or more hosts, a first host of which is an organic

optical compound having the following structure:

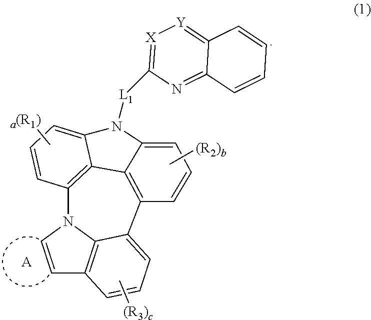

However, neither does it disclose or teach a special advantage when the organic optical compound is used with a phosphorescent material having a particular structure, nor does it further studies a special advantage when another compound having a similar carbazole-fused macrocyclic structure is used with the phosphorescent material having the particular structure.

To meet the increasing requirements of the industry for various aspects of performance of electroluminescent devices, such as emitted color, color saturation of luminescence, driving voltage, luminescence efficiency and device lifetime, researches related to phosphorescent devices are still urgently needed. In the researches on the phosphorescent devices, it is very important to use a phosphorescent material in combination with a host material, and how to combine and select the phosphorescent material and the host material directly relates to the luminescence performance of the devices. Therefore, how to select and optimize a combination of the phosphorescent material and the host material is an important part of the related researches of the industry.

### SUMMARY

The present disclosure aims to provide an electroluminescent device having a new material combination to solve at least part of the above problems. An organic layer of the electroluminescent device comprises a new material combination consisting of a first compound having a structure of H-L-E and a second compound comprising a ligand Lₐ having a structure of Formula C. Such a novel material combination can be used in a light-emitting layer of the electroluminescent device. Such a novel material combination can obtain higher efficiency in the device, significantly extend a lifetime, and provide better device performance.

According to an embodiment of the present disclosure, disclosed is an electroluminescent device comprising an anode, a cathode and an organic layer disposed between the anode and the cathode, wherein the organic layer comprises at least a first compound and a second compound;
wherein the first compound has a structure of H-L-E, wherein the H has a structure represented by Formula A:

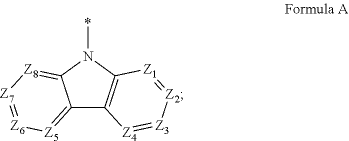

wherein in Formula A,

Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;

L is selected from a single bond, substituted or unsubstituted arylene having 6 to 30 carbon atoms, substituted or unsubstituted heteroarylene having 3 to 30 carbon atoms or a combination thereof;

E is selected from substituted or unsubstituted aryl having 6 to 30 carbon atoms or substituted or unsubstituted heteroaryl having 3 to 30 carbon atoms;

R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

R_{z2} is, at each occurrence identically or differently, selected from the group consisting of: hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof; and

adjacent substituents R_{z1}, R_{z2} can be optionally joined to form a ring;

wherein the second compound is a metal complex, wherein the metal is selected from a metal with a relative atomic mass greater than 40, and the metal complex comprises a ligand Lₐ which has a structure represented by Formula C:

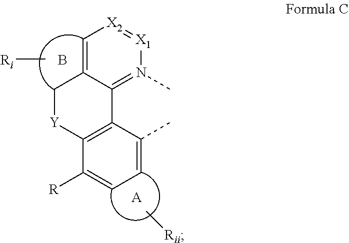

wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;

Rᵢ represents, at each occurrence identically or differently, mono-substitution, multiple substitutions or non-substitution; and Rᵢᵢ, represents, at each occurrence identically or differently, mono-substitution, multiple substitutions or non-substitution;

Y is selected from SiR_{y}R_{y}, GeR_{y}R_{y}, NR_{y}, PR_{y}, O, S or Se;

when two R_{y} are present, the two R_{y} may be identical or different;

X₁ and X₂ are, at each occurrence identically or differently, selected from CRₓ or N;

R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

adjacent substituents Rᵢ, Rₓ, R_{y}, R and Rᵢᵢ can be optionally joined to form a ring.

According to another embodiment of the present disclosure, further disclosed is a display assembly comprising the electroluminescent device described above.

According to another embodiment of the present disclosure, further disclosed is a compound combination including at least a first compound and a second compound;

wherein the first compound has a structure of H-L-E, wherein the H has a structure represented by Formula A:

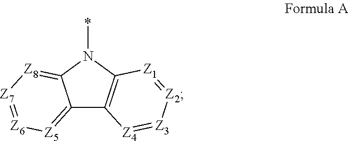

wherein in Formula A,

Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;

L is selected from a single bond, substituted or unsubstituted arylene having 6 to 30 carbon atoms, substituted or unsubstituted heteroarylene having 3 to 30 carbon atoms or a combination thereof;

E is selected from substituted or unsubstituted aryl having 6 to 30 carbon atoms or substituted or unsubstituted heteroaryl having 3 to 30 carbon atoms;

R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

R_{z2} is, at each occurrence identically or differently, selected from the group consisting of: hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof; and

adjacent substituents R_{z1}, R_{z2} can be optionally joined to form a ring;

wherein the second compound is a metal complex, wherein the metal is selected from a metal with a relative atomic mass greater than 40, and the metal complex comprises a ligand Lₐ which has a structure represented by Formula C:

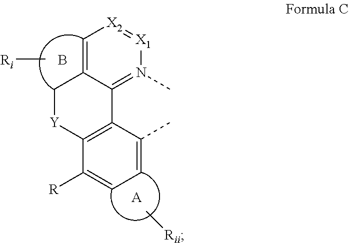

wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;

Rᵢ represents, at each occurrence identically or differently, mono-substitution, multiple substitutions or non-substitution; and Rᵢᵢ represents, at each occurrence identically or differently, mono-substitution, multiple substitutions or non-substitution;

Y is selected from SiR_{y}R_{y}, GeR_{y}R_{y}, NR_{y}, PR_{y}, O, S or Se;

when two R_{y} are present, the two R_{y} may be identical or different;

X₁ and X₂ are, at each occurrence identically or differently, selected from CRₓ or N;

R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof; and

adjacent substituents Rᵢ, Rₓ, R_{y}, R and Rₐ can be optionally joined to form a ring.

The present disclosure discloses a new electroluminescent device. The electroluminescent device uses the novel material combination consisting of the first compound having the structure of H-L-E and the second compound comprising the ligand Lₐ having the structure of Formula C. Such a novel material combination can be used in the light-emitting layer of the electroluminescent device. Such a novel material combination can enable the novel electroluminescent device to obtain the higher efficiency, can significantly extend the lifetime, and can provide the better device performance.

### BRIEF DESCRIPTION OF DRAWINGS

FIG. 1 is a schematic diagram of an organic light-emitting apparatus that may include an electroluminescent device disclosed herein.

FIG. 2 is a schematic diagram of another organic light-emitting apparatus that may include an electroluminescent device disclosed herein.

### DETAILED DESCRIPTION

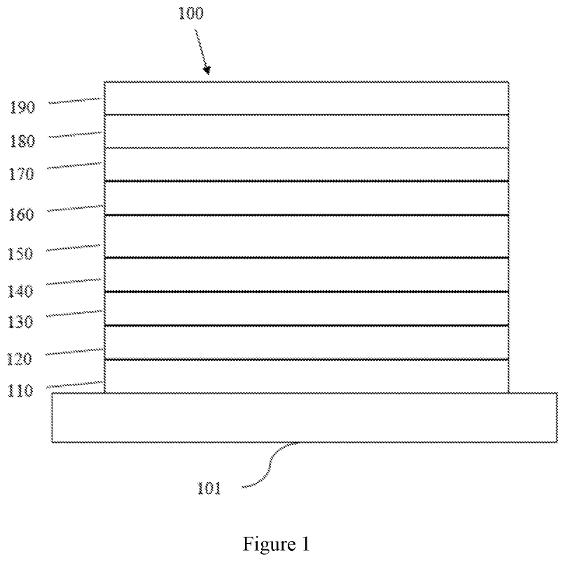

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

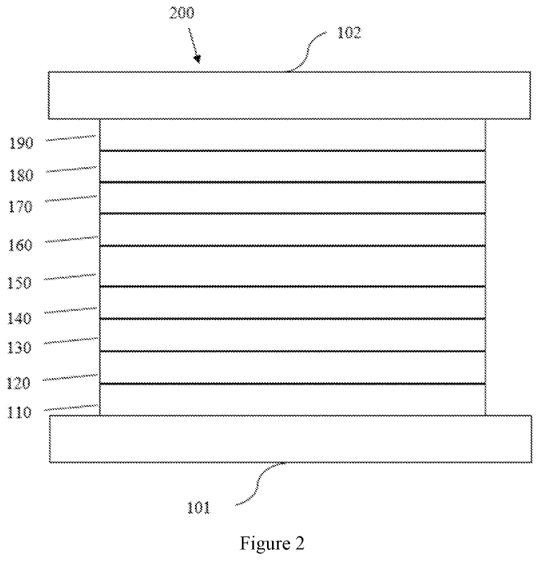

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, an n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethyl silyl, dimethyl ethyl silyl, dimethylisopropylsilyl, t-butyldimethyl silyl, tri ethyl silyl, triisopropylsilyl, trimethyl silylmethyl, trimethyl silyl ethyl, and trimethylsilylisopropyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, propenyl, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocyclic ring—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, wherein at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indenoazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, betanaphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group.

Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with at least one aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

The term “aza” in azadibenzofuran, azadibenzothiophene, etc. means that one or more of the C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogues are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted amino group, substituted acyl, substituted carbonyl, substituted carboxylic acid group, substituted ester group, substituted sulfinyl, substituted sulfonyl and substituted phosphino is used, it means that any group of alkyl, cycloalkyl, heteroalkyl, arylalkyl, alkoxy, aryloxy, alkenyl, alkynyl, aryl, heteroaryl, alkylsilyl, arylsilyl, amino group, acyl, carbonyl, carboxylic acid group, ester group, sulfinyl, sulfonyl and phosphino may be substituted with one or more groups selected from the group consisting of deuterium, a halogen, an unsubstituted alkyl group having 1 to 20 carbon atoms, an unsubstituted cycloalkyl group having 3 to 20 ring carbon atoms, an unsubstituted heteroalkyl group having 1 to 20 carbon atoms, an unsubstituted heterocyclic group having 3 to 20 ring atoms, an unsubstituted arylalkyl group having 7 to 30 carbon atoms, an unsubstituted alkoxy group having 1 to 20 carbon atoms, an unsubstituted aryloxy group having 6 to 30 carbon atoms, an unsubstituted alkenyl group having 2 to 20 carbon atoms, an unsubstituted alkynyl group having 2 to 20 carbon atoms, an unsubstituted aryl group having 6 to 30 carbon atoms, an unsubstituted heteroaryl group having 3 to 30 carbon atoms, an unsubstituted alkylsilyl group having 3 to 20 carbon atoms, an unsubstituted arylsilyl group having 6 to 20 carbon atoms, an unsubstituted amino group having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group and a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, the hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When a substitution in the compounds mentioned in the present disclosure represents multiple substitutions (including di, tri, tetra substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot connect to form a ring unless otherwise explicitly defined, for example, adjacent substituents can be optionally joined to form a ring. In the compounds mentioned in the present disclosure, adjacent substituents can be optionally joined to form a ring, including both the case where adjacent substituents can be joined to form a ring, and the case where adjacent substituents are not joined to form a ring. When adjacent substituents can be optionally joined to form a ring, the ring formed may be monocyclic or polycyclic, as well as alicyclic, heteroalicyclic, aromatic or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

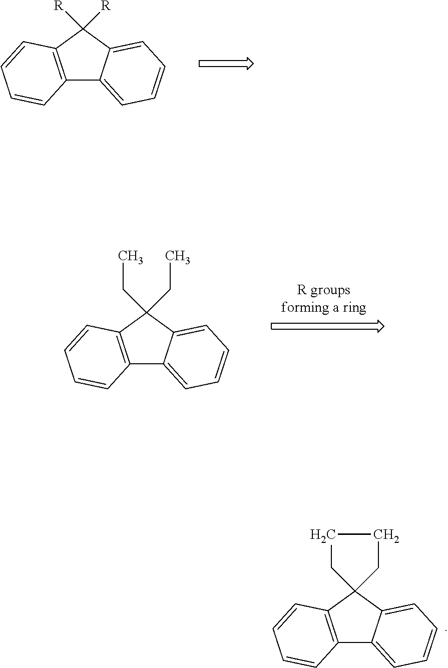

The expression that adjacent substituents can be optionally joined to form a ring is also intended to mean that two substituents bonded to carbon atoms which are directly bonded to each other are joined to each other via a chemical bond to form a ring, which can be exemplified by the following formula:

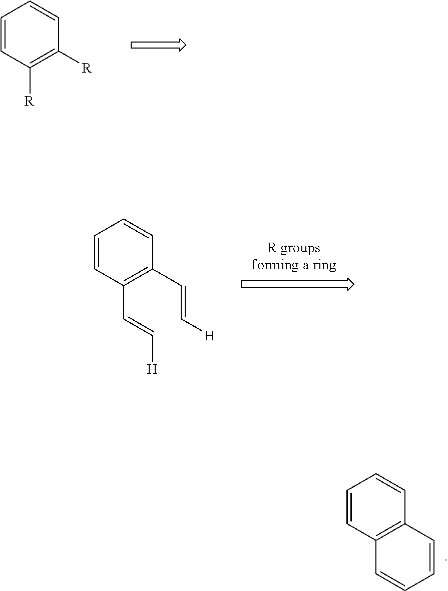

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

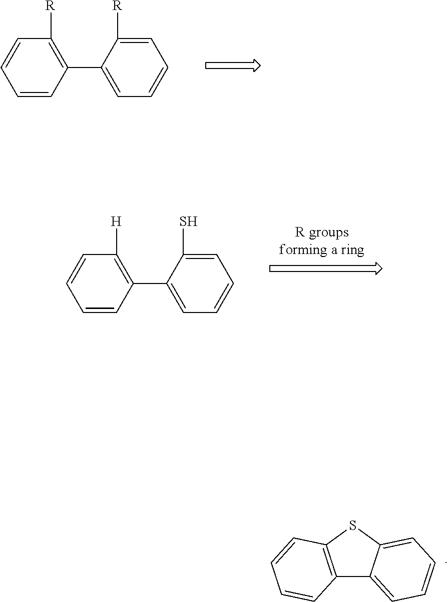

According to an embodiment of the present disclosure, disclosed is an electroluminescent device comprising:

an anode,

a cathode, and

an organic layer disposed between the anode and the cathode, wherein the organic layer comprises at least a first compound and a second compound;

wherein the first compound has a structure of H-L-E, wherein the H has a structure represented by Formula A:

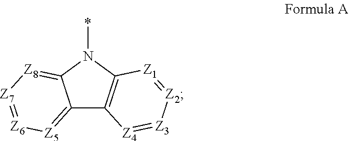

wherein in Formula A,

Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;

L is selected from a single bond, substituted or unsubstituted arylene having 6 to 30 carbon atoms, substituted or unsubstituted heteroarylene having 3 to 30 carbon atoms or a combination thereof;

E is selected from substituted or unsubstituted aryl having 6 to 30 carbon atoms or substituted or unsubstituted heteroaryl having 3 to 30 carbon atoms;

R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

R_{z2} is, at each occurrence identically or differently, selected from the group consisting of: hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof, and

adjacent substituents R_{z1}, R_{z2} can be optionally joined to form a ring;

wherein the second compound is a metal complex, wherein the metal is selected from a metal with a relative atomic mass greater than 40, and the metal complex comprises a ligand Lₐ which has a structure represented by Formula C:

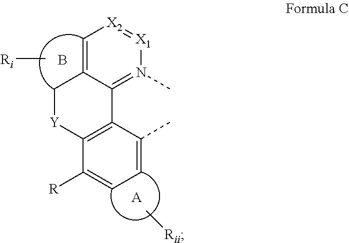

wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;

Rᵢ represents, at each occurrence identically or differently, mono-substitution, multiple substitutions or non-substitution; and Rᵢᵢ represents, at each occurrence identically or differently, mono-substitution, multiple substitutions or non-substitution;

Y is selected from SiR_{y}R_{y}, GeR_{y}R_{y}, NR_{y}, PR_{y}, O, S or Se;

when two R_{y} are present, the two R_{y} may be identical or different;

X₁ and X₂ are, at each occurrence identically or differently, selected from CRₓ or N;

R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents Rᵢ, Rₓ, R_{y}, R and Rᵢᵢ can be optionally joined to form a ring.

In this embodiment, the expression that “adjacent substituents R_{z1}, R_{z2} can be optionally joined to form a ring” is intended to mean that any one or more of groups of adjacent substituents, such as adjacent substituents R_{z1} in Z₁ to Z₃, adjacent substituents R_{z1} in Z₆ to Z₈, substituent R_{z1} in Z₃ and substituent R_{z2} in Z₄, substituent R_{z1} in Z₃ and substituent R_{z2} in Z₅, substituent R_{z1} in Z₆ and substituent R_{z2} in Z₄, and substituent R_{z1} in Z₆ and substituent R_{z2} in Z₅, can be joined to form a ring. Obviously, it is possible that none of these groups of adjacent substituents are joined to form a ring.

In the present disclosure, the expression that “adjacent substituents Rᵢ, Rₓ, R_{y}, R and Rᵢᵢ can be optionally joined to form a ring” is intended to mean that any one or more of groups of adjacent substituents, such as two substituents Rᵢ, two substituents Rᵢᵢ, two substituents R_{y}, two substituents Rₓ, substituents Rᵢ and Rₓ, substituents R and R_{y}, and substituents Rᵢᵢ and R, can be joined to form a ring. Obviously, it is possible that none of these substituents are joined to form a ring.

According to an embodiment of the present disclosure, wherein, in Formula A, the two substituents R_{z2} in Z₄ and Z₅ are joined to form a ring, and the ring has at least six ring atoms.

According to an embodiment of the present disclosure, wherein, in Formula A, the two substituents R_{z2} in Z₄ and Z₅ are joined to form a ring, and the ring has at least seven ring atoms.

According to an embodiment of the present disclosure, wherein, in the first compound, the H has a structure represented by any one of Formula A-1 to Formula A-8:

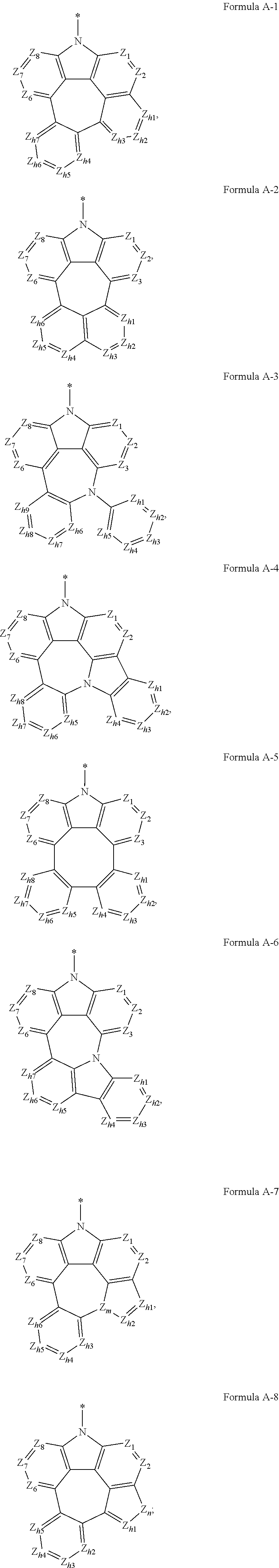

wherein in Formula A-1 to Formula A-8,

Z₁ to Z₃ and Z₆ to Z₈ are, at each occurrence identically or differently, selected from CR_{z1} or N;

Zₕ₁ to Zₕ₈ are, at each occurrence identically or differently, selected from CR_{zh} or N;

Zₘ is selected from CR_{zm} or N;

Zₙ is selected from CR_{zn}R_{zn}, O, S or NR_{zn}; wherein when Zₙ is selected from CR_{zn}R_{zn}, two R_{zn} may be identical or different;

R_{z1}, R_{zh}, R_{zm} and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents R_{z1}, R_{zh}, R_{zm}, R_{zn} can be optionally joined to form a ring.

In the present disclosure, the expression that “adjacent substituents R_{z1}, R_{zh}, R_{zm}, R_{zn} can be optionally joined to form a ring” is intended to mean that any one or more of groups of adjacent substituents, such as adjacent substituents R_{z1} in Z₁ to Z₃, adjacent substituents R_{z1} in Z₆ to Z₈, adjacent substituents R_{zh}, adjacent substituents R_{zh} and R_{zm}, adjacent substituents R_{zn}, and adjacent substituents R_{zh} and R_{zn}, can be joined to form a ring. Obviously, it is possible that none of these groups of adjacent substituents are joined to form a ring.

According to an embodiment of the present disclosure, wherein, in Formula A-1 to Formula A-8, R_{z1}, R_{zh}, R_{zm} and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted amino having 0 to 20 carbon atoms, cyano, isocyano, hydroxyl, a sulfanyl group and combinations thereof, and

adjacent substituents R_{z1}, R_{zh}, R_{zm}, R_{zn} can be optionally joined to form a ring.

According to an embodiment of the present disclosure, wherein, in the first compound, the H has a structure represented by any one of Formula A-1 to Formula A-8:

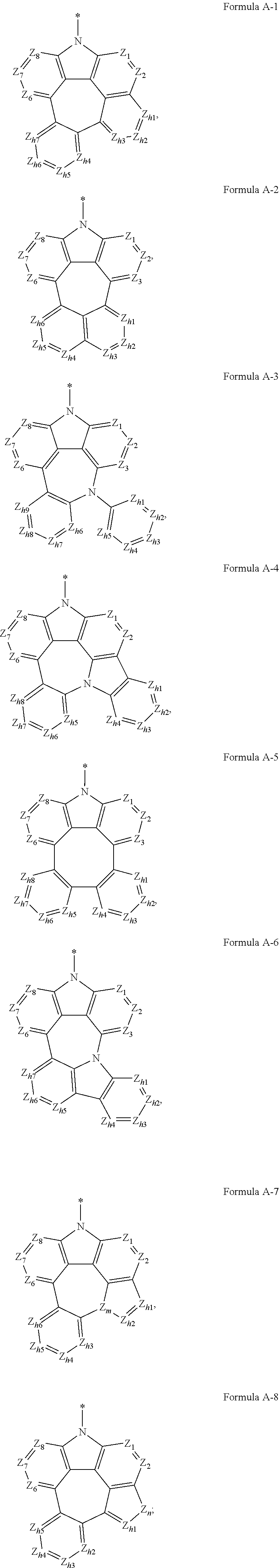

wherein in Formula A-1 to Formula A-8,

Z₁ to Z₃ and Z₆ to Z₈ are, at each occurrence identically or differently, selected from CR_{z1};

Zₕ₁ to Zₕ₈ are, at each occurrence identically or differently, selected from CR_{zh} or N;

Zₘ is selected from N;

Zₙ is selected from O, S or NR_{zn};

R_{z1}, R_{zh}, and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents R_{z1}, R_{zh}, R_{zn} can be optionally joined to form a ring.

In the present disclosure, the expression that “adjacent substituents R_{z1}, R_{zh}, R_{zn} can be optionally joined to form a ring” is intended to mean that any one or more of groups of adjacent substituents, such as adjacent substituents R_{z1} in Z₁ to Z₃, adjacent substituents R_{z1} in Z₆ to Z₈, adjacent substituents R_{zh}, adjacent substituents R_{zn}, and adjacent substituents R_{zh} and R_{zn}, can be joined to form a ring. Obviously, it is possible that none of these groups of adjacent substituents are joined to form a ring.

According to an embodiment of the present disclosure, wherein, in the first compound, the H has a structure represented by any one of Formula A-1 to Formula A-8:

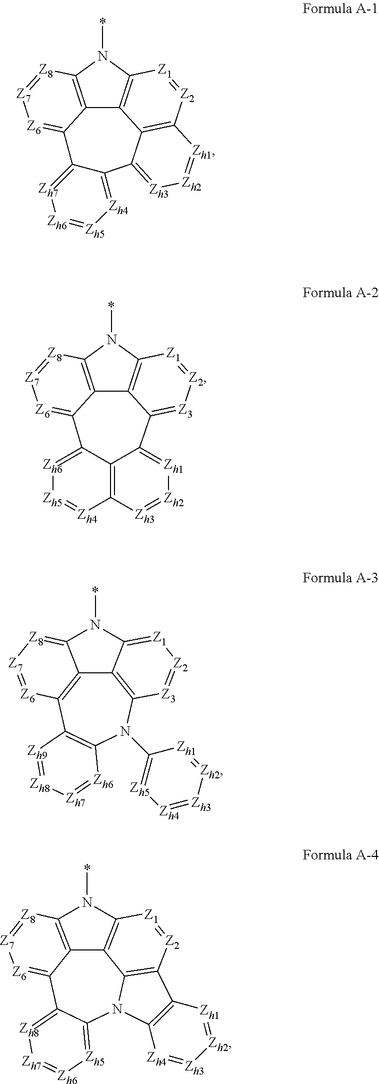

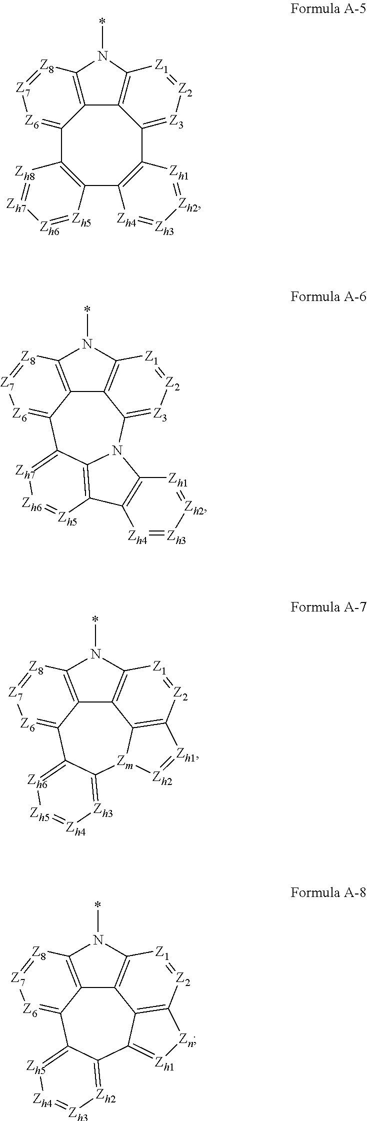

wherein in Formula A-1 to Formula A-8,

Z₁ to Z₃ and Z₆ to Z₈ are, at each occurrence identically or differently, selected from CR_{z1};

Zₕ₁ to Zₕ₈ are, at each occurrence identically or differently, selected from CR_{zh} or N;

Zₘ is selected from N;

Zₙ is selected from O, S or NR_{zn};

R_{z1}, R_{zh} and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, cyano, isocyano, hydroxyl, a sulfanyl group and combinations thereof, and

adjacent substituents R_{z1}, R_{zh}, R_{zn} can be optionally joined to form a ring.

According to an embodiment of the present disclosure, wherein, in the first compound, the H is selected from the group consisting of the following structures:

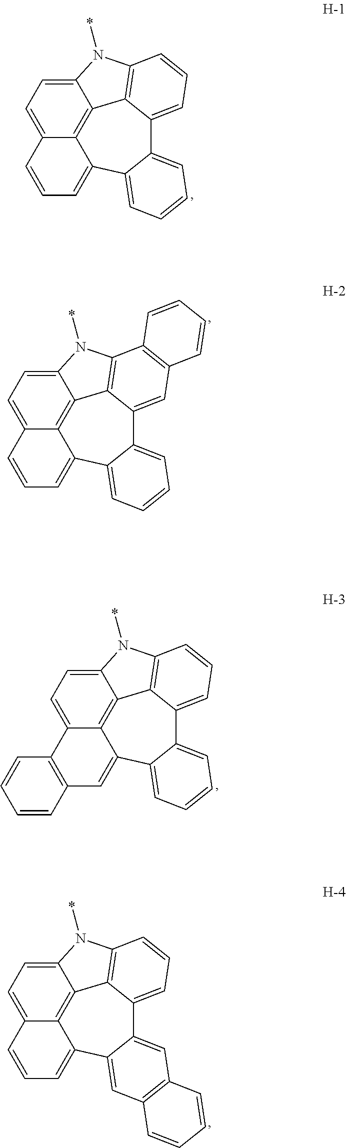 [SMILES: *n1c2cccc3c2c2c4c(cccc4ccc21)-c1ccccc1-3 ; *n1c2ccc3cccc4c3c2c2c(cc3ccccc3c21)-c1ccccc1-4 ; *n1c2cccc3c2c2c4c(cc5ccccc5c4ccc21)-c1ccccc1-3 ; *n1c2cccc3c2c2c4c(cccc4ccc21)-c1cc2ccccc2cc1-3]

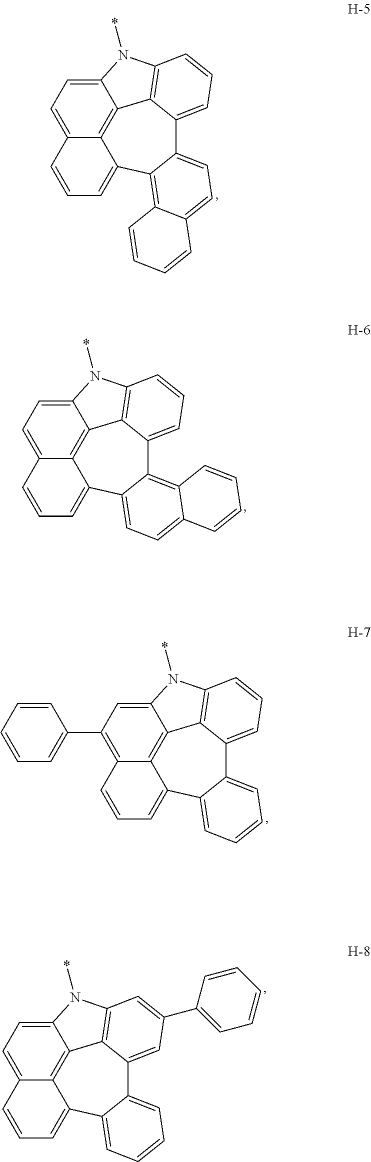 [SMILES: *n1c2cccc3c2c2c4c(cccc4ccc21)-c1c-3ccc2ccccc12 ; *n1c2cccc3c2c2c4c(cccc4ccc21)-c1ccc2ccccc2c1-3 ; *n1c2cccc3c2c2c4c(cccc4c(-c4ccccc4)cc21)-c1ccccc1-3 ; *n1c2cc(-c3ccccc3)cc3c2c2c4c(cccc4ccc21)-c1ccccc1-3]

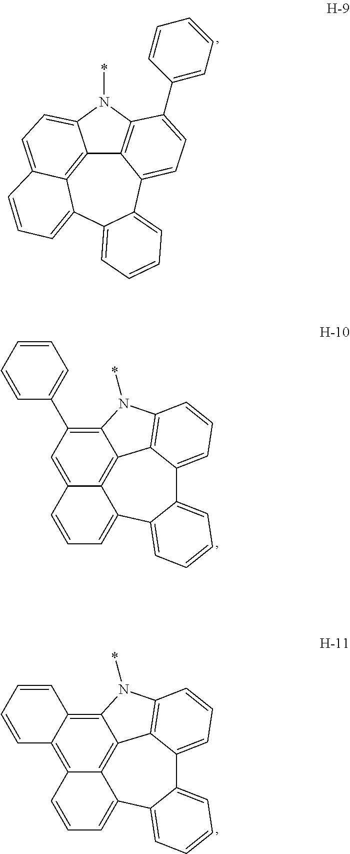 [SMILES: *n1c2ccc3cccc4c3c2c2c(ccc(-c3ccccc3)c21)-c1ccccc1-4 ; *n1c2cccc3c2c2c4c(cccc4cc(-c4ccccc4)c21)-c1ccccc1-3 ; *n1c2cccc3c2c2c4c(cccc4c4ccccc4c21)-c1ccccc1-3]

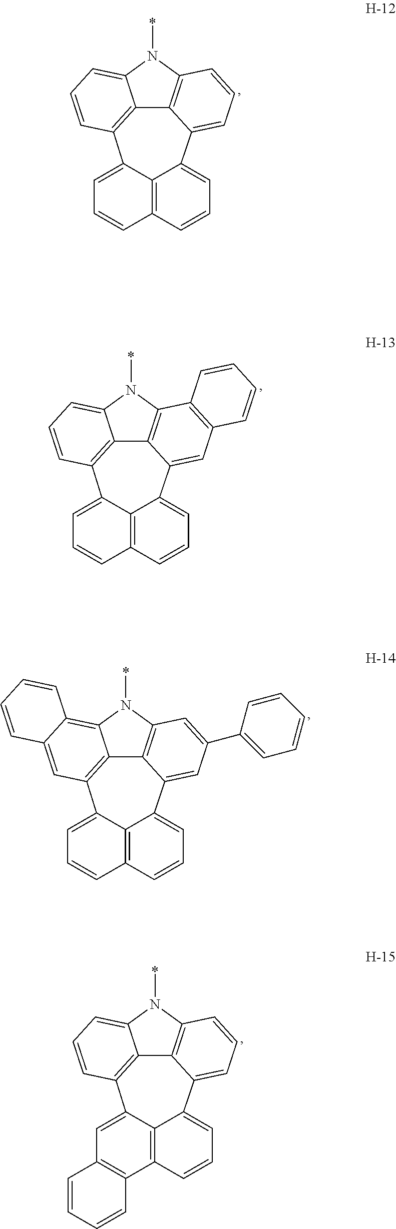 [SMILES: *n1c2cccc3c2c2c(cccc21)-c1cccc2cccc-3c12 ; *n1c2cccc3c2c2c(cc4ccccc4c21)-c1cccc2cccc-3c12 ; *n1c2cc(-c3ccccc3)cc3c2c2c(cc4ccccc4c21)-c1cccc2cccc-3c12 ; *n1c2cccc3c2c2c(cccc21)-c1cc2ccccc2c2cccc-3c12]

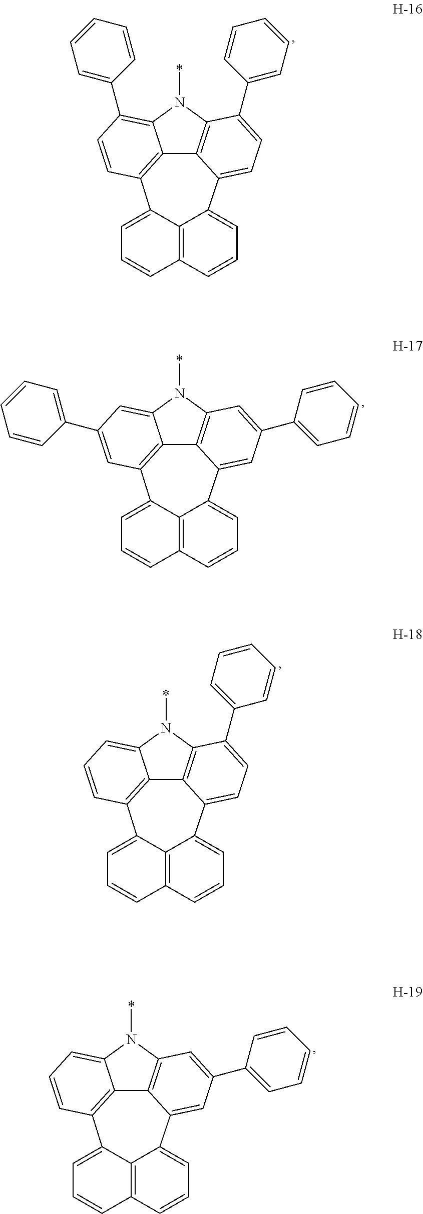 [SMILES: *n1c2c(-c3ccccc3)ccc3c2c2c(ccc(-c4ccccc4)c21)-c1cccc2cccc-3c12 ; *n1c2cc(-c3ccccc3)cc3c2c2c(cc(-c4ccccc4)cc21)-c1cccc2cccc-3c12 ; *n1c2cccc3c2c2c(ccc(-c4ccccc4)c21)-c1cccc2cccc-3c12 ; *n1c2cccc3c2c2c(cc(-c4ccccc4)cc21)-c1cccc2cccc-3c12]

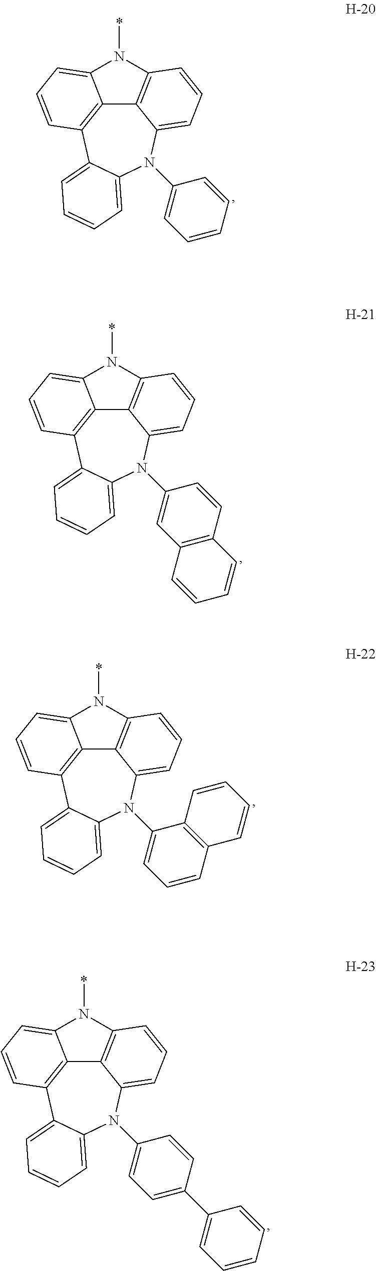 [SMILES: *n1c2cccc3c4ccccc4n(-c4ccccc4)c4cccc1c4c32 ; *n1c2cccc3c4ccccc4n(-c4ccc5ccccc5c4)c4cccc1c4c32 ; *n1c2cccc3c4ccccc4n(-c4cccc5ccccc45)c4cccc1c4c32 ; *n1c2cccc3c4ccccc4n(-c4ccc(-c5ccccc5)cc4)c4cccc1c4c32]

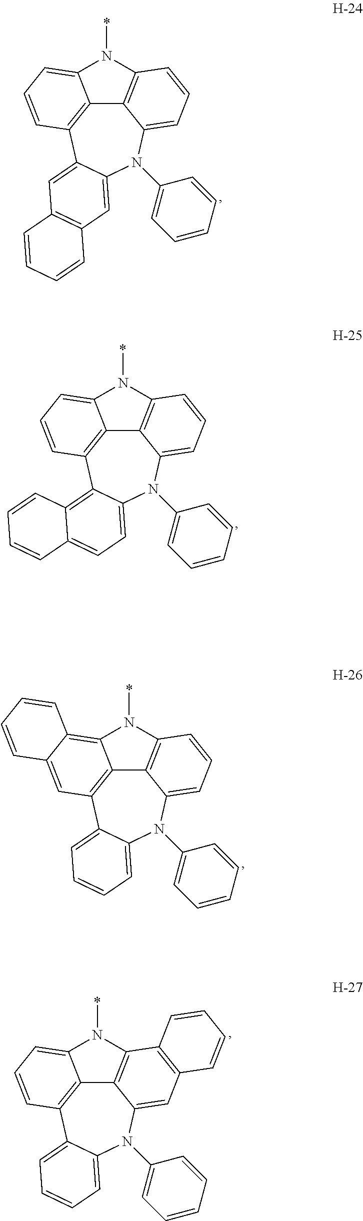 [SMILES: *n1c2cccc3c4cc5ccccc5cc4n(-c4ccccc4)c4cccc1c4c32 ; *n1c2cccc3c2c2c1cccc2n(-c1ccccc1)c1ccc2ccccc2c31 ; *n1c2cccc3c2c2c(cc4ccccc4c21)c1ccccc1n3-c1ccccc1 ; *n1c2cccc3c4ccccc4n(-c4ccccc4)c4cc5ccccc5c1c4c32]

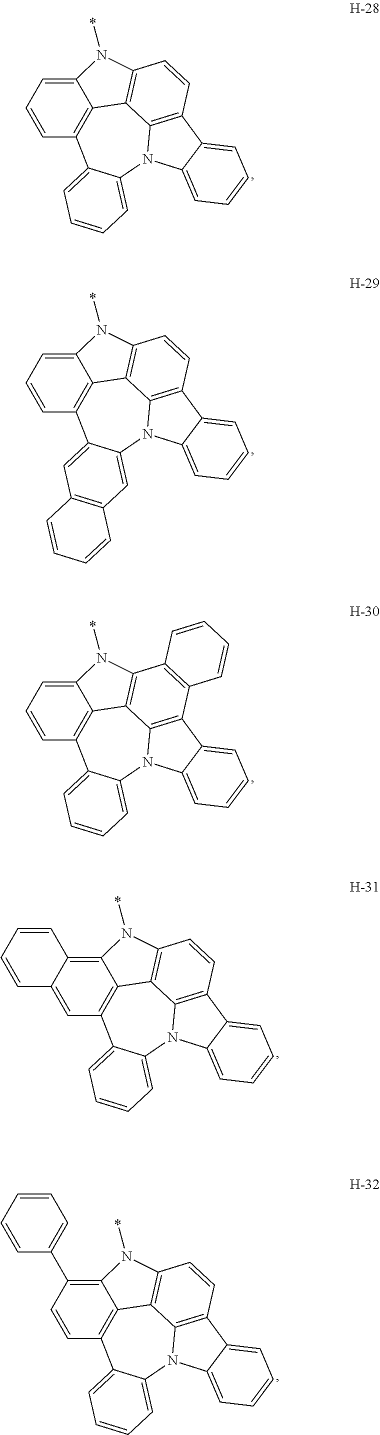 [SMILES: *n1c2cccc3c4ccccc4n4c5ccccc5c5ccc1c(c32)c54 ; *n1c2cccc3c4cc5ccccc5cc4n4c5ccccc5c5ccc1c(c32)c54 ; *n1c2cccc3c4ccccc4n4c5ccccc5c5c6ccccc6c1c(c32)c54 ; *n1c2ccc3c4ccccc4n4c5ccccc5c5cc6ccccc6c1c5c2c34 ; *n1c2ccc3c4ccccc4n4c5ccccc5c5ccc(-c6ccccc6)c1c5c2c34]

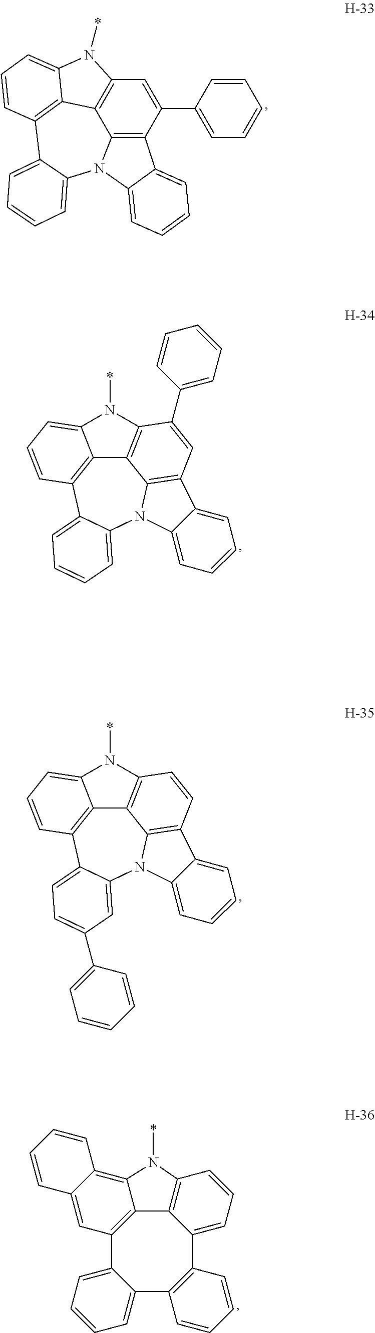 [SMILES: *n1c2cccc3c4ccccc4n4c5ccccc5c5c(-c6ccccc6)cc1c(c32)c54 ; *n1c2cccc3c4ccccc4n4c5ccccc5c5cc(-c6ccccc6)c1c(c32)c54 ; *n1c2cccc3c4ccc(-c5ccccc5)cc4n4c5ccccc5c5ccc1c(c32)c54 ; *n1c2cccc3c4ccccc4c4ccccc4c4cc5ccccc5c1c4c32]

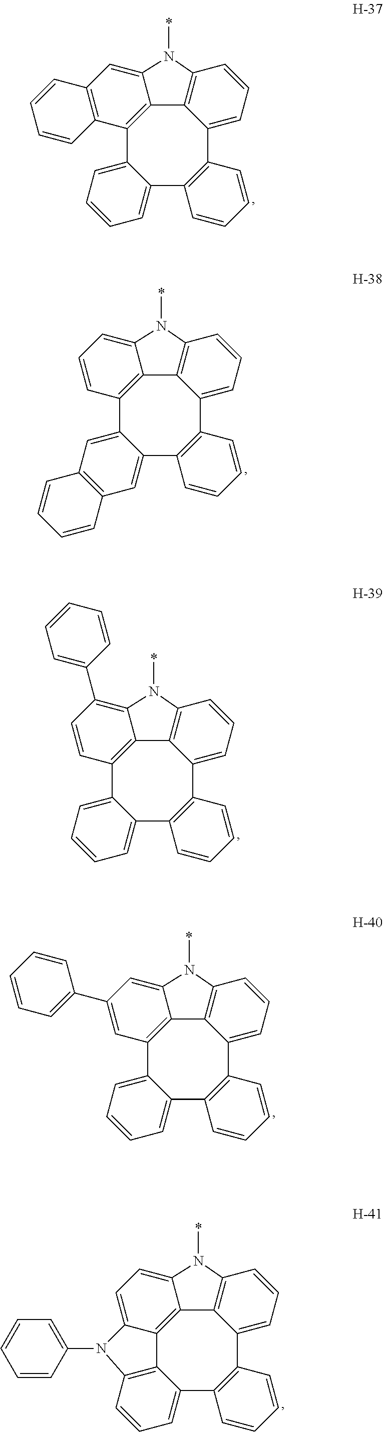 [SMILES: *n1c2cccc3c4ccccc4c4ccccc4c4c5ccccc5cc1c4c32 ; *n1c2cccc3c4ccccc4c4cc5ccccc5cc4c4cccc1c4c32 ; *n1c2cccc3c4ccccc4c4ccccc4c4ccc(-c5ccccc5)c1c4c32 ; *n1c2cccc3c4ccccc4c4ccccc4c4cc(-c5ccccc5)cc1c4c32 ; *n1c2cccc3c4ccccc4c4cccc5c4c4c(c32)c1ccc4n5-c1ccccc1]

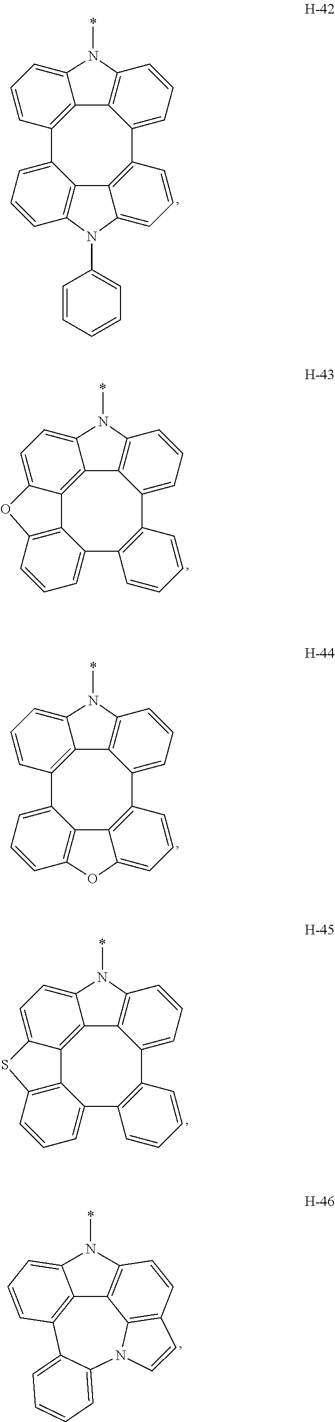 [SMILES: *n1c2cccc3c4cccc5c4c4c(cccc4n5-c4ccccc4)c4cccc1c4c32 ; *n1c2cccc3c4ccccc4c4cccc5oc6ccc1c(c6c54)c32 ; *n1c2cccc3c4cccc5oc6cccc(c7cccc1c7c32)c6c54 ; *n1c2cccc3c4ccccc4c4cccc5sc6ccc1c(c6c54)c32 ; *n1c2cccc3c4ccccc4n4ccc5ccc1c(c32)c54]

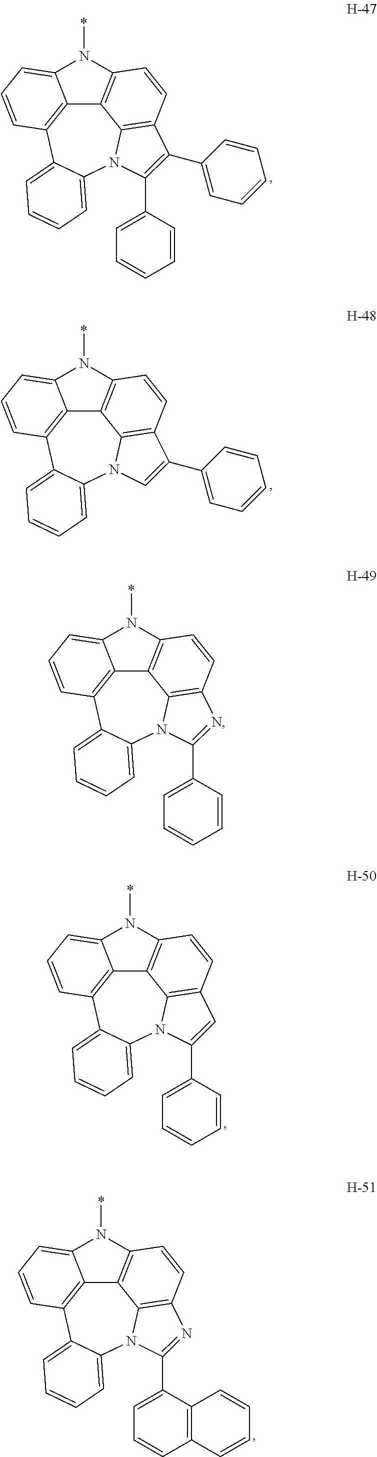 [SMILES: *n1c2cccc3c4ccccc4n4c(-c5ccccc5)c(-c5ccccc5)c5ccc1c(c32)c54 ; *n1c2cccc3c4ccccc4n4cc(-c5ccccc5)c5ccc1c(c32)c54 ; *n1c2cccc3c4ccccc4n4c(-c5ccccc5)nc5ccc1c(c32)c54 ; *n1c2cccc3c4ccccc4n4c(-c5ccccc5)cc5ccc1c(c32)c54 ; *n1c2cccc3c4ccccc4n4c(-c5cccc6ccccc56)nc5ccc1c(c32)c54]

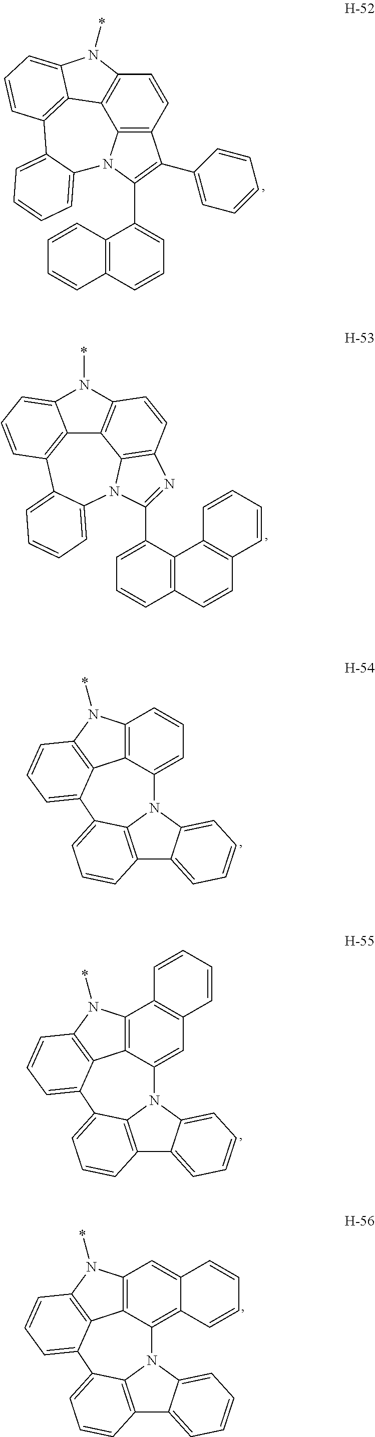 [SMILES: *n1c2cccc3c4ccccc4n4c(-c5cccc6ccccc56)c(-c5ccccc5)c5ccc1c(c32)c54 ; *n1c2cccc3c4ccccc4n4c(-c5cccc6ccc7ccccc7c56)nc5ccc1c(c32)c54 ; *n1c2cccc3c4cccc5c6ccccc6n(c6cccc1c6c32)c45 ; *n1c2cccc3c4cccc5c6ccccc6n(c6cc7ccccc7c1c6c32)c45 ; *n1c2cccc3c4cccc5c6ccccc6n(c45)c4c5ccccc5cc1c4c32]

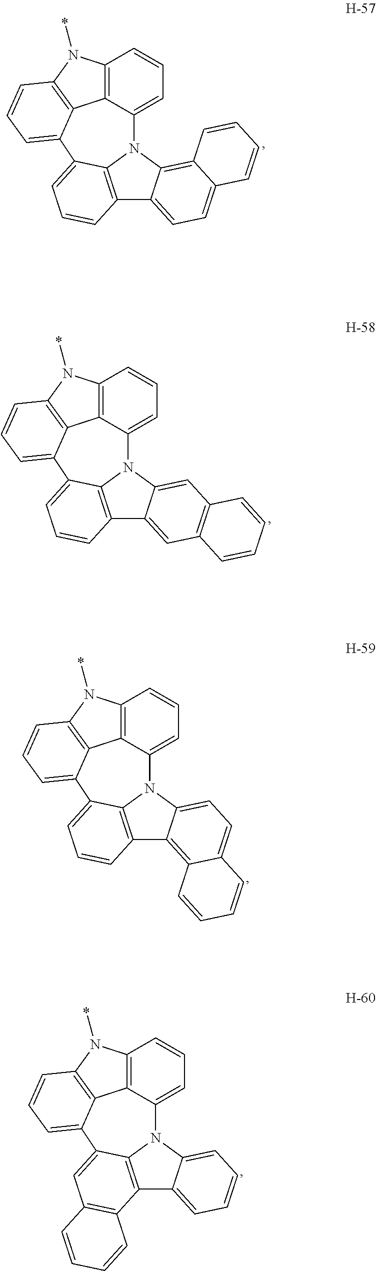 [SMILES: *n1c2cccc3c4cccc5c6ccc7ccccc7c6n(c6cccc1c6c32)c45 ; *n1c2cccc3c4cccc5c6cc7ccccc7cc6n(c6cccc1c6c32)c45 ; *n1c2cccc3c4cccc5c6c7ccccc7ccc6n(c6cccc1c6c32)c45 ; *n1c2cccc3c4cc5ccccc5c5c6ccccc6n(c6cccc1c6c32)c45]

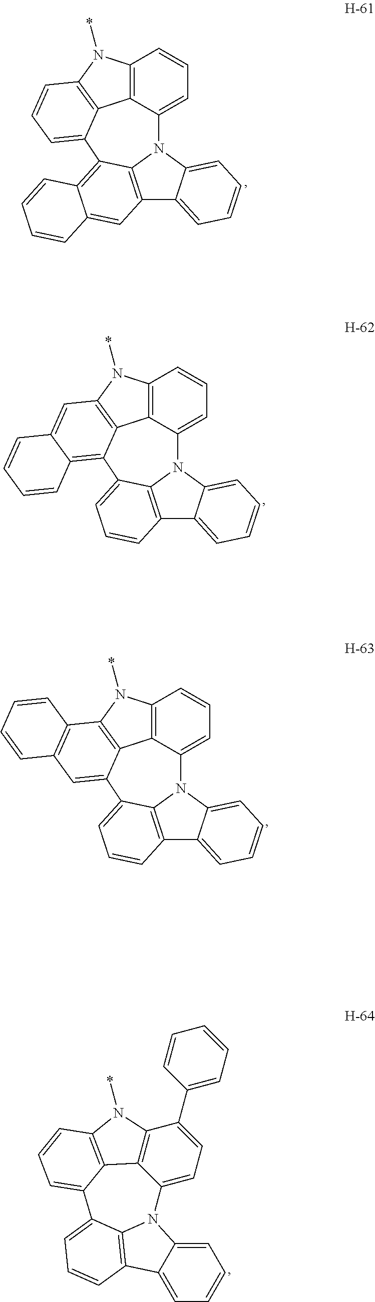 [SMILES: *n1c2cccc3c2c2c1cccc2n1c2ccccc2c2cc4ccccc4c3c21 ; *n1c2cc3ccccc3c3c4cccc5c6ccccc6n(c6cccc1c6c32)c54 ; *n1c2cccc3c2c2c(cc4ccccc4c21)c1cccc2c4ccccc4n3c12 ; *n1c2cccc3c4cccc5c6ccccc6n(c6ccc(-c7ccccc7)c1c6c32)c45]

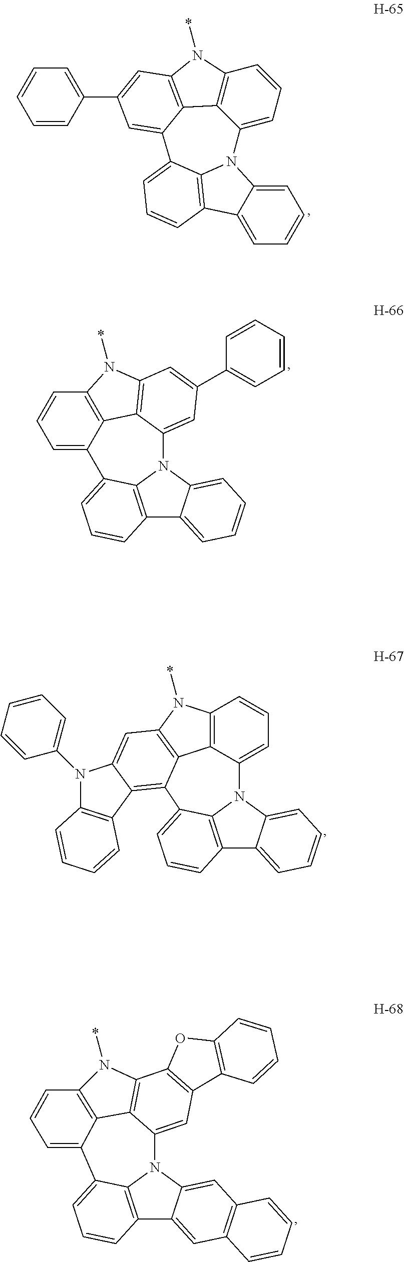 [SMILES: *n1c2cc(-c3ccccc3)cc3c4cccc5c6ccccc6n(c6cccc1c6c32)c45 ; *n1c2cccc3c4cccc5c6ccccc6n(c6cc(-c7ccccc7)cc1c6c32)c45 ; *n1c2cc3c(c4ccccc4n3-c3ccccc3)c3c4cccc5c6ccccc6n(c6cccc1c6c32)c54 ; *n1c2cccc3c4cccc5c6cc7ccccc7cc6n(c6cc7c8ccccc8oc7c1c6c32)c45]

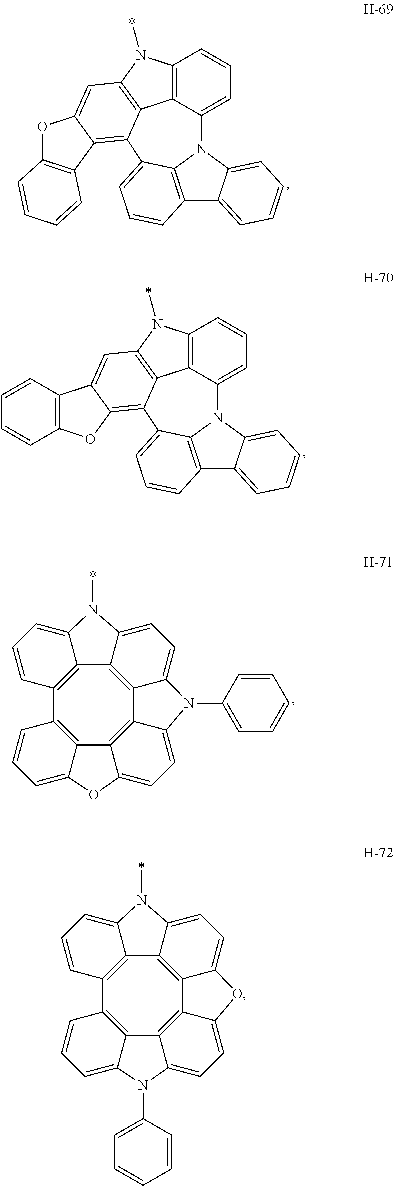 [SMILES: *n1c2cc3oc4ccccc4c3c3c4cccc5c6ccccc6n(c6cccc1c6c32)c54 ; *n1c2cc3c4ccccc4oc3c3c4cccc5c6ccccc6n(c6cccc1c6c32)c54 ; *n1c2cccc3c4cccc5oc6ccc7c(c6c54)c4c(c32)c1ccc4n7-c1ccccc1 ; *n1c2cccc3c4cccc5c4c4c6c(ccc4n5-c4ccccc4)oc4ccc1c(c46)c32]

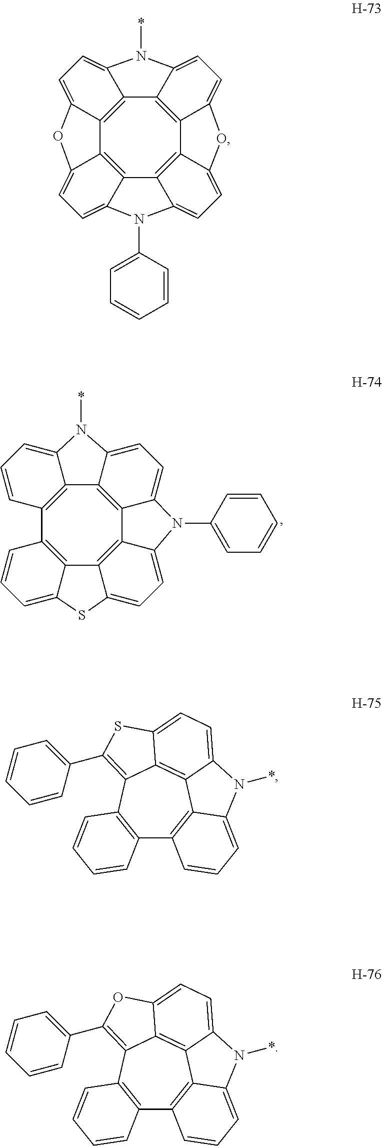

In this embodiment, “*” represents a position where the structure of H is joined to the L.

According to an embodiment of the present disclosure, wherein, hydrogen in the structures of H-1 to H-76 can be partially or fully substituted with deuterium.

According to an embodiment of the present disclosure, wherein, in the first compound, the E has a structure represented by Formula E-a or Formula E-b:

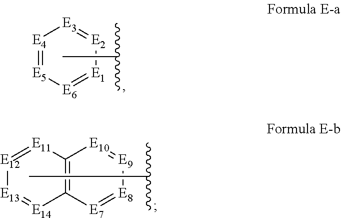

wherein in Formula E-a and Formula E-b,

E₁ to E₁₄ are, at each occurrence identically or differently, selected from C, CRₑ or N;

Rₑ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents Rₑ can be optionally joined to form a ring.

In this embodiment, “ [symbol] ” represents a position where the structure of E is joined to the L.

In this embodiment, in Formula E-a, one of E₁ to E₆ is C, and the C is joined to the L; in Formula E-b, one of E₇ to E₁₄ is C, and the C is joined to the L.

In this embodiment, the expression that “adjacent substituents Rₑ can be optionally joined to form a ring” is intended to mean that any adjacent substituents Rₑ can be joined to form a ring. Obviously, it is possible that any adjacent substituents Rₑ are not joined to form a ring.

According to an embodiment of the present disclosure, wherein, in Formula E-a, at least two of E₁ to E₆ are N; in Formula E-b, at least two of E₇ to E₁₄ are N.

According to an embodiment of the present disclosure, wherein, in Formula E-a, three of E₁ to E₆ are N; in Formula E-b, two of E₇ to E₁₀ are N.

According to an embodiment of the present disclosure, wherein, in the first compound, the E has a structure represented by any one of Formula E-1 to Formula E-10:

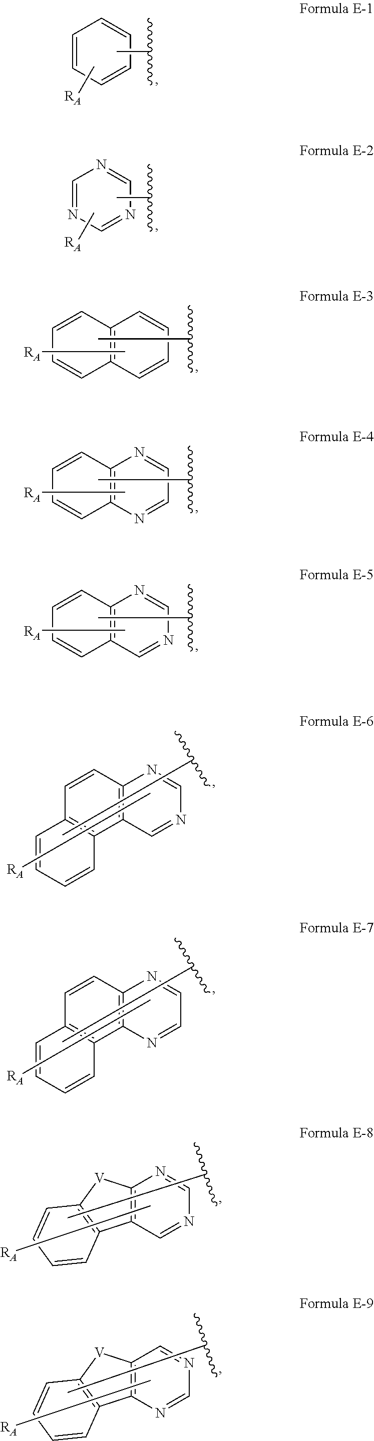

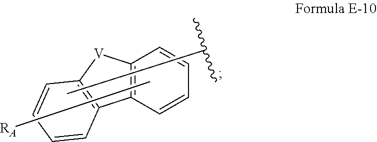

R_{A} represents, at each occurrence identically or differently, mono-substitution, multiple substitutions or non-substitution;

V is selected from O, S or Se;

R_{A} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents R_{A} can be optionally joined to form a ring.

In the present disclosure, the expression that “adjacent substituents R_{A} can be optionally joined to form a ring” is intended to mean that any adjacent substituents R_{A} can be joined to form a ring. Obviously, it is possible that any adjacent substituents R_{A} are not joined to form a ring.

In this embodiment, “ [symbol] ” represents a position where the structure of E is joined to the L.

According to an embodiment of the present disclosure, wherein, in the first compound, the E has a structure represented by any one of Formula E-11 to Formula E-21:

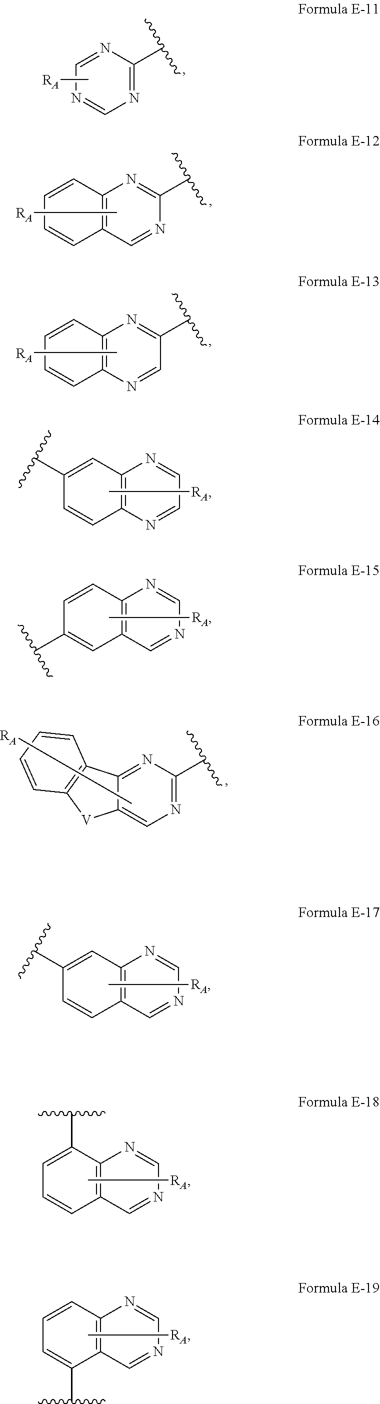

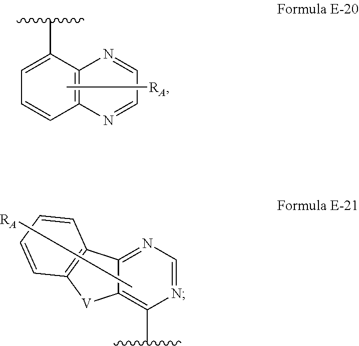

R_{A} represents, at each occurrence identically or differently, mono-substitution, multiple substitutions or non-substitution;

V is selected from O, S or Se;

R_{A} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof, and

adjacent substituents R_{A} can be optionally joined to form a ring.

In this embodiment, “ [symbol] ” represents a position where the structure of E is joined to the L.

According to an embodiment of the present disclosure, wherein, in Formula E-1 to Formula E-21, R_{A} is, at each occurrence identically or differently, selected from the group consisting of: hydrogen, deuterium, halogen, cyano, hydroxyl, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof, and

adjacent substituents R_{A} can be optionally joined to form a ring.

According to an embodiment of the present disclosure, wherein, in Formula E-1 to Formula E-21, R_{A} is, at each occurrence identically or differently, selected from the group consisting of: hydrogen, deuterium, fluorine, cyano, hydroxyl, a sulfanyl group, methyl, trideuteriomethyl, vinyl, phenyl, biphenyl, naphthyl, 4-cyanophenyl, dibenzofuranyl, dibenzothienyl, triphenylene, carbazolyl, 9-phenylcarbazolyl, 9,9-dimethylfluorenyl, pyridyl, phenylpyridyl and combinations thereof, and

adjacent substituents R_{A} can be optionally joined to form a ring.

According to an embodiment of the present disclosure, wherein, in Formula E-1 to Formula E-21, at least one R_{A} is present, and the at least one R_{A} is, at each occurrence identically or differently, selected from the group consisting of: deuterium, halogen, cyano, hydroxyl, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof, and

adjacent substituents R_{A} can be optionally joined to form a ring.

According to an embodiment of the present disclosure, wherein, in Formula E-1 to Formula E-21, at least one R_{A} is present, and the at least one R_{A} is, at each occurrence identically or differently, selected from the group consisting of: deuterium, halogen, cyano, substituted or unsubstituted alkyl having 1 to 20 carbon atoms, substituted or unsubstituted aryl having 6 to 30 carbon atoms, substituted or unsubstituted heteroaryl having 3 to 30 carbon atoms and combinations thereof, and

adjacent substituents R_{A} can be optionally joined to form a ring.

According to an embodiment of the present disclosure, wherein, in Formula E-1 to Formula E-21, at least one R_{A} is present, and the at least one R_{A} is, at each occurrence identically or differently, selected from the group consisting of: deuterium, fluorine, cyano, methyl, trideuteriomethyl, phenyl, biphenyl, naphthyl, 4-cyanophenyl, dibenzofuranyl, dibenzothienyl, triphenylene, carbazolyl, 9-phenylcarbazolyl, 9,9-dimethylfluorenyl, pyridyl, phenylpyridyl and combinations thereof, and adjacent substituents R_{A} can be optionally joined to form a ring.

According to an embodiment of the present disclosure, wherein, in the first compound, the E is selected from the group consisting of the following structures:

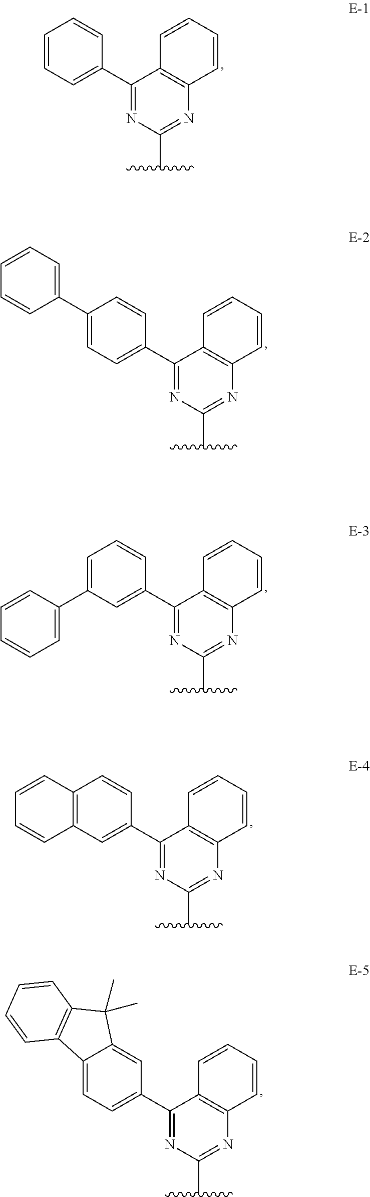 [SMILES: *c1nc(-c2ccccc2)c2ccccc2n1 ; *c1nc(-c2ccc(-c3ccccc3)cc2)c2ccccc2n1 ; *c1nc(-c2cccc(-c3ccccc3)c2)c2ccccc2n1 ; *c1nc(-c2ccc3ccccc3c2)c2ccccc2n1 ; *c1nc(-c2ccc3c(c2)C(C)(C)c2ccccc2-3)c2ccccc2n1]

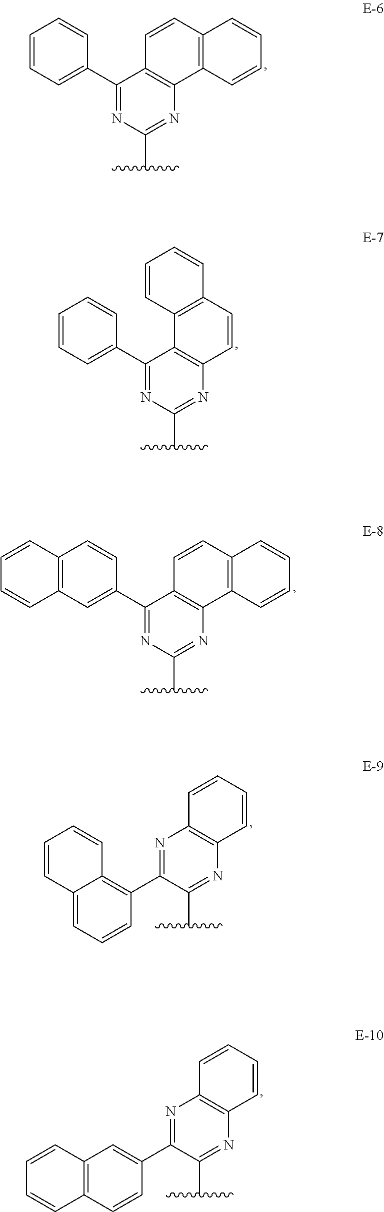 [SMILES: *c1nc(-c2ccccc2)c2ccc3ccccc3c2n1 ; *c1nc(-c2ccccc2)c2c(ccc3ccccc32)n1 ; *c1nc(-c2ccc3ccccc3c2)c2ccc3ccccc3c2n1 ; *c1nc2ccccc2nc1-c1cccc2ccccc12 ; *c1nc2ccccc2nc1-c1ccc2ccccc2c1]

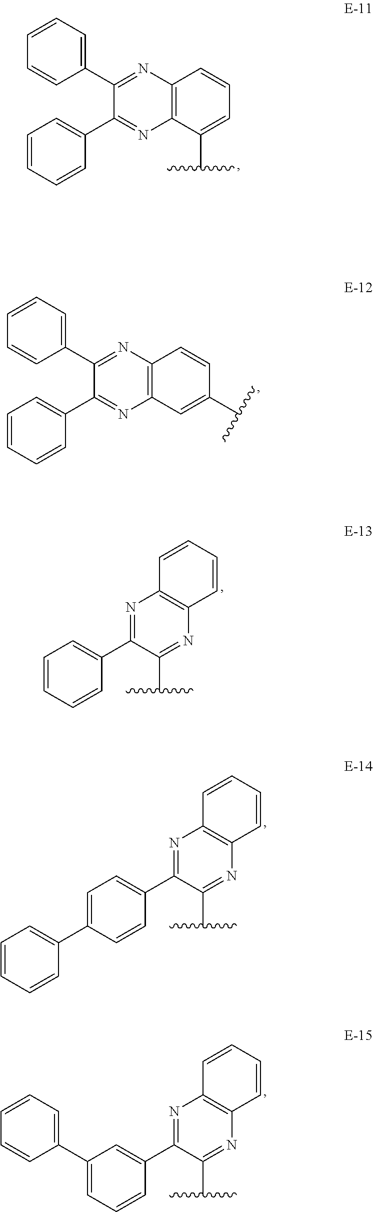 [SMILES: *c1cccc2nc(-c3ccccc3)c(-c3ccccc3)nc12 ; *c1ccc2nc(-c3ccccc3)c(-c3ccccc3)nc2c1 ; *c1nc2ccccc2nc1-c1ccccc1 ; *c1nc2ccccc2nc1-c1ccc(-c2ccccc2)cc1 ; *c1nc2ccccc2nc1-c1cccc(-c2ccccc2)c1]

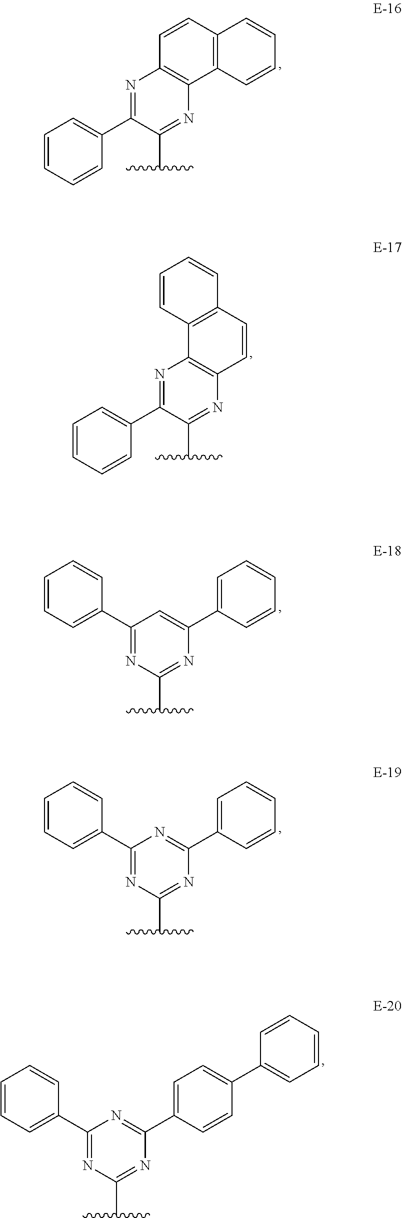 [SMILES: *c1nc2c(ccc3ccccc32)nc1-c1ccccc1 ; *c1nc2ccc3ccccc3c2nc1-c1ccccc1 ; *c1nc(-c2ccccc2)cc(-c2ccccc2)n1 ; *c1nc(-c2ccccc2)nc(-c2ccccc2)n1 ; *c1nc(-c2ccccc2)nc(-c2ccc(-c3ccccc3)cc2)n1]

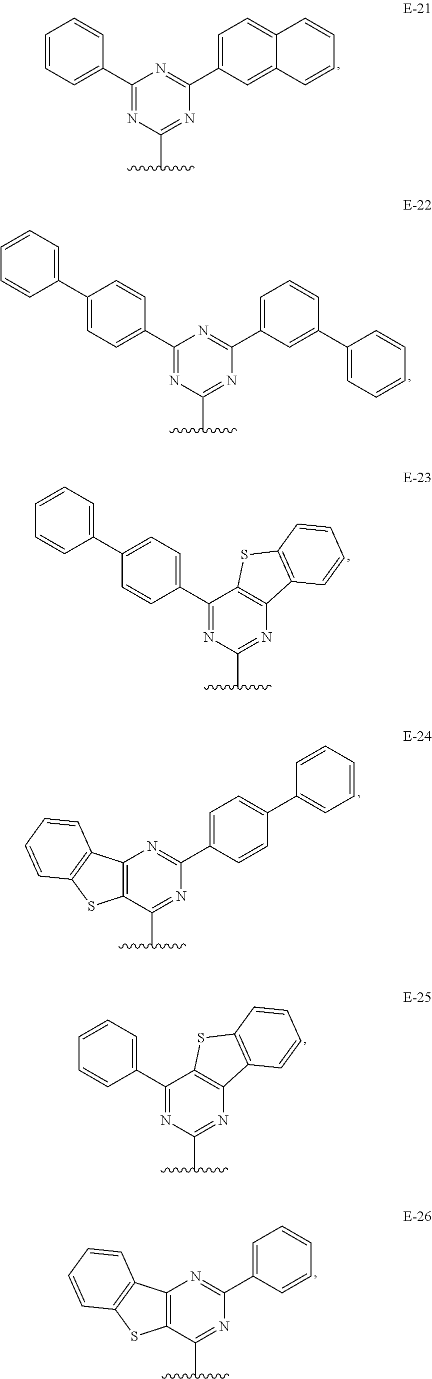 [SMILES: *c1nc(-c2ccccc2)nc(-c2ccc3ccccc3c2)n1 ; *c1nc(-c2ccc(-c3ccccc3)cc2)nc(-c2cccc(-c3ccccc3)c2)n1 ; *c1nc(-c2ccc(-c3ccccc3)cc2)c2sc3ccccc3c2n1 ; *c1nc(-c2ccc(-c3ccccc3)cc2)nc2c1sc1ccccc12 ; *c1nc(-c2ccccc2)c2sc3ccccc3c2n1 ; *c1nc(-c2ccccc2)nc2c1sc1ccccc12]

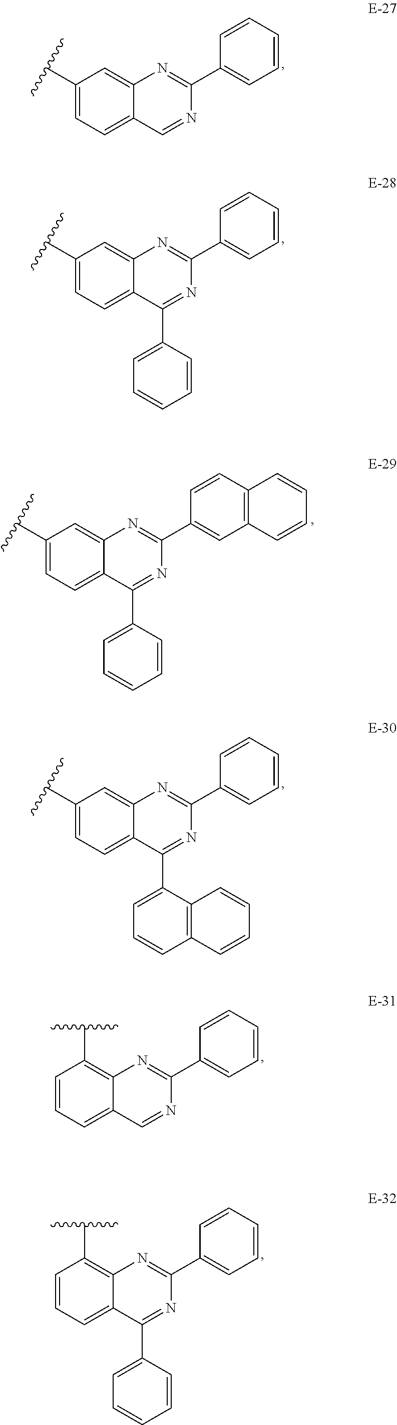 [SMILES: *c1ccc2cnc(-c3ccccc3)nc2c1 ; *c1ccc2c(-c3ccccc3)nc(-c3ccccc3)nc2c1 ; *c1ccc2c(-c3ccccc3)nc(-c3ccc4ccccc4c3)nc2c1 ; *c1ccc2c(-c3cccc4ccccc34)nc(-c3ccccc3)nc2c1 ; *c1cccc2cnc(-c3ccccc3)nc12 ; *c1cccc2c(-c3ccccc3)nc(-c3ccccc3)nc12]

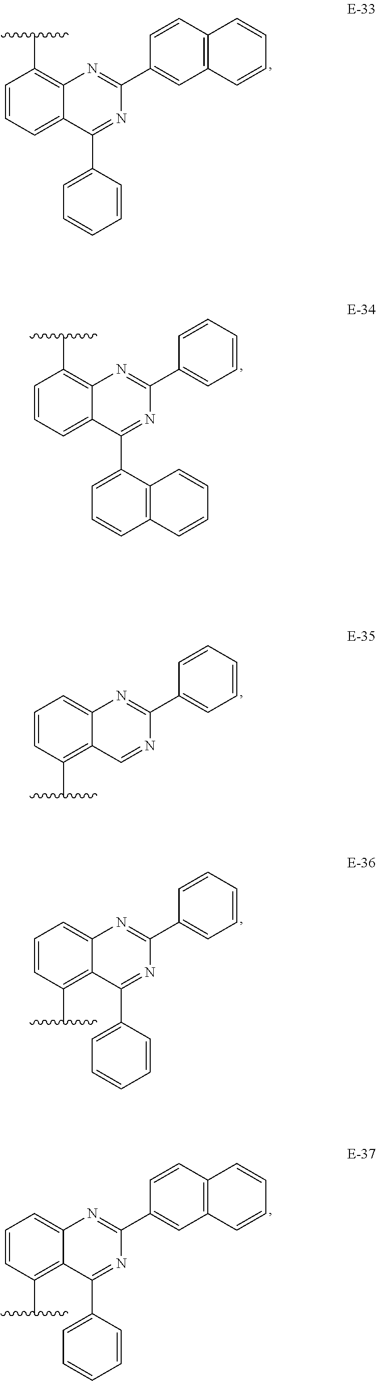 [SMILES: *c1cccc2c(-c3ccccc3)nc(-c3ccc4ccccc4c3)nc12 ; *c1cccc2c(-c3cccc4ccccc34)nc(-c3ccccc3)nc12 ; *c1cccc2nc(-c3ccccc3)ncc12 ; *c1cccc2nc(-c3ccccc3)nc(-c3ccccc3)c12 ; *c1cccc2nc(-c3ccc4ccccc4c3)nc(-c3ccccc3)c12]

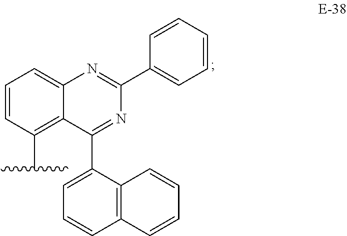 [SMILES: *c1cccc2nc(-c3ccccc3)nc(-c3cccc4ccccc34)c12]

wherein “ [symbol] ” represents a position where the structure of E is joined to the L.

According to an embodiment of the present disclosure, wherein, the L is selected from the group consisting of: a single bond, phenylene, naphthylene, biphenylene, terphenylene, triphenylenylene, pyridylene, dibenzothienylene, dibenzofuranylene and thienylene.

According to an embodiment of the present disclosure, wherein, the L is selected from the group consisting of the following structures:

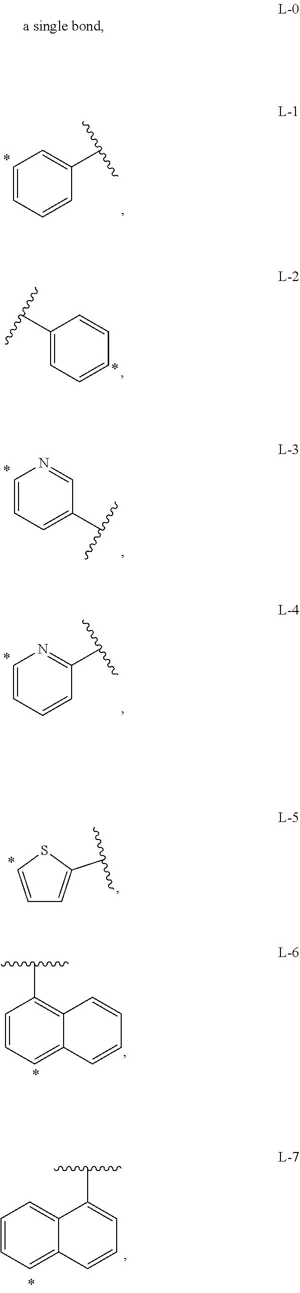

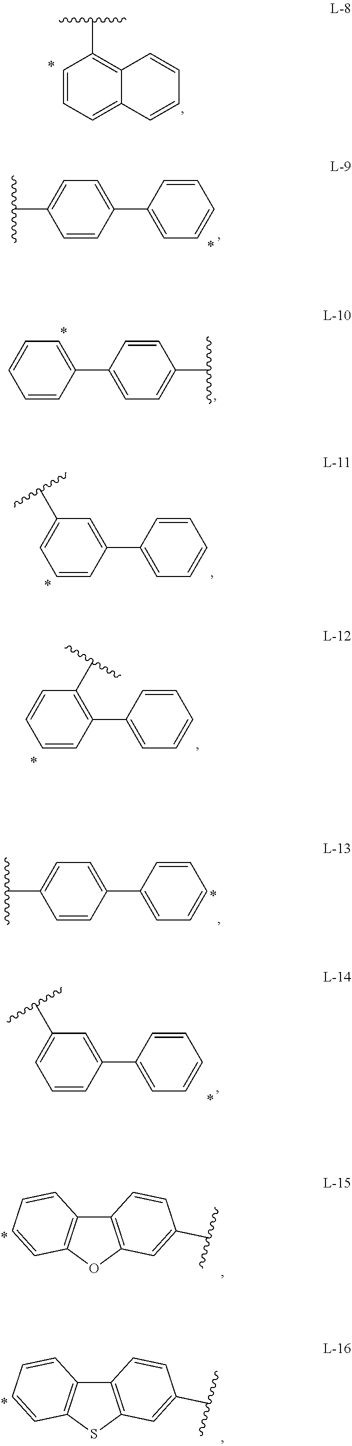

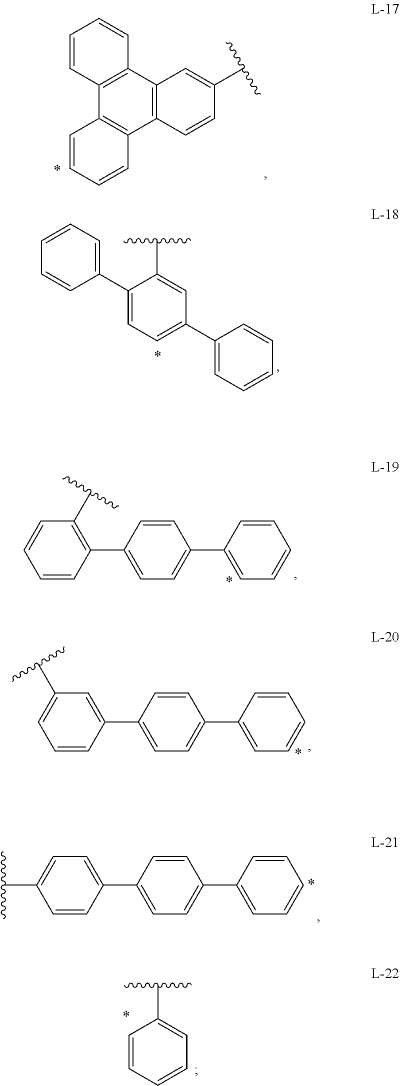 [SMILES: *c1ccc2c3ccccc3c3ccccc3c2c1 ; *c1cc(-c2ccccc2)ccc1-c1ccccc1 ; *c1ccccc1-c1ccc(-c2ccccc2)cc1 ; *c1cccc(-c2ccc(-c3ccccc3)cc2)c1 ; *c1ccc(-c2ccc(-c3ccccc3)cc2)cc1 ; *c1ccccc1]

wherein “*” represents a position where the structure of L is joined to the H, and “ [symbol] ” represents a position where the structure of L is joined to the E.

According to an embodiment of the present disclosure, wherein, hydrogen in the structures of L-1 to L-22 can be partially or fully substituted with deuterium.

According to an embodiment of the present disclosure, wherein, the first compound has the structure of H-L-E, wherein the H is selected from any one of the group consisting of H-1 to H-76, the L is selected from any one of the group consisting of L-0 to L-22, and the E is selected from any one of the group consisting of E-1 to E-38. Optionally, hydrogen in the structure of the first compound can be partially or fully substituted with deuterium.

According to an embodiment of the present disclosure, wherein, the first compound is selected from the group consisting of Compound 1-1 to Compound 1-249, wherein the specific structures of Compound 1-1 to Compound 1-249 are referred to claim 12.

According to an embodiment of the present disclosure, wherein, hydrogen in Compound 1-1 to Compound 1-249 can be partially or fully substituted with deuterium.

According to an embodiment of the present disclosure, wherein, in the second compound, in Formula C, the ring A and/or the ring B are each independently selected from a five-membered unsaturated carbocyclic ring, an aromatic ring having 6 to 18 carbon atoms or a heteroaromatic ring having 3 to 18 carbon atoms.

According to an embodiment of the present disclosure, wherein, in the second compound, in Formula C, the ring A and/or the ring B are each independently selected from a five-membered unsaturated carbocyclic ring, an aromatic ring having 6 to 10 carbon atoms or a heteroaromatic ring having 3 to 10 carbon atoms.

According to an embodiment of the present disclosure, wherein, in the second compound, the Lₐ has a structure represented by any one of Formula 2-1 to Formula 2-19:

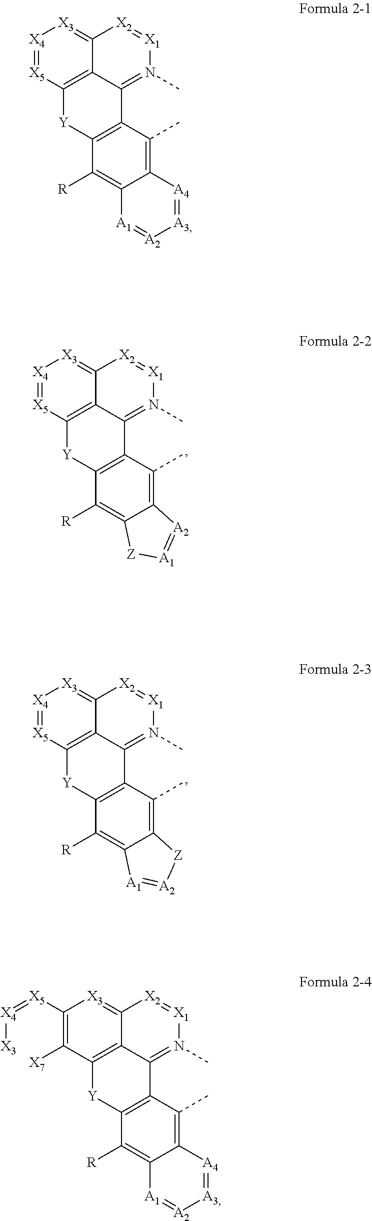

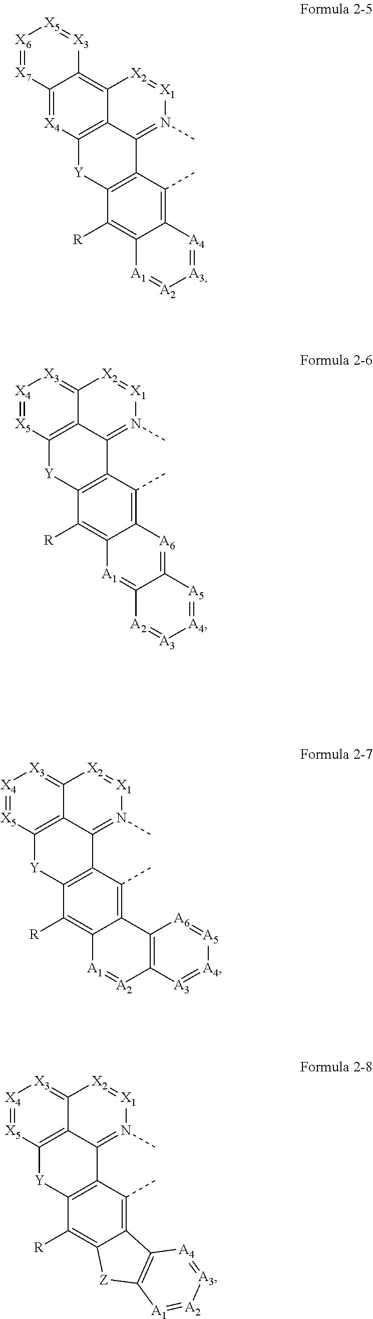

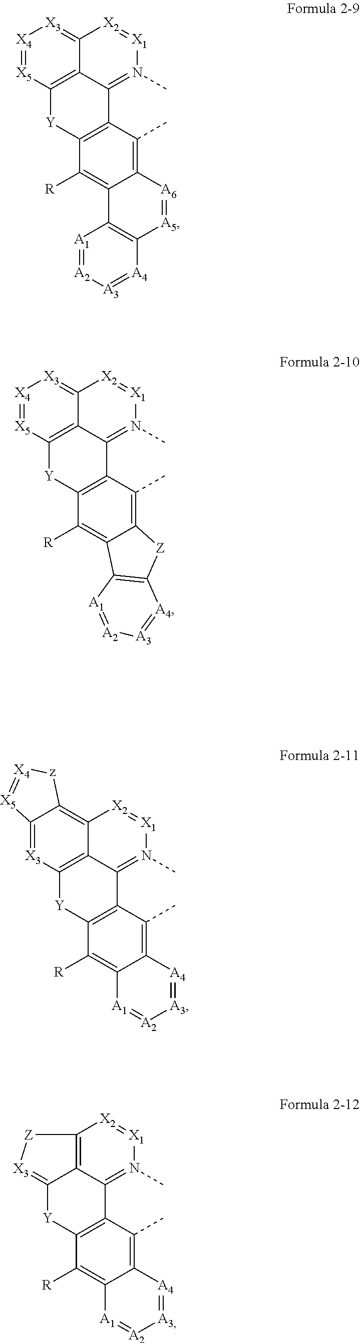

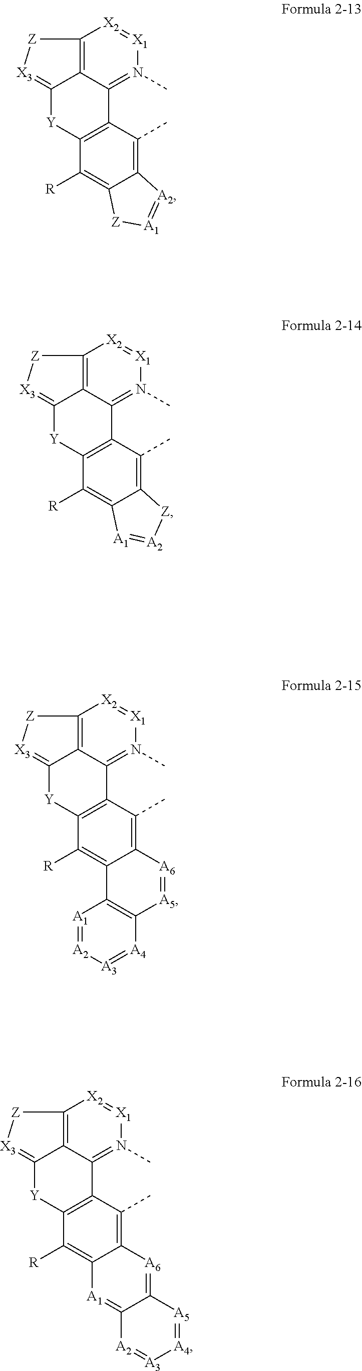

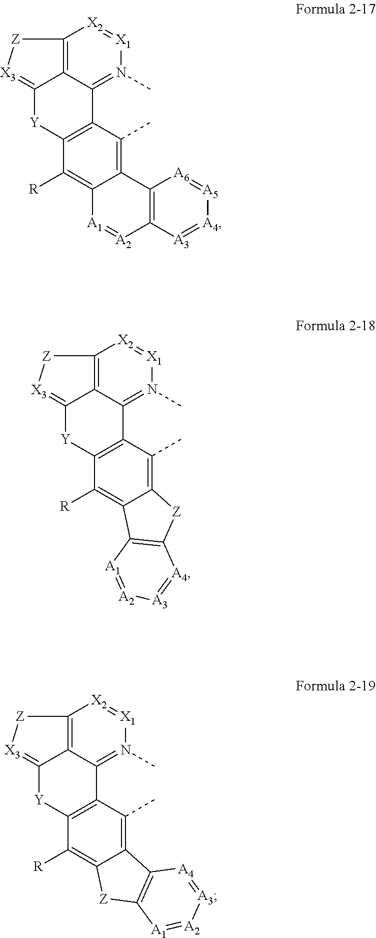

wherein

in Formula 2-1 to Formula 2-19, X₁ and X₂ are, at each occurrence identically or differently, selected from CRₓ or N, X₃ to X₇ are, at each occurrence identically or differently, selected from CRᵢ or N, and A₁ to A₆ are, at each occurrence identically or differently, selected from CRᵢᵢ or N;

Z is, at each occurrence identically or differently, selected from CRᵢᵢᵢRᵢᵢᵢ, SiRᵢᵢᵢRᵢᵢᵢ, PRᵢᵢᵢ, O, S or NRᵢᵢᵢ; wherein when two Rᵢᵢᵢ are present, the two Rᵢᵢᵢ are identical or different;

Y is selected from SiR_{y}R_{y}, NR_{y}, PR_{y}, O, S or Se; wherein when two R_{y} are present, the two R_{y} are identical or different;

R, Rₓ, R_{y}, Rᵢ, Rᵢᵢ and Rᵢᵢᵢ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents R, Rₓ, R_{y}, Rᵢ, Rᵢᵢ and Rᵢᵢᵢ can be optionally joined to form a ring.

In the present disclosure, the expression that “adjacent substituents R, Rₓ, R_{y}, Rᵢ, Rᵢᵢ and Rᵢᵢᵢ can be optionally joined to form a ring” is intended to mean that any one or more of groups of adjacent substituents, such as two substituents Rᵢ, two substituents Rᵢᵢ, two substituents Rₓ, two substituents R_{y}, two substituents Rᵢᵢᵢ, substituents Rᵢ and Rₓ, substituents Rᵢᵢ and Rᵢᵢᵢ, substituents R and R_{y}, substituents R_{y} and Rᵢᵢᵢ, substituents Rₓ and Rᵢᵢᵢ, and substituents R and Rᵢᵢᵢ, can be joined to form a ring. Obviously, it is possible that none of these substituents are joined to form a ring.

According to an embodiment of the present disclosure, Lₐ is selected from a structure represented by any one of Formula 2-1, Formula 2-5, Formula 2-8, Formula 2-10, Formula 2-11 or Formula 2-12.

According to an embodiment of the present disclosure, Lₐ is selected from a structure represented by Formula 2-1.

According to an embodiment of the present disclosure, wherein, in Formula 2-1 to Formula 2-19, at least one of X₁ to Xₙ and/or A₁ to Aₘ is selected from N, wherein the Xₙ corresponds to one of X₁ to X₇ that has the largest number in any one of Formula 2-1 to Formula 2-19, and the Aₘ corresponds to one of A₁ to A₆ that has the largest number in any one of Formula 2-1 to Formula 2-19. For example, in Formula 2-1, the Xₙ corresponds to X₅ of X₁ to X₇ that has the largest number in Formula 2-1, and the Aₘ corresponds to A₄ of A₁ to A₆ that has the largest number in Formula 2-1, that is, in Formula 2-1, at least one of X₁ to X₅ and/or A₁ to A₄ is selected from N. In another example, in Formula 2-12, the Xₙ corresponds to X₃ of X₁ to X₇ that has the largest number in Formula 2-12, and the Aₘ corresponds to A₄ of A₁ to A₆ that has the largest number in Formula 2-12, that is, in Formula 2-12, at least one of X₁ to X₃ and/or A₁ to A₄ is selected from N.

According to an embodiment of the present disclosure, in Formula 2-1 to Formula 2-19, at least one of X₁ to Xₙ is selected from N, wherein the Xₙ corresponds to one of X₁ to X₇ that has the largest number in any one of Formula 2-1 to Formula 2-19.

According to an embodiment of the present disclosure, in Formula 2-1 to Formula 2-19, X₂ is N.

According to an embodiment of the present disclosure, wherein, in Formula 2-1 to Formula 2-19, X₁ and X₂ are each independently selected from CRₓ, X₃ to X₇ are each independently selected from CRᵢ, A₁ to A₆ are each independently selected from CRᵢᵢ, and adjacent substituents Rₓ, Rᵢ, Rᵢᵢ can be optionally joined to form a ring.

In this embodiment, the expression that “adjacent substituents Rₓ, Rᵢ, Rᵢᵢ can be optionally joined to form a ring” is intended to mean that any one or more of groups of adjacent substituents, such as two substituents Rᵢ, two substituents Rᵢᵢ, two substituents Rₓ, and substituents Rᵢ and Rₓ, can be joined to form a ring. Obviously, it is possible that none of these substituents are joined to form a ring.

According to an embodiment of the present disclosure, the Rₓ, Rᵢ and Rᵢᵢ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, cyano and combinations thereof.

According to an embodiment of the present disclosure, at least two or three of the Rₓ, Rᵢ and Rᵢᵢ are, at each occurrence identically or differently, selected from the group consisting of:
deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, cyano and combinations thereof.

In this embodiment, the expression that at least two or three of Rₓ, Rᵢ and Rᵢᵢ are, at each occurrence identically or differently, selected from the group of substituents is intended to mean that at least two or three substituents in the group consisting of two substituents Rₓ, all substituents Rᵢ and all substituents Rᵢᵢ are, at each occurrence identically or differently, selected from the group of substituents.

According to an embodiment of the present disclosure, wherein, in Formula 2-1 to Formula 2-11, X₄ and/or X₅ is selected from CRᵢ, and in Formula 2-12 to Formula 2-19, X₃ is selected from CRᵢ; and

the Rᵢ is, at each occurrence identically or differently, selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, in Formula 2-1 to Formula 2-11, X₄ and/or X₅ is selected from CRᵢ, and in Formula 2-12 to Formula 2-19, X₃ is selected from CRᵢ; and the Rᵢ is, at each occurrence identically or differently, selected from the group consisting of: hydrogen, deuterium, fluorine, methyl, ethyl, isopropyl, isobutyl, t-butyl, neopentyl, cyclopentyl, cyclopentylmethyl, cyclohexyl, norbornyl, adamantyl, trimethylsilyl, isopropyldimethylsilyl, phenyldimethylsilyl, trifluoromethyl, cyano and combinations thereof.

According to an embodiment of the present disclosure, wherein, in Formula 2-1 to Formula 2-19, the R is selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, in Formula 2-1 to Formula 2-19, the R is selected from hydrogen, deuterium, fluorine, methyl, ethyl, isopropyl, isobutyl, t-butyl, cyclopentyl, cyclopentylmethyl, cyclohexyl, neopentyl, deuterated methyl, deuterated ethyl, deuterated isopropyl, deuterated isobutyl, deuterated t-butyl, deuterated cyclopentyl, deuterated cyclopentylmethyl, deuterated cyclohexyl, deuterated neopentyl, trimethylsilyl or a combination thereof.

According to an embodiment of the present disclosure, wherein, in Formula 2-1 to Formula 2-19, Y is selected from O or S.

According to an embodiment of the present disclosure, wherein, in Formula 2-1 to Formula 2-19, Y is O.

According to an embodiment of the present disclosure, wherein, in Formula 2-1 to Formula 2-19, X₁ and X₂ are each independently selected from CRₓ.

According to an embodiment of the present disclosure, wherein, in Formula 2-1 to Formula 2-19, X₁ and X₂ are each independently selected from CRₓ, wherein the Rₓ is, at each occurrence identically or differently, selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, in Formula 2-1 to Formula 2-19, X₁ is selected from CRₓ, and X₂ is N.

According to an embodiment of the present disclosure, wherein, in Formula 2-1 to Formula 2-19, X₁ is selected from CRₓ, and X₂ is N, wherein the Rₓ is selected from hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, in the second compound, the ligand Lₐ has a structure represented by Formula 2-20 or Formula 2-21:

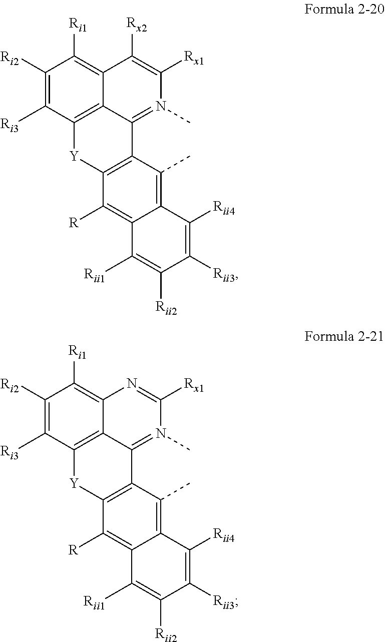

wherein in Formula 2-20 and Formula 2-21,

Y is selected from O or S;

Rₓ₁, Rₓ₂, Rᵢ₁, Rᵢ₂, Rᵢ₃, Rᵢᵢ₁, Rᵢᵢ₂, Rᵢᵢ₃ and Rᵢᵢ₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof;

R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms and combinations thereof.

According to an embodiment of the present disclosure, wherein, the ligand Lₐ has a structure represented by Formula 2-20 or Formula 2-21:

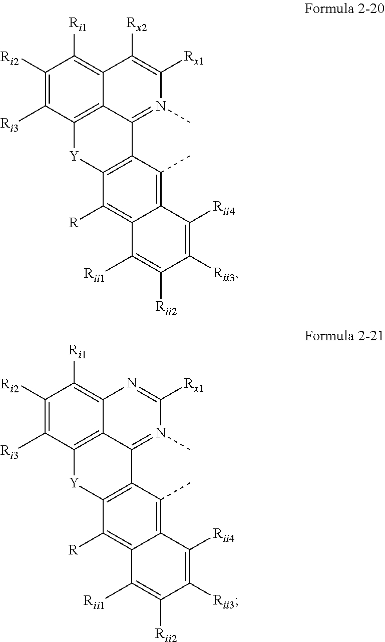

wherein in Formula 2-20 and Formula 2-21,

Y is selected from O or S; and

at least one or two of Rₓ₁, Rₓ₂, Rᵢ₁, Rᵢ₂ and Rᵢ₃ and/or at least one or two of Rᵢᵢ₁, Rᵢᵢ₂, Rᵢᵢ₃ and Rᵢᵢ₄ are, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, R is selected from halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, the ligand Lₐ has a structure represented by Formula 2-20 or Formula 2-21:

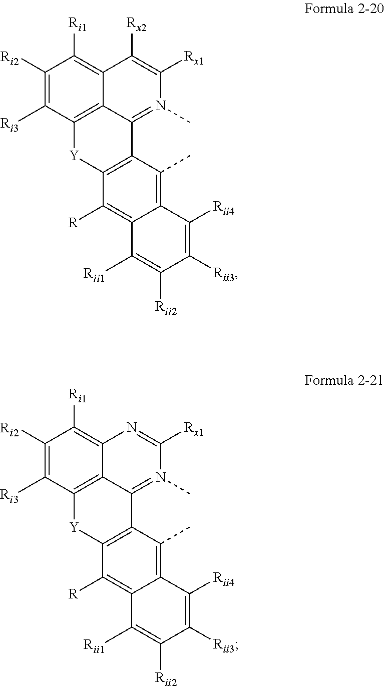

wherein in Formula 2-20 and Formula 2-21,

Y is selected from O or S; and

at least one or two of Rₓ₁, Rₓ₂, Rᵢ₁, Rᵢ₂ and Rᵢ₃ and/or at least one or two of Rᵢᵢ₁, Rᵢᵢ₂, Rᵢᵢ₃ and Rᵢᵢ₄ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof; R is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, the ligand Lₐ has a structure represented by Formula 2-20 or Formula 2-21:

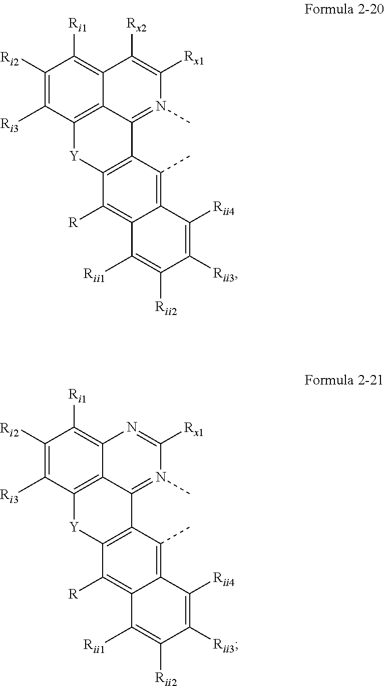

wherein in Formula 2-20 and Formula 2-21,

Y is selected from O or S;

Rᵢ₂ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and

R is selected from halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and at least one or two of Rᵢᵢ₁, Rᵢᵢ₂, Rᵢᵢ₃ and Rᵢᵢ₄ are, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, the ligand Lₐ has a structure represented by Formula 2-20 or Formula 2-21:

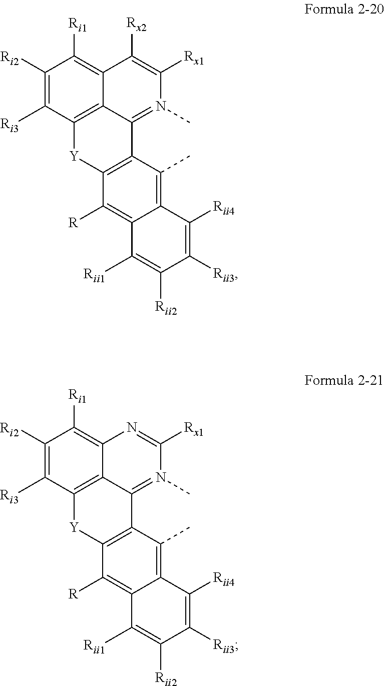

wherein in Formula 2-20 and Formula 2-21,

Y is selected from O or S;

Rᵢ₂ is selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and

R is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and at least one or two of Rᵢᵢ₁, Rᵢᵢ₂, Rᵢᵢ₃ and Rᵢᵢ₄ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

According to an embodiment of the present disclosure, wherein, in Formula 2-20 and Formula 2-21, at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group consisting of: substituted or unsubstituted alkyl having 3 to 20 carbon atoms, substituted or unsubstituted cycloalkyl having 3 to 20 ring carbon atoms, substituted or unsubstituted alkylsilyl having 3 to 20 carbon atoms and combinations thereof.

In this embodiment, the expression that at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group of substituents is intended to mean that at least one of Rₓ₁ and Rₓ₂ is, at each occurrence identically or differently, selected from the group of substituents and/or that at least one of Rᵢ₁, Rᵢ₂ and Rᵢ₃ is, at each occurrence identically or differently, selected from the group of substituents and/or that at least one of Rᵢᵢ₁, Rᵢᵢ₂, Rᵢᵢ₃ and Rᵢᵢ₄ is, at each occurrence identically or differently, selected from the group of substituents and/or that R is selected from the group of substituents.

According to an embodiment of the present disclosure, wherein, in Formula 2-20 and Formula 2-21, at least one of Rᵢ₂, Rᵢ₃, Rᵢᵢ₁, Rᵢᵢ₂, Rᵢᵢ₃ and R is, at each occurrence identically or differently, selected from the group consisting of: substituted or unsubstituted alkyl having 3 to 20 carbon atoms, substituted or unsubstituted cycloalkyl having 3 to 20 ring carbon atoms, substituted or unsubstituted alkylsilyl having 3 to 20 carbon atoms and combinations thereof.

In this embodiment, the expression that at least one of Rᵢ₂, Rᵢ₃, Rᵢᵢ₁, Rᵢᵢ₂, Rᵢᵢ₃ and R is, at each occurrence identically or differently, selected from the group of substituents is intended to mean that at least one of Rᵢ₂ and Rᵢ₃ is, at each occurrence identically or differently, selected from the group of substituents and/or that at least one of Rᵢᵢ₁, Rᵢᵢ₂ and Rᵢᵢ₃ is, at each occurrence identically or differently, selected from the group of substituents and/or that R is selected from the group of substituents.

According to an embodiment of the present disclosure, wherein, in Formula 2-20 and Formula 2-21, at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group consisting of: substituted or unsubstituted alkyl having 3 to 10 carbon atoms, substituted or unsubstituted cycloalkyl having 3 to 10 ring carbon atoms and combinations thereof.

In this embodiment, the expression that at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group of substituents is intended to mean that at least one of Rₓ₁ and Rₓ₂ is, at each occurrence identically or differently, selected from the group of substituents and/or that at least one of Rᵢ₁, Rᵢ₂ and Rᵢ₃ is, at each occurrence identically or differently, selected from the group of substituents and/or that at least one of Rᵢᵢ₁, Rᵢᵢ₂, Rᵢᵢ₃ and Rᵢᵢ₄ is, at each occurrence identically or differently, selected from the group of substituents and/or that R is selected from the group of substituents.

According to an embodiment of the present disclosure, wherein, the Lₐ is selected from the group consisting of Lₐ₁ to Lₐ₁₈₈:

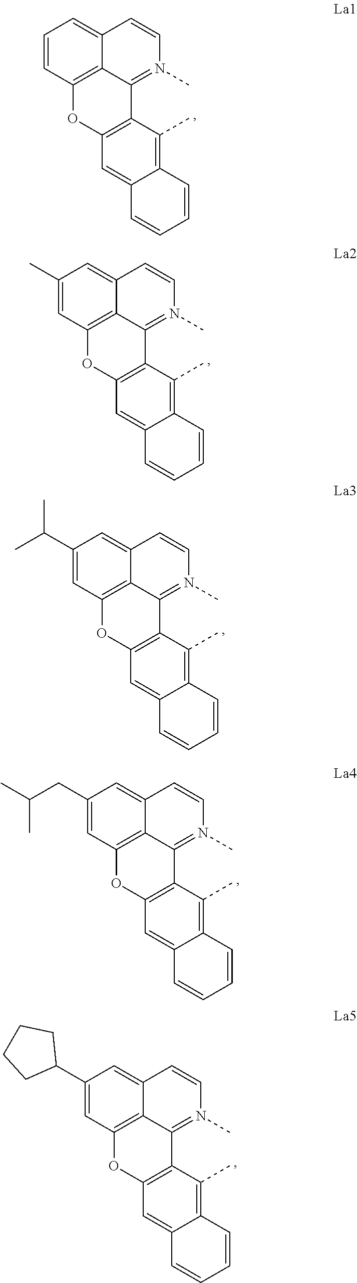

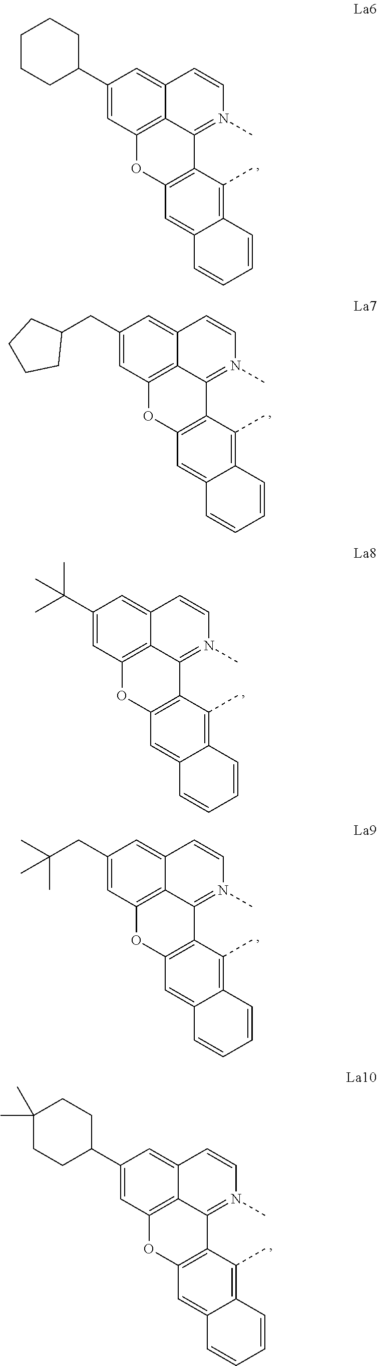

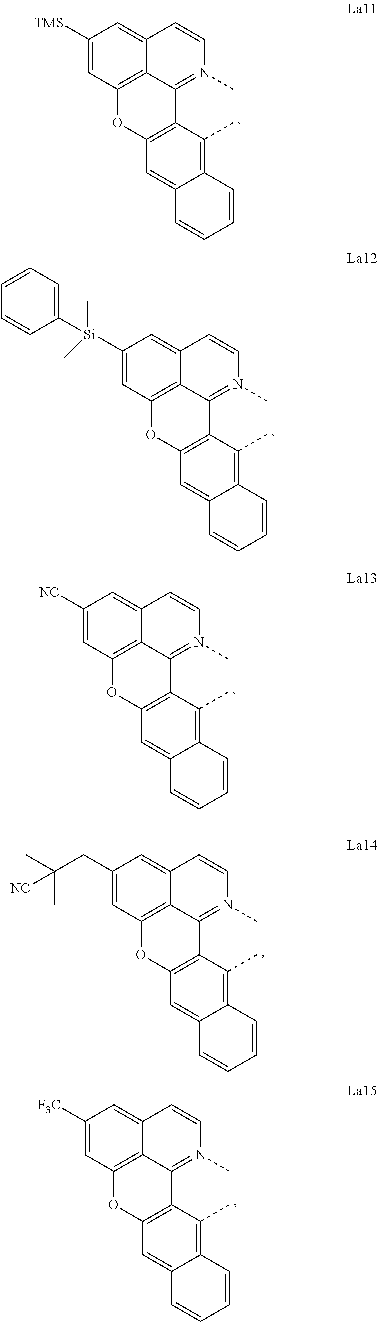

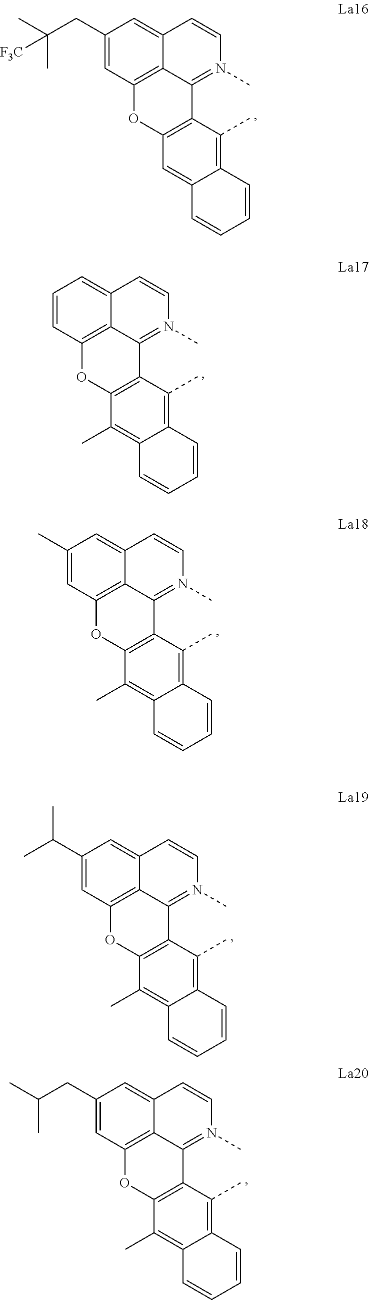

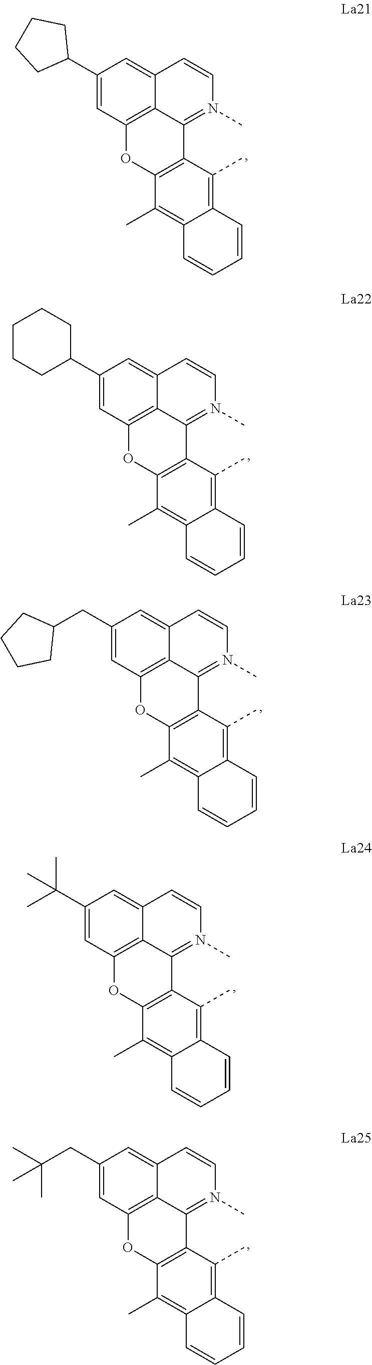

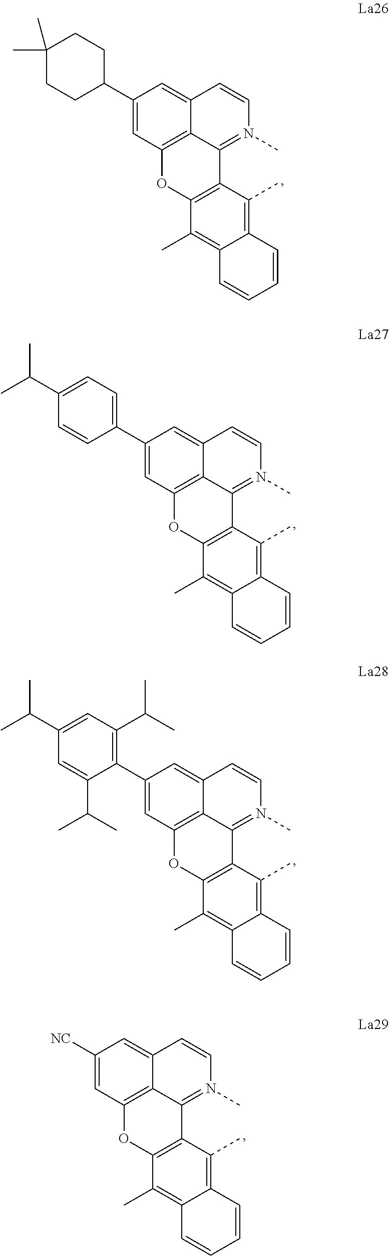

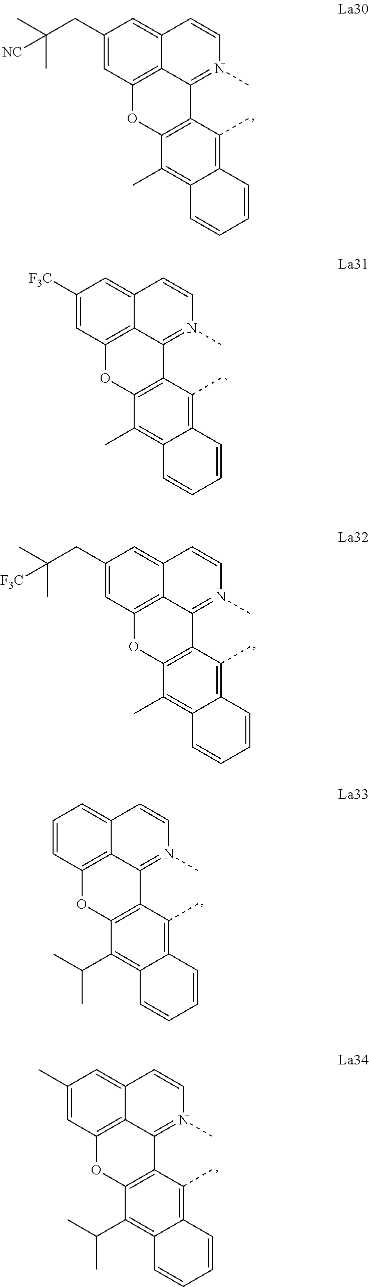

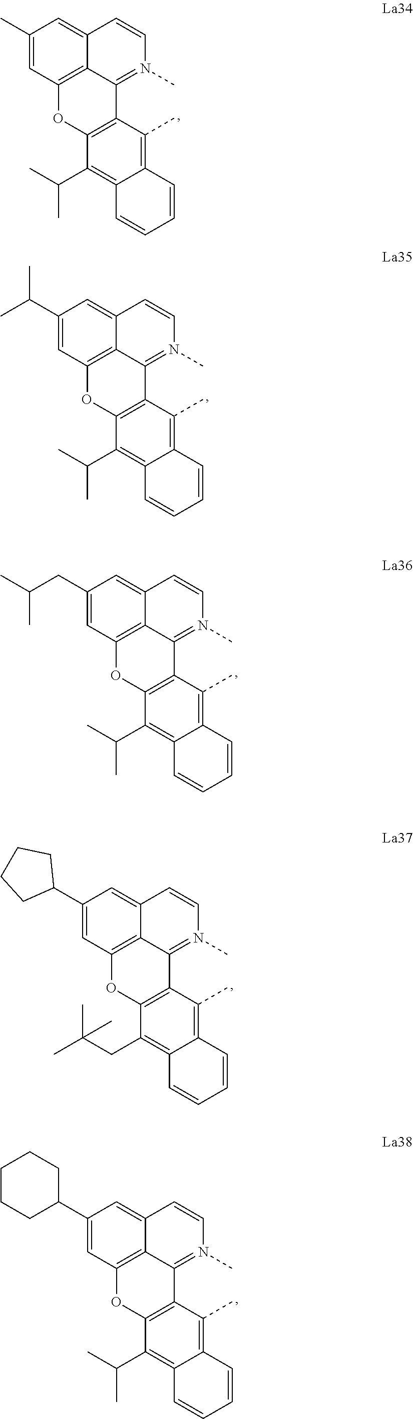

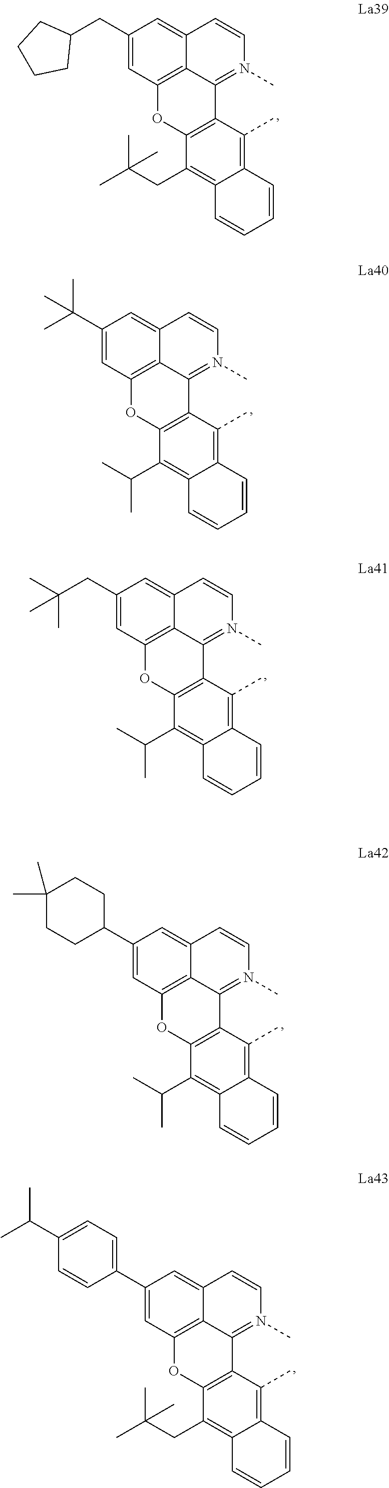

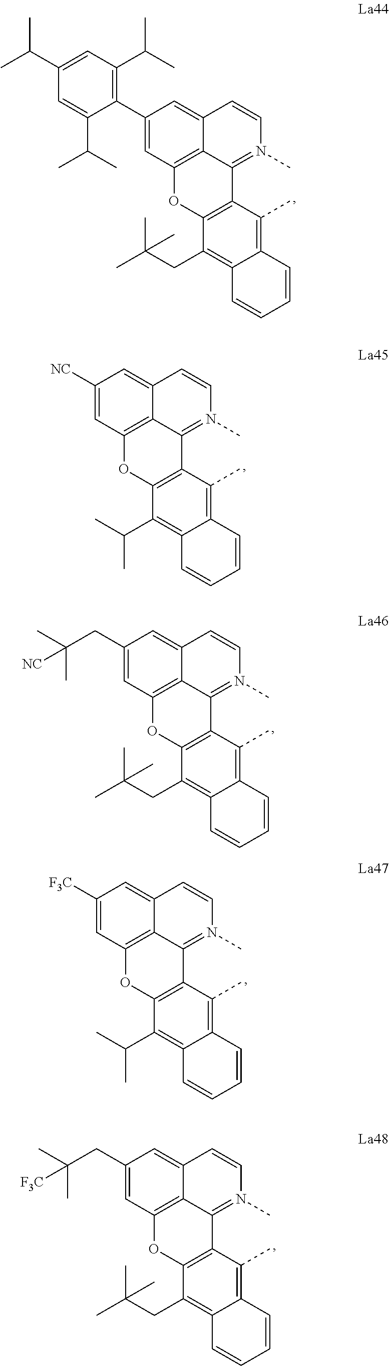

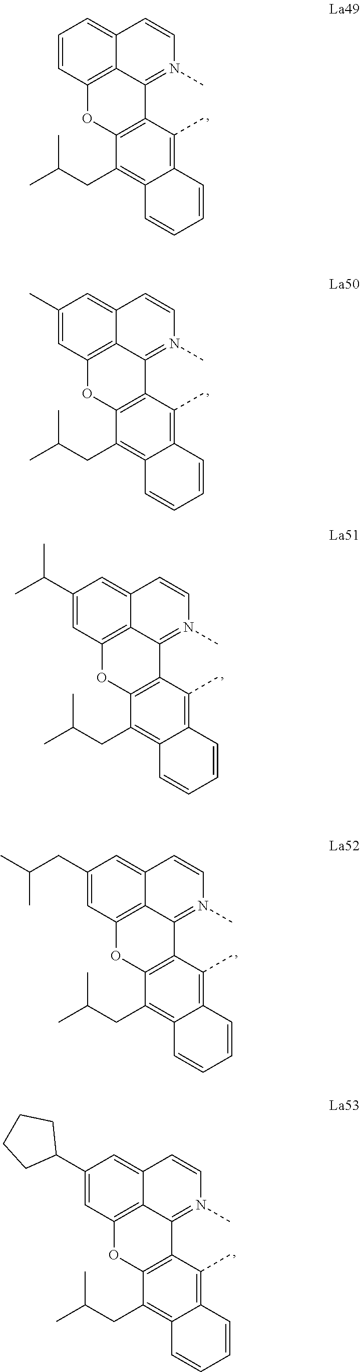

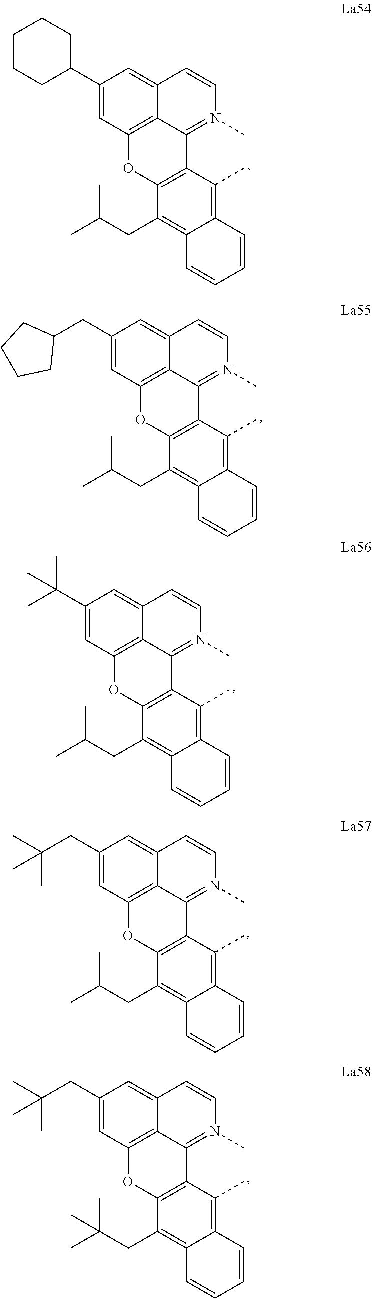

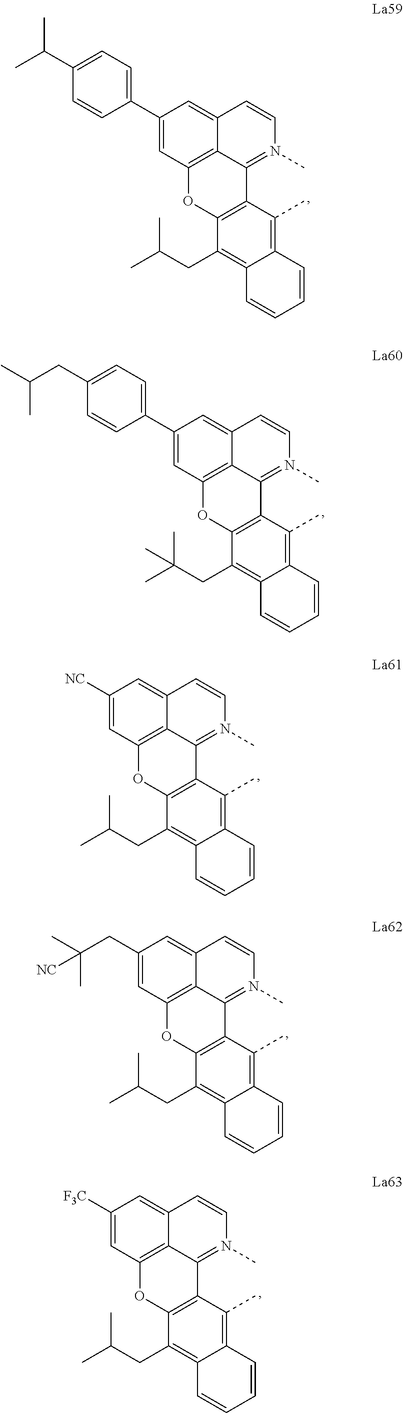

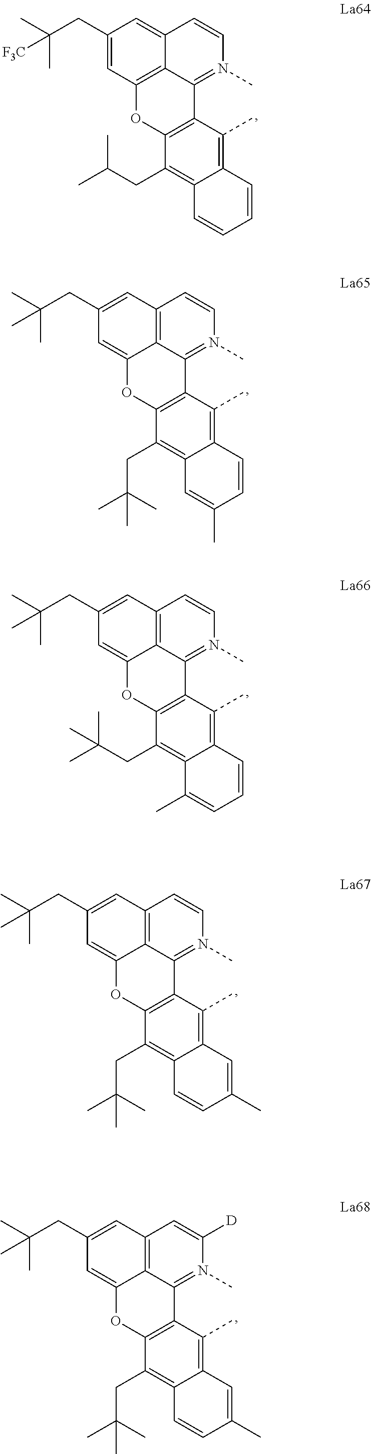

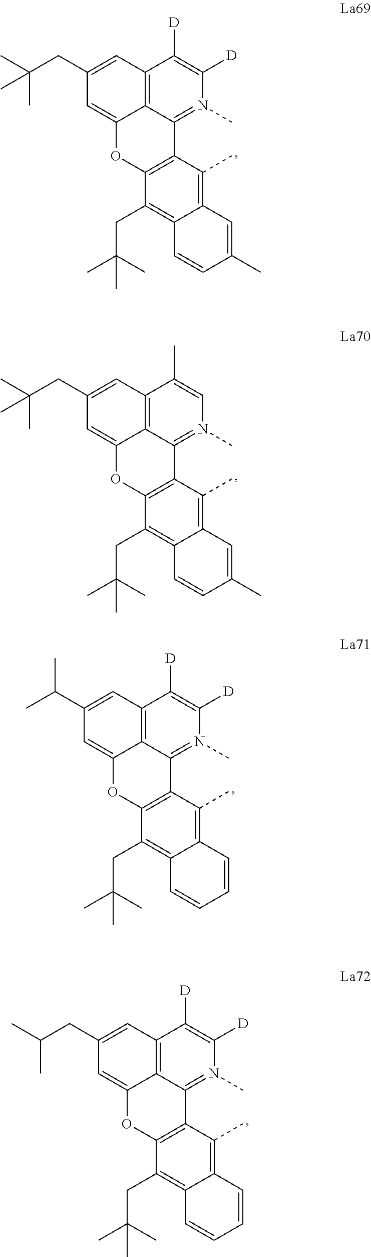

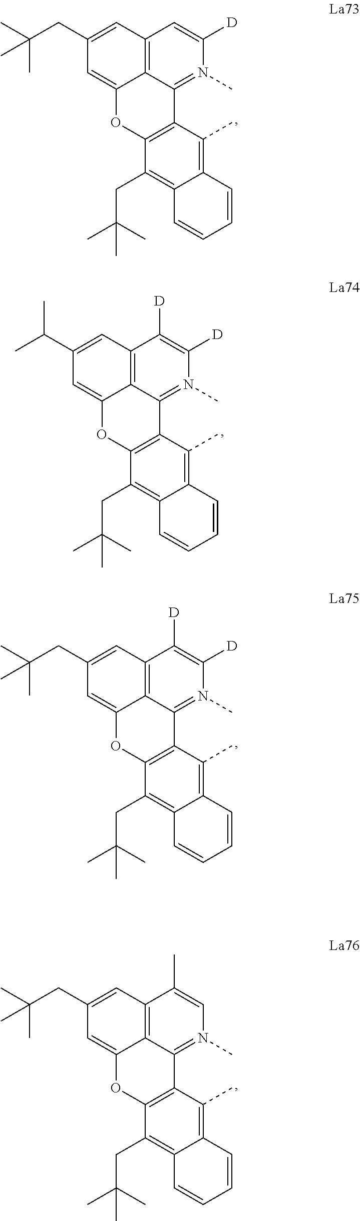

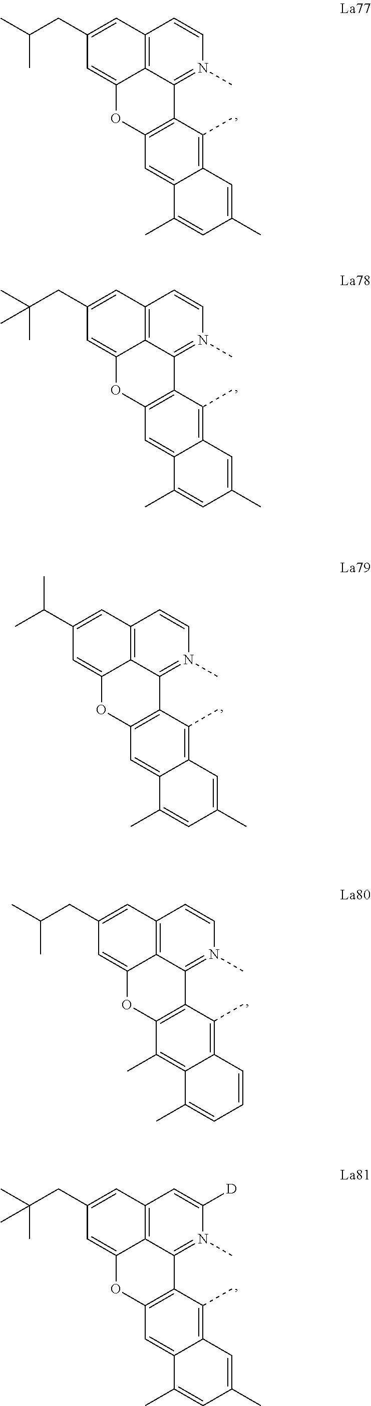

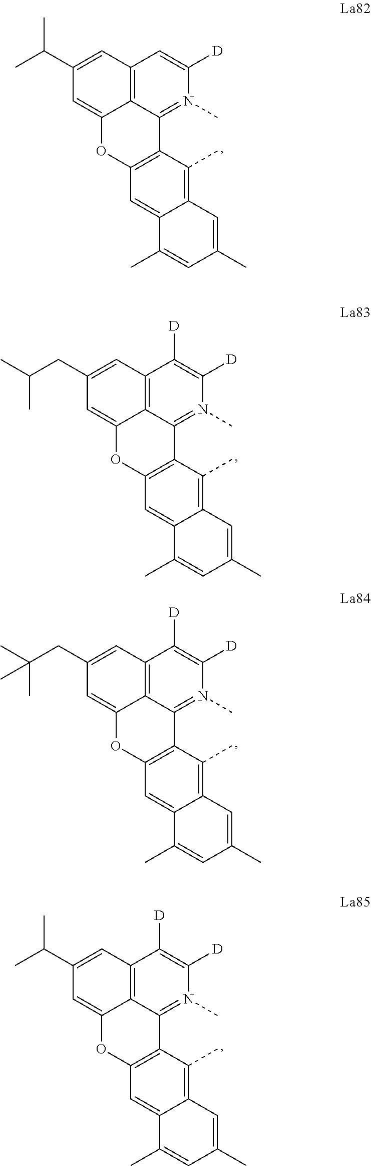

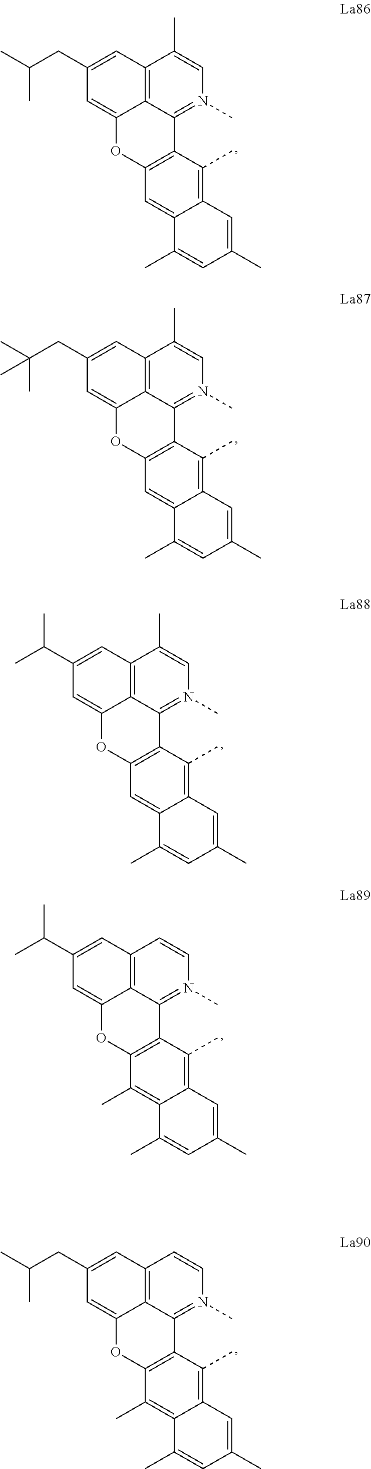

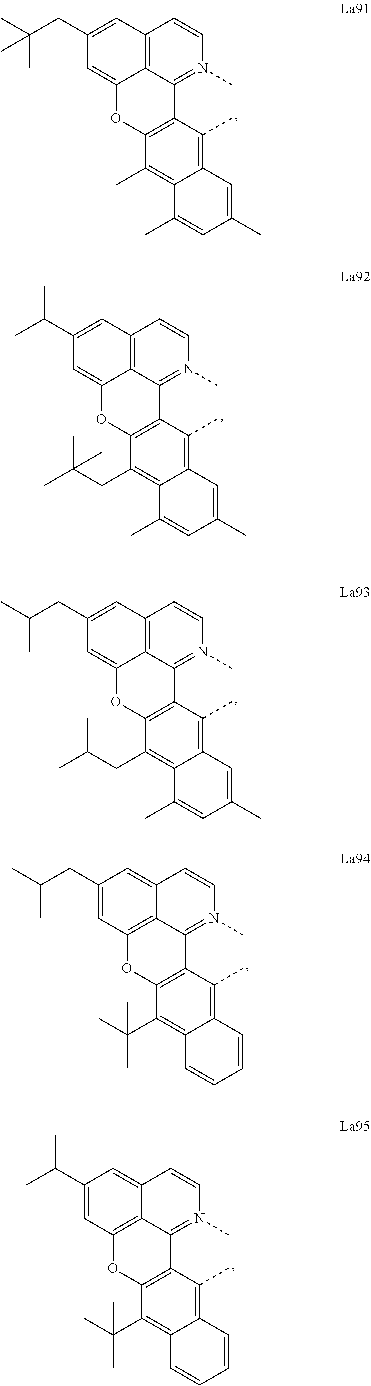

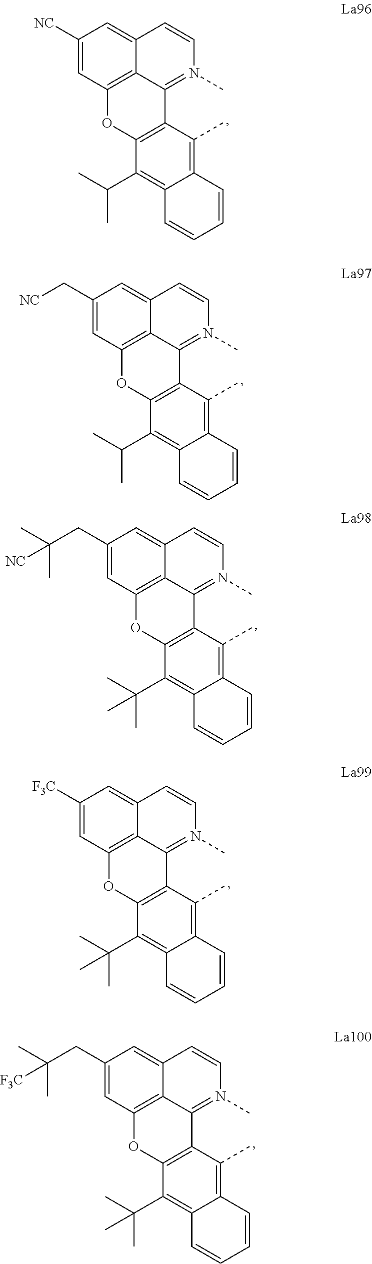

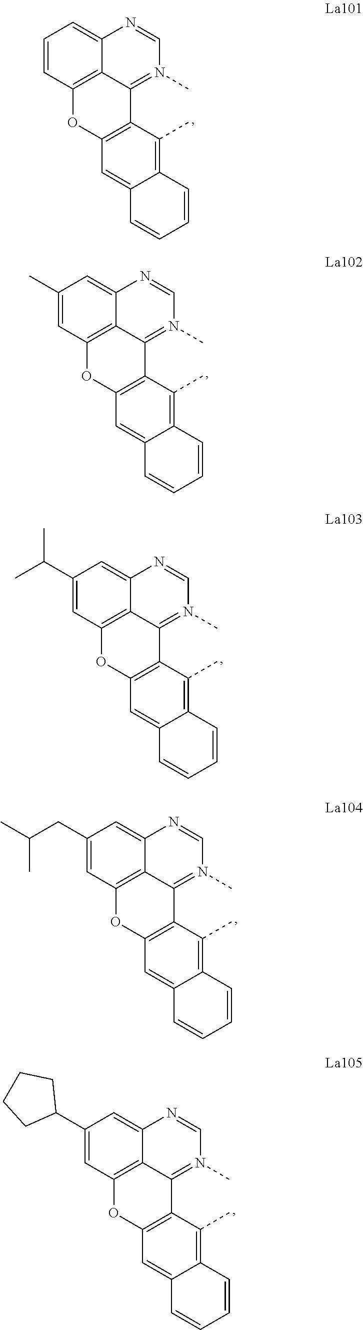

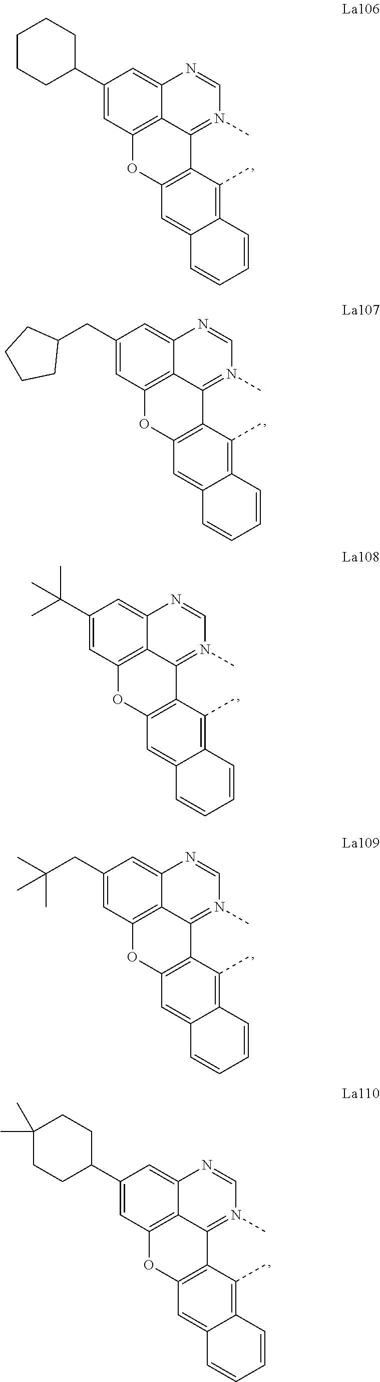

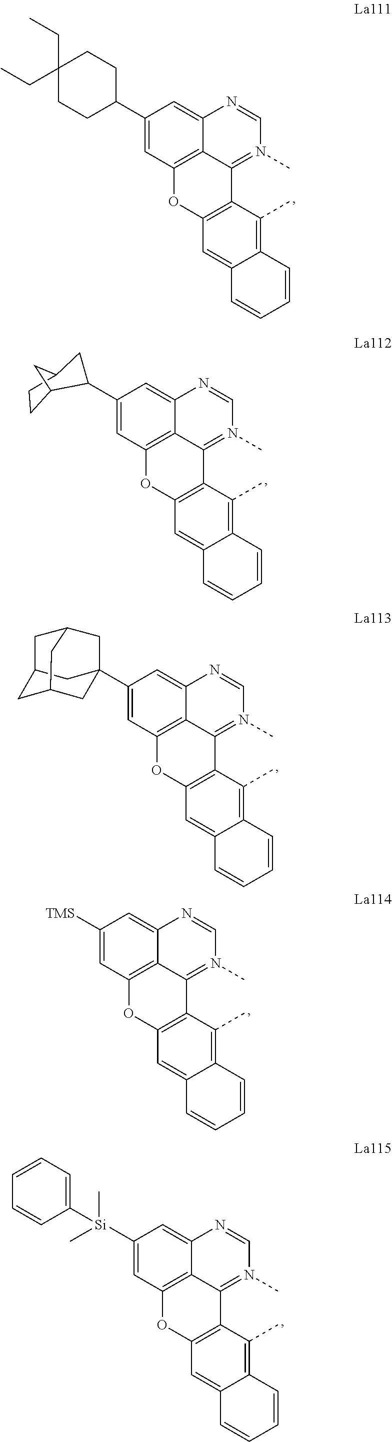

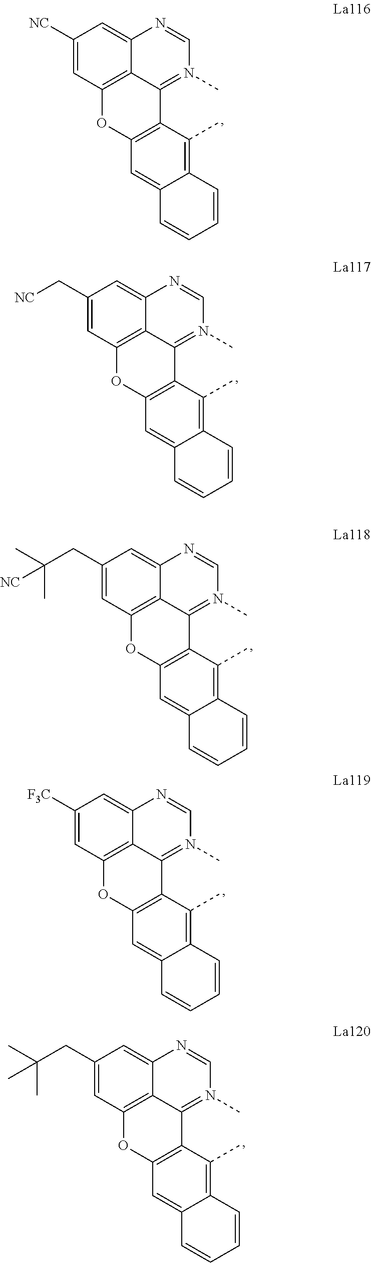

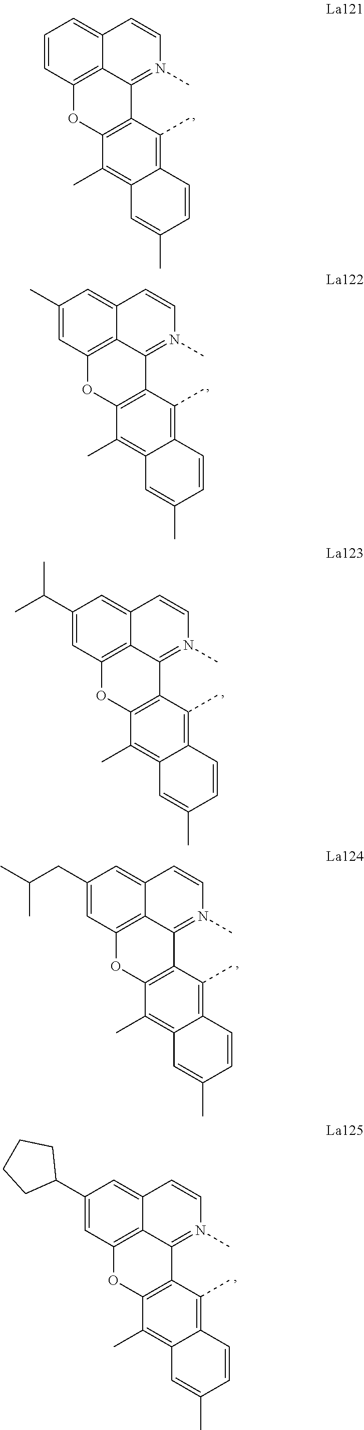

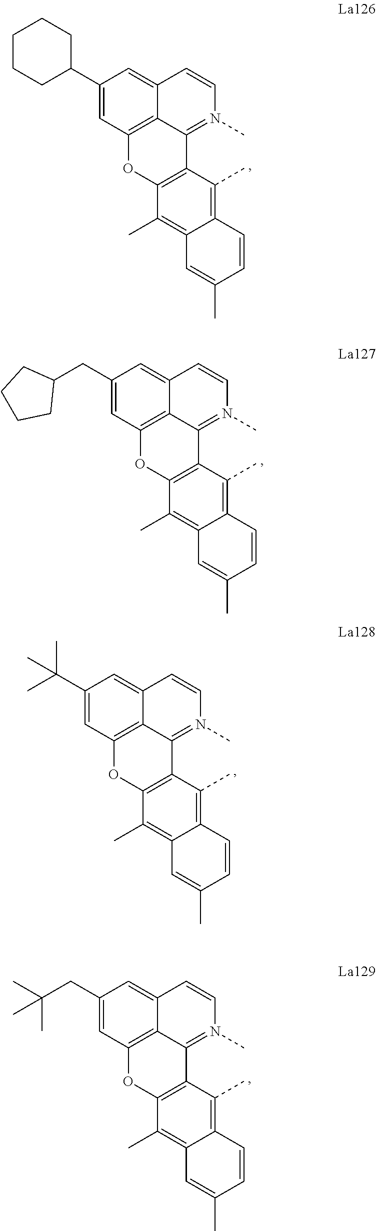

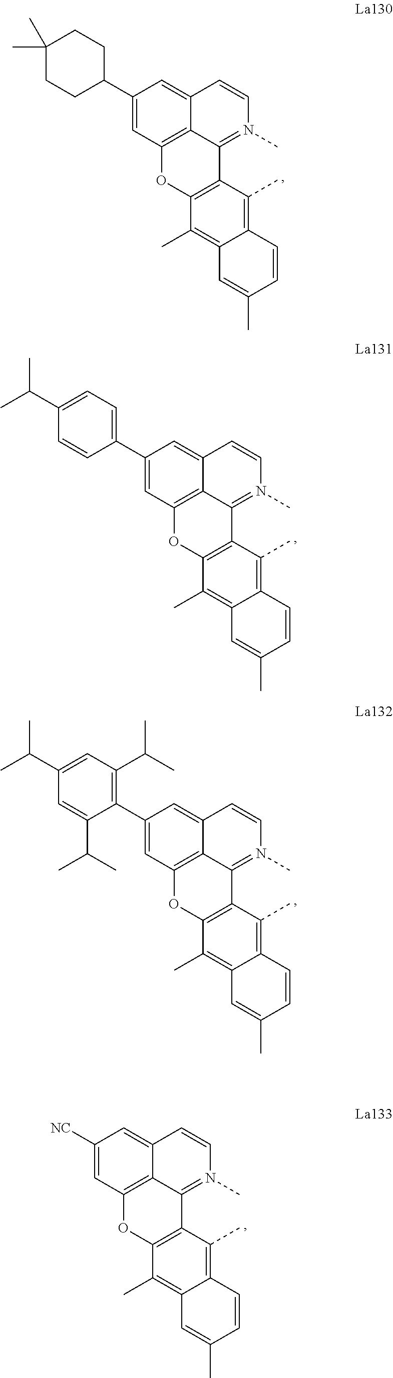

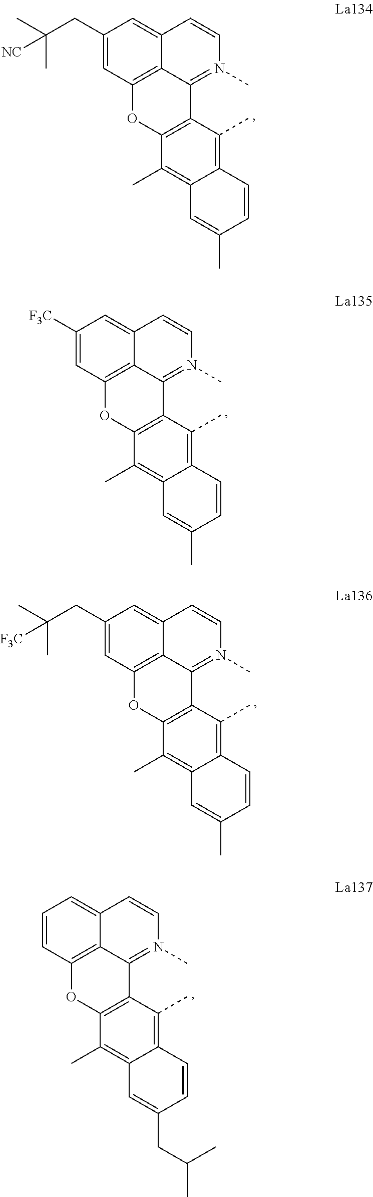

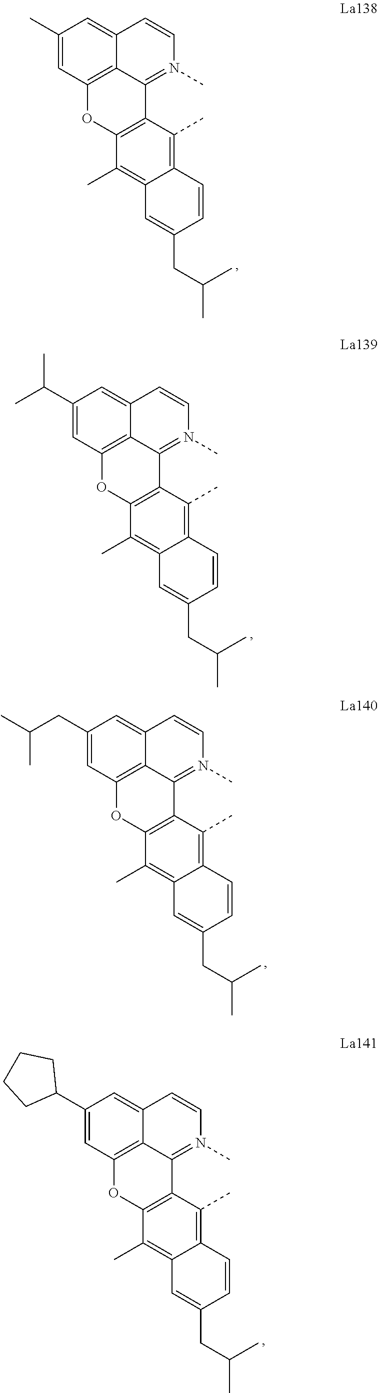

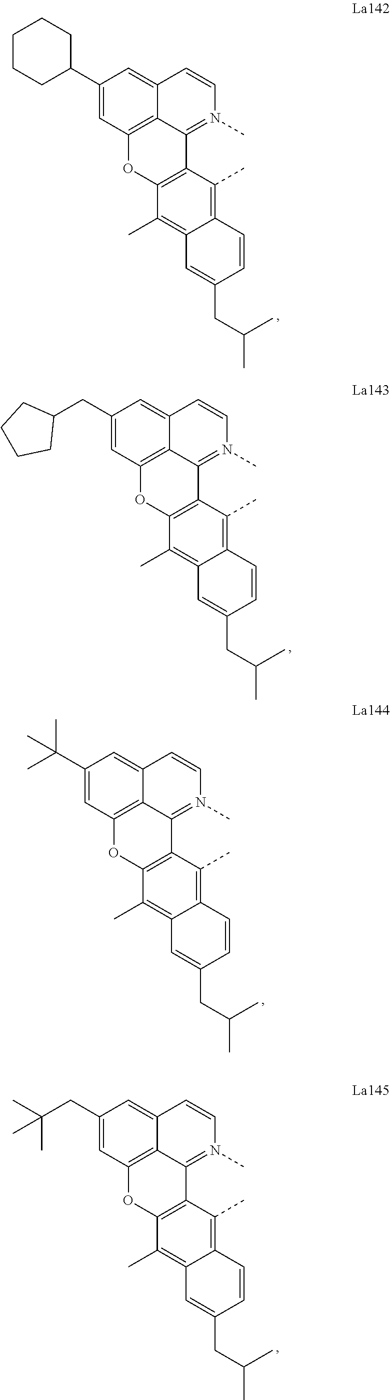

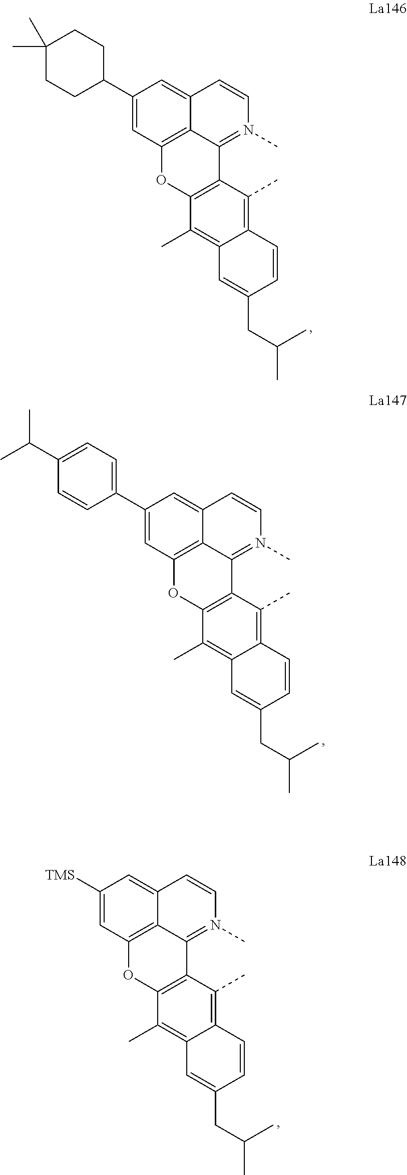

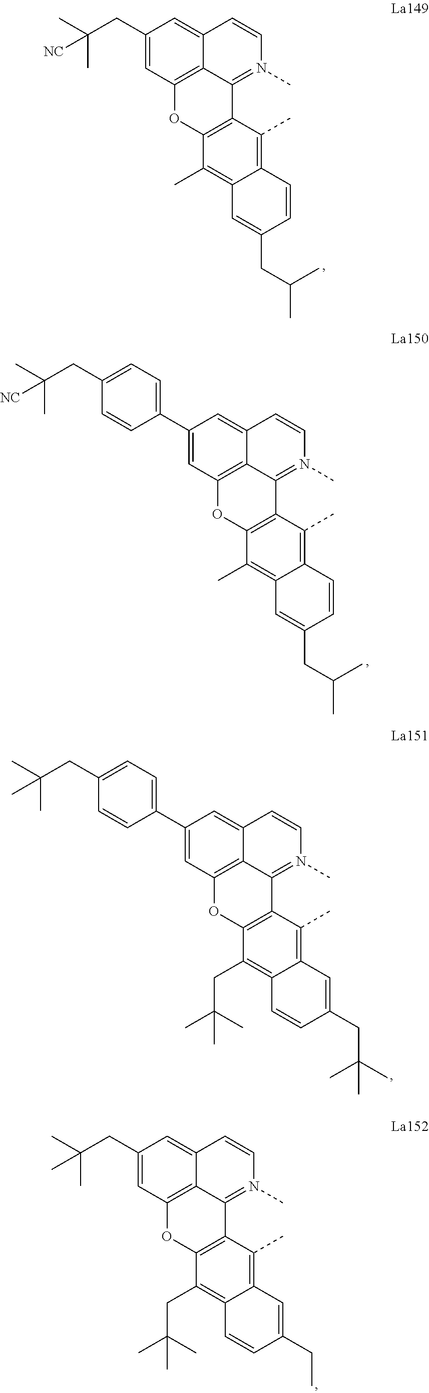

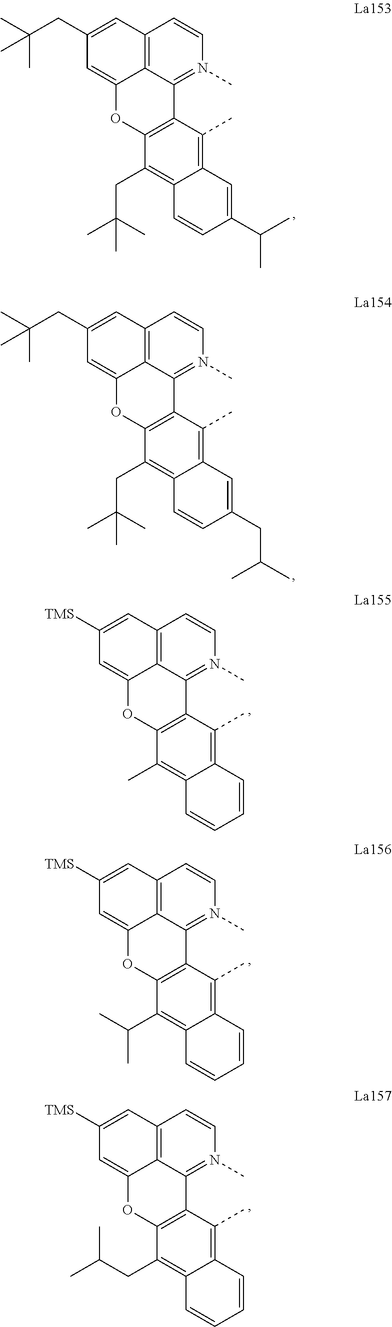

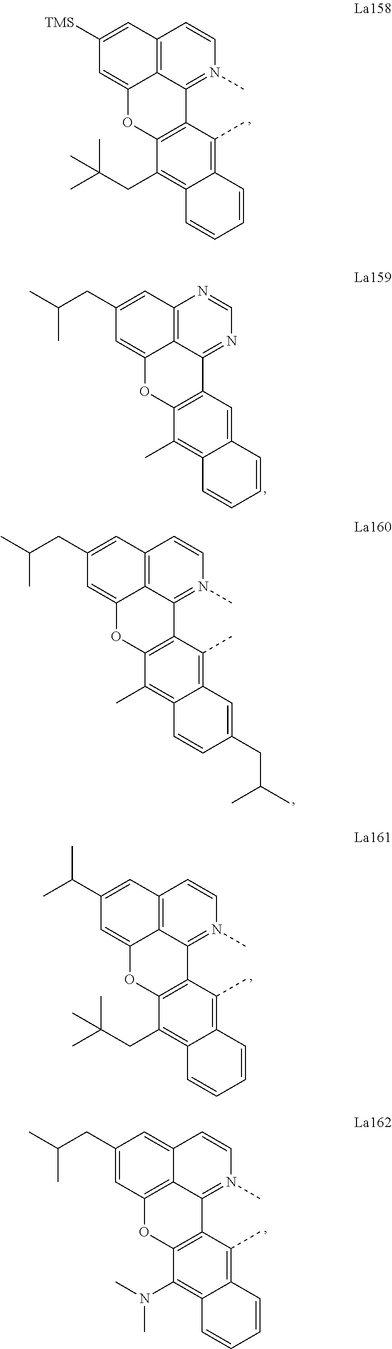

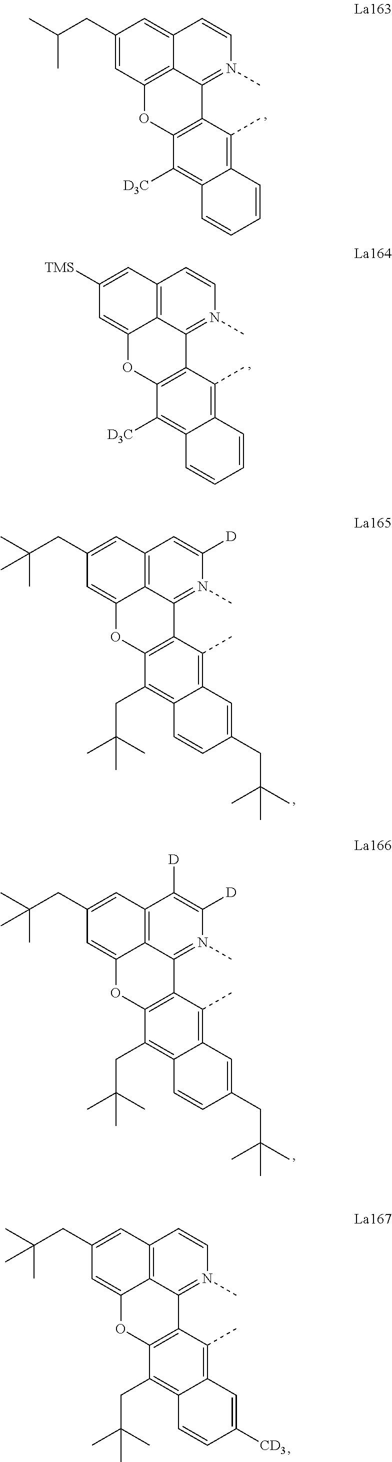

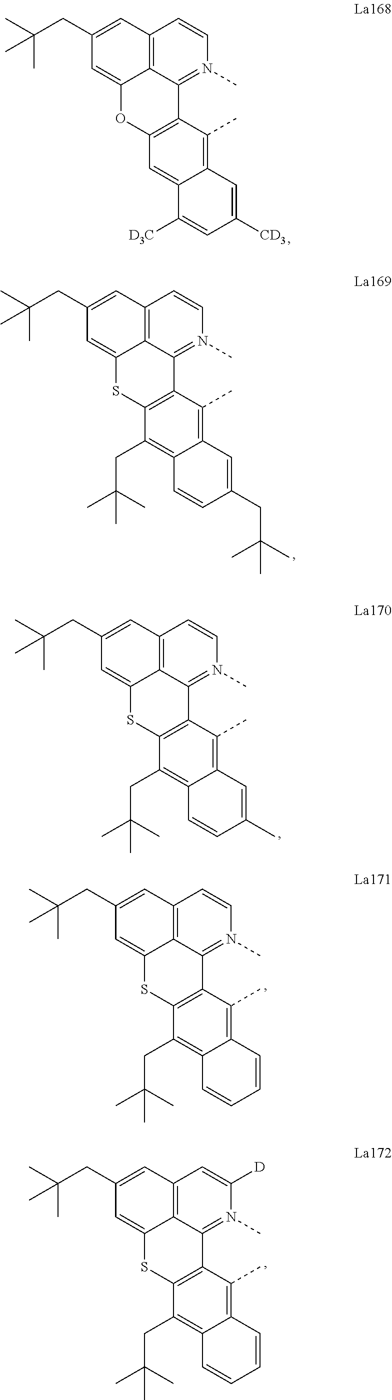

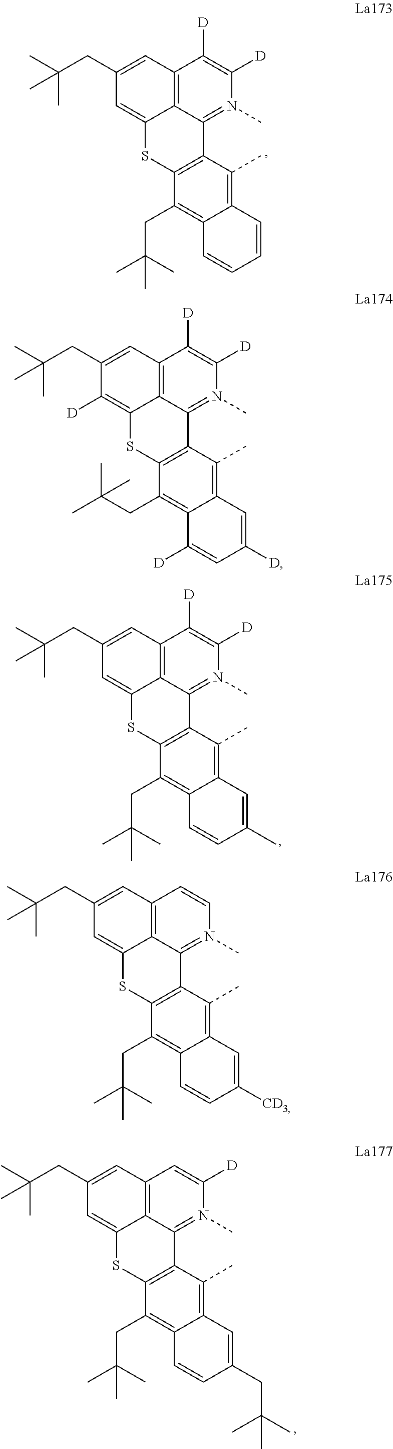

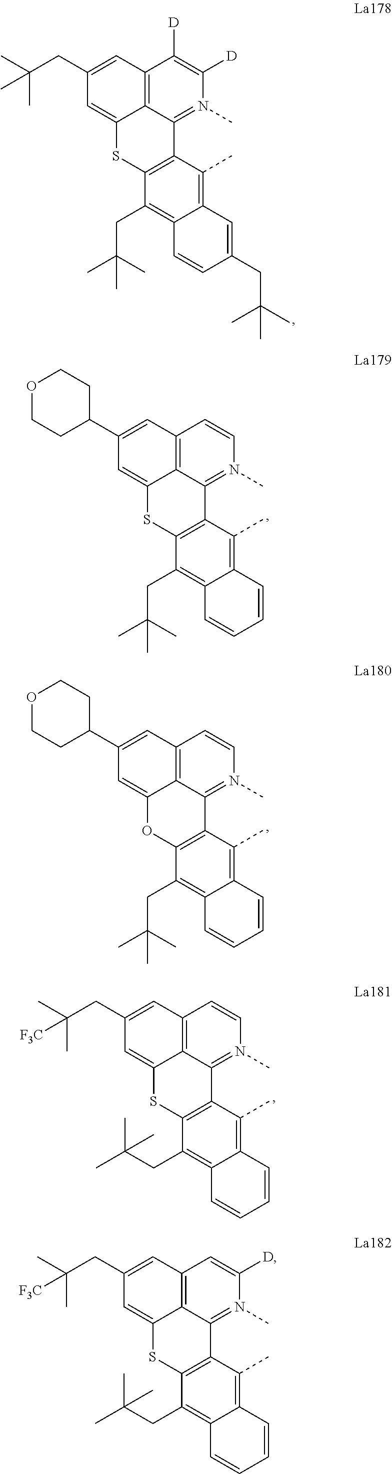

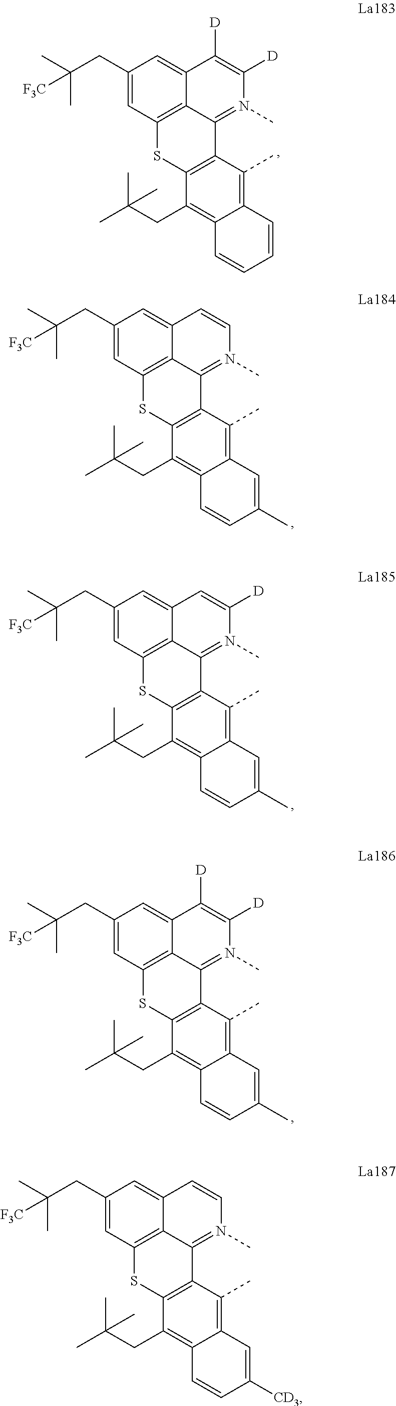

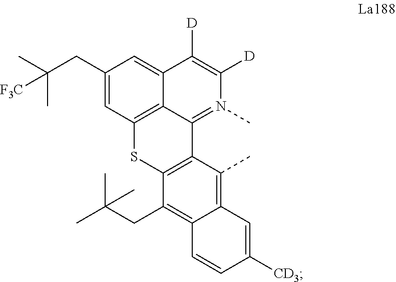

wherein in the above structures, TMS represents trimethylsilyl.

According to an embodiment of the present disclosure, wherein, hydrogen in the structures of Lₐ₁ to Lₐ₁₈₈ can be partially or fully substituted with deuterium.

According to an embodiment of the present disclosure, wherein, the second compound has a structure of M(Lₐ)ₘ(L_{b})ₙ(L_{c})_{q}; wherein

the metal M is selected from a metal with a relative atomic mass greater than 40; Lₐ, L_{b} and L_{c}, are a first ligand, a second ligand and a third ligand of the complex, respectively; m is 1, 2 or 3, n is 0, 1 or 2, q is 0, 1 or 2, and m+n+q equals to an oxidation state of the metal M; wherein when m is greater than 1, a plurality of Lₐ are identical or different; when n is 2, two L_{b} are identical or different; when q is 2, two L_{c} are identical or different;

Lₐ, L_{b} and L_{c} can be optionally joined to form a multidentate ligand; for example, Lₐ, L_{b} and L_{c} can be optionally joined to form a tetradentate ligand or a hexadentate ligand; it is possible that Lₐ, L_{b} and L_{c} are not joined so that no multidentate ligand is formed;

L_{b} and L_{c} are, at each occurrence identically or differently, selected from the group consisting of the following structures:

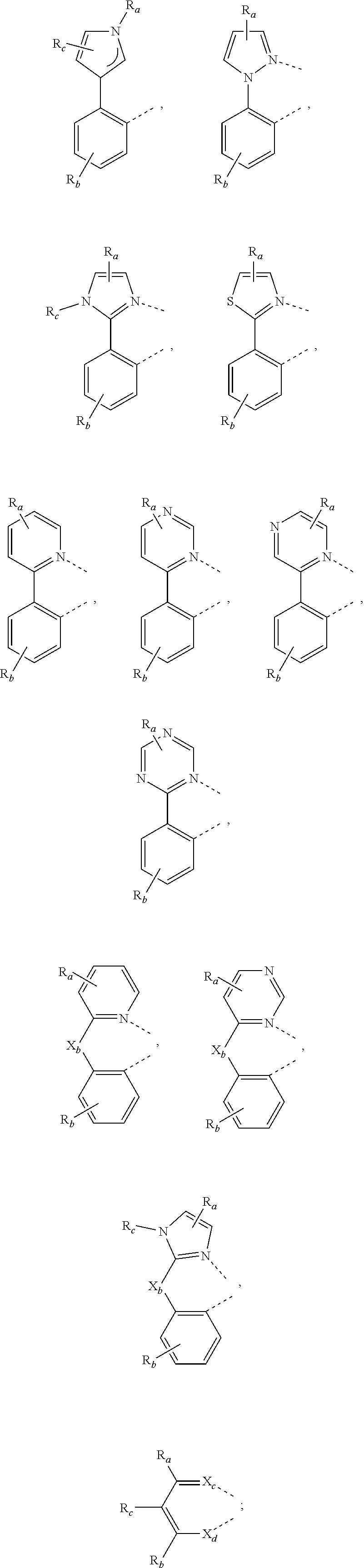

wherein Rₐ, R_{b} and R_{c} represent, at each occurrence identically or differently, mono-substitution, multiple substitutions or non-substitution;

X_{b} is, at each occurrence identically or differently, selected from the group consisting of: O, S, Se, NR_{N1} and CR_{C1}R_{C2};

X_{c} and X_{d} are, at each occurrence identically or differently, selected from the group consisting of: O, S, Se and NR_{N2};

Rₐ, R_{b}, R_{c}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, a sulfanyl group, a sulfinyl group, a sulfonyl group, a phosphino group and combinations thereof; and

adjacent substituents Rₐ, R_{b}, R_{c}, R_{N1}, R_{N2}, R_{C1} and R_{C2} can be optionally joined to form a ring.

In this embodiment, the expression that “adjacent substituents Rₐ, R_{b}, R_{c}, R_{N1}, R_{N2}, R_{C1} and R_{C2} can be optionally joined to form a ring” is intended to mean that any one or more of groups of adjacent substituents, such as two substituents Rₐ, two substituents R_{b}, two substituents R_{c}, substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{c2}, substituents Rₐ and R_{N2}, substituents R_{b} and R_{N2}, and substituents R_{C1} and R_{C2}, can be joined to form a ring. Obviously, it is possible that none of these substituents are joined to form a ring.

In this embodiment, the expression that “Lₐ, L_{b} and L_{c} can be optionally joined to form a multidentate ligand” is intended to mean that any two or three of Lₐ, L_{b} and L_{c} can be joined to form a tetradentate ligand or a hexadentate ligand. Obviously, it is possible that none of Lₐ, L_{b} and L_{c} are joined to form a multidentate ligand.

According to an embodiment of the present disclosure, wherein, in the second compound, the metal M is selected from Ir, Rh, Re, Os, Pt, Au or Cu.

According to an embodiment of the present disclosure, wherein, in the second compound, the metal M is selected from Ir, Pt or Os.

According to an embodiment of the present disclosure, wherein, in the second compound, the metal M is Ir.

According to an embodiment of the present disclosure, in the device, the second compound is an Ir complex and has a structure represented by any one of Ir(Lₐ)(L_{b})(L_{c}), Ir(Lₐ)₂(L_{b}), Ir(Lₐ)₂(L_{c}) or Ir(Lₐ)(L_{c})₂.

According to an embodiment of the present disclosure, wherein, L_{b} is, at each occurrence identically or differently, selected from the following structure:

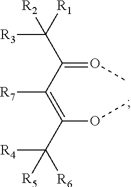

wherein R₁ to R₇ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, a sulfanyl group, a sulfinyl group, a sulfonyl group, a phosphino group and combinations thereof.

According to an embodiment of the present disclosure, wherein, L_{b} is, at each occurrence identically or differently, selected from the following structure:

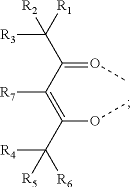

wherein at least one of R₁ to R₃ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one of R₄ to R₆ is substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, wherein, L_{b} is, at each occurrence identically or differently, selected from the following structure:

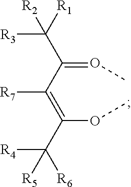

wherein at least two of R₁ to R₃ are selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof; and/or at least one of R₄ to R₆ is substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms or a combination thereof.

According to an embodiment of the present disclosure, wherein, L_{b} is, at each occurrence identically or differently, selected from the following structure:

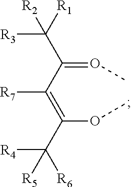

wherein at least two of R₁ to R₃ are selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof, and/or at least two of R₄ to R₆ are selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms or a combination thereof.

According to an embodiment of the present disclosure, wherein, in the second compound, L_{b} is, at each occurrence identically or differently, selected from the group consisting of L_{b1} to L_{b322}, wherein the specific structures of L_{bl} to L_{b322} are referred to claim 23.

According to an embodiment of the present disclosure, wherein, in the second compound, L_{c} is, at each occurrence identically or differently, selected from the group consisting of L_{c1} to L_{c231}, wherein the specific structures of L_{c1} to L_{c231} are referred to claim 23.

According to an embodiment of the present disclosure, in the device, the second compound is an Ir complex and has a structure represented by any one of Ir(Lₐ)(L_{b})(L_{c}), Ir(Lₐ)₂(L_{b}), Ir(Lₐ)₂(L_{c}) and Ir(Lₐ)(L_{c})₂; when the second compound has a structure of Ir(Lₐ)(L_{b})(L_{c}), Lₐ is selected from any one of the group consisting of Lₐ₁ to Lₐ₁₈₈, L_{b} is selected from any one of the group consisting of L_{bl} to L_{b322}, and L_{c} is selected from any one of the group consisting of L_{c1} to L_{c231}; when the second compound has a structure of Ir(Lₐ)₂L_{b}, Lₐ is selected from any one or any two of the group consisting of Lₐ₁ to Lₐ₁₈₈, and L_{b} is selected from any one of the group consisting of L_{b1} to L_{b322}; when the second compound has a structure of Ir(Lₐ)₂(L_{c}), Lₐ is selected from any one or any two of the group consisting of Lₐ₁ to Lₐ₁₈₈, and L_{c} is selected from any one of the group consisting of L_{c1} to L_{c231}; when the second compound has a structure of Ir(Lₐ)(L_{c})₂, Lₐ is selected from any one of the group consisting of Lₐ₁ to Lₐ₁₈₈, and L_{c} is selected from any one or any two of the group consisting of L_{c1} to L_{c231}.

According to an embodiment of the present disclosure, wherein, the second compound is selected from the group consisting of Compound C₁ to Compound C₁₇₃:

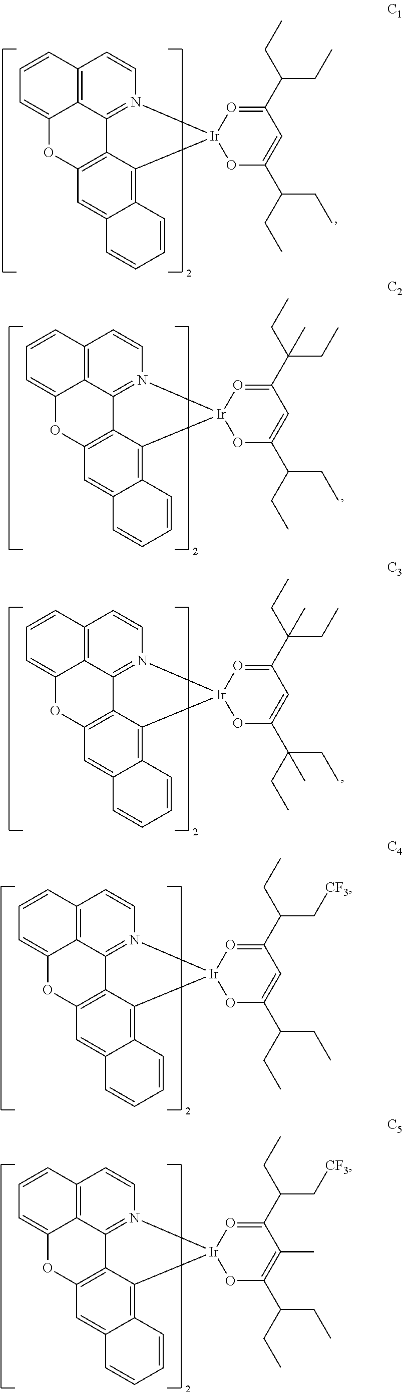

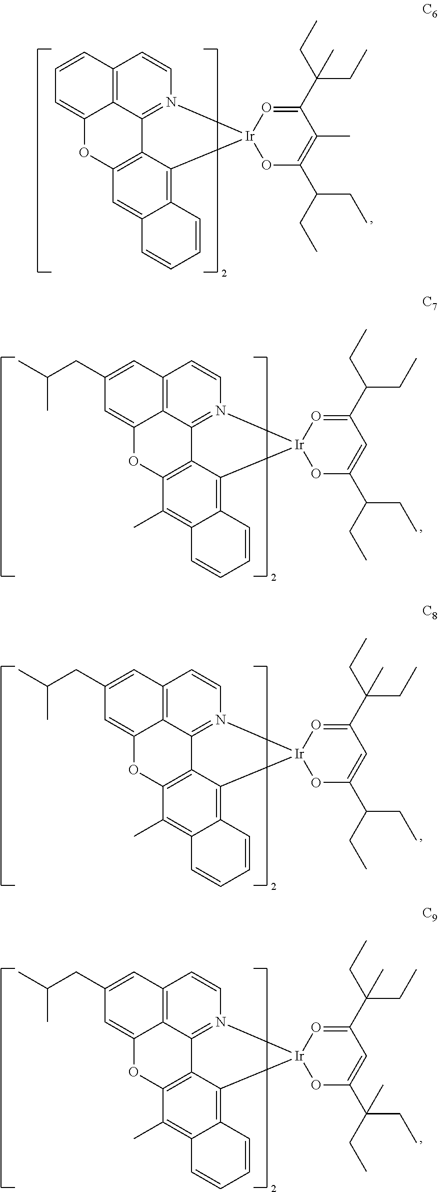

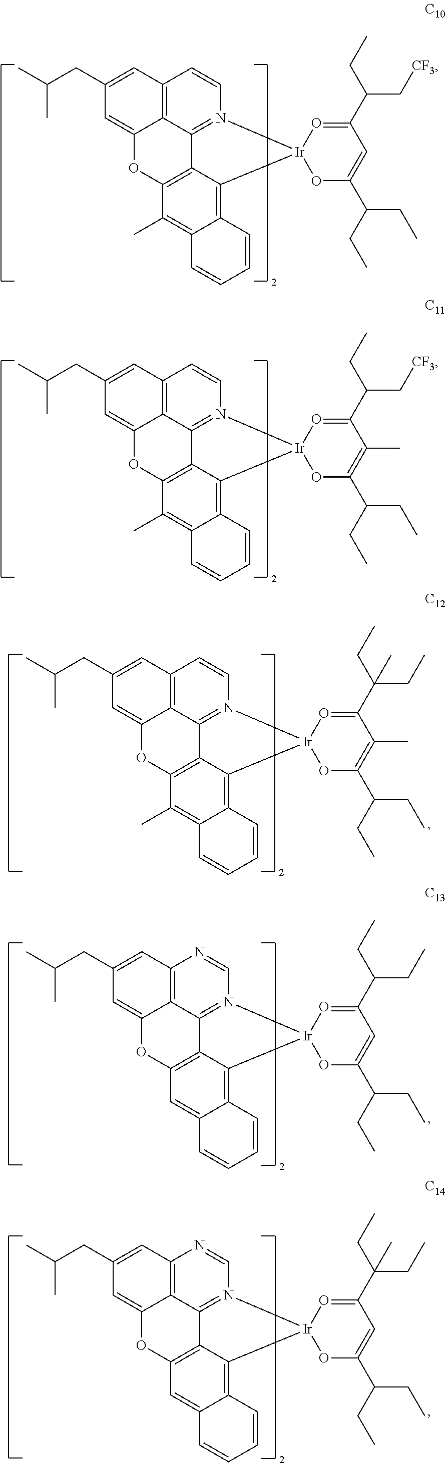

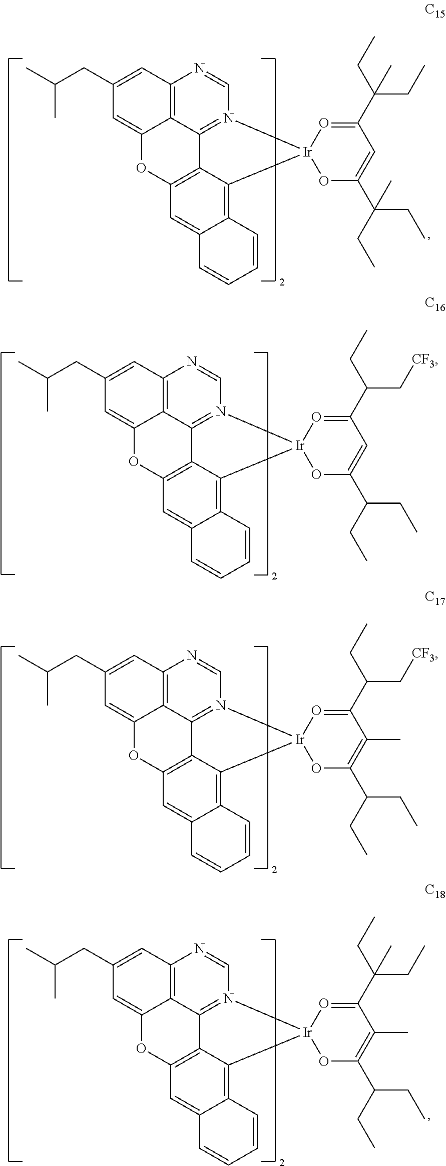

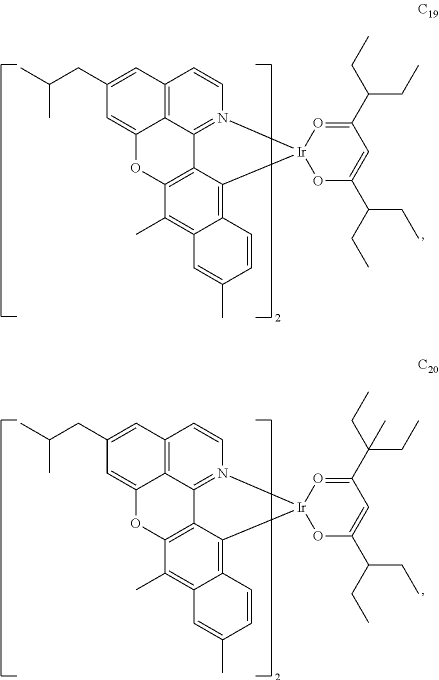

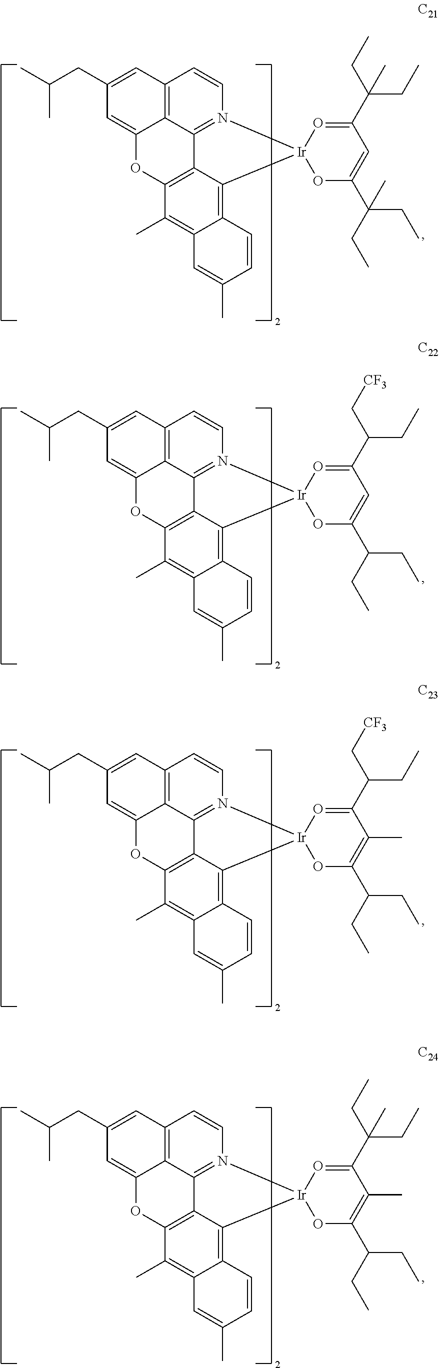

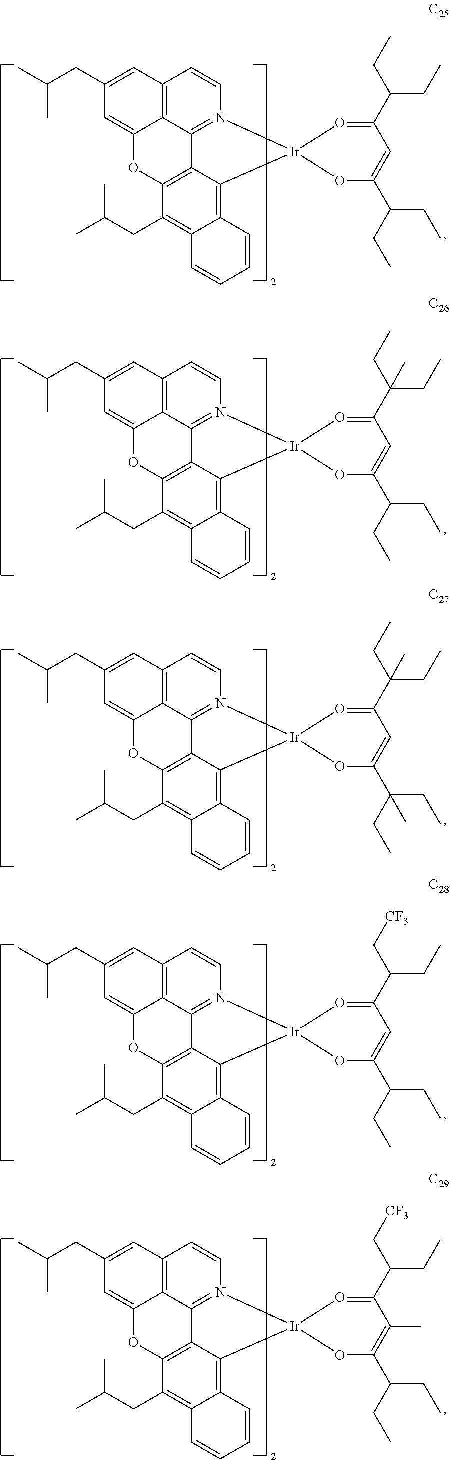

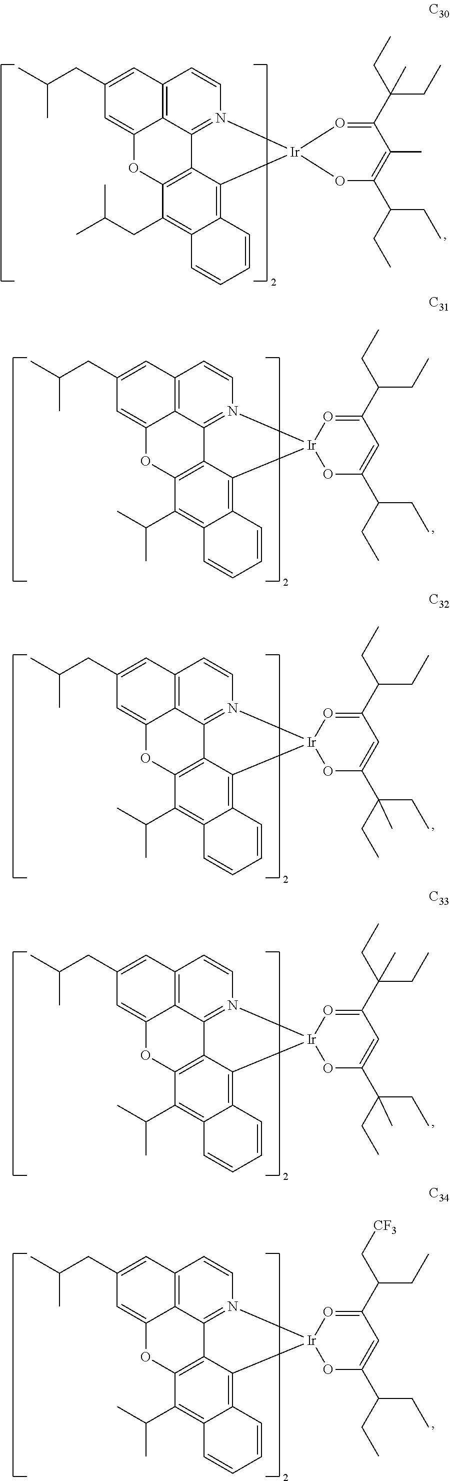

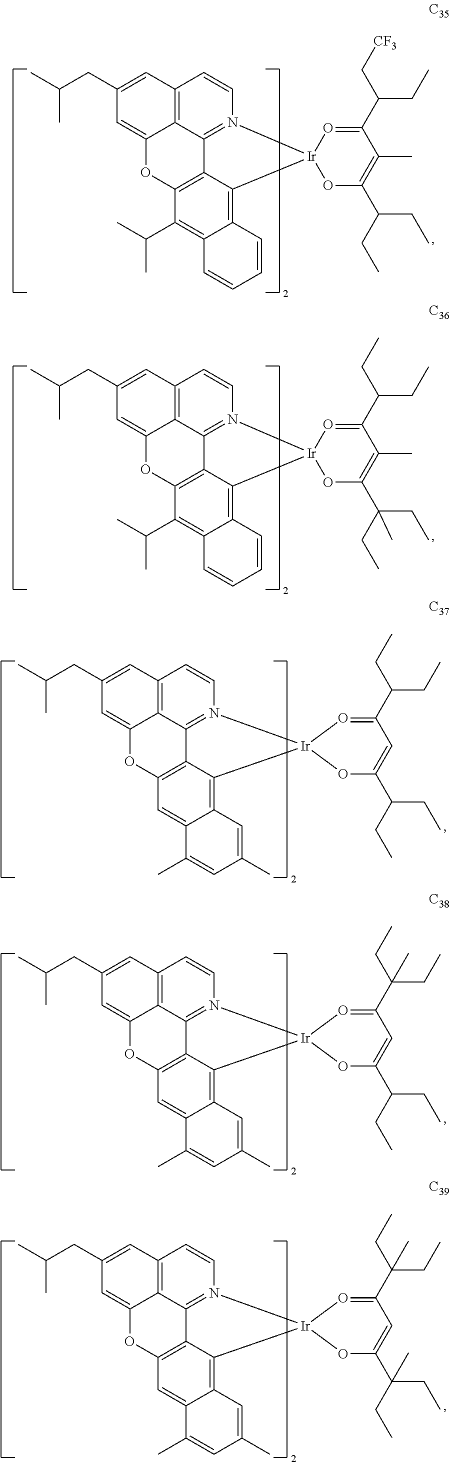

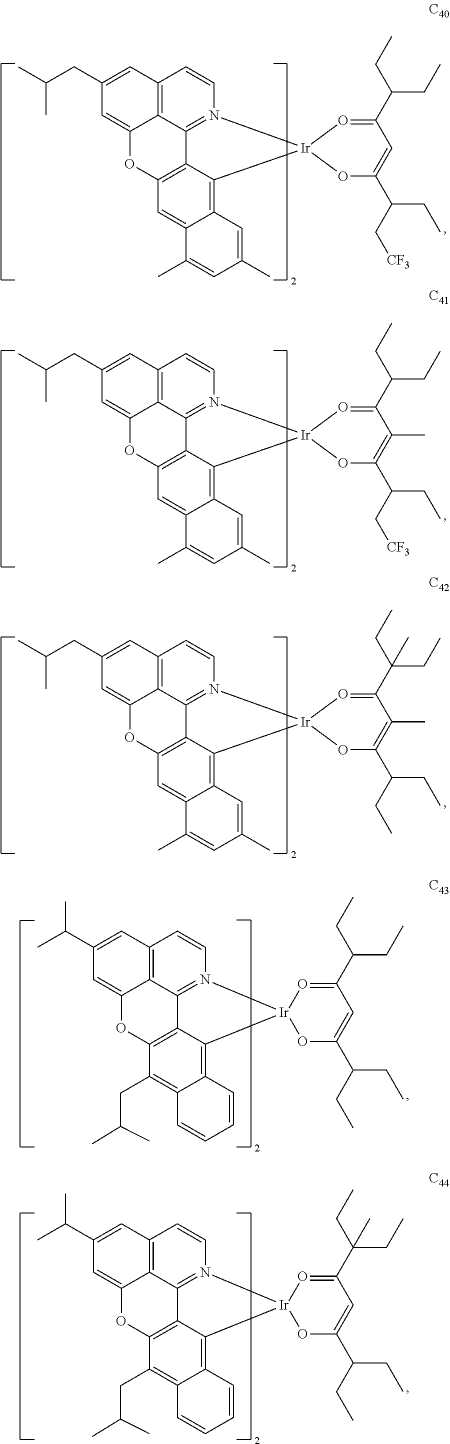

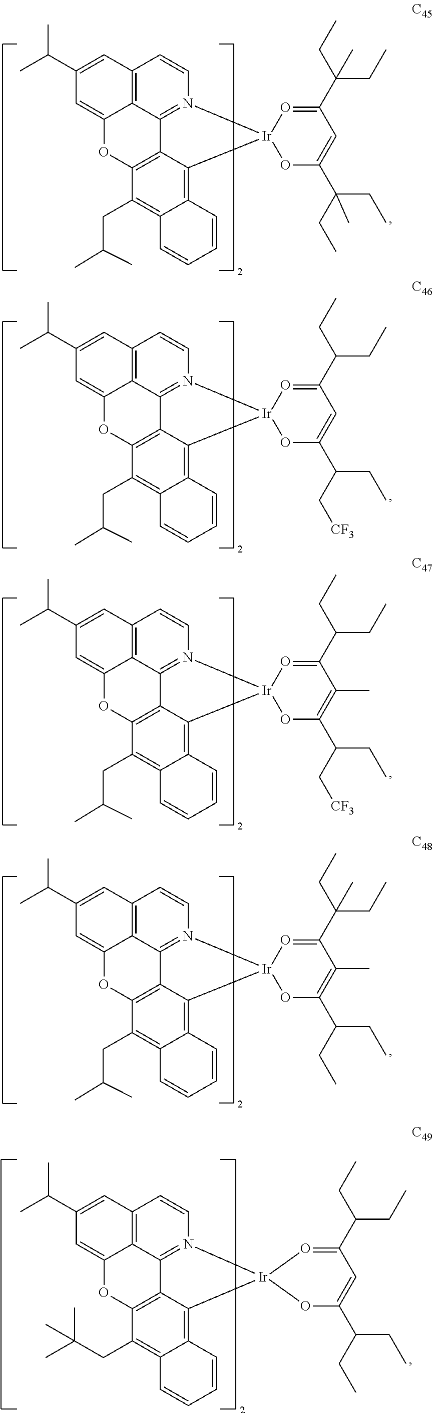

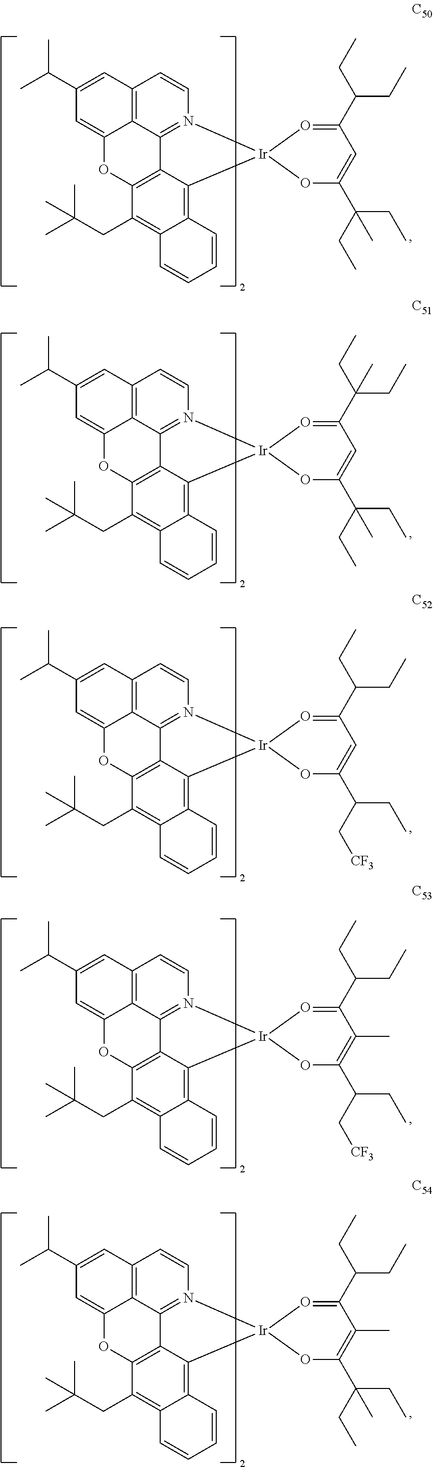

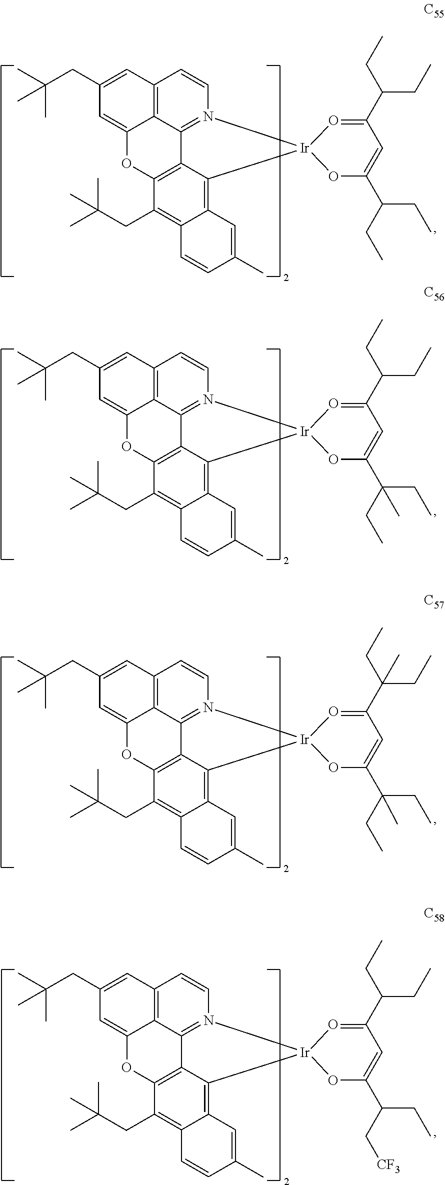

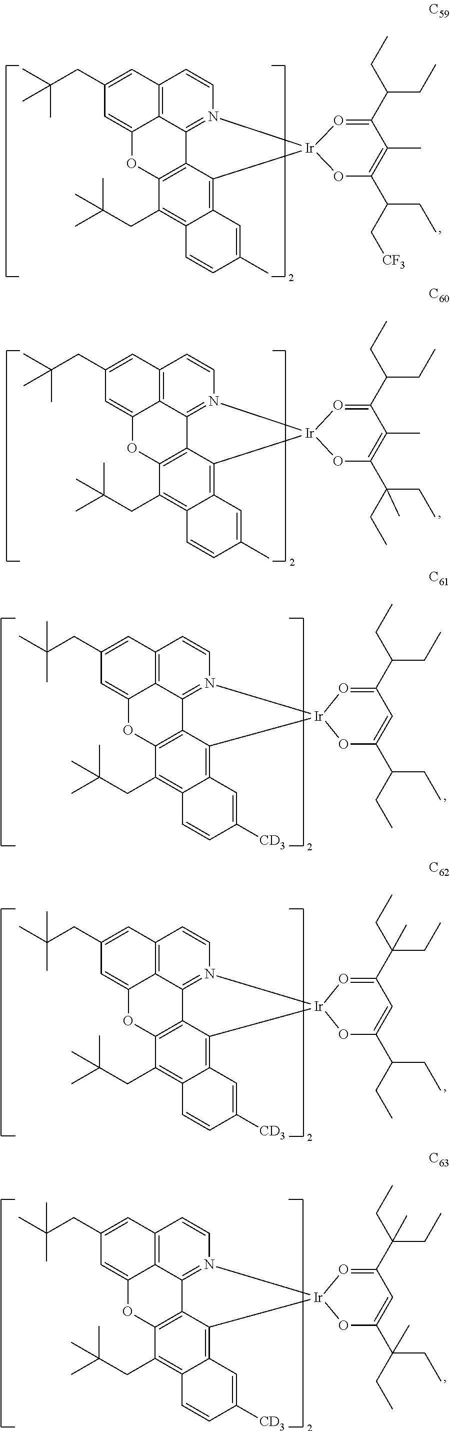

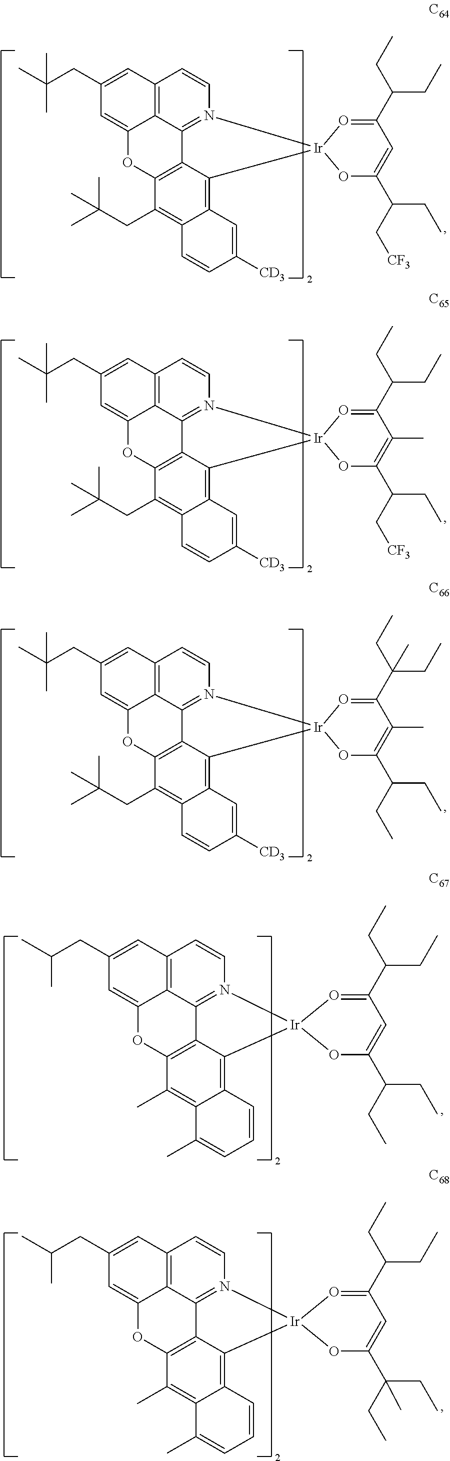

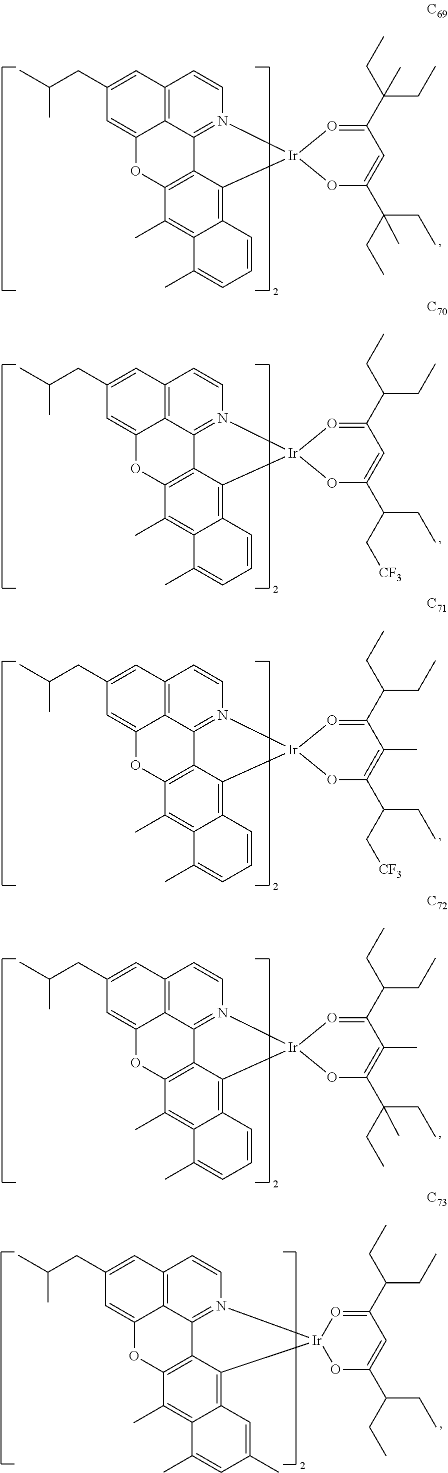

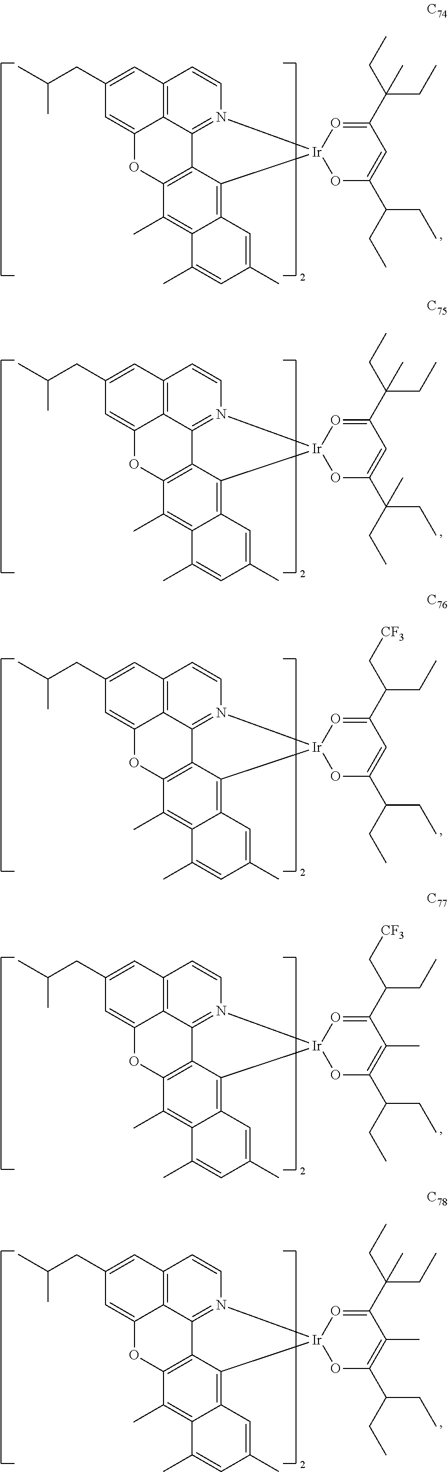

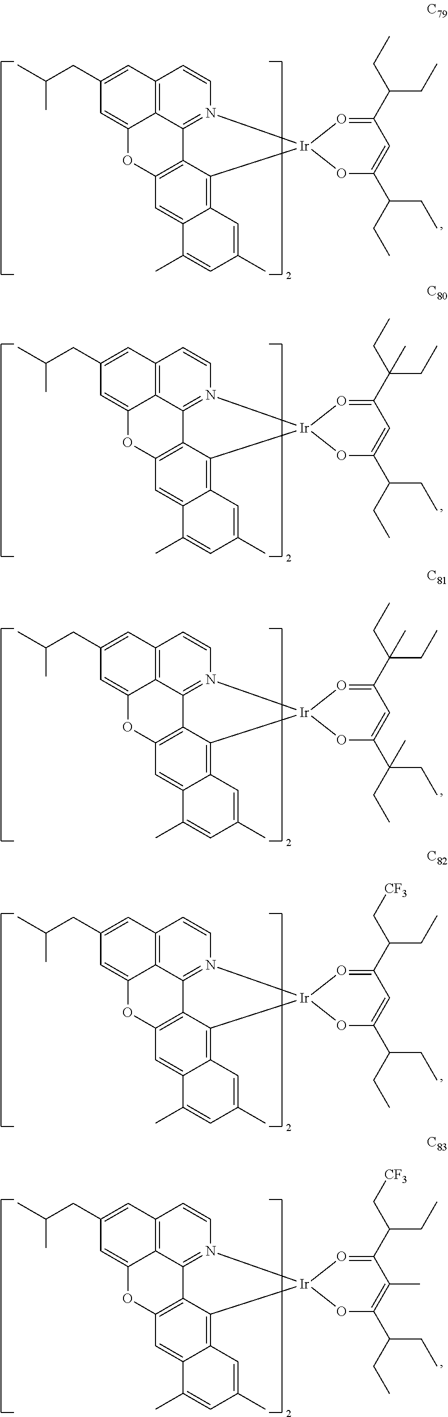

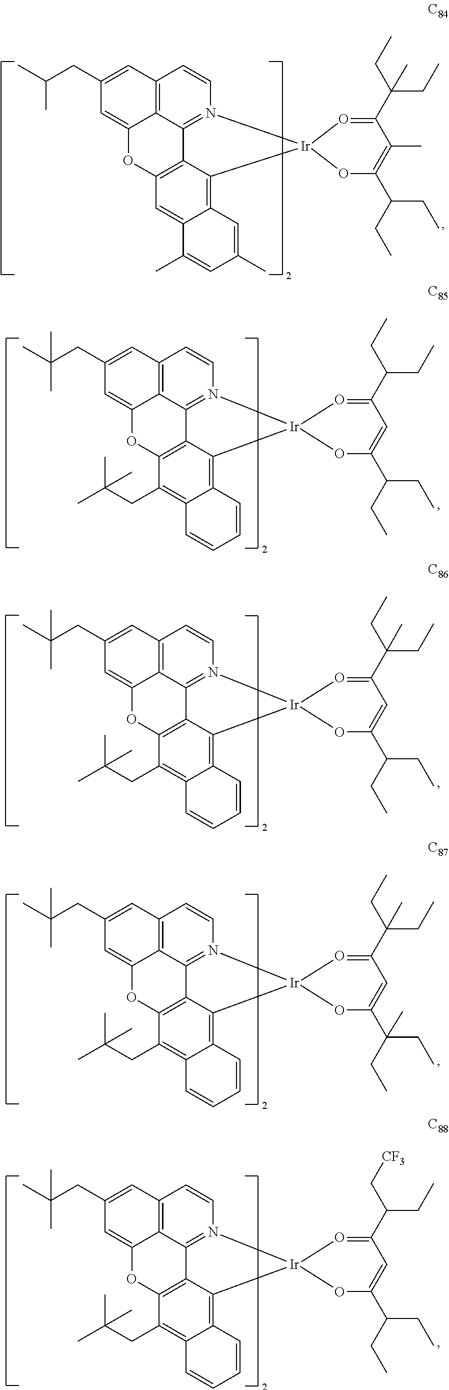

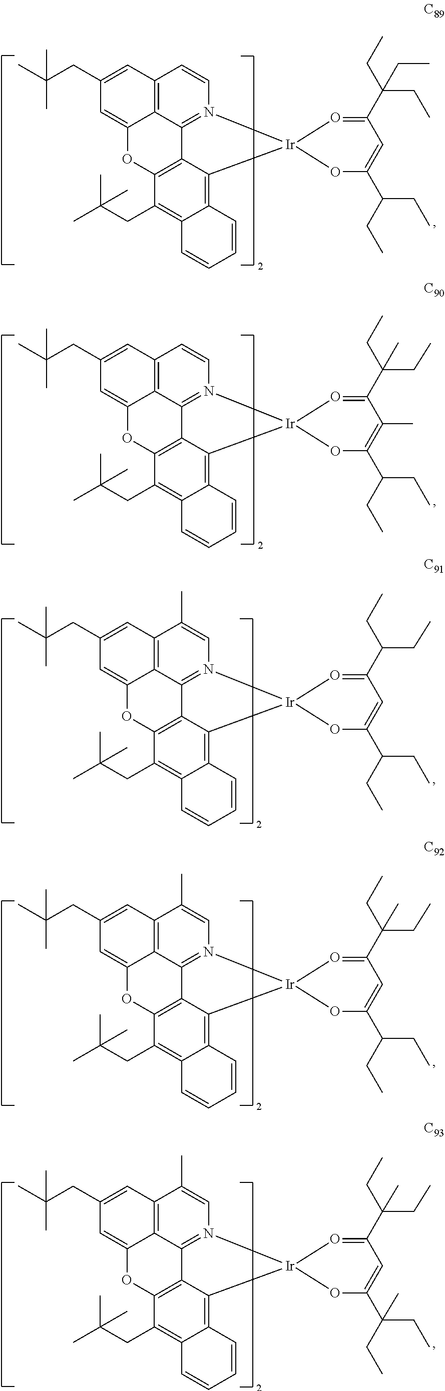

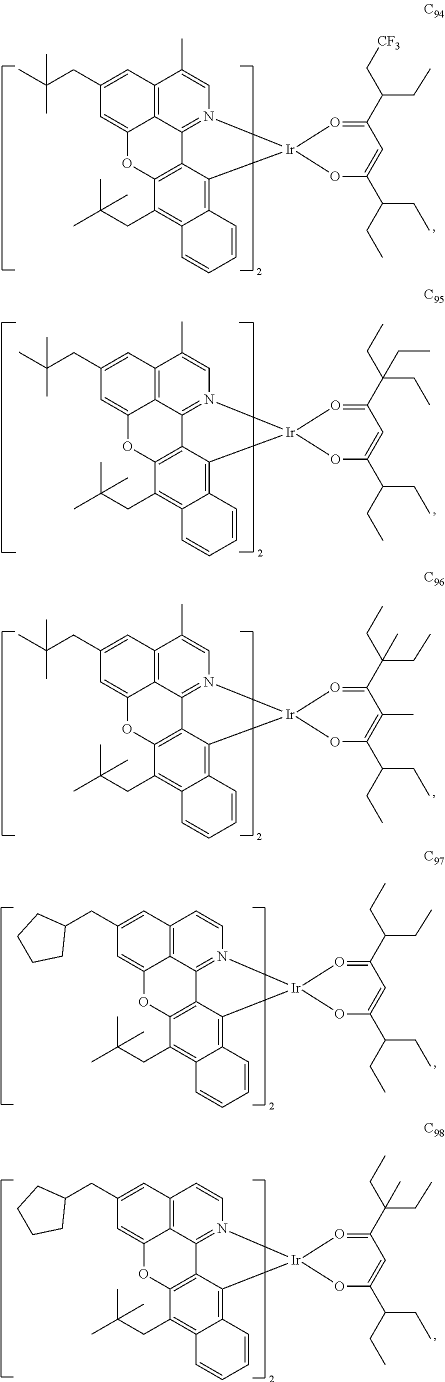

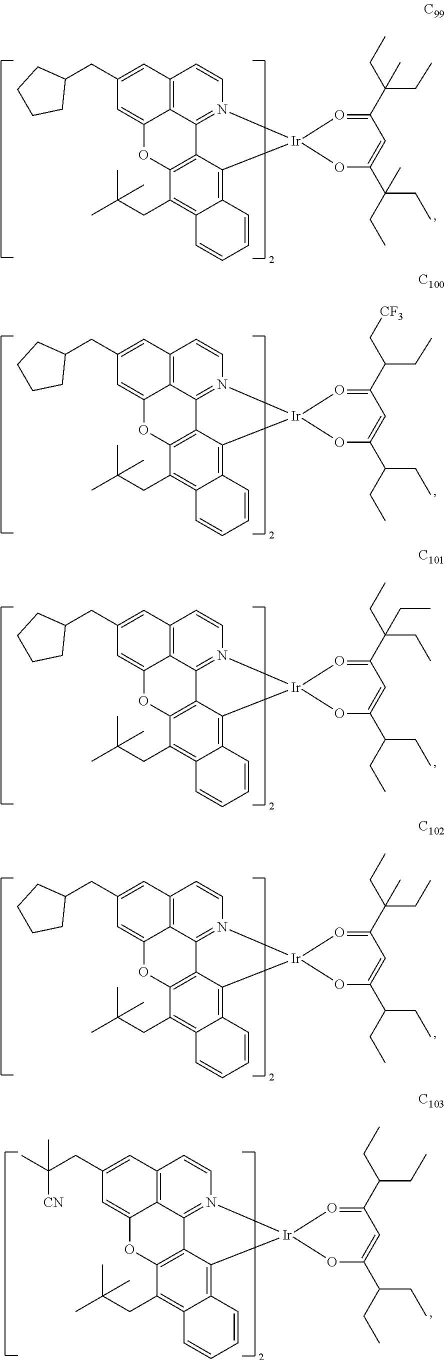

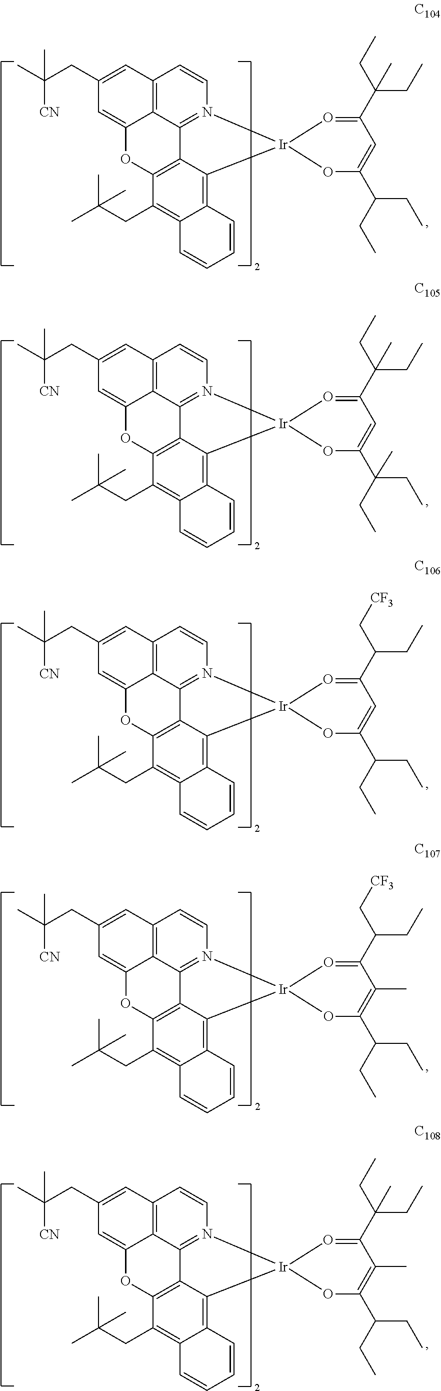

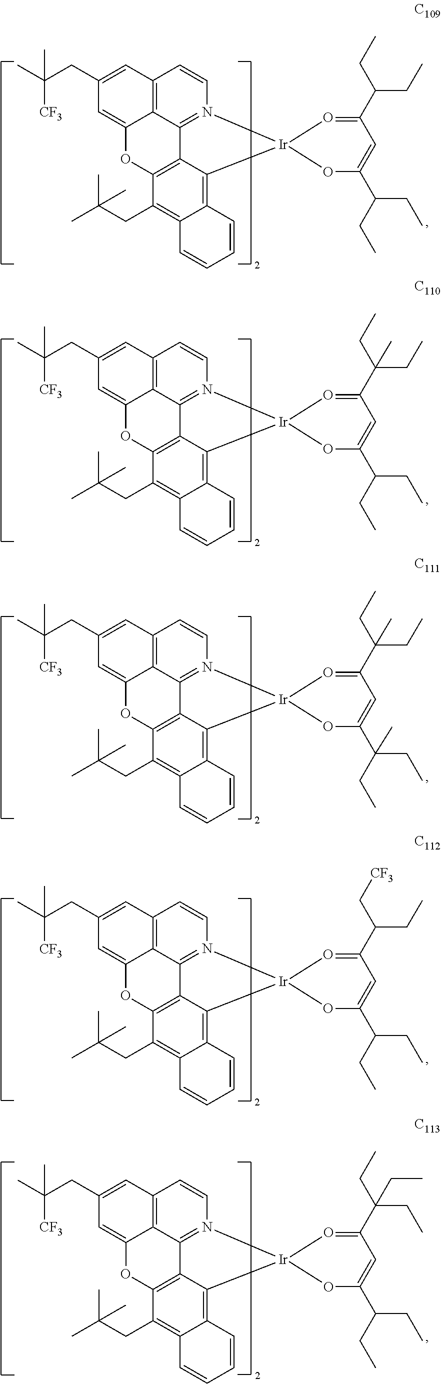

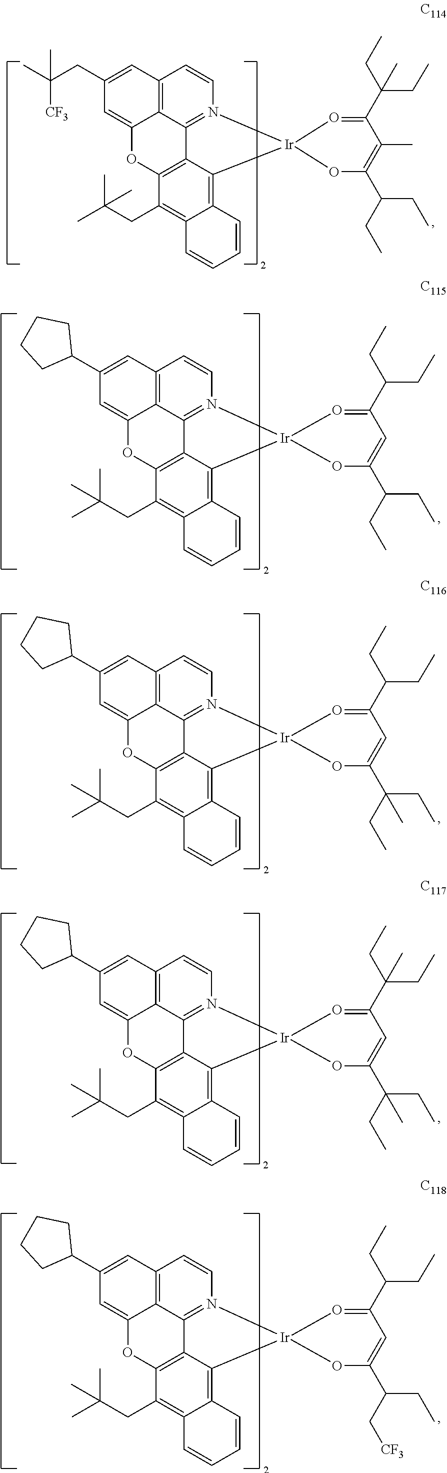

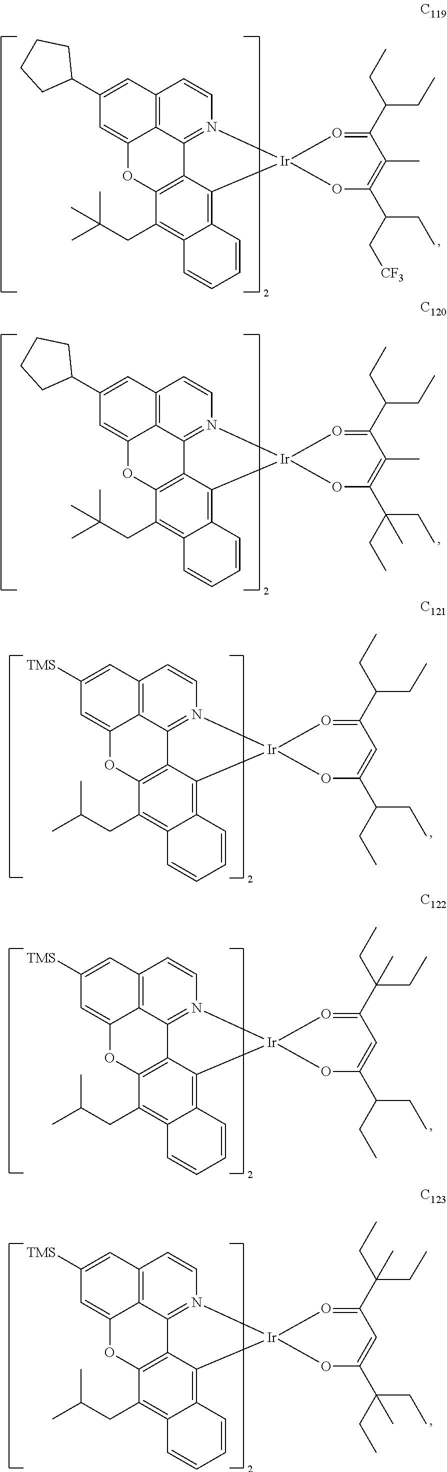

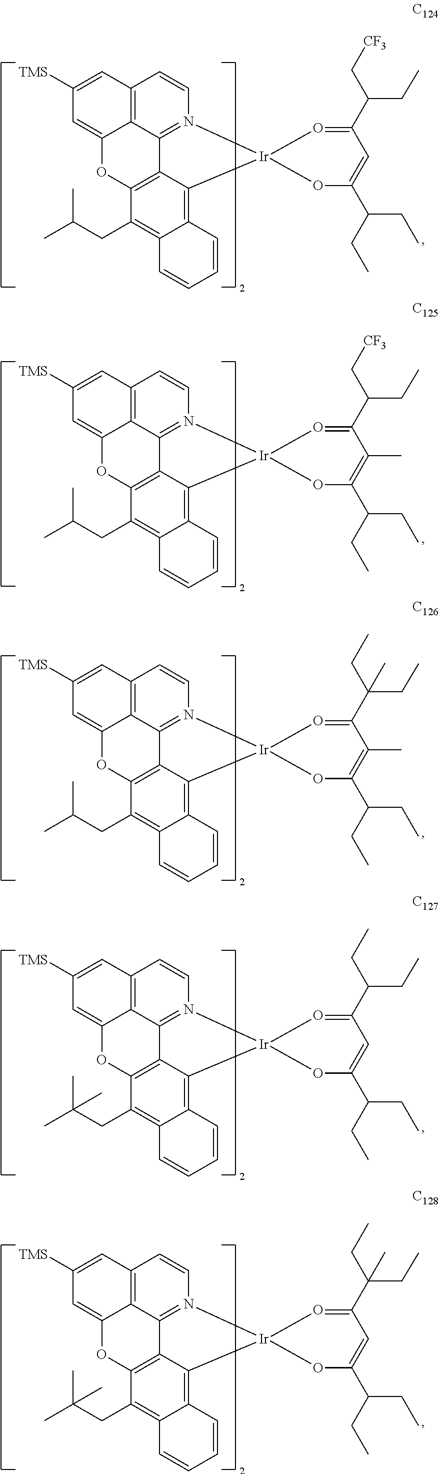

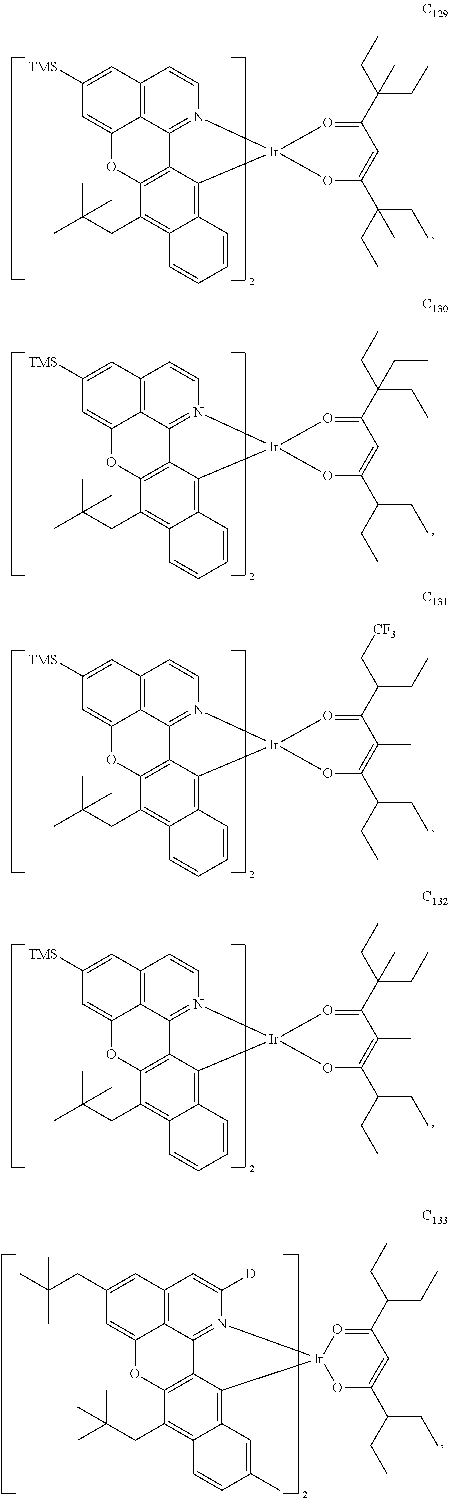

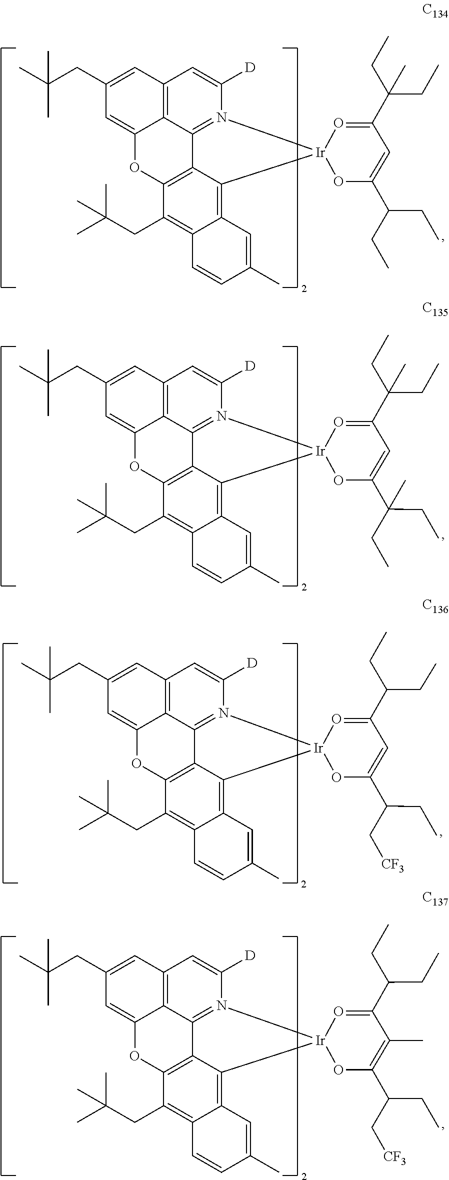

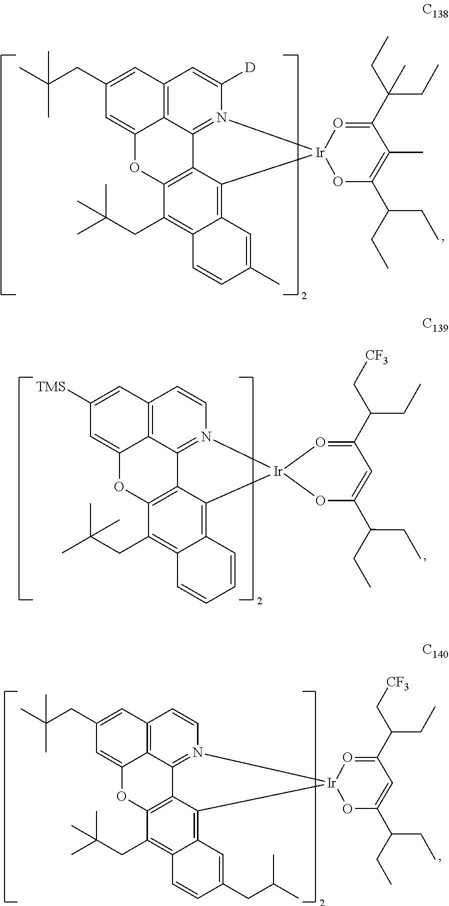

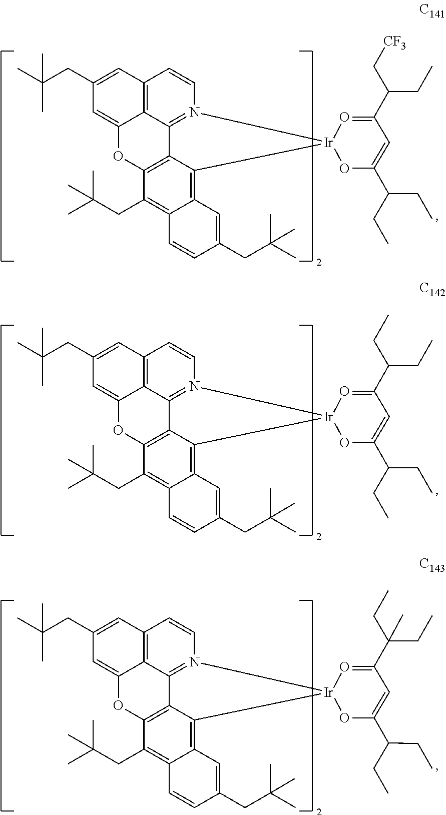

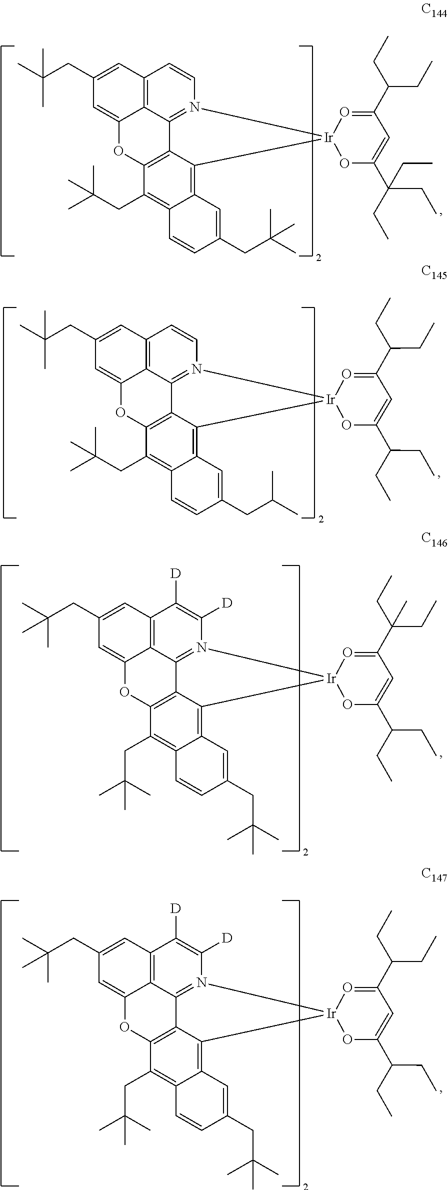

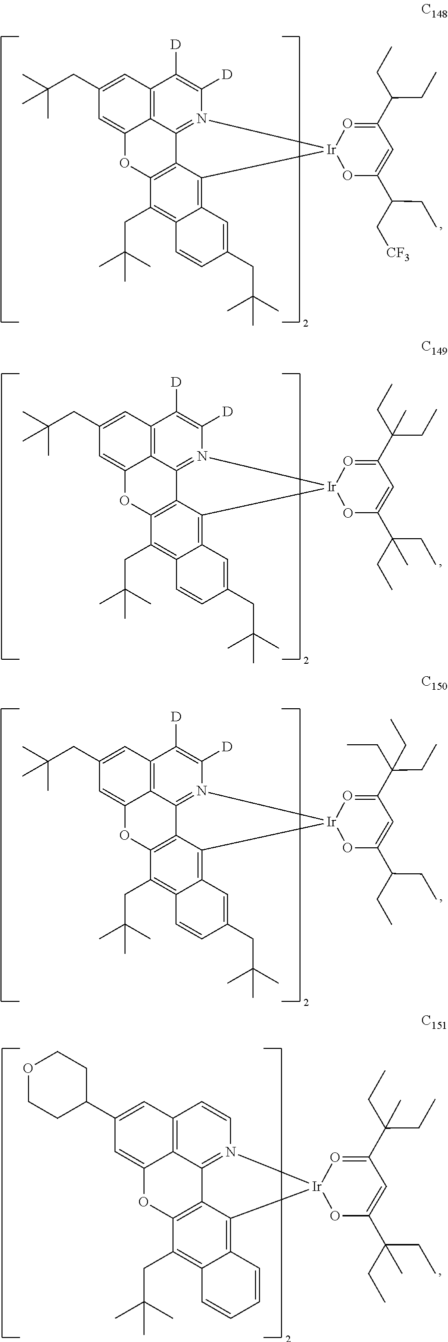

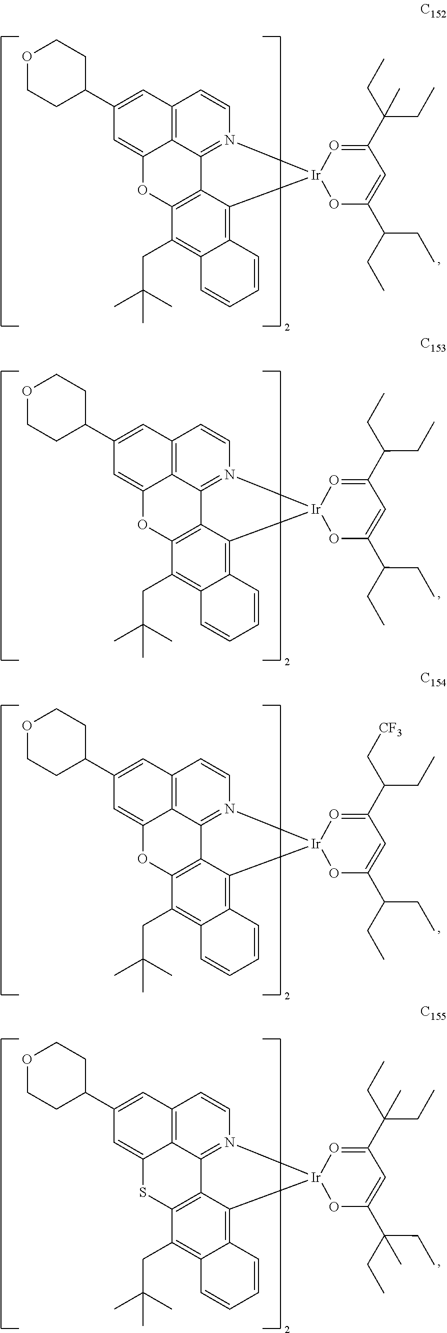

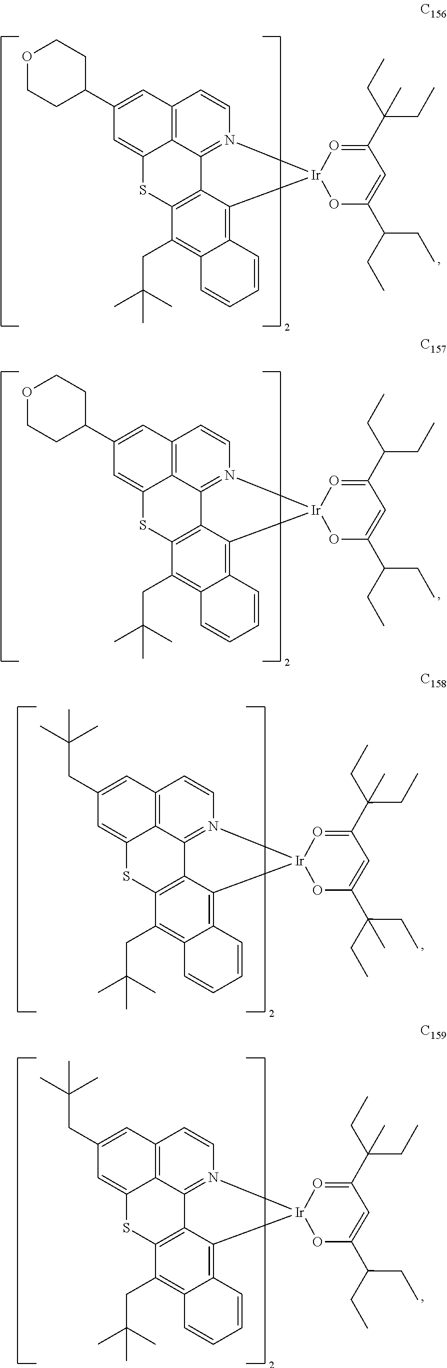

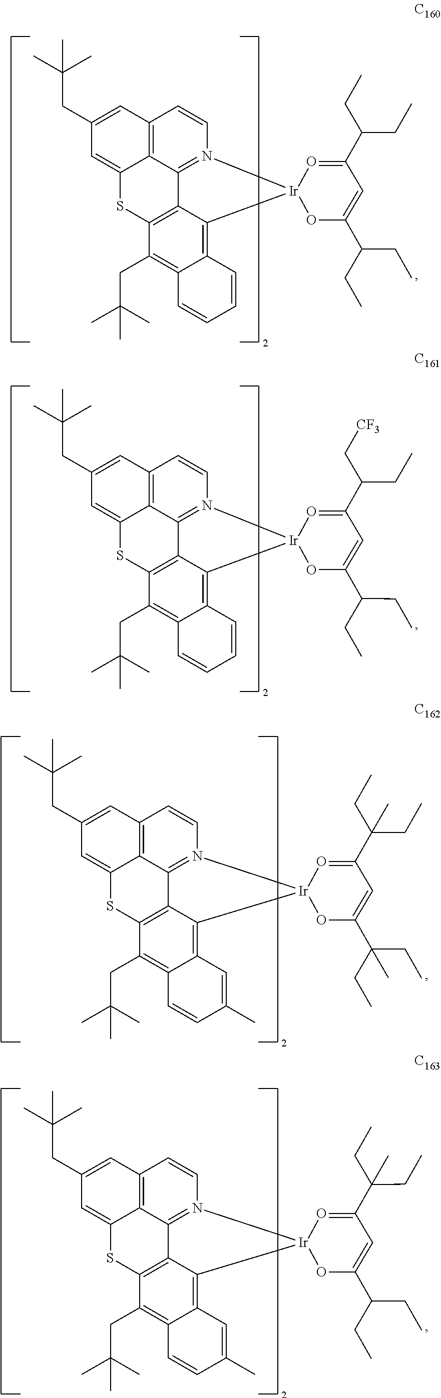

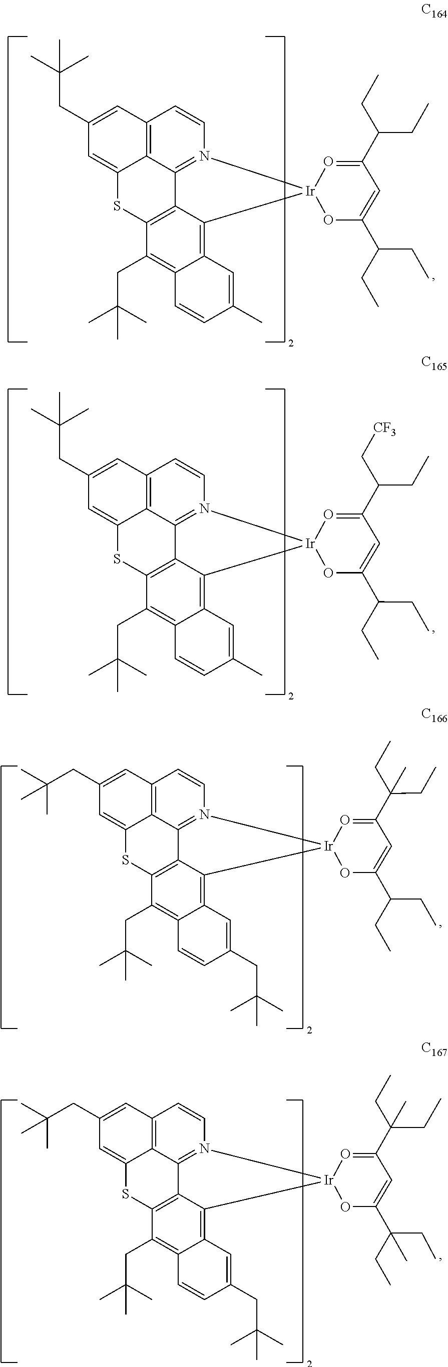

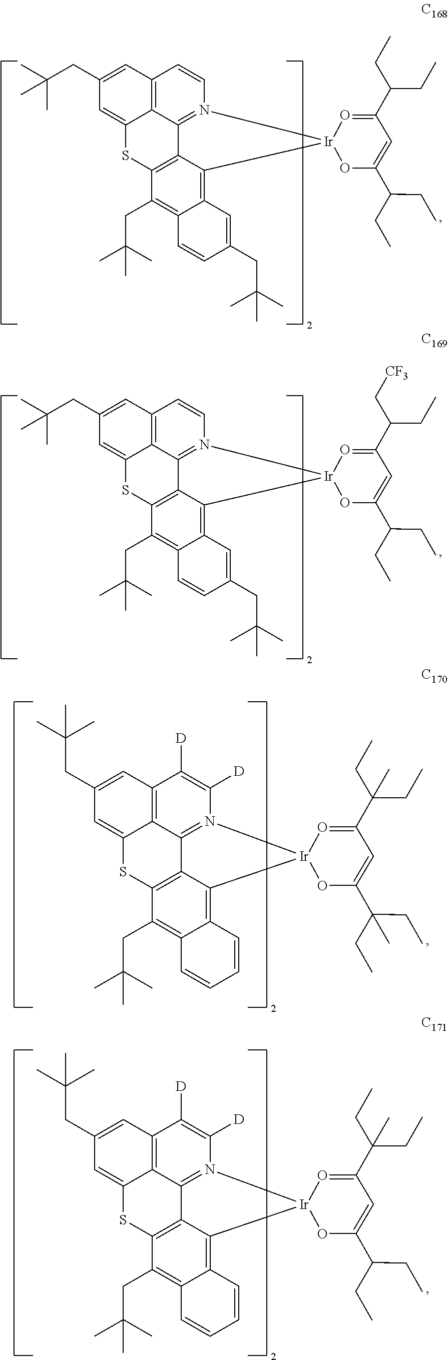

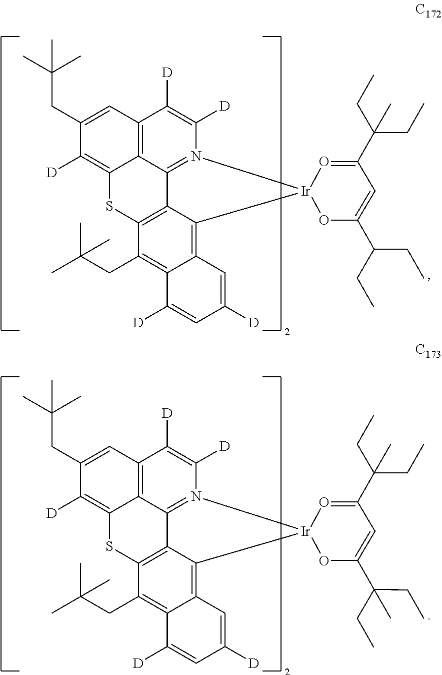

According to an embodiment of the present disclosure, wherein, the organic layer is a light-emitting layer, the first compound is a host material, and the second compound is a light-emitting material.

According to an embodiment of the present disclosure, wherein, the device emits red light or white light.

According to another embodiment of the present disclosure, further disclosed is a display assembly comprising an electroluminescent device, wherein the specific structure of the electroluminescent device is shown in any one of the preceding embodiments.

According to another embodiment of the present disclosure, further disclosed is a compound combination comprising at least a first compound and a second compound;

wherein the first compound has a structure of H-L-E, wherein the H has a structure represented by Formula A:

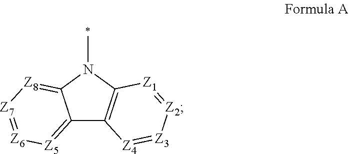

wherein in Formula A,

Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;

L is selected from a single bond, substituted or unsubstituted arylene having 6 to 30 carbon atoms, substituted or unsubstituted heteroarylene having 3 to 30 carbon atoms or a combination thereof;

E is selected from substituted or unsubstituted aryl having 6 to 30 carbon atoms or substituted or unsubstituted heteroaryl having 3 to 30 carbon atoms;

R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;

R_{z2} is, at each occurrence identically or differently, selected from the group consisting of: hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof, and

adjacent substituents R_{z1}, R_{z2} can be optionally joined to form a ring;

wherein the second compound is a metal complex, wherein the metal is selected from a metal with a relative atomic mass greater than 40, and the metal complex comprises a ligand Lₐ which has a structure represented by Formula C:

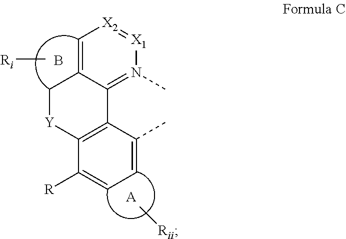

wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;

Rᵢ represents, at each occurrence identically or differently, mono-substitution, multiple substitutions or non-substitution; and Rᵢᵢ represents, at each occurrence identically or differently, mono-substitution, multiple substitutions or non-substitution;

Y is selected from SiR_{y}R_{y}, GeR_{y}R_{y}, NR_{y}, PR_{y}, O, S or Se;

when two R_{y} are present, the two R_{y} may be identical or different;

X₁ and X₂ are, at each occurrence identically or differently, selected from CRₓ or N;

R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and

adjacent substituents Rᵢ, Rₓ, R_{y}, R and Rᵢᵢ can be optionally joined to form a ring.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described in the present disclosure for a particular layer in an organic light emitting device can be used in combination with various other materials present in the device. For example, a combination of the first compound and the second compound disclosed herein may be used in combination with a wide variety of emissive dopants, hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The first compound and the second compound used in the present disclosure may be obtained with reference to preparation methods in the related art or may also be easily prepared with reference to patent applications of Publication Nos. or Application Nos. US20180337340A1, CN111868210A, CN202010285016.7, CN202010268985.1, CN202010285026.0, CN202010720191.4, CN202010825242.X, CN202011219604.7, CN202110348602.6 and so on, which is not repeated here. The method for preparing an electroluminescent device is not limited. The preparation methods in the following examples are merely examples and not to be construed as limitations. Those skilled in the art can make reasonable improvements on the preparation methods in the following examples based on the related art. Exemplarily, the proportions of various materials in a light-emitting layer are not particularly limited. Those skilled in the art can reasonably select the proportions within a certain range based on the related art. For example, taking the total weight of the materials in the light-emitting layer as reference, a host material may account for 80% to 99% and a light-emitting material may account for 1% to 20%; or the host material may account for 90% to 98% and the light-emitting material may account for 2% to 10%. Further, the host material may include one material or two materials, where a ratio of two host materials may be 100:0 to 1:99; or the ratio may be 80:20 to 20:80; or the ratio may be 60:40 to 40:60. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Device Example 1

First, a glass substrate having an indium tin oxide (ITO) anode with a thickness of 120 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second under a vacuum degree of about 10⁻⁸ torr. Compound HI was used as a hole injection layer (HIL) with a thickness of 100 Å. Compound HT was used as a hole transporting layer (HTL) with a thickness of 400 Å. Compound EB was used as an electron blocking layer (EBL) with a thickness of 50 Å. Then, Compound C₈ as a dopant material and Compound 1-34 as a host material were co-deposited (at a weight ratio of 2:98) as an emissive layer (EML) with a thickness of 400 Å. Compound HB was used as a hole blocking layer (HBL) with a thickness of 50 Å. On the HBL, Compound ET and 8-hydroxyquinolinolato-lithium (Liq) were co-deposited as an electron transporting layer (ETL) with a thickness of 350 Å. Finally, Liq was deposited as an electron injection layer with a thickness of 1 nm, and A1 was deposited as a cathode with a thickness of 120 nm. The device was transferred back to the glovebox and encapsulated with a glass lid and a moisture getter to complete the device.

### Device Example 2

The implementation mode in Device Example 2 was the same as that in Device Example 1, except that in the EML, Compound 1-34 was replaced with Compound 1-35 as the host material.

### Device Example 3

The implementation mode in Device Example 3 was the same as that in Device Example 1, except that in the EML, Compound C₈ was replaced with Compound C₂₇ as the dopant material.

### Device Example 4

The implementation mode in Device Example 4 was the same as that in Device Example 3, except that in the EML, Compound 1-34 was replaced with Compound 1-36 as the host material.

### Device Example 5

The implementation mode in Device Example 5 was the same as that in Device Example 3, except that in the EML, Compound 1-34 was replaced with Compound 1-35 as the host material.

### Device Example 6

The implementation mode in Device Example 6 was the same as that in Device Example 1, except that in the EML, Compound C₈ was replaced with Compound C₅₆ as the dopant material, and in the EML, the doping weight ratio of the dopant material to the host material was adjusted to 3:97.

### Device Example 7

The implementation mode in Device Example 7 was the same as that in Device Example 6, except that in the EML, Compound C₅₆ was replaced with Compound C₅₈ as the dopant material.

### Device Example 8

The implementation mode in Device Example 8 was the same as that in Device Example 6, except that in the EML, Compound C₅₆ was replaced with Compound C₈₇ as the dopant material.

### Device Example 9

The implementation mode in Device Example 9 was the same as that in Device Example 6, except that in the EML, Compound C₅₆ was replaced with Compound C₁₃₉ as the dopant material.

### Device Example 10

The implementation mode in Device Example 10 was the same as that in Device Example 6, except that in the EML, Compound C₅₆ was replaced with Compound C₁₄₀ as the dopant material.

### Device Example 11

The implementation mode in Device Example 11 was the same as that in Device Example 6, except that in the EML, Compound C₅₆ was replaced with Compound C₁₄₁ as the dopant material.

### Device Example 12

The implementation mode in Device Example 12 was the same as that in Device Example 6, except that in the EML, Compound C₅₆ was replaced with Compound C₁₄₄ as the dopant material.

### Device Example 13

The implementation mode in Device Example 13 was the same as that in Device Example 6, except that in the EML, Compound C₅₆ was replaced with Compound C₁₄₃ as the dopant material.

### Device Example 14

The implementation mode in Device Example 14 was the same as that in Device Example 6, except that in the EML, Compound C₅₆ was replaced with Compound C₁₄₆ as the dopant material.

### Device Example 15

The implementation mode in Device Example 15 was the same as that in Device Example 6, except that in the EML, Compound C₅₆ was replaced with Compound C₁₅₀ as the dopant material.

### Device Example 16

The implementation mode in Device Example 16 was the same as that in Device Example 1, except that in the EML, Compound C₈ was replaced with Compound C₁₅₈ as the dopant material.

### Device Example 17

The implementation mode in Device Example 17 was the same as that in Device Example 6, except that in the EML, Compound C₅₆ was replaced with Compound C₁₆₃ as the dopant material.

### Device Example 18

The implementation mode in Device Example 18 was the same as that in Device Example 6, except that in the EML, Compound C₅₆ was replaced with Compound C₁₆₆ as the dopant material.

### Device Example 19

The implementation mode in Device Example 19 was the same as that in Device Example 6, except that in the EML, Compound C₅₆ was replaced with Compound C₁₆₇ as the dopant material.

### Device Example 20

The implementation mode in Device Example 20 was the same as that in Device Example 6, except that in the EML, Compound C₅₆ was replaced with Compound C₁₇₃ as the dopant material.

### Device Comparative Example 1

The implementation mode in Device Comparative Example 1 was the same as that in Device Example 1, except that in the EML, Compound 1-34 was replaced with Compound H1 as the host material, and Compound C₈ was replaced with Compound RD-A as the dopant material.

### Device Comparative Example 2

The implementation mode in Device Comparative Example 2 was the same as that in Device Comparative Example 1, except that in the EML, Compound H1 was replaced with Compound 1-34 as the host material.

### Device Comparative Example 3

The implementation mode in Device Comparative Example 3 was the same as that in Device Comparative Example 1, except that in the EML, Compound H1 was replaced with Compound 1-36 as the host material.

### Device Comparative Example 4

The implementation mode in Device Comparative Example 4 was the same as that in Device Comparative Example 1, except that in the EML, Compound H1 was replaced with Compound 1-35 as the host material.

### Device Comparative Example 5

The implementation mode in Device Comparative Example 5 was the same as that in Device Example 1, except that in the EML, Compound 1-34 was replaced with Compound H1 as the host material.

### Device Comparative Example 6

The implementation mode in Device Comparative Example 6 was the same as that in Device Example 3, except that in the EML, Compound 1-34 was replaced with Compound H1 as the host material.

### Device Comparative Example 7

The implementation mode in Device Comparative Example 7 was the same as that in Device Example 6, except that in the EML, Compound 1-34 was replaced with Compound H1 as the host material.

### Device Comparative Example 8

The implementation mode in Device Comparative Example 8 was the same as that in Device Example 7, except that in the EML, Compound 1-34 was replaced with Compound H1 as the host material.

### Device Comparative Example 9

The implementation mode in Device Comparative Example 9 was the same as that in Device Example 8, except that in the EML, Compound 1-34 was replaced with Compound H1 as the host material.

### Device Comparative Example 10

The implementation mode in Device Comparative Example 10 was the same as that in Device Example 9, except that in the EML, Compound 1-34 was replaced with Compound H1 as the host material.

### Device Comparative Example 11

The implementation mode in Device Comparative Example 11 was the same as that in Device Example 10, except that in the EML, Compound 1-34 was replaced with Compound H1 as the host material.

### Device Comparative Example 12

The implementation mode in Device Comparative Example 12 was the same as that in Device Example 11, except that in the EML, Compound 1-34 was replaced with Compound H1 as the host material.

### Device Comparative Example 13

The implementation mode in Device Comparative Example 13 was the same as that in Device Example 12, except that in the EML, Compound 1-34 was replaced with Compound H1 as the host material.

The structures and thicknesses of layers of the devices are shown in the following table. A layer using more than one material is obtained by doping different compounds at their weight ratio as recorded.

TABLE 1
Device structures in device examples and device comparative examples

| Device ID | HIL | HTL | EBL | EML | HBL | ETL |
|---|---|---|---|---|---|---|
| Comparative | Compound | Compound | Compound | Compound | Compound | Compound |
| Example 1 | HI | HT | EB | H1:Compound | HB | ET:Liq (40:60) |
| | (100 Å) | (400 Å) | (50 Å) | RD-A (98:2) | (50 Å) | (350 Å) |
| | | | | (400 Å) | | |
| Comparative | Compound | Compound | Compound | Compound | Compound | Compound |
| Example 2 | HI | HT | EB | l-34:Compound | HB | ET:Liq (40:60) |
| | (100 Å) | (400 Å) | (50 Å) | RD-A (98:2) | (50 Å) | (350 Å) |
| | | | | (400 Å) | | |
| Comparative | Compound | Compound | Compound | Compound | Compound | Compound |
| Example 3 | HI | HT | EB | l-36:Compound | HB | ET:Liq (40:60) |
| | (100 Å) | (400 Å) | (50 Å) | RD-A (98:2) | (50 Å) | (350 Å) |
| | | | | (400 Å) | | |
| Comparative | Compound | Compound | Compound | Compound | Compound | Compound |
| Example 4 | HI | HT | EB | 1-35:Compound | HB | ET:Liq (40:60) |
| | (100 Å) | (400 Å) | (50 Å) | RD-A (98:2) | (50 Å) | (350 Å) |
| | | | | (400 Å) | | |
| Comparative | Compound | Compound | Compound | Compound | Compound | Compound |
| Example 5 | HI | HT | EB | H1:Compound C₈ | HB | ET:Liq (40:60) |
| | (100 Å) | (400 Å) | (50 Å) | (98:2) | (50 Å) | (350 Å) |
| | | | | (400 Å) | | |
| Comparative | Compound | Compound | Compound | Compound | Compound | Compound |
| Example 6 | HI | HT | EB | H1:Compound C₂₇ | HB | ET:Liq (40:60) |
| | (100 Å) | (400 Å) | (50 Å) | (98:2) | (50 Å) | (350 Å) |
| | | | | (400 Å) | | |
| Comparative | Compound | Compound | Compound | Compound | Compound | Compound |
| Example 7 | HI | HT | EB | H1:Compound C₅₆ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 8 | HI | HT | EB | H1:Compound C₅₈ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 9 | HI | HT | EB | H1:Compound C₈₇ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 10 | HI | HT | EB | H1:Compound C₁₃₉ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 11 | HI | HT | EB | H1:Compound C₁₄₀ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 12 | HI | HT | EB | H1:Compound C₁₄₁ | HB | ET:Liq (40:60) |
| | (100 Å) | (400 Å) | (50 Å) | (97:3) | (50 Å) | (350 Å) |
| | | | | (400 Å) | | |
| Comparative | Compound | Compound | Compound | Compound | Compound | Compound |
| Example 13 | HI | HT | EB | H1:Compound C₁₄₄ | HB | ET:Liq (40:60) |
| | (100 Å) | (400 Å) | (50 Å) | (97:3) | (50 Å) | (350 Å) |
| | | | | (400 Å) | | |
| Example 1 | Compound | Compound | Compound | Compound | Compound | Compound |
| | HI | HT | EB | 1-34:Compound C₈ | HB | ET:Liq (40:60) |
| | (100 Å) | (400 Å) | (50 Å) | (98:2) | (50 Å) | (350 Å) |
| | | | | (400 Å) | | |
| Example 2 | Compound | Compound | Compound | Compound | Compound | Compound |
| | HI | HT | EB | 1-35:Compound C₈ | HB | ET:Liq (40:60) |
| | (100 Å) | (400 Å) | (50 Å) | (98:2) | (50 Å) | (350 Å) |
| | | | | (400 Å) | | |
| Example 3 | Compound | Compound | Compound | Compound | Compound | Compound |
| | HI | HT | EB | 1-34:Compound C₂₇ | HB | ET:Liq (40:60) |
| | (100 Å) | (400 Å) | (50 Å) | (98:2) | (50 Å) | (350 Å) |
| | | | | (400 Å) | | |
| Example 4 | Compound | Compound | Compound | Compound | Compound | Compound |
| | HI | HT | EB | 1-36:Compound C₂₇ | HB | ET:Liq (40:60) |
| | (100 Å) | (400 Å) | (50 Å) | (98:2) | (50 Å) | (350 Å) |
| | | | | (400 Å) | | |
| Example 5 | Compound | Compound | Compound | Compound | Compound | Compound |
| | HI | HT | EB | 1-35:Compound C₂₇ | HB | ET:Liq (40:60) |
| | (100 Å) | (400 Å) | (50 Å) | (98:2) | (50 Å) | (350 Å) |
| | | | | (400 Å) | | |
| Example 6 | Compound | Compound | Compound | Compound | Compound | Compound |
| | HI | HT | EB | 1-34:Compound C₅₆ | HB | ET:Liq (40:60) |
| | (100 Å) | (400 Å) | (50 Å) | (97:3) | (50 Å) | (350 Å) |
| | | | | (400 Å) | | |
| Example 7 | Compound | Compound | Compound | Compound | Compound | Compound |
| | HI | HT | EB | 1-34:Compound C₅₈ | HB | ET:Liq (40:60) |
| | (100 Å) | (400 Å) | (50 Å) | (97:3) | (50 Å) | (350 Å) |
| | | | | (400 Å) | | |
| Example 8 | Compound | Compound | Compound | Compound | Compound | Compound |
| | HI | HT | EB | 1-34:Compound C₈₇ | HB | ET:Liq (40:60) |
| | (100 Å) | (400 Å) | (50 Å) | (97:3) | (50 Å) | (350 Å) |
| | | | | (400 Å) | | |
| Example 9 | Compound | Compound | Compound | Compound | Compound | Compound |
| | HI | HT | EB | l-34:Compound C₁₃₉ | HB | ET:Liq (40:60) |
| | (100 Å) | (400 Å) | (50 Å) | (97:3) | (50 Å) | (350 Å) |
| | | | | (400 Å) | | |
| Example 10 | Compound | Compound | Compound | Compound | Compound | Compound |
| | HI | HT | EB | l-34:Compound C₁₄₀ | HB | ET:Liq (40:60) |
| | (100 Å) | (400 Å) | (50 Å) | (97:3) | (50 Å) | (350 Å) |
| | | | | (400 Å) | | |
| Example 11 | Compound | Compound | Compound | Compound | Compound | Compound |
| | HI | HT | EB | l-34:Compound C₁₄₁ | HB | ET:Liq (40:60) |
| | (100 Å) | (400 Å) | (50 Å) | (97:3) | (50 Å) | (350 Å) |
| | | | | (400 Å) | | |
| Example 12 | Compound | Compound | Compound | Compound | Compound | Compound |
| | HI | HT | EB | l-34:Compound C₁₄₄ | HB | ET:Liq (40:60) |
| | (100 Å) | (400 Å) | (50 Å) | (97:3) | (50 Å) | (350 Å) |
| | | | | (400 Å) | | |
| Example 13 | Compound | Compound | Compound | Compound | Compound | Compound |
| | HI | HT | EB | l-34:Compound C₁₄₃ | HB | ET:Liq (40:60) |
| | (100 Å) | (400 Å) | (50 Å) | (97:3) | (50 Å) | (350 Å) |
| | | | | (400 Å) | | |
| Example 14 | Compound | Compound | Compound | Compound | Compound | Compound |
| | HI | HT | EB | l-34:Compound C₁₄₆ | HB | ET:Liq (40:60) |
| | (100 Å) | (400 Å) | (50 Å) | (97:3) | (50 Å) | (350 Å) |
| | | | | (400 Å) | | |
| Example 15 | Compound | Compound | Compound | Compound | Compound | Compound |
| | HI | HT | EB | l-34:Compound C₁₅₀ | HB | ET:Liq (40:60) |
| | (100 Å) | (400 Å) | (50 Å) | (97:3) | (50 Å) | (350 Å) |
| | | | | (400 Å) | | |
| Example 16 | Compound | Compound | Compound | Compound | Compound | Compound |
| | HI | HT | EB | l-34:Compound C₁₅₈ | HB | ET:Liq (40:60) |
| | (100 Å) | (400 Å) | (50 Å) | (98:2) | (50 Å) | (350 Å) |
| | | | | (400 Å) | | |
| Example 17 | Compound | Compound | Compound | Compound | Compound | Compound |
| | HI | HT | EB | l-34:Compound C₁₆₃ | HB | ET:Liq (40:60) |
| | (100 Å) | (400 Å) | (50 Å) | (97:3) | (50 Å) | (350 Å) |
| | | | | (400 Å) | | |
| Example 18 | Compound | Compound | Compound | Compound | Compound | Compound |
| | HI | HT | EB | l-34:Compound C₁₆₆ | HB | ET:Liq (40:60) |
| | (100 Å) | (400 Å) | (50 Å) | (97:3) | (50 Å) | (350 Å) |
| | | | | (400 Å) | | |
| Example 19 | Compound | Compound | Compound | Compound | Compound | Compound |
| | HI | HT | EB | l-34:Compound C₁₆₇ | HB | ET:Liq (40:60) |
| | (100 Å) | (400 Å) | (50 Å) | (97:3) | (50 Å) | (350 Å) |
| | | | | (400 Å) | | |
| Example 20 | Compound | Compound | Compound | Compound | Compound | Compound |
| | HI | HT | EB | l-34:Compound C₁₇₃ | HB | ET:Liq (40:60) |
| | (100 Å) | (400 Å) | (50 Å) | (97:3) | (50 Å) | (350 Å) |
| | | | | (400 Å) | | |

The structures of the materials used in the devices are shown as follows:

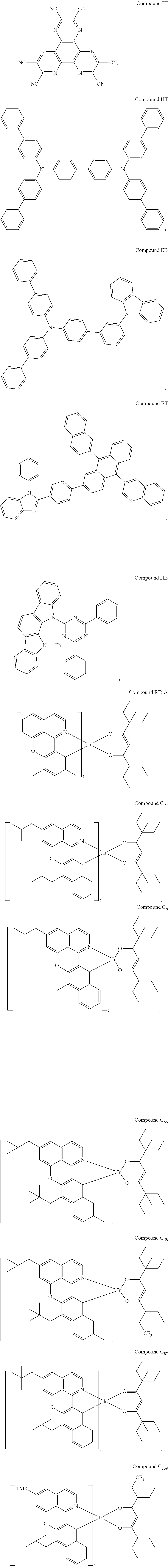

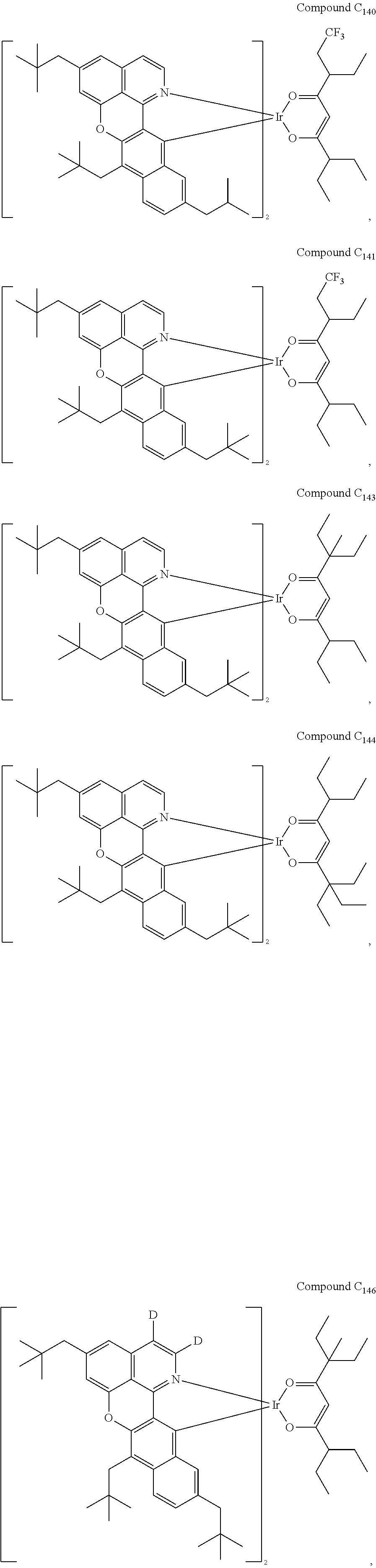

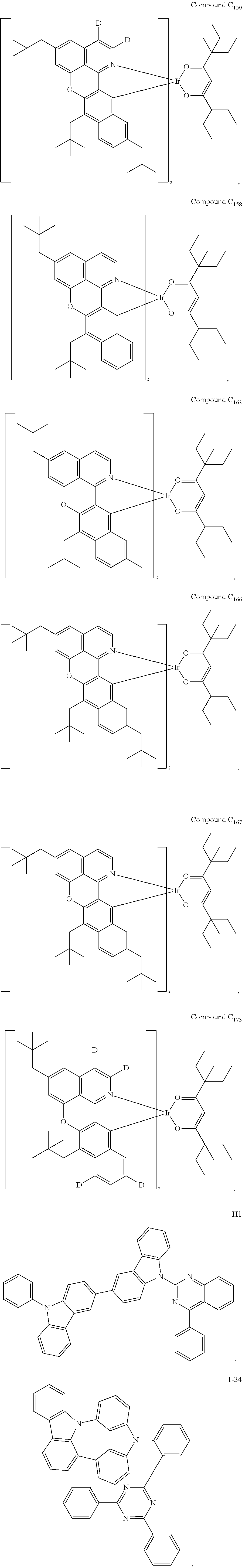

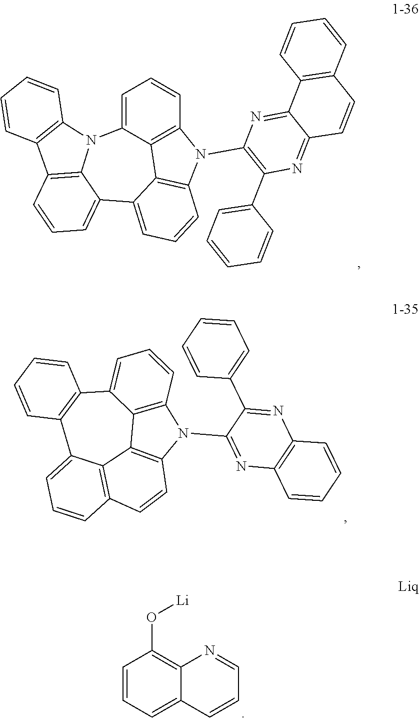

Current-voltage-luminance (IVL) characteristics and lifetime characteristics of the devices were measured. Table 2 shows external quantum efficiency (EQE) data measured at a current density of 15 mA/cm² and lifetime (LT97) data measured at initial brightness of 5000 cd/m²

TABLE 2
Device data

| Device ID | EQE (%) | LT97 (h) |
|---|---|---|
| Example 1 | 24.37 | 1234 |
| Example 2 | 24.10 | 795 |
| Example 3 | 25.50 | 1323 |
| Example 4 | 25.21 | 689 |
| Example 5 | 25.31 | 942 |
| Example 6 | 25.08 | 1930 |
| Example 7 | 24.57 | 2492 |
| Example 8 | 25.94 | 1648 |
| Example 9 | 23.53 | 821 |
| Example 10 | 24.24 | 3184 |
| Example 11 | 24.06 | 1071 |
| Example 12 | 25.58 | 2332 |
| Example 13 | 24.24 | 2016 |
| Example 14 | 24.58 | 2504 |
| Example 15 | 24.16 | 2107 |
| Example 16 | 24.97 | 1169 |
| Example 17 | 24.57 | 1803 |
| Example 18 | 24.58 | 805 |
| Example 19 | 24.43 | 774 |
| Example 20 | 26.10 | 1603 |
| Comparative | 21.30 | 246 |
| Example 1 | | |
| Comparative | 5.64 | 4 |
| Example 2 | | |
| Comparative | 4.35 | 1 |
| Example 3 | | |
| Comparative | 4.76 | 2 |
| Example 4 | | |
| Comparative | 22.45 | 702 |
| Example 5 | | |
| Comparative | 22.68 | 665 |
| Example 6 | | |
| Comparative | 23.63 | 1242 |
| Example 7 | | |
| Comparative | 23.06 | 1199 |
| Example 8 | | |
| Comparative | 21.94 | 505 |
| Example 9 | | |
| Comparative | 21.25 | 225 |
| Example 10 | | |
| Comparative | 22.25 | 435 |
| Example 11 | | |
| Comparative | 21.70 | 367 |
| Example 12 | | |
| Comparative | 22.95 | 792 |
| Example 13 | | |

From the data comparison between Examples 1 and 2 and Comparative Example 5, it can be seen that a combination of the first compound and the second compound of the present disclosure can further improve the EQE of the device from 22.45% in Comparative Example 5, which has already reached a very high level, to 24.10% and 24.37%, respectively, which are significantly improved by 7.3% and 8.5%, respectively; more importantly, the compound combination of the present disclosure significantly extends the device lifetime from 702 hours in Comparative Example 5 to 1234 hours and 795 hours, respectively. It proves that the combination of the first compound and the second compound of the present disclosure has excellent performance that can significantly improve the EQE of the device and significantly extend the device lifetime. From the data comparison between Examples 3 to 5 and Comparative Example 6, it can be seen that the combination of the first compound and the second compound of the present disclosure can further improve the EQE of the device from 22.68% in Comparative Example 6, which has already reached a very high level, to 25.50%, 25.21% and 25.31%, respectively, which are significantly improved by 12.4%, 9.8% and 11.6%, respectively; more importantly, the compound combination of the present disclosure significantly extends the lifetime from 665 hours in Comparative Example 6 to 1323 hours, 689 hours and 942 hours in Examples 3 to 5, respectively. It proves that the combination of the first compound and the second compound of the present disclosure has the excellent performance that can significantly improve the EQE of the device and significantly extend the device lifetime. Similarly, from the data comparison between Example 6 and Comparative Example 7, the comparison between Example 7 and Comparative Example 8, the comparison between Example 8 and Comparative Example 9, the comparison between Example 9 and Comparative Example 10, the comparison between Example 10 and Comparative Example 11, the comparison between Example 11 and Comparative Example 12 and the comparison between Example 12 and Comparative Example 13, it again proves that the combination of the first compound and the second compound of the present disclosure can significantly improve the EQE of the device and significantly extend the device lifetime. Moreover, the comparison of these nine sets of data also proves that the combination of the first compound and the second compound of the present disclosure generally has excellent performance on improving device performance.

These results show that the combination of the first compound and the second compound of the present disclosure can significantly improve the device performance and achieves a far better device effect than a commercially available host material, and these results fully prove the superiority of the combination of the first compound and the second compound of the present disclosure.

Compared to Comparative Example 2, the device performance in Examples 1 and 3 has obvious advantages: compared to that in Comparative Example 2, the EQE in Examples 1 and 3 is improved by 332.1% and 352.1%, respectively, and compared to that in Comparative Example 2, the lifetimes are improved by several hundred times, reaching more than 1200 hours, which achieve very significant improvements. From the comparison between Examples 13 to 20 and Comparative Example 2, it can also be seen that the device performance in the examples has obvious advantages. Compared to that in Comparative Example 2, the device efficiency in Examples 13 to 20 is generally improved by several times, the device lifetimes in Examples 13 to 20 are generally improved by several hundred times, Example 20, in particular, has ultra-high device efficiency of 26.10% in the case of a long lifetime of 1603 h, indicating that the combination of the first compound and the second compound selected specifically in the present disclosure can significantly improve the device performance. Again, these results prove the superiority of the combination of the first compound and the second compound of the present disclosure.

From the comparison between Comparative Example 1 and Comparative Examples 2 to 4, it can be found that when the same dopant Compound RD-A is used, the device performance in Comparative Examples 2 to 4 in which the first compound selected in the present disclosure is used as a host is significantly reduced compared to that in Comparative Example 1 in which the commercially available host material is used. According to the comparison between Examples 1 to 5 and Comparative Examples 5 and 6, it can be found that when the second compound selected in the present disclosure is used as the dopant material, the device performance in Examples 1 to 5 is significantly improved compared to that in Comparative Examples 5 and 6 in which the commercially available host material is used. From the above comparison, it can be found that although Compound RD-A and the second compound selected in the present disclosure are similar in structure, the combination of the second compound selected in the present disclosure and the first compound selected in the present disclosure unexpectedly and significantly improves the device performance. The completely different variation in device performance exhibited when such similar compound structures are combined with different compounds further shows the unpredictable superiority of the combination of the first compound and the second compound of the present disclosure.

To conclude, the combination of the first compound and the second compound selected in the present disclosure can exhibit excellent device performance in the device, obtain higher EQE, and significantly extend the device lifetime. It proves that the combination of the first compound and the second compound selected in the present disclosure has an excellent application prospect.

It is to be understood that various embodiments described herein are merely illustrative and not intended to limit the scope of the present disclosure. Therefore, it is apparent to the persons skilled in the art that the present disclosure as claimed may include variations of specific embodiments and preferred embodiments described herein. Many of the materials and structures described herein may be replaced with other materials and structures without departing from the spirit of the present disclosure. It is to be understood that various theories as to why the present disclosure works are not intended to be limiting.

What is claimed is:

1. An electroluminescent device, comprising:
an anode,
a cathode, and
an organic layer disposed between the anode and the cathode, wherein the organic layer contains at least a first compound and a second compound;
wherein the first compound has a structure of H-L-E, wherein the H has a structure represented by Formula A: 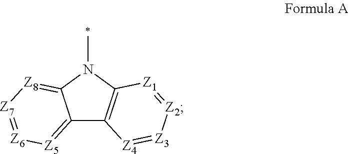
wherein in Formula A,
Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;
L is selected from a single bond, substituted or unsubstituted arylene having 6 to 30 carbon atoms, substituted or unsubstituted heteroarylene having 3 to 30 carbon atoms or a combination thereof,
E is selected from substituted or unsubstituted aryl having 6 to 30 carbon atoms or substituted or unsubstituted heteroaryl having 3 to 30 carbon atoms;
R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof,
R_{z2} is, at each occurrence identically or differently, selected from the group consisting of: hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof, and
adjacent substituents R_{z1}, R_{z2} can be optionally joined to form a ring;
wherein the second compound is a metal complex, wherein the metal is selected from a metal with a relative atomic mass greater than 40, and the metal complex comprises a ligand Lₐ which has a structure represented by Formula C: 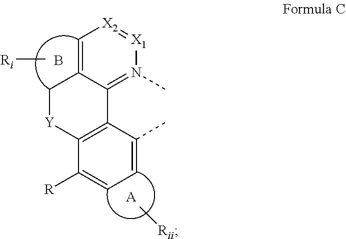
wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;
Rᵢ represents, at each occurrence identically or differently, mono-substitution, multiple substitutions or non-substitution; and Rᵢᵢ represents, at each occurrence identically or differently, mono-substitution, multiple substitutions or non-substitution;
Y is selected from SiR_{y}R_{y}, GeR_{y}R_{y}, NR_{y}, PR_{y}, O, S or Se;
when two R_{y} are present, the two R_{y} may be identical or different;
X₁ and X₂ are, at each occurrence identically or differently, selected from CRₓ or N;
R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and
adjacent substituents Rᵢ, Rₓ, R_{y}, R and Rᵢᵢ can be optionally joined to form a ring.

2. The electroluminescent device according to claim 1, wherein in Formula A, the two substituents R_{z2} in Z₄ and Z₅ are joined to form a ring, and the ring has at least six ring atoms;
preferably, the two substituents R_{z2} in Z₄ and Z₅ are joined to form a ring, and the ring has at least seven ring atoms.

3. The electroluminescent device according to claim 1, wherein in the first compound, the H has a structure represented by any one of Formula A-1 to Formula A-8: 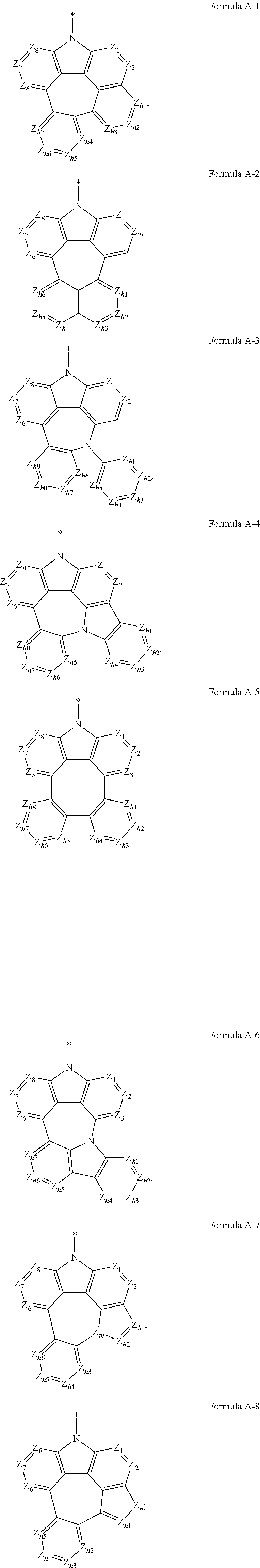
wherein in Formula A-1 to Formula A-8,
Z₁ to Z₃ and Z₆ to Z₈ are, at each occurrence identically or differently, selected from CR_{z1} or N;
Zₕ₁ to Zₕ₈ are, at each occurrence identically or differently, selected from CR_{zh} or N;
Zₘ is selected from CR_{zm} or N;
Zₙ is selected from CR_{zn}R_{zn}, O, S or NR_{zn}; wherein when Zₙ is selected from CR_{zn}R_{zn}, two R_{zn} may be identical or different;
R_{z1}, R_{zh}, R_{zm} and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and
adjacent substituents R_{z1}, R_{zh}, R_{zm}, R_{zn} can be optionally joined to form a ring.

4. The electroluminescent device according to claim 3, wherein in Formula A-1 to Formula A-8, R_{z1}, R_{zh}, R_{zm} and R_{zn} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted amino having 0 to 20 carbon atoms, cyano, isocyano, hydroxyl, a sulfanyl group and combinations thereof, and
adjacent substituents R_{z1}, R_{zh}h, R_{zm}, R_{zn} can be optionally joined to form a ring.

5. The electroluminescent device according to claim 1, wherein the H is selected from the group consisting of the following structures: 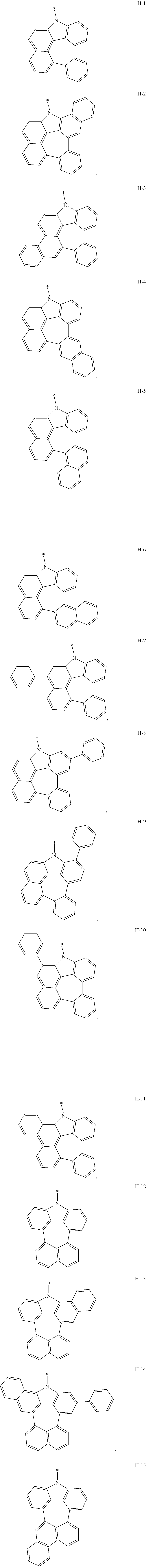 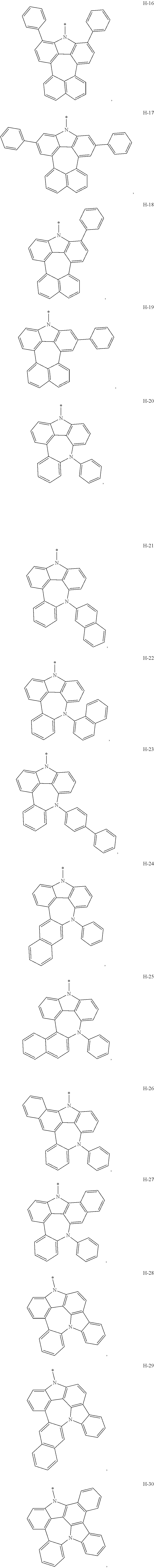 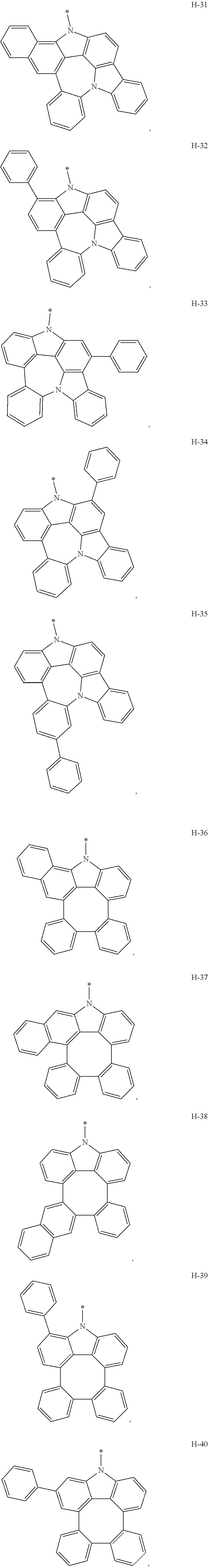 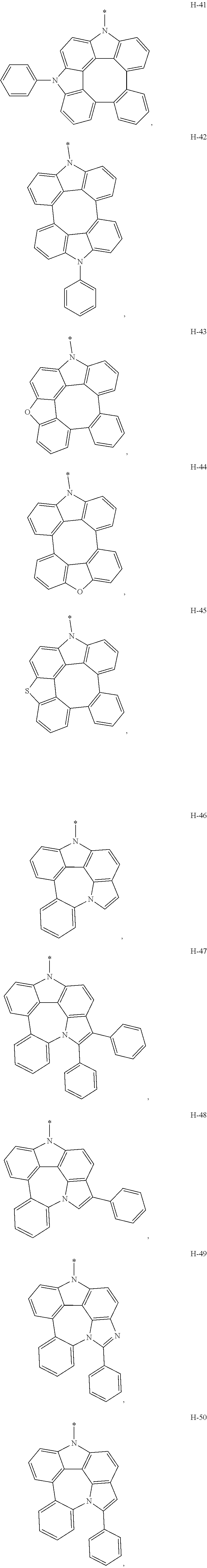 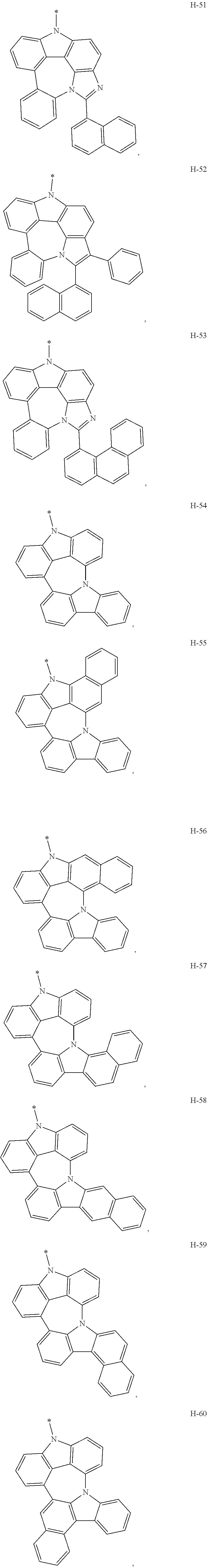 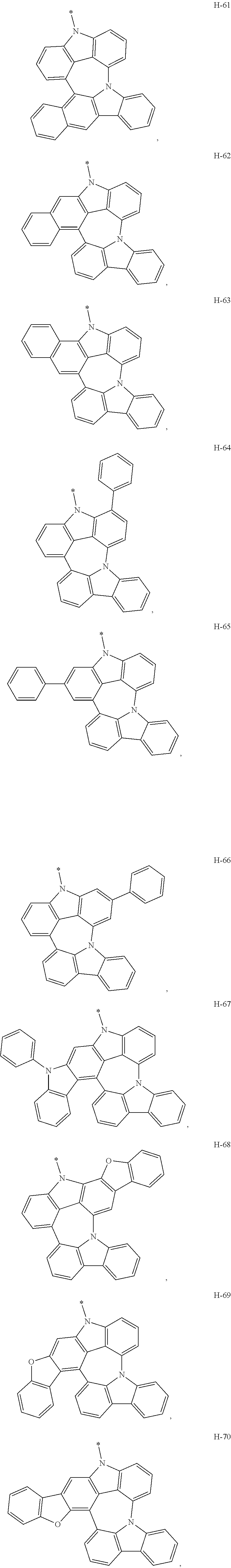 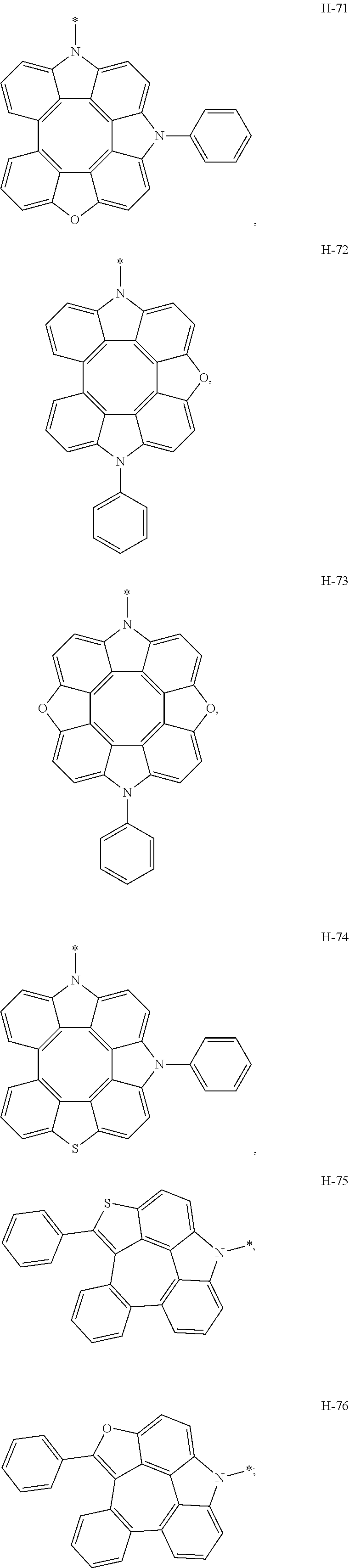
wherein optionally, hydrogen in the structures of H-1 to H-76 can be partially or fully substituted with deuterium.

6. The electroluminescent device according to claim 1, wherein in the first compound, the E has a structure represented by Formula E-a or Formula E-b: 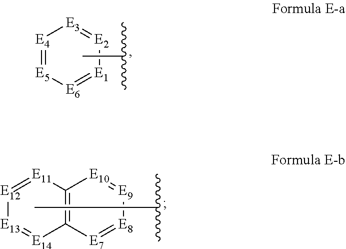
wherein in Formula E-a and Formula E-b,
E₁ to E₁₄ are, at each occurrence identically or differently, selected from C, CRₑ or N;
Rₑ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof,
adjacent substituents Rₑ can be optionally joined to form a ring;
preferably, in Formula E-a, at least two of E₁ to E₆ are N; in Formula E-b, at least two of E₇ to E₁₄ are N;
more preferably, in Formula E-a, three of E₁ to E₆ are N; in Formula E-b, two of E₇ to E₁₀ are N.

7. The electroluminescent device according to claim 1, wherein in the first compound, the E has a structure represented by any one of Formula E-1 to Formula E-10: 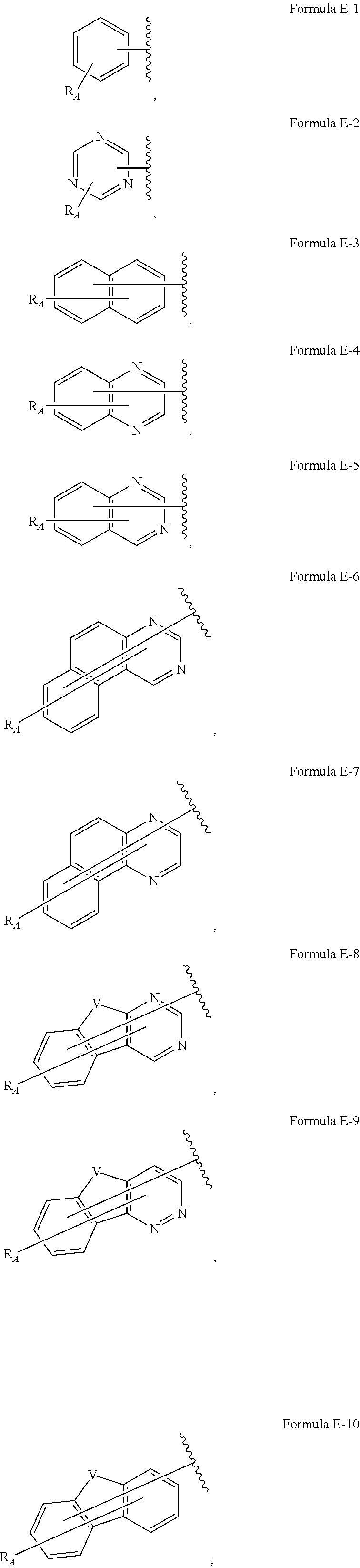
wherein in Formula E-1 to Formula E-10,
R_{A} represents, at each occurrence identically or differently, mono-substitution, multiple substitutions or non-substitution;
V is selected from O, S or Se;
R_{A} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and
adjacent substituents R_{A} can be optionally joined to form a ring;
preferably, the E has a structure represented by any one of Formula E-11 to Formula E-21: 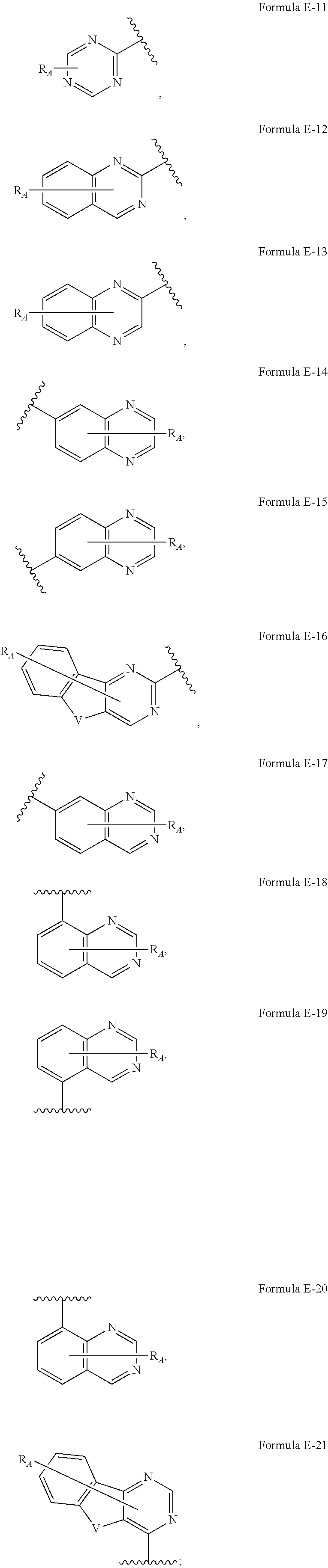
wherein R_{A} represents, at each occurrence identically or differently, mono-substitution, multiple substitutions or non-substitution;
V is selected from O, S or Se; and
R_{A} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof.

8. The electroluminescent device according to claim 7, wherein in Formula E-1 to Formula E-21, R_{A} is, at each occurrence identically or differently, selected from the group consisting of: hydrogen, deuterium, halogen, cyano, hydroxyl, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof,
adjacent substituents R_{A} can be optionally joined to form a ring;
preferably, R_{A} is, at each occurrence identically or differently, selected from the group consisting of: hydrogen, deuterium, fluorine, cyano, hydroxyl, a sulfanyl group, methyl, trideuteriomethyl, vinyl, phenyl, biphenyl, naphthyl, 4-cyanophenyl, dibenzofuranyl, dibenzothienyl, triphenylene, carbazolyl, 9-phenylcarbazolyl, 9,9-dimethylfluorenyl, pyridyl, phenylpyridyl and combinations thereof.

9. The electroluminescent device according to claim 7, wherein in Formula E-1 to Formula E-21, at least one R_{A} is present, and the at least one R_{A} is, at each occurrence identically or differently, selected from the group consisting of: deuterium, halogen, cyano, hydroxyl, a sulfanyl group, substituted or unsubstituted alkyl having 1 to 20 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms and combinations thereof,
adjacent substituents R_{A} can be optionally joined to form a ring;
preferably, the at least one R_{A} is, at each occurrence identically or differently, selected from the group consisting of: deuterium, halogen, cyano, substituted or unsubstituted alkyl having 1 to 20 carbon atoms, substituted or unsubstituted aryl having 6 to 30 carbon atoms, substituted or unsubstituted heteroaryl having 3 to 30 carbon atoms and combinations thereof,
more preferably, the at least one R_{A} is, at each occurrence identically or differently, selected from the group consisting of: deuterium, fluorine, cyano, methyl, trideuteriomethyl, phenyl, biphenyl, naphthyl, 4-cyanophenyl, dibenzofuranyl, dibenzothienyl, triphenylene, carbazolyl, 9-phenylcarbazolyl, 9,9-dimethylfluorenyl, pyridyl, phenylpyridyl and combinations thereof.

10. The electroluminescent device according to claim 1, wherein in the first compound, the E is selected from the group consisting of the following structures: 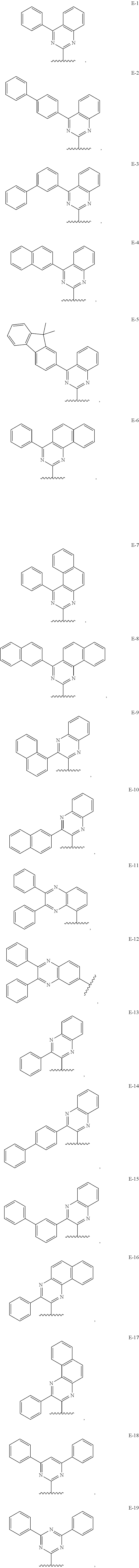 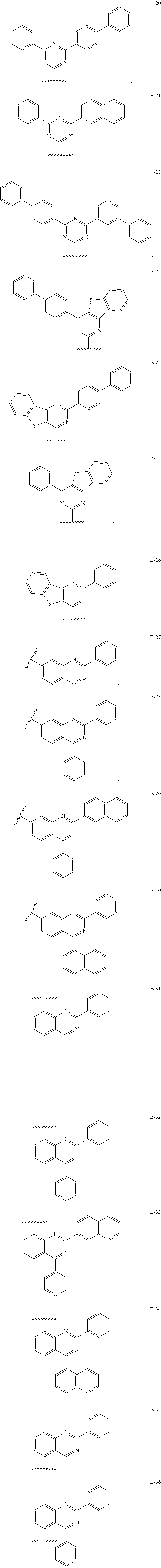 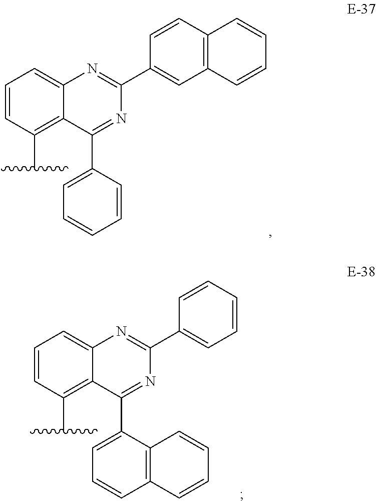 [SMILES: *c1cccc2nc(-c3ccc4ccccc4c3)nc(-c3ccccc3)c12 ; *c1cccc2nc(-c3ccccc3)nc(-c3cccc4ccccc34)c12]
wherein “ [symbol] ” represents a position where the structure of E is joined to the L.

11. The electroluminescent device according to claim 1, wherein the L is selected from the group consisting of: a single bond, phenylene, naphthylene, biphenylene, terphenylene, triphenylenylene, pyridylene, dibenzothienylene, dibenzofuranylene and thienylene; optionally, hydrogen in the above groups can be partially or fully substituted with deuterium;
preferably, the L is selected from the group consisting of the following structures: a single bond 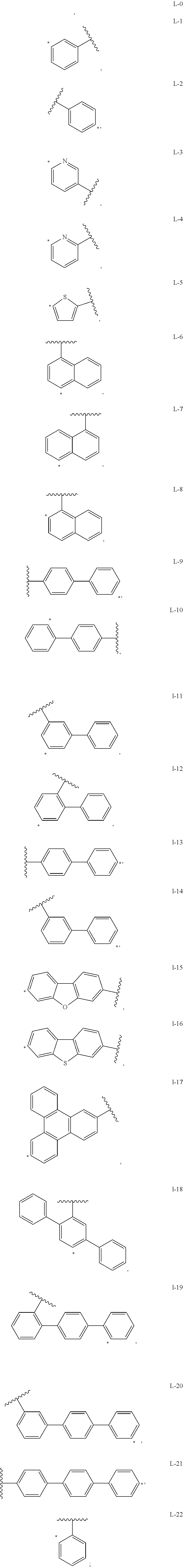
wherein “*” represents a position where the structure of L is joined to the H, and “ [symbol] ” represents a position where the structure of L is joined to the E;
optionally, hydrogen in the above structures of L-1 to L-22 can be partially or fully substituted with deuterium.

12. The electroluminescent device according to claim 1, wherein the first compound has the structure of H-L-E, wherein the H is selected from any one of the group consisting of H-1 to H-76, the L is selected from any one of the group consisting of L-0 to L-22, and the E is selected from any one of the group consisting of E-1 to E-38; optionally, hydrogen in the structure of the first compound can be partially or fully substituted with deuterium;
preferably, the first compound is selected from the group consisting of the following structures: 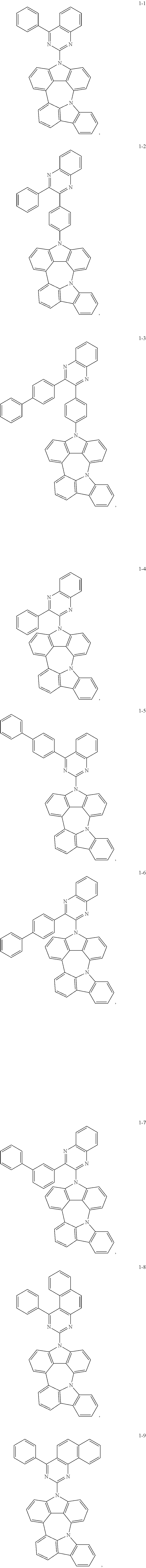 [chemical structure] [chemical structure] [chemical structure] [chemical structure] [chemical structure] [chemical structure] [chemical structure] [chemical structure] [chemical structure] [chemical structure] 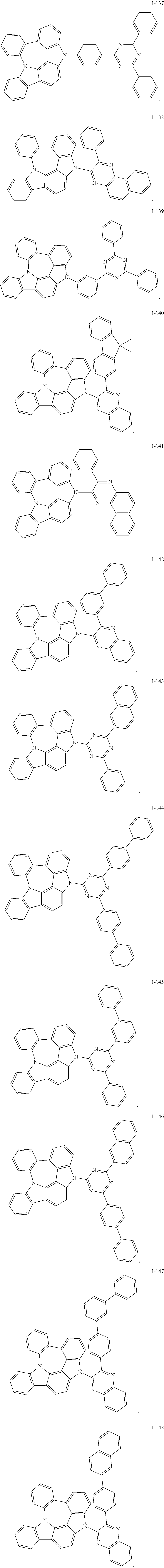 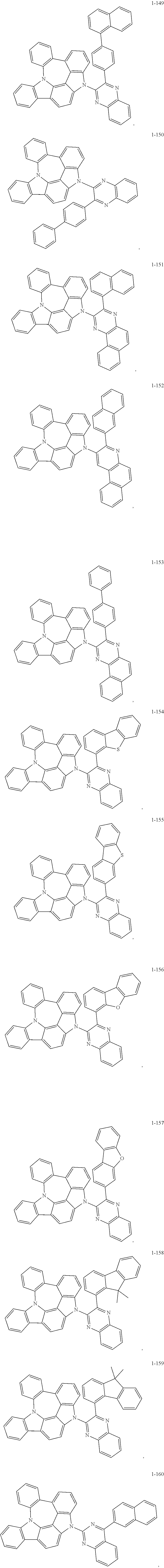 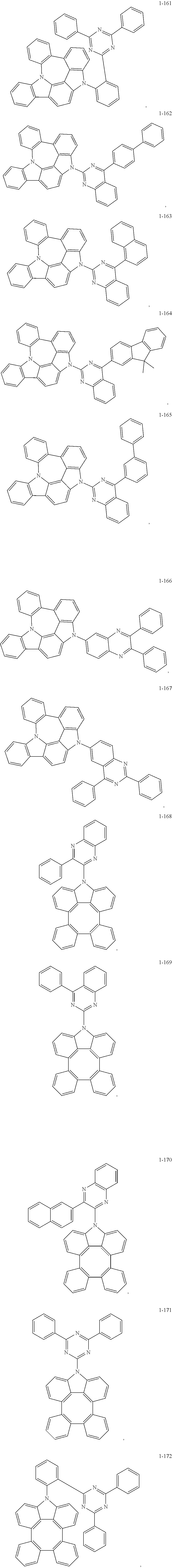 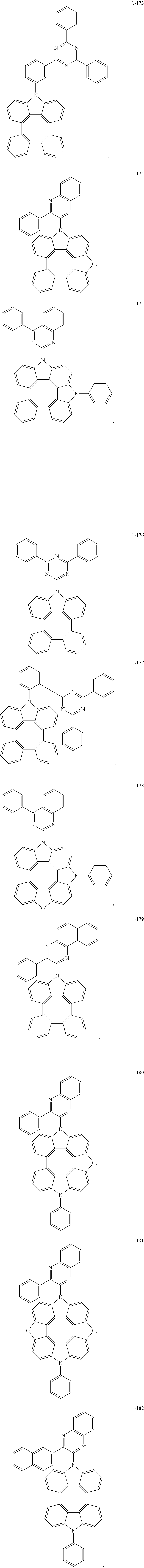 [chemical structure] [chemical structure] [chemical structure] 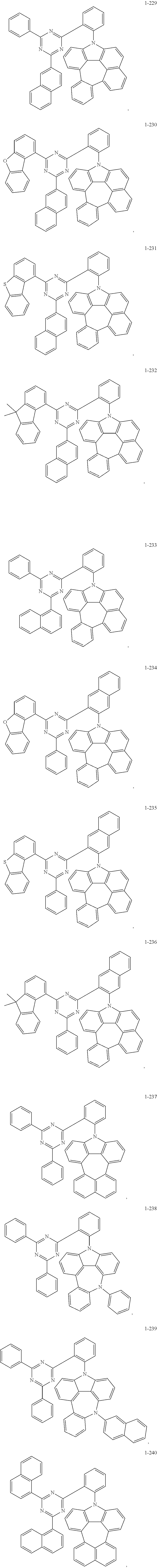 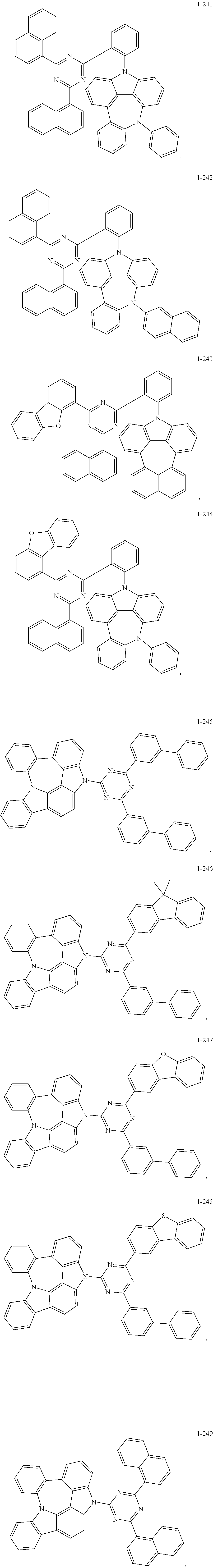
wherein optionally, hydrogen in the above structures can be partially or fully substituted with deuterium.

13. The electroluminescent device according to claim 1, wherein in Formula C, the ring A and/or the ring B are each independently selected from a five-membered unsaturated carbocyclic ring, an aromatic ring having 6 to 18 carbon atoms or a heteroaromatic ring having 3 to 18 carbon atoms; and
preferably, the ring A and/or the ring B are each independently selected from a five-membered unsaturated carbocyclic ring, an aromatic ring having 6 to 10 carbon atoms or a heteroaromatic ring having 3 to 10 carbon atoms.

14. The electroluminescent device according to claim 1, wherein in the second compound, the Lₐ has a structure represented by any one of Formula 2-1 to Formula 2-19: 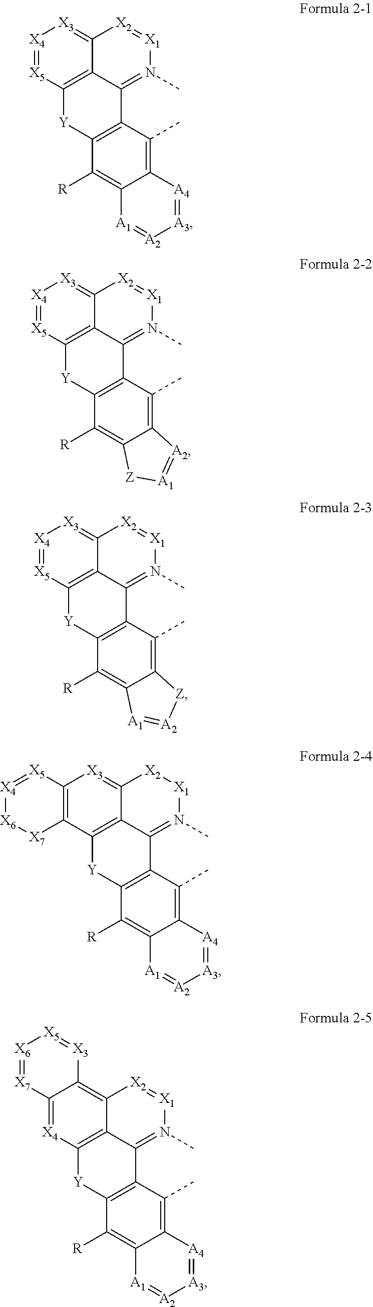 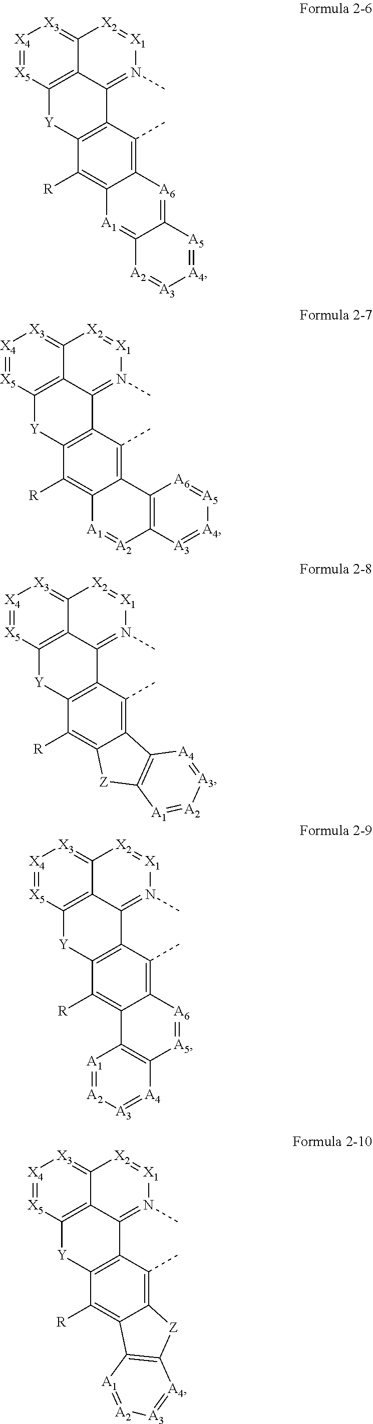 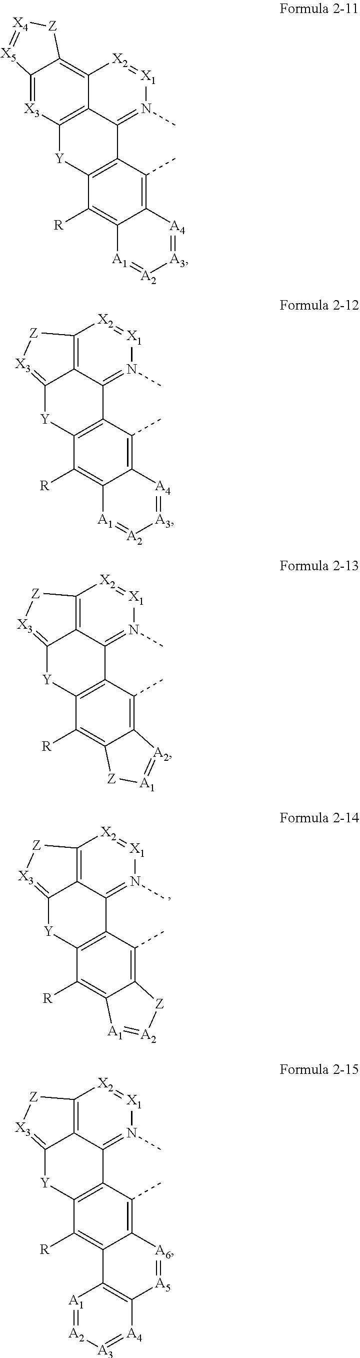 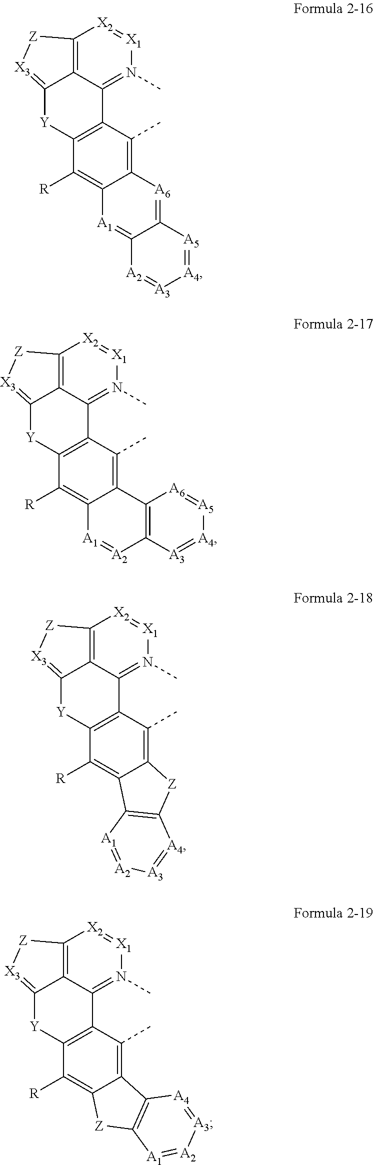
wherein
in Formula 2-1 to Formula 2-19, X₁ and X₂ are, at each occurrence identically or differently, selected from CRₓ or N, X₃ to X₇ are, at each occurrence identically or differently, selected from CRᵢ or N, and A₁ to A₆ are, at each occurrence identically or differently, selected from CRᵢᵢ or N;
Z is, at each occurrence identically or differently, selected from CRᵢᵢᵢRᵢᵢᵢ, SiRᵢᵢᵢRᵢᵢᵢ, PRᵢᵢᵢ, O, S or NRᵢᵢᵢ; wherein when two Rᵢᵢᵢ are present, the two Rᵢᵢᵢ are identical or different;
Y is selected from SiR_{y}R_{y}, NR_{y}, PR_{y}, O, S or Se; wherein when two R_{y} are present, the two R_{y} are identical or different;
R, Rₓ, R_{y}, Rᵢ, Rᵢᵢ and Rᵢᵢᵢ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof;
adjacent substituents R, Rₓ, R_{y}, Rᵢ, Rᵢᵢ and Rᵢᵢᵢ can be optionally joined to form a ring;
preferably, Lₐ is selected from a structure represented by any one of Formula 2-1, Formula 2-5, Formula 2-8, Formula 2-10, Formula 2-11 or Formula 2-12;
more preferably, Lₐ is selected from a structure represented by Formula 2-1.

15. The electroluminescent device according to claim 14, wherein in Formula 2-1 to Formula 2-19, at least one of X₁ to Xₙ and/or A₁ to Aₘ is selected from N, wherein the Xₙ corresponds to one of X₁ to X₇ that has the largest number in any one of Formula 2-1 to Formula 2-19, and the Aₘ corresponds to one of A₁ to A₆ that has the largest number in any one of Formula 2-1 to Formula 2-19;
preferably, in Formula 2-1 to Formula 2-19, at least one of X₁ to Xₙ is selected from N, wherein the Xₙ corresponds to one of X₁ to X₇ that has the largest number in any one of Formula 2-1 to Formula 2-19;
more preferably, X₂ is N.

16. The electroluminescent device according to claim 14, wherein in Formula 2-1 to Formula 2-19, X₁ and X₂ are each independently selected from CRₓ, X₃ to X₇ are each independently selected from CRᵢ, and A₁ to A₆ are each independently selected from CRᵢᵢ;
adjacent substituents Rₓ, Rᵢ, Rᵢᵢ can be optionally joined to form a ring;
preferably, Rₓ, Rᵢ and Rᵢᵢ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, cyano and combinations thereof,
more preferably, at least two or three of Rₓ, Rᵢ and Rᵢᵢ are, at each occurrence identically or differently, selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, cyano and combinations thereof.

17. The electroluminescent device according to claim 1, wherein in the second compound, the ligand Lₐ has a structure represented by Formula 2-20 or Formula 2-21: 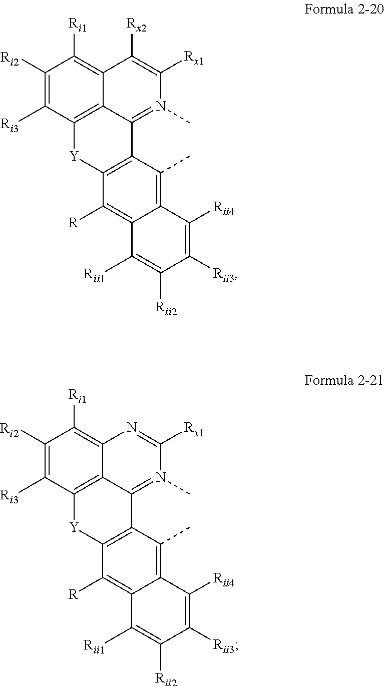
wherein in Formula 2-20 and Formula 2-21,
Y is selected from O or S;
Rₓ₁, Rₓ₂, Rᵢ₁, Rᵢ₂, Rᵢ₃, Rᵢᵢ₁, Rᵢᵢ₂, Rᵢᵢ₃ and Rᵢᵢ₄ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms and combinations thereof,
R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms and combinations thereof,
preferably, at least one or two of Rₓ₁, Rₓ₂, Rᵢ₁, Rᵢ₂ and Rᵢ₃ and/or at least one or two of Rᵢᵢ₁, Rᵢᵢ₂, Rᵢᵢ₃ and Rᵢᵢ₄ are, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, R is selected from halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof,
more preferably, at least one or two of Rₓ₁, Rₓ₂, Rᵢ₁, Rᵢ₂ and Rᵢ₃ and/or at least one or two of Rᵢᵢ₁, Rᵢᵢ₂, Rᵢᵢ₃ and Rᵢᵢ₄ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof; R is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

18. The electroluminescent device according to claim 17, wherein Rᵢ₂ is selected from the group consisting of: deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof; R is selected from halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and at least one or two of Rᵢᵢ₁, Rᵢᵢ₂, Rᵢᵢ₃ and Rᵢᵢ₄ are, at each occurrence identically or differently, selected from deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof,
preferably, Rᵢ₂ is selected from the group consisting of: substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, R is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof, and at least one or two of Rᵢᵢ₁, Rᵢᵢ₂, Rᵢᵢ₃ and Rᵢᵢ₄ are, at each occurrence identically or differently, selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms or a combination thereof.

19. The electroluminescent device according to claim 17, wherein in Formula 2-20 and Formula 2-21, at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group consisting of: substituted or unsubstituted alkyl having 3 to 20 carbon atoms, substituted or unsubstituted cycloalkyl having 3 to 20 ring carbon atoms, substituted or unsubstituted alkylsilyl having 3 to 20 carbon atoms and combinations thereof, and
preferably, at least one of Rₓ₁, Rₓ₂, Rᵢ₁, Rᵢ₂, Rᵢ₃, Rᵢᵢ₁, Rᵢᵢ₂, Rᵢᵢ₃, Rᵢᵢ₄ and R is, at each occurrence identically or differently, selected from the group consisting of: substituted or unsubstituted alkyl having 3 to 10 carbon atoms, substituted or unsubstituted cycloalkyl having 3 to 10 ring carbon atoms and combinations thereof.

20. The electroluminescent device according to claim 1, wherein the Lₐ is, at each occurrence identically or differently, selected from the group consisting of the following structures: 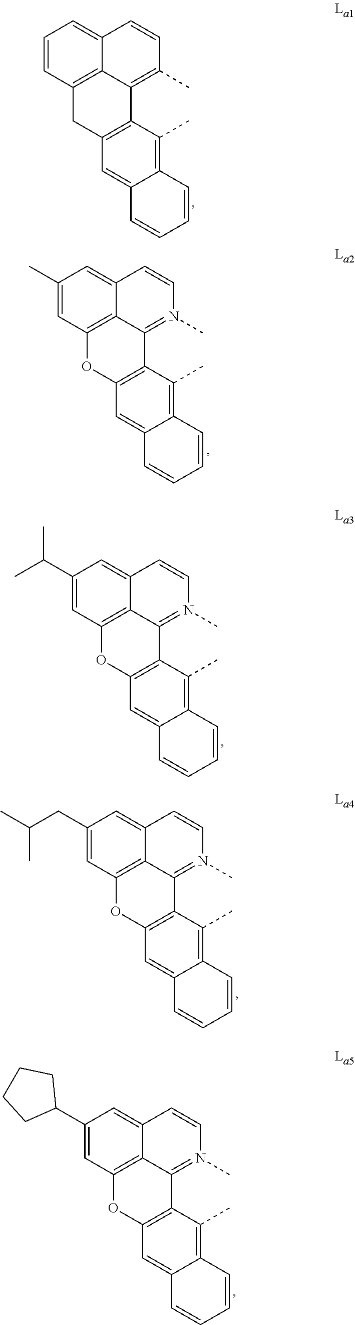 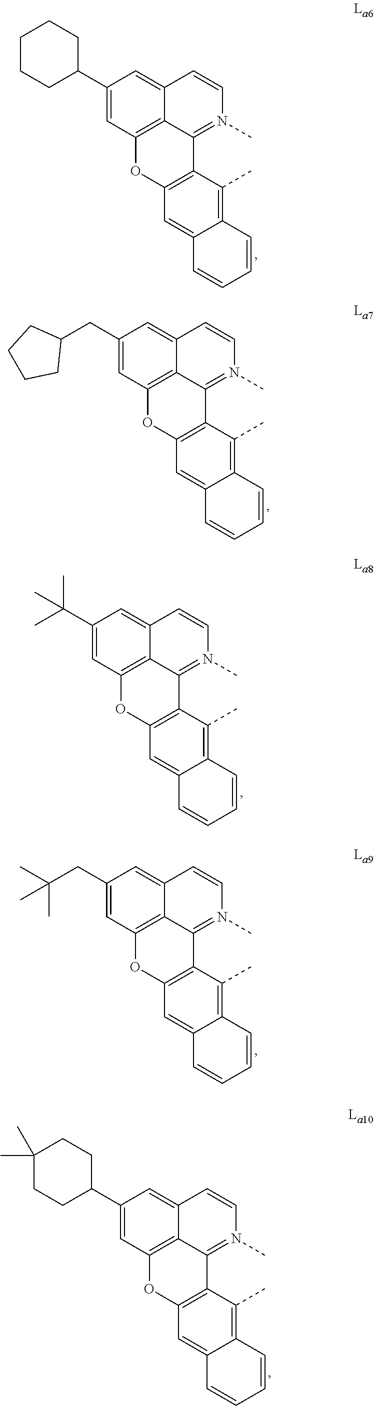 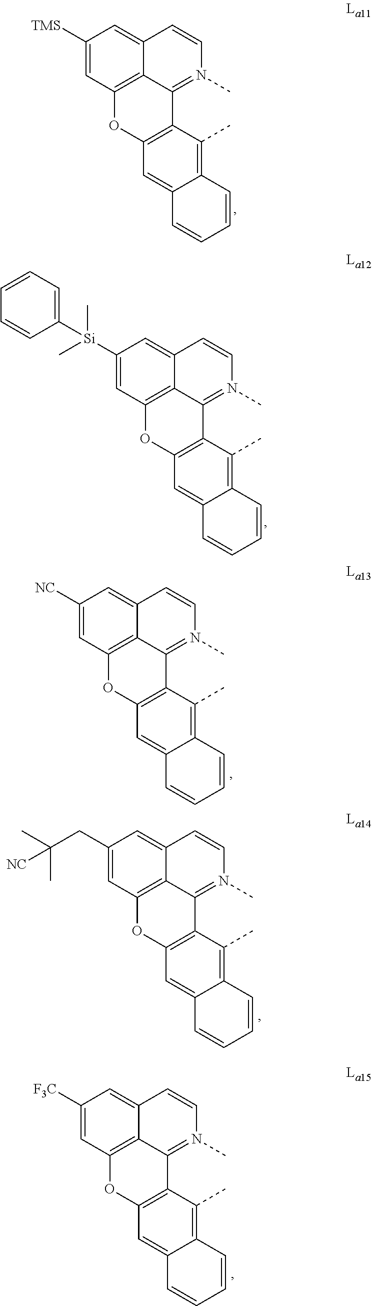 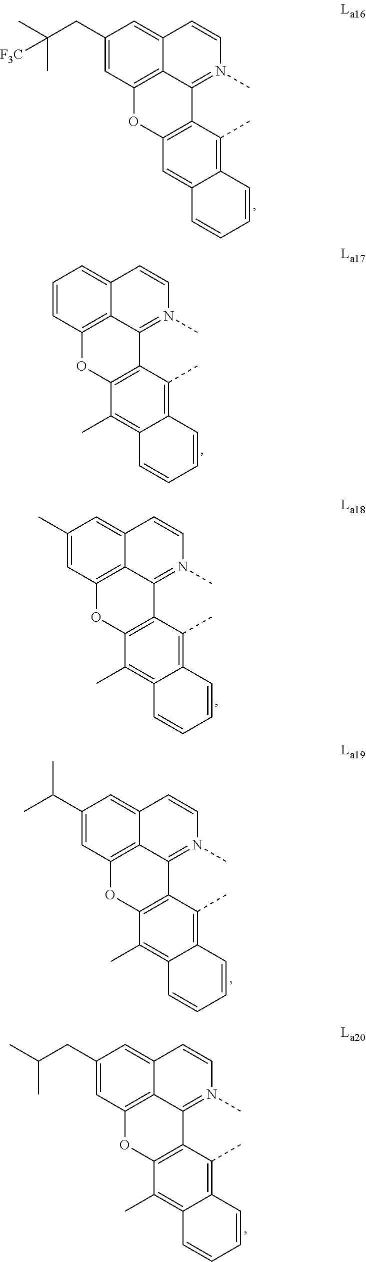 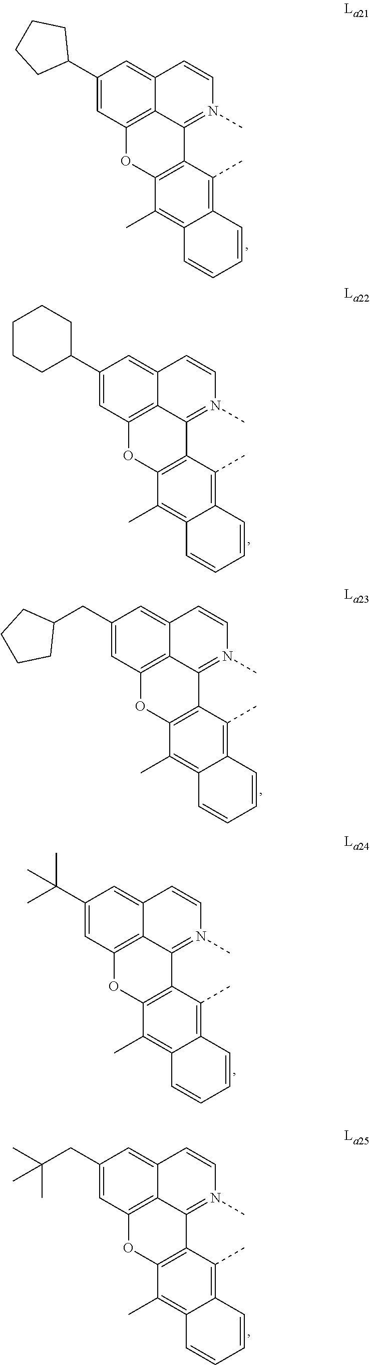 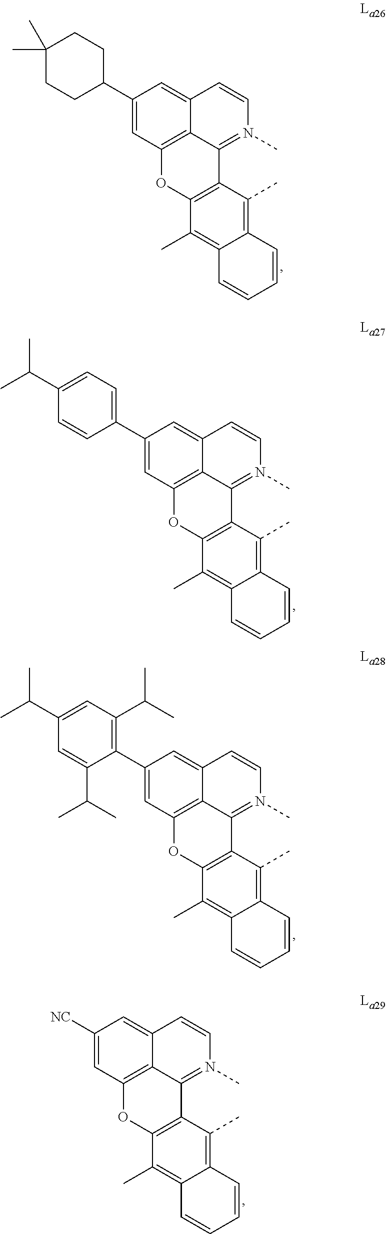 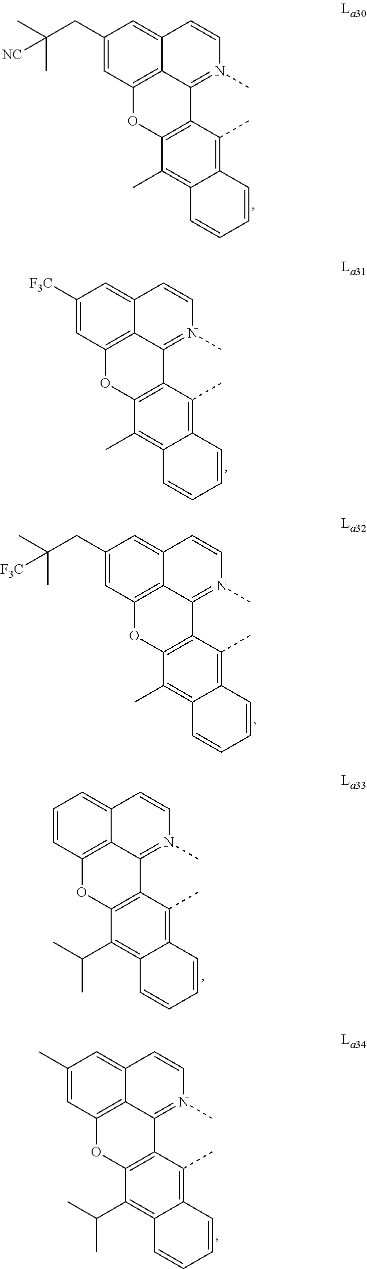 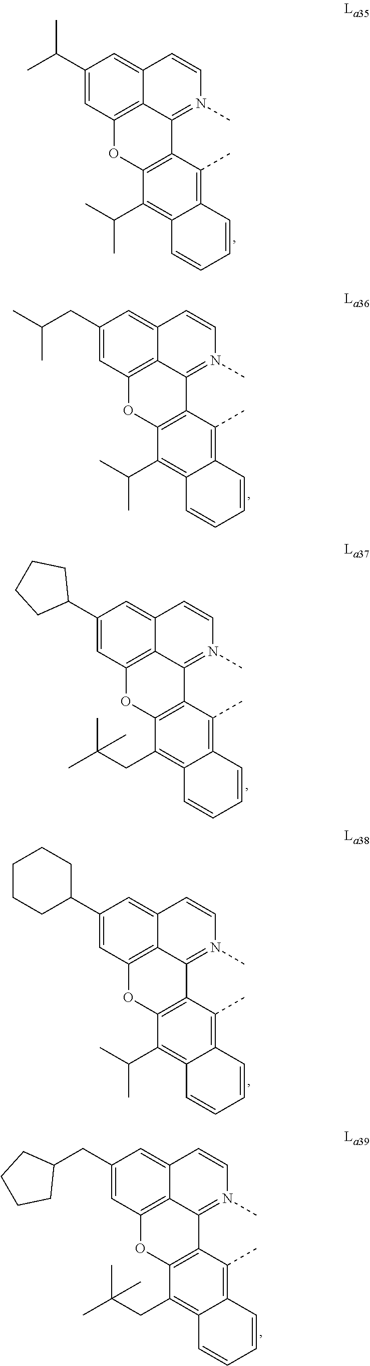 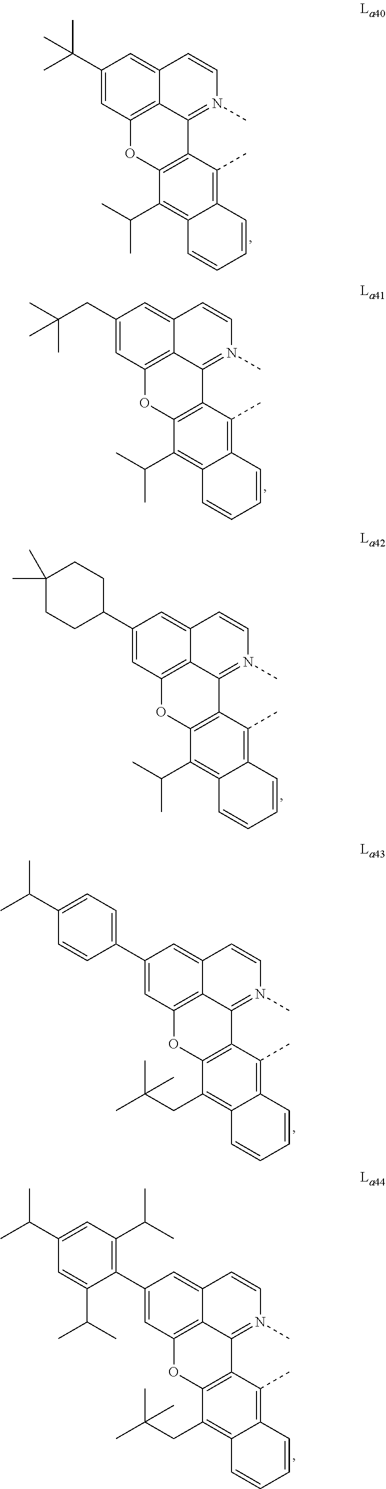 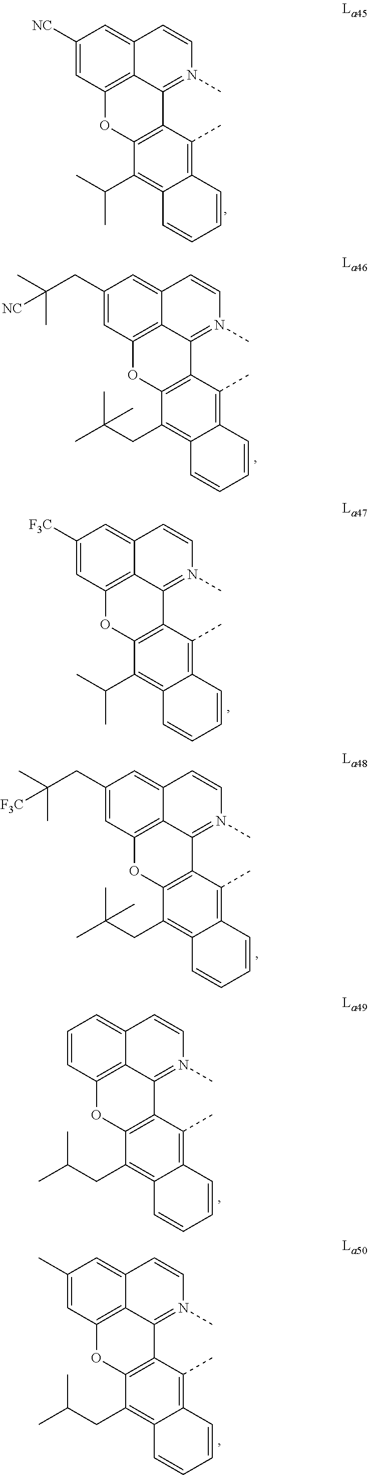 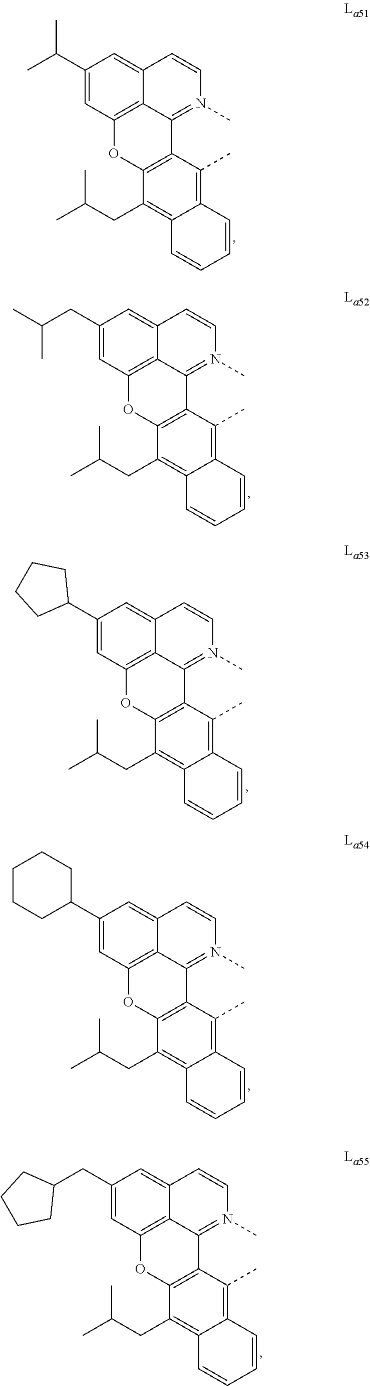 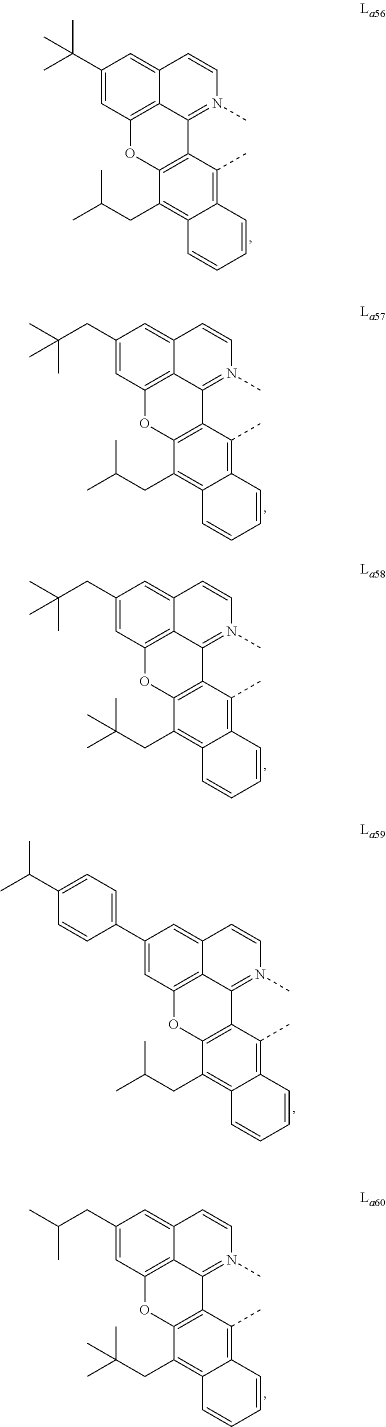 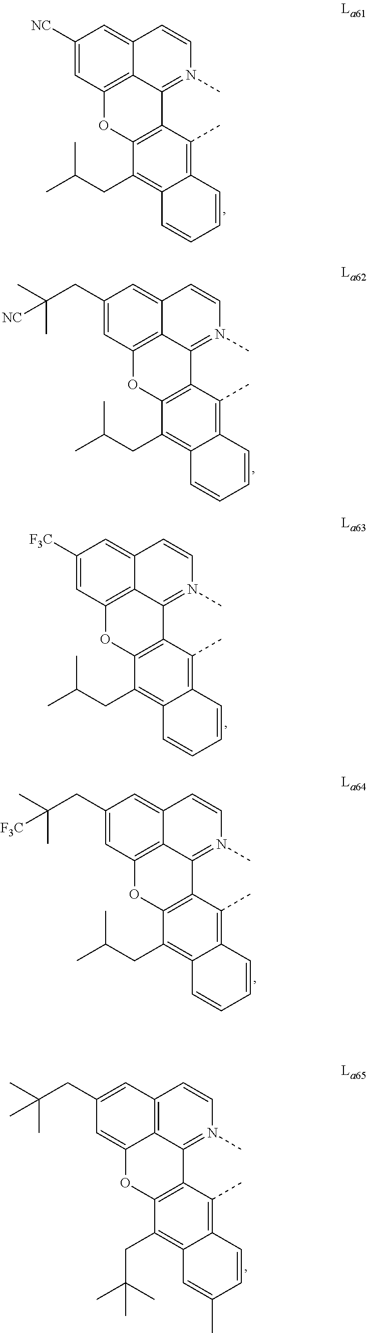 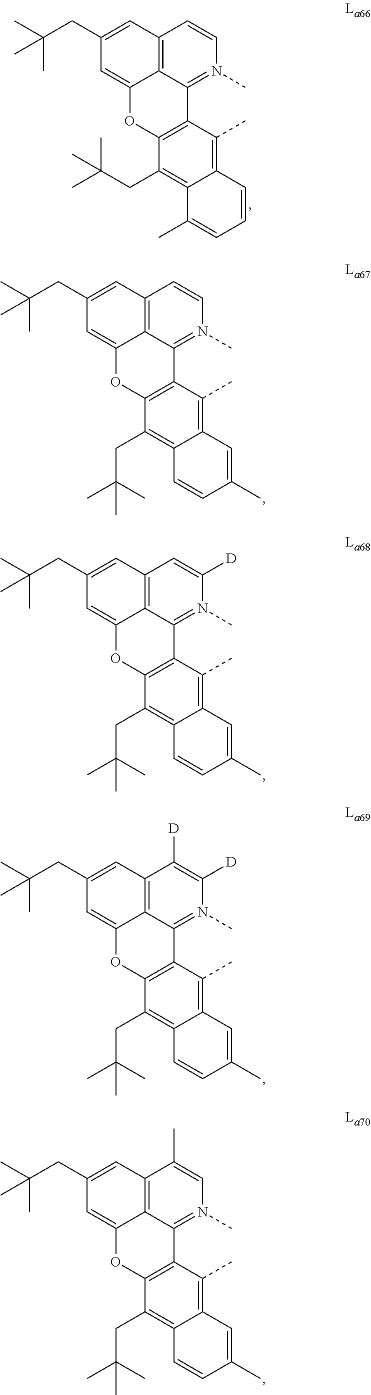 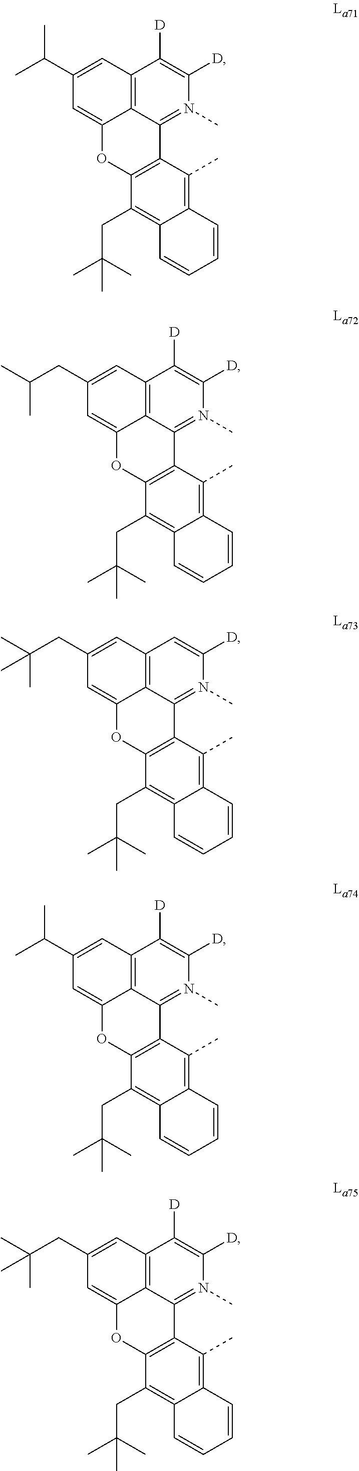 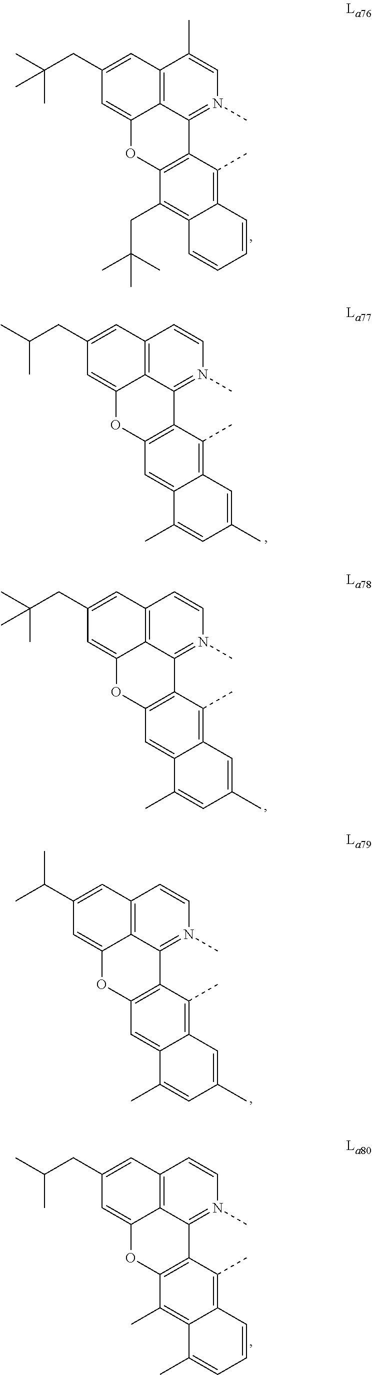 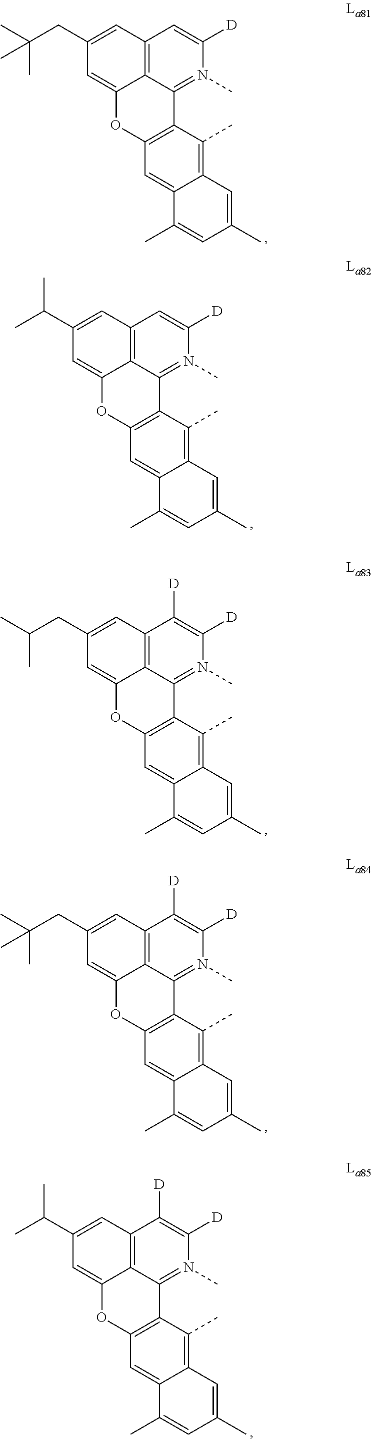 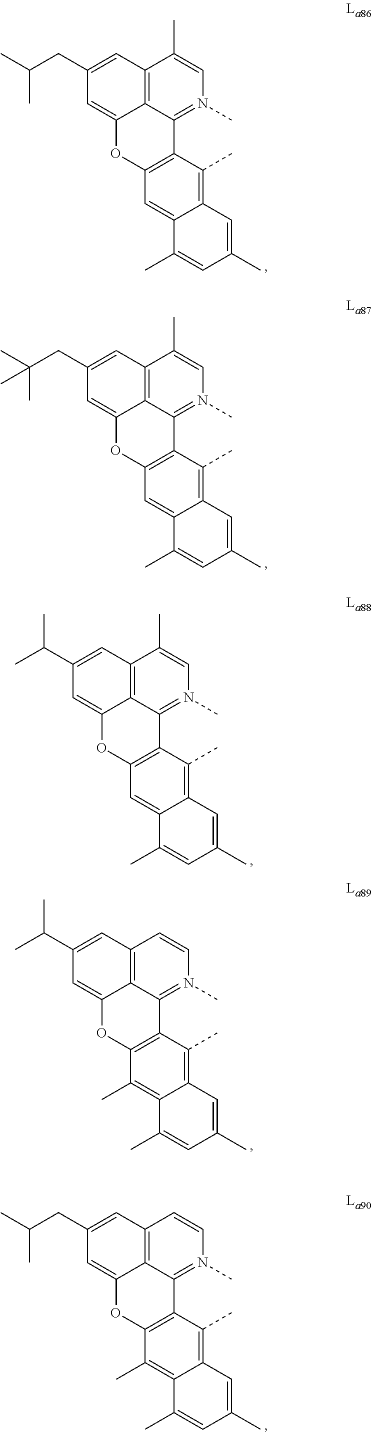 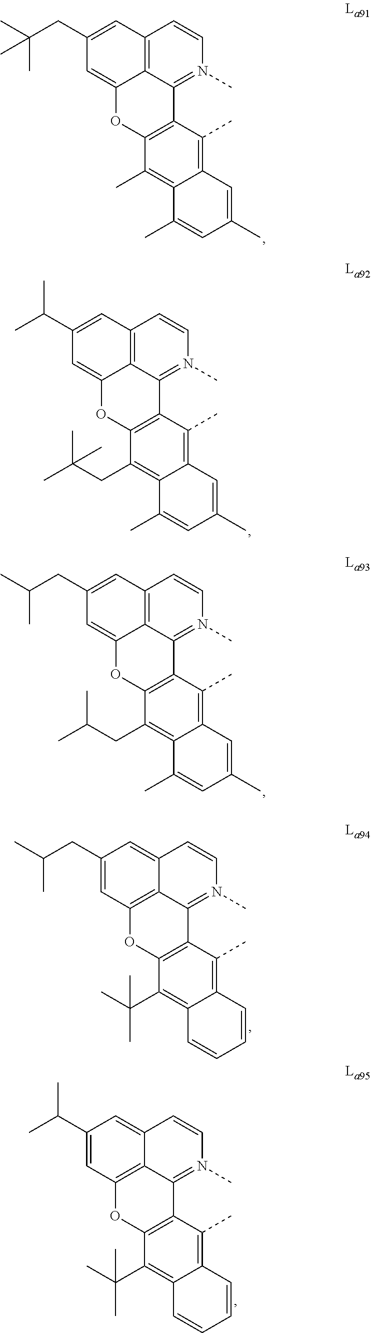 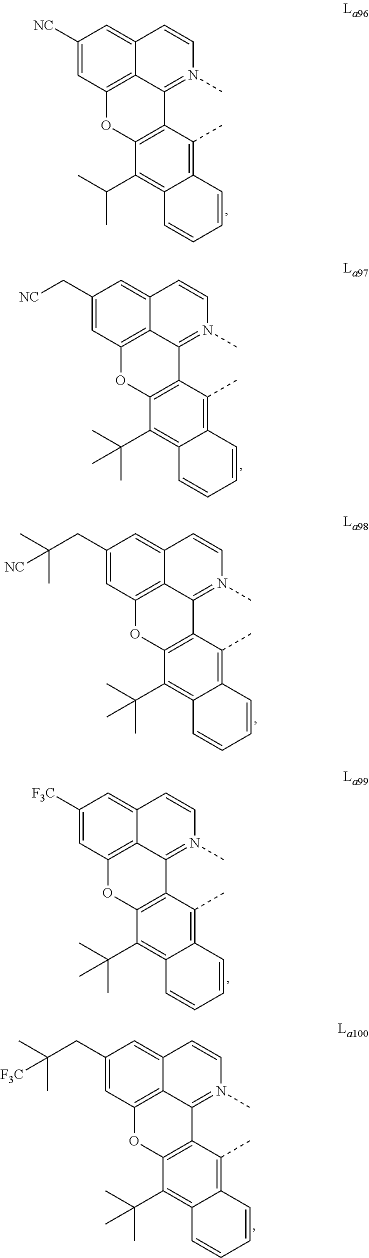 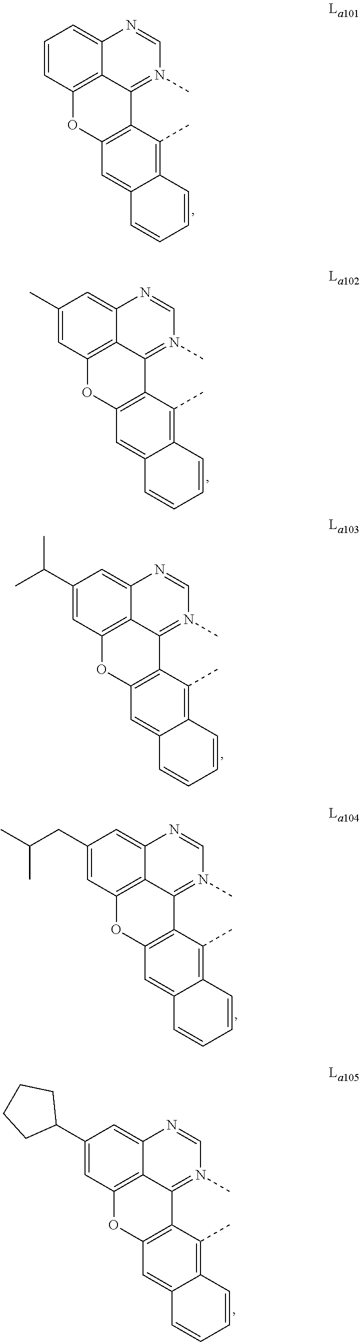 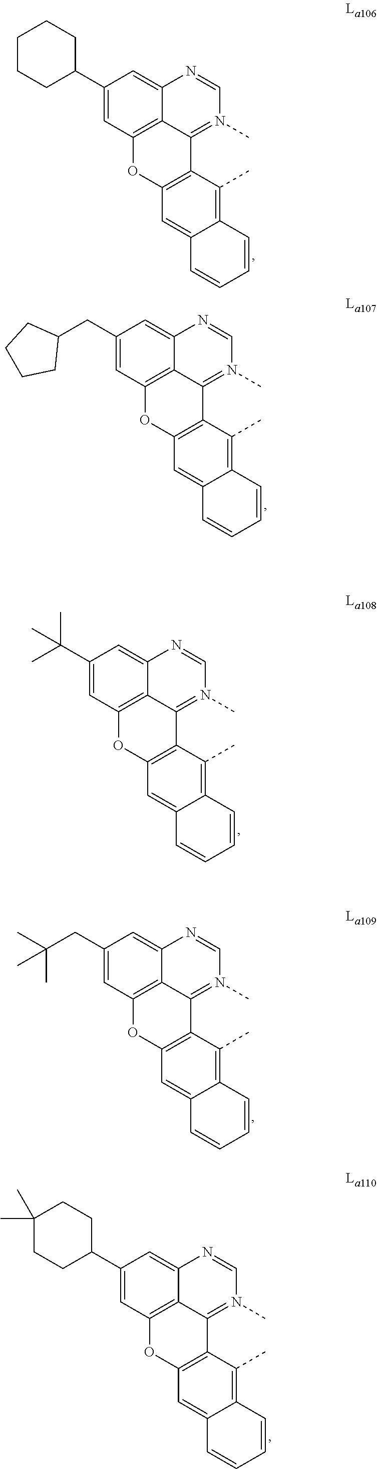 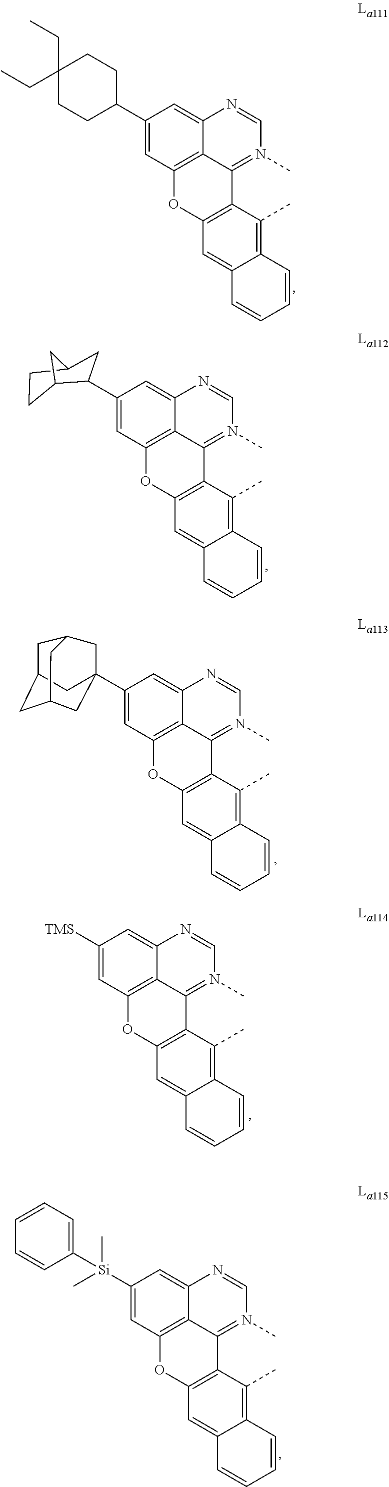 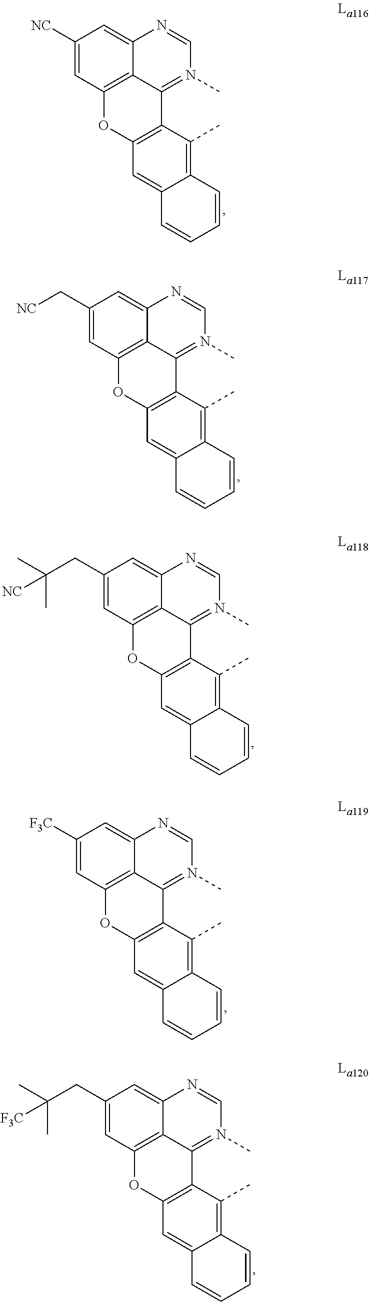 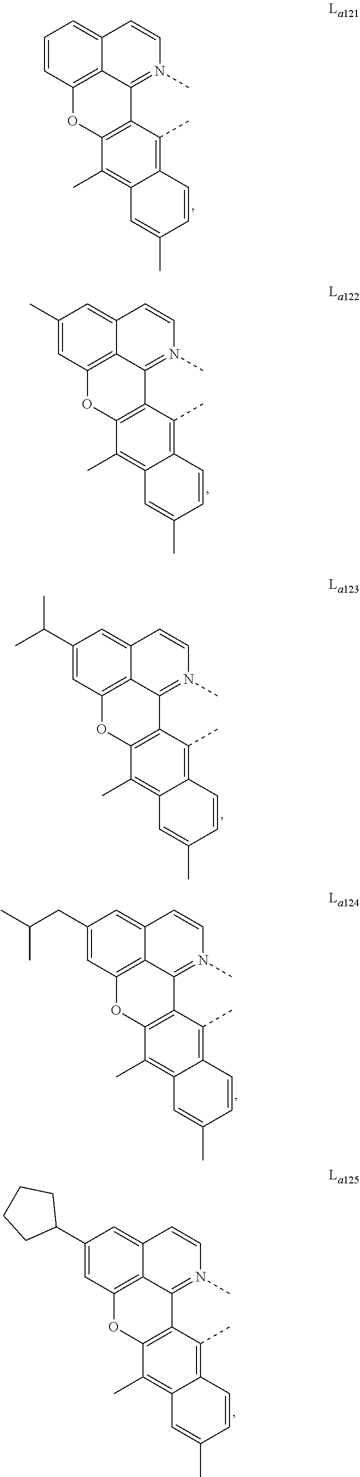 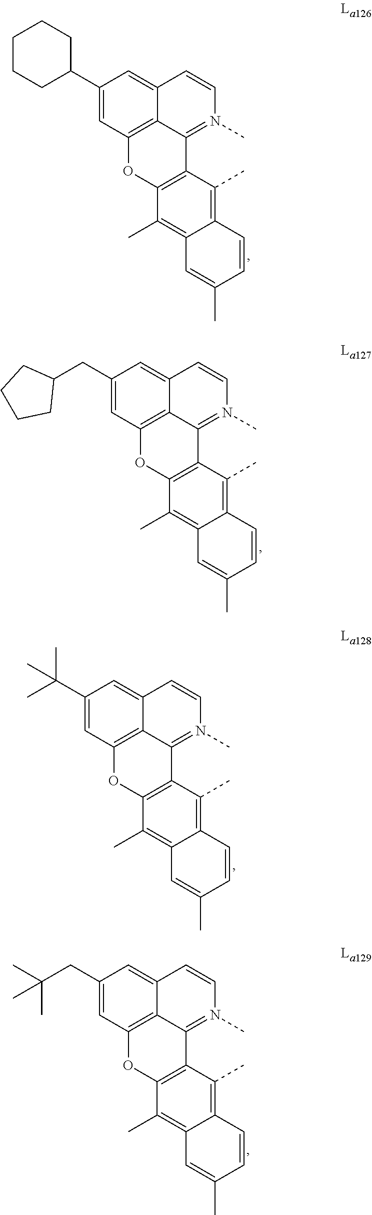 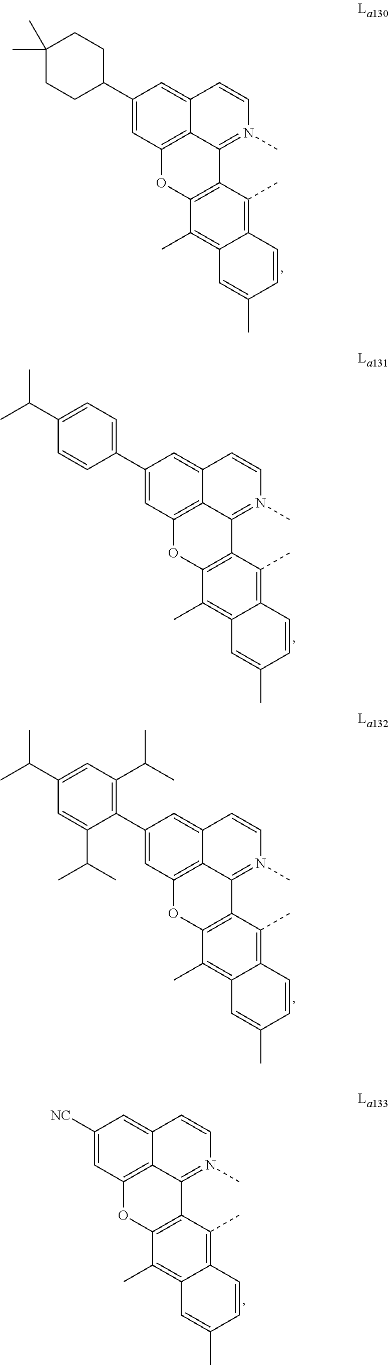 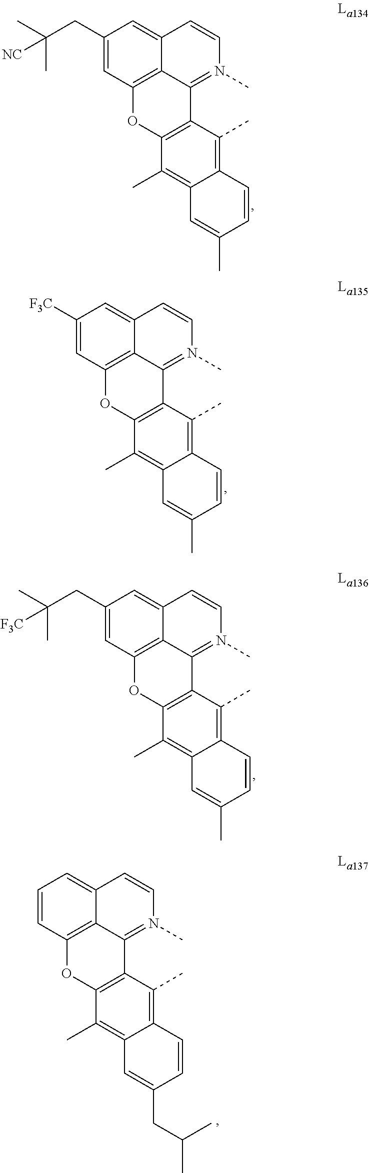 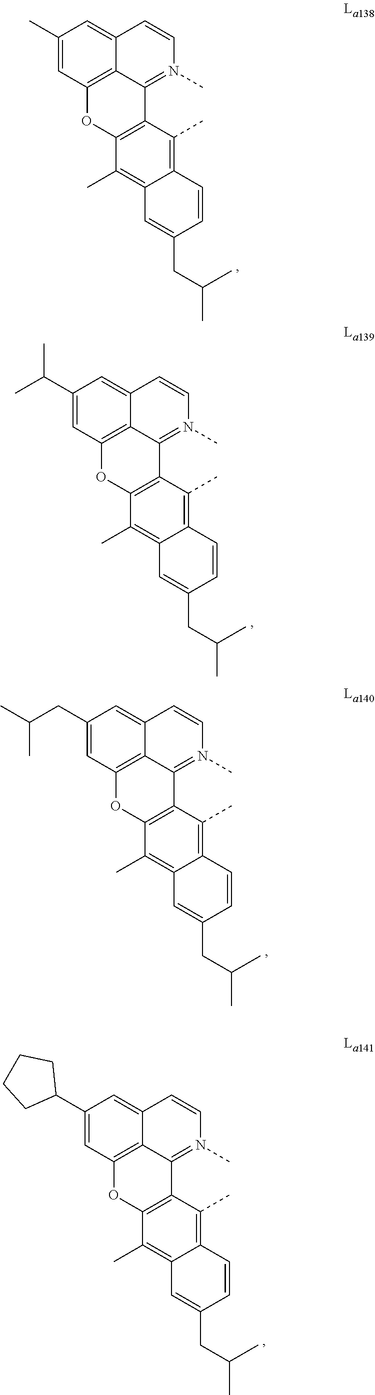 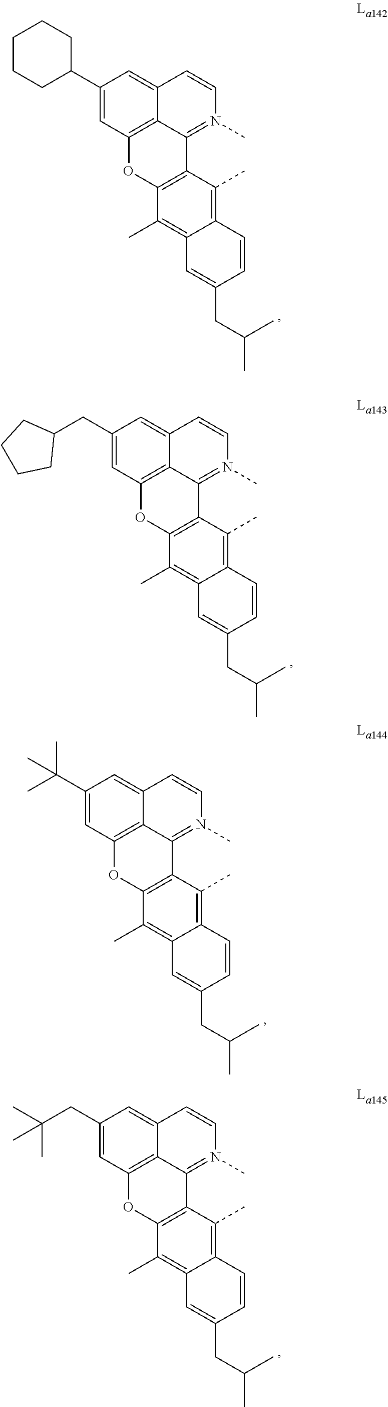 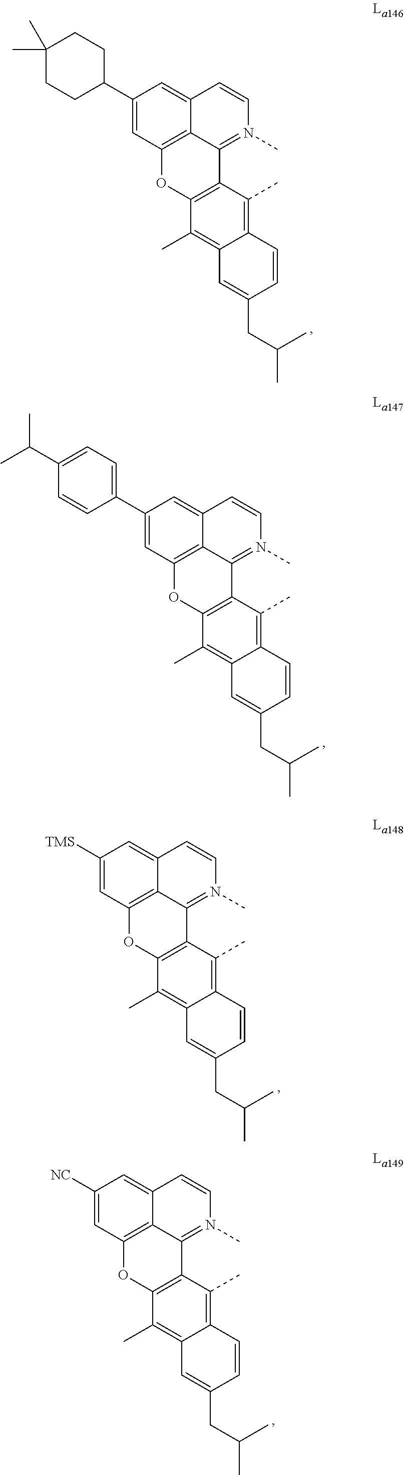 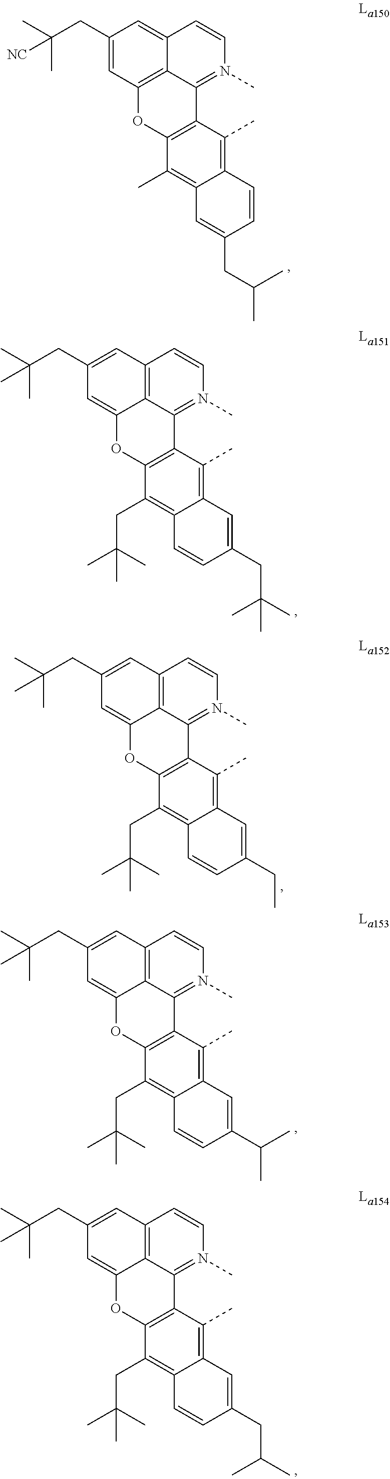 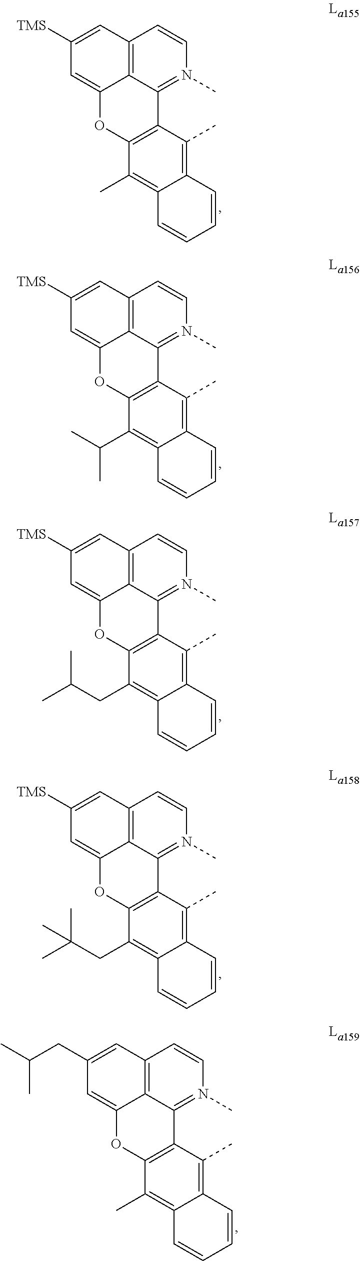 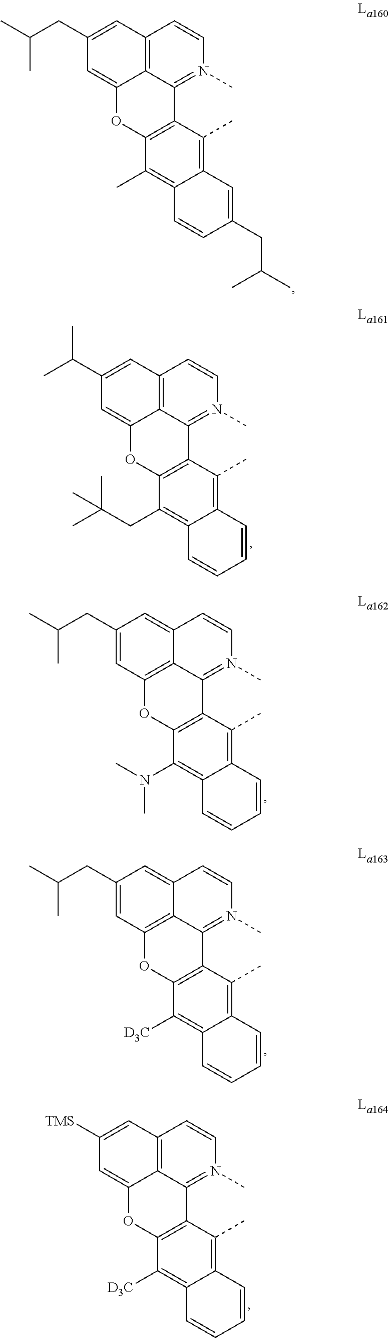 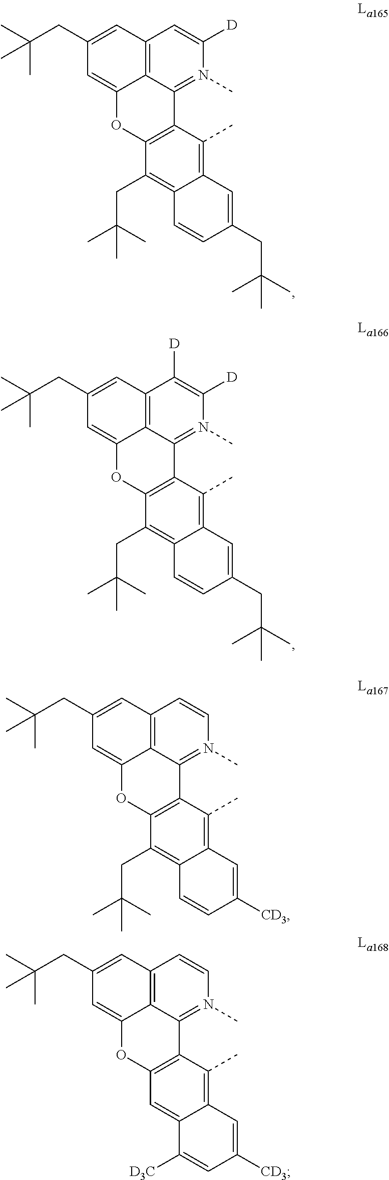
wherein in the above structures, TMS represents trimethylsilyl;
optionally, hydrogen in the structures of Lₐ₁ to Lₐ₁₆₈ can be partially or fully substituted with deuterium.

21. The electroluminescent device according to claim 1, wherein the second compound has a general formula of M(Lₐ)ₘ(L_{b})ₙ(L_{c})_{q}, wherein the metal M is selected from a metal with a relative atomic mass greater than 40, and Lₐ, L_{b} and L_{c} are a first ligand, a second ligand and a third ligand of the complex, respectively;
m is 1, 2 or 3, n is 0, 1 or 2, q is 0, 1 or 2, and m+n+q equals to an oxidation state of the metal M; wherein when m is greater than 1, a plurality of Lₐ are identical or different; when n is 2, two L_{b} are identical or different; when q is 2, two L_{c} are identical or different;
Lₐ, L_{b} and L_{c} can be optionally joined to form a multidentate ligand;
L_{b} and L_{c} are, at each occurrence identically or differently, selected from the group consisting of the following structures: 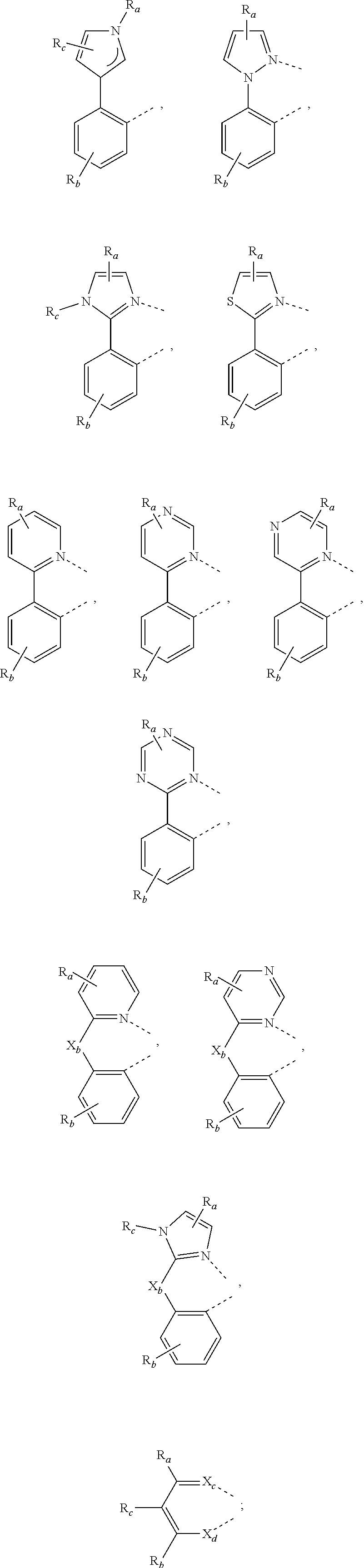
wherein Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2},
Rₐ, R_{b}, R_{c}, R_{N1}, R_{N2}, R_{C1} and R_{c2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and
adjacent substituents Rₐ, R_{b}, R_{c}, R_{N1}, R_{N2}, R_{C1} and R_{C2} can be optionally joined to form a ring.

22. The electroluminescent device according to claim 21, wherein the metal M is selected from Ir, Rh, Re, Os, Pt, Au or Cu; preferably, M is selected from Ir or Pt; more preferably, M is Ir.

23. The electroluminescent device according to claim 21, wherein L_{b} is, at each occurrence identically or differently, selected from the group consisting of the following structures: 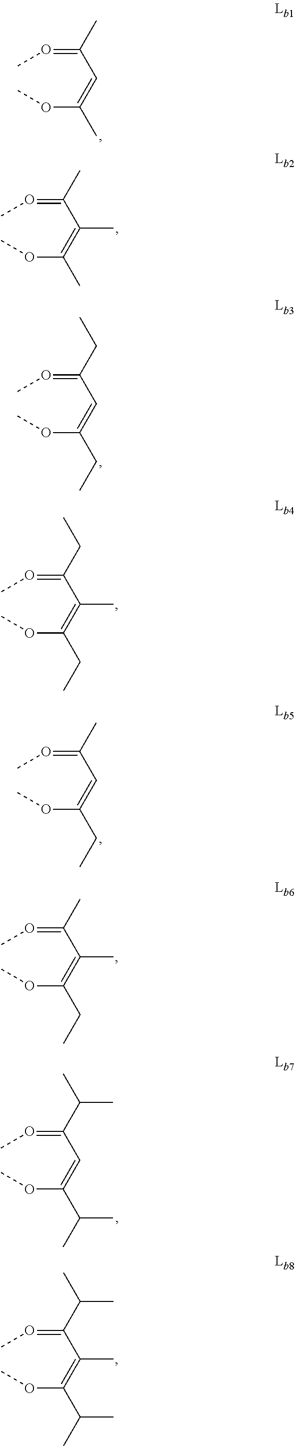 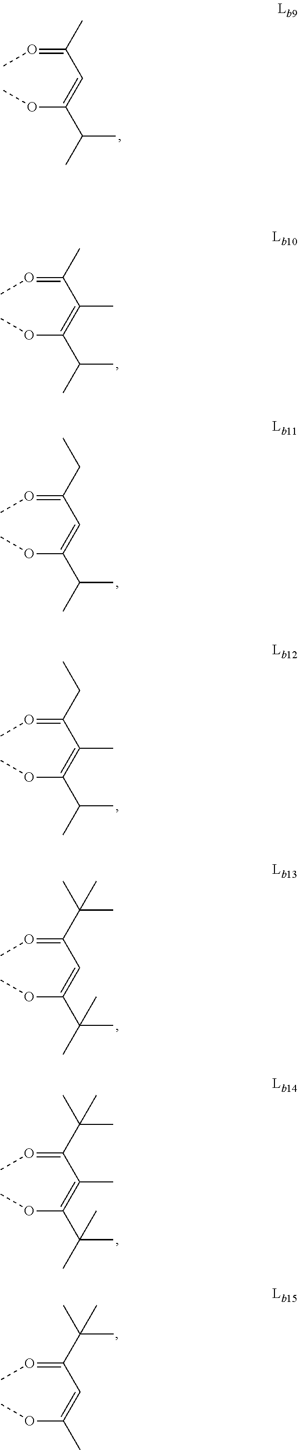 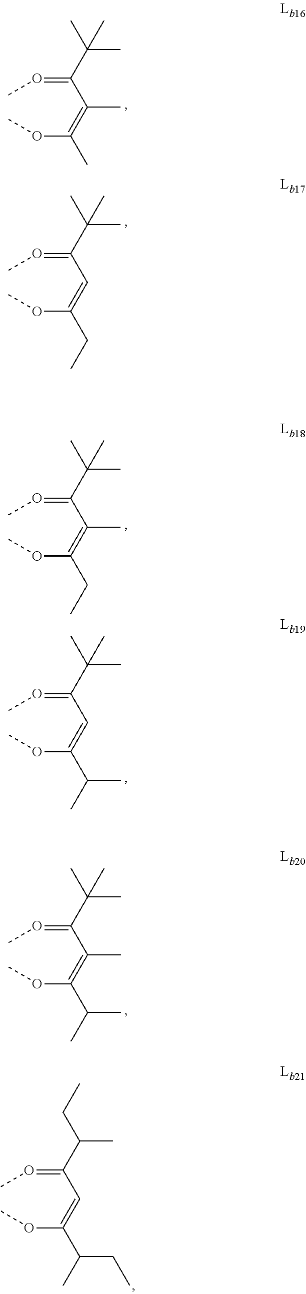 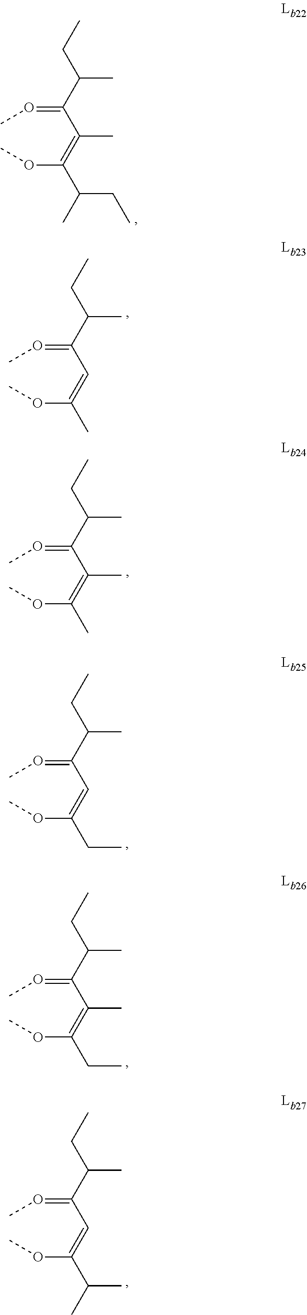 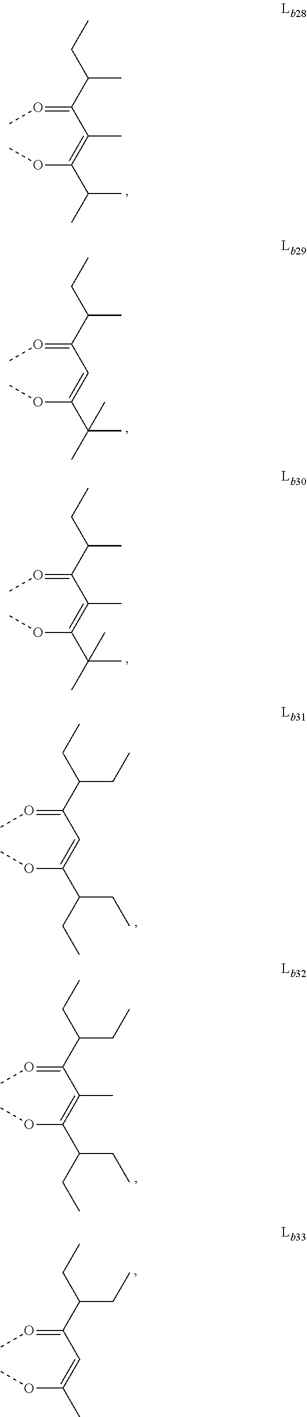 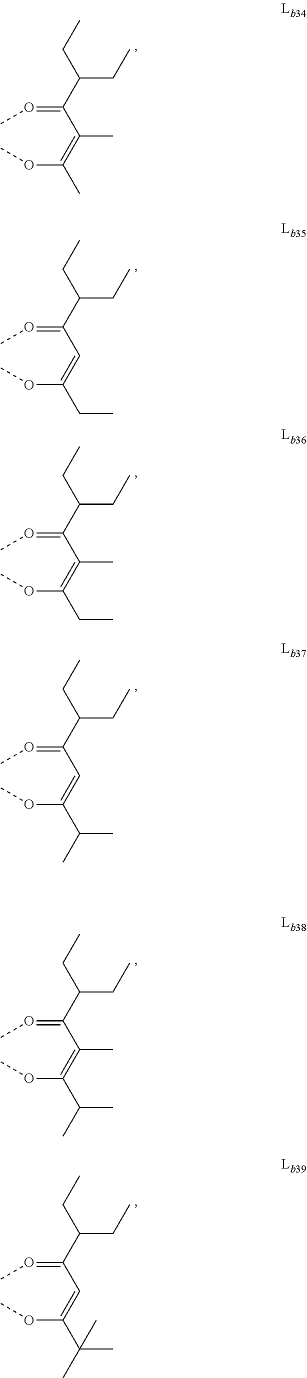 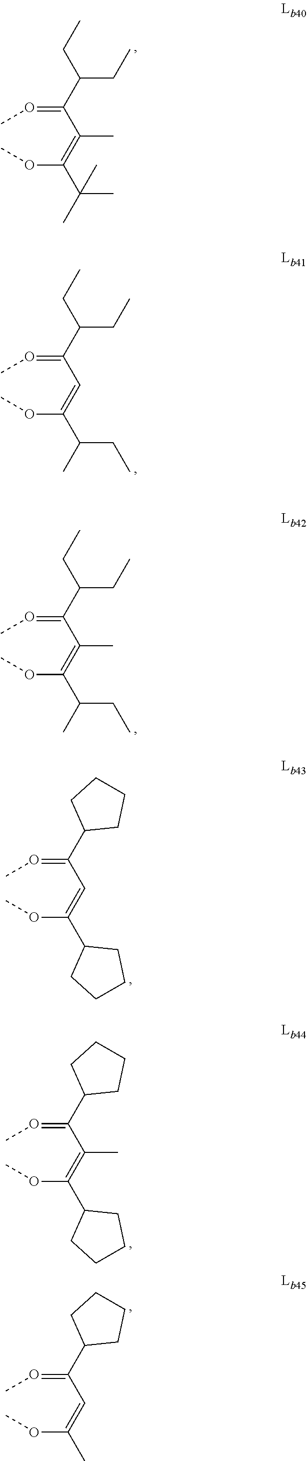 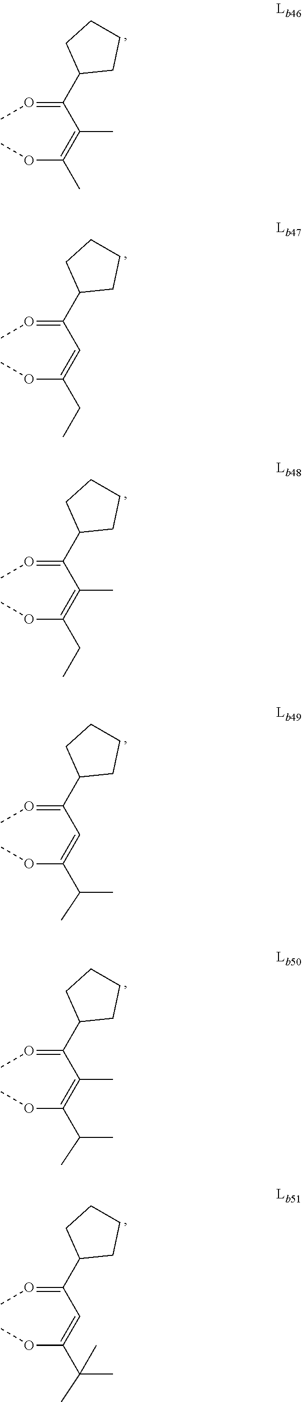 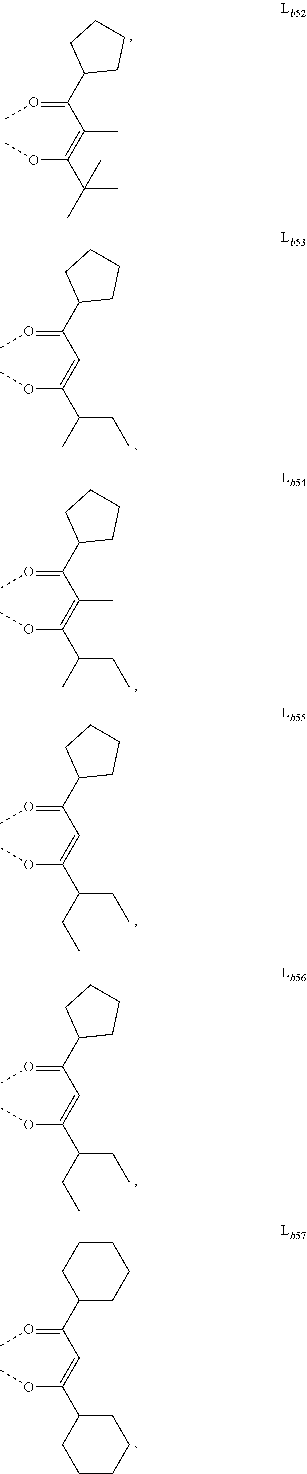 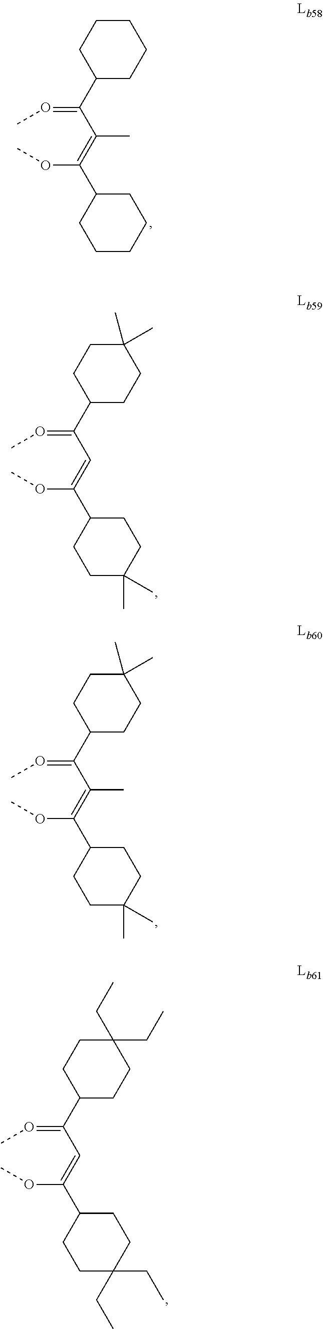 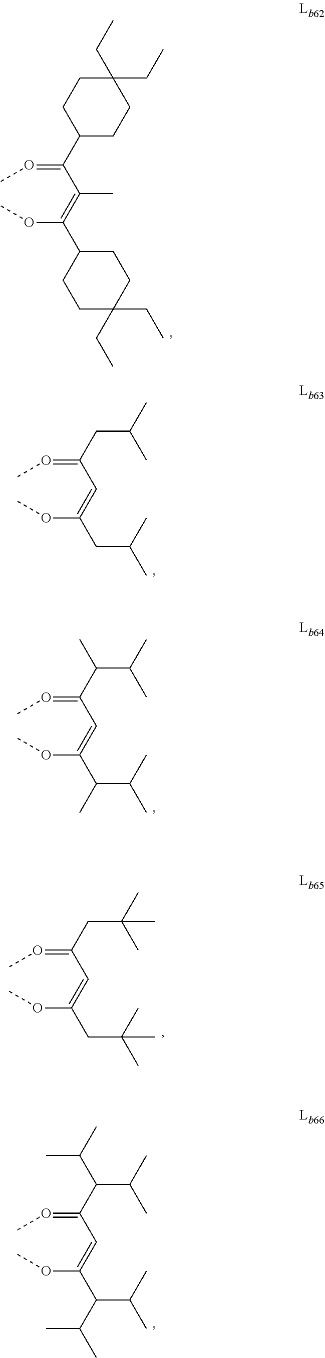 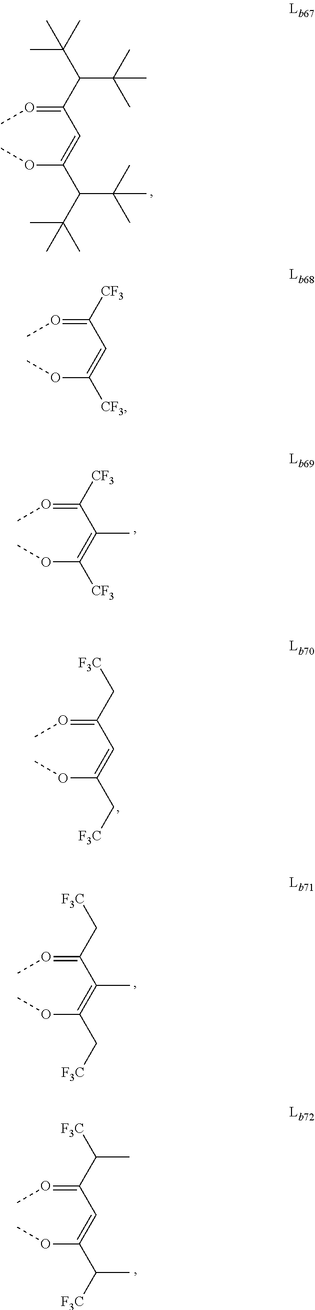 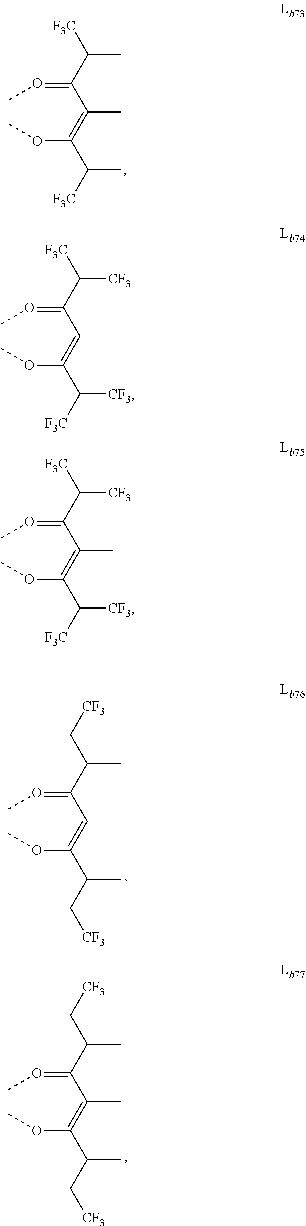 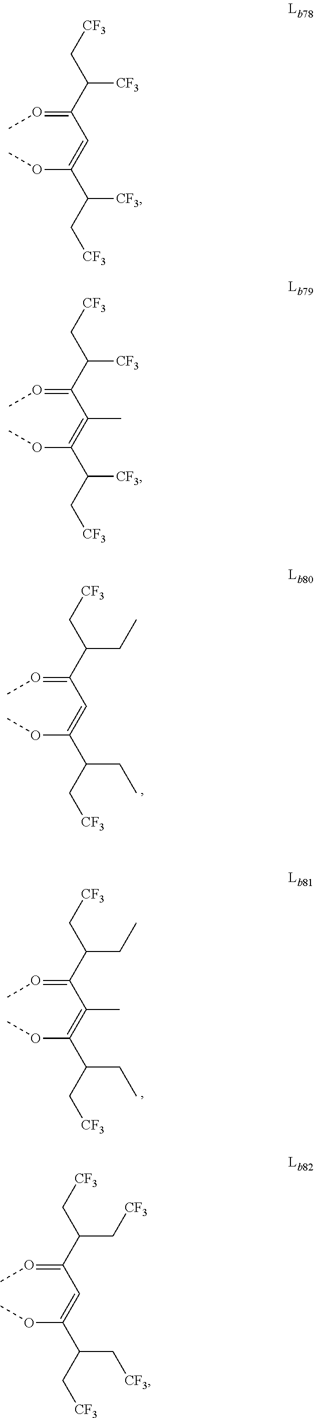 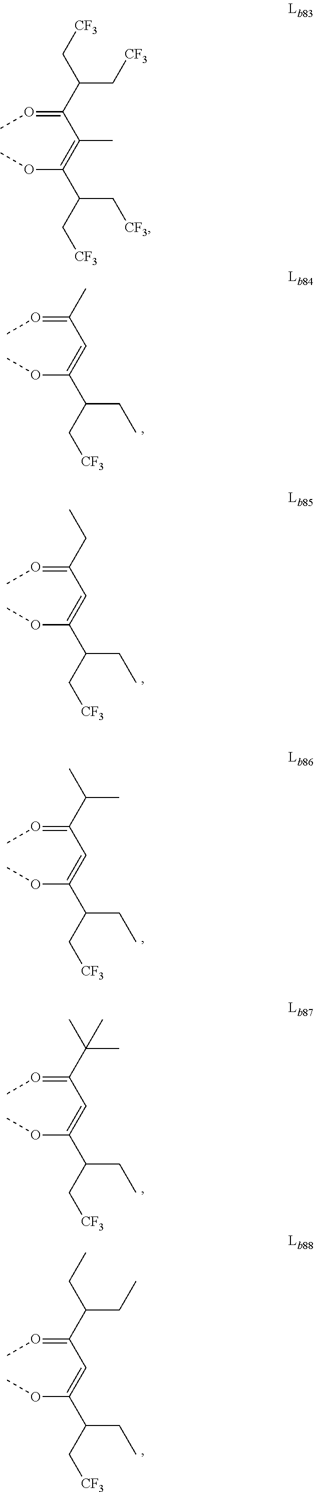 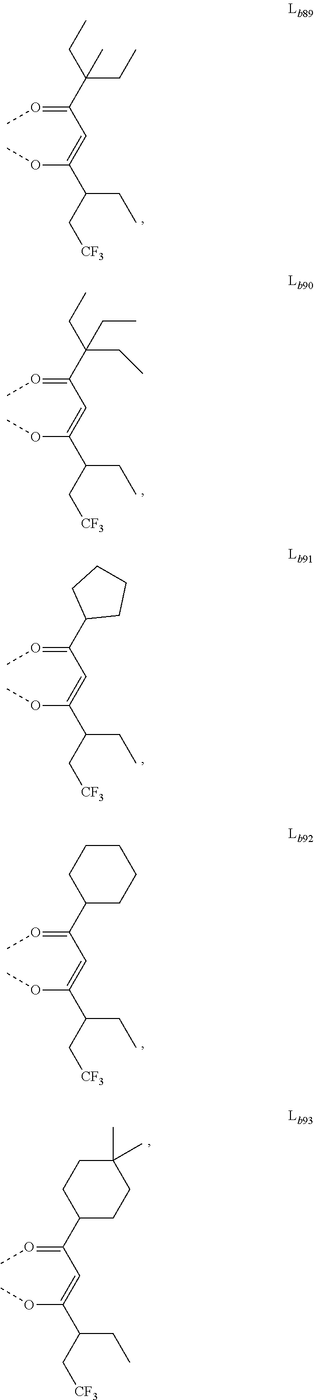 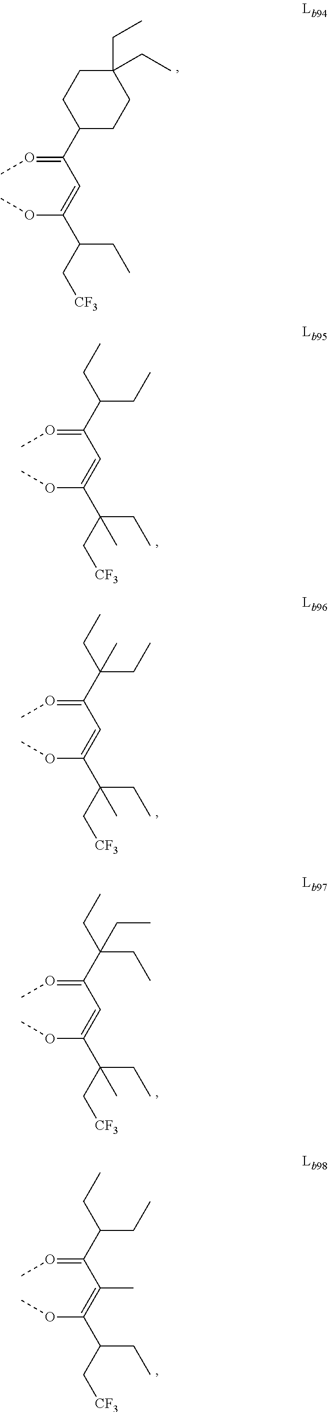 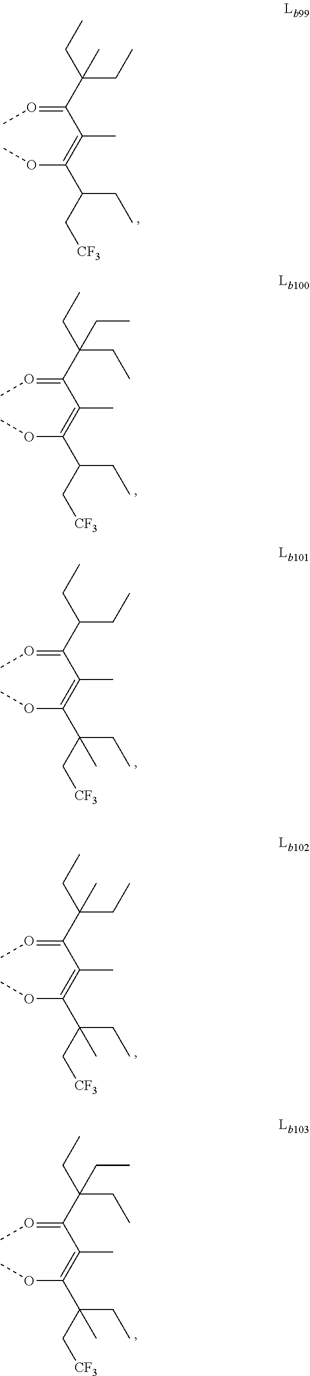 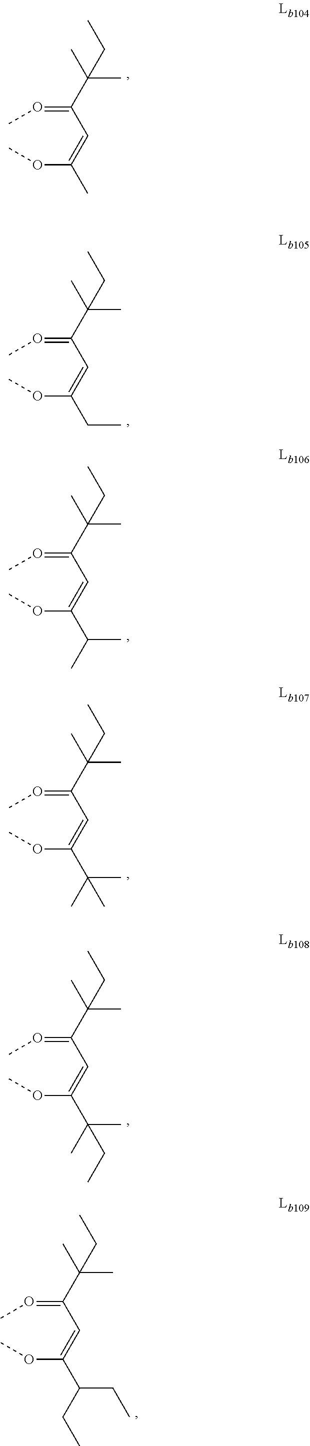 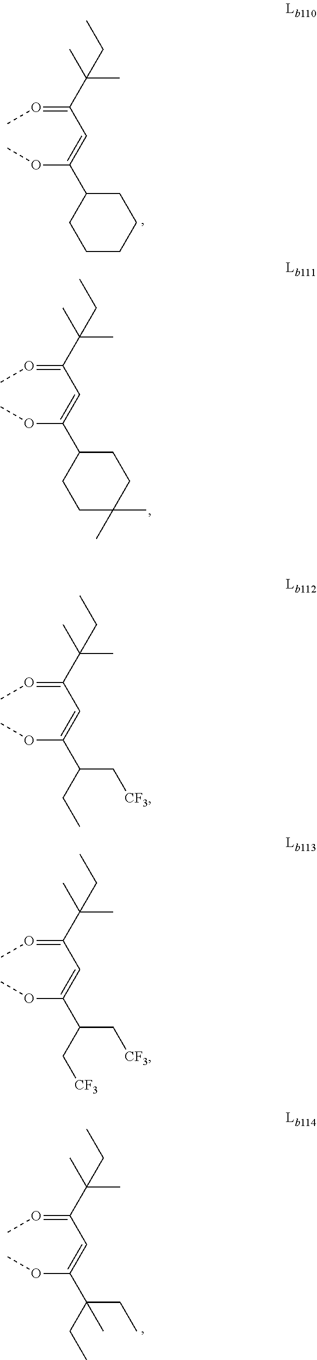 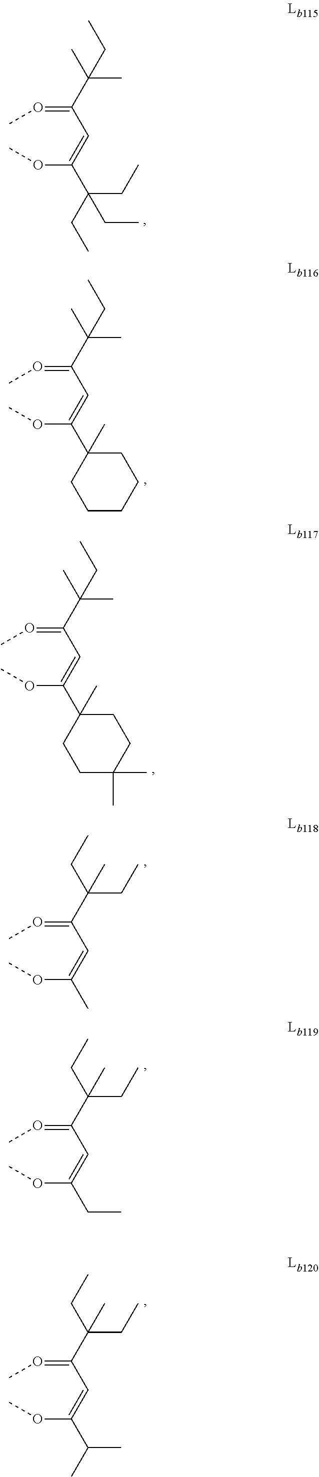 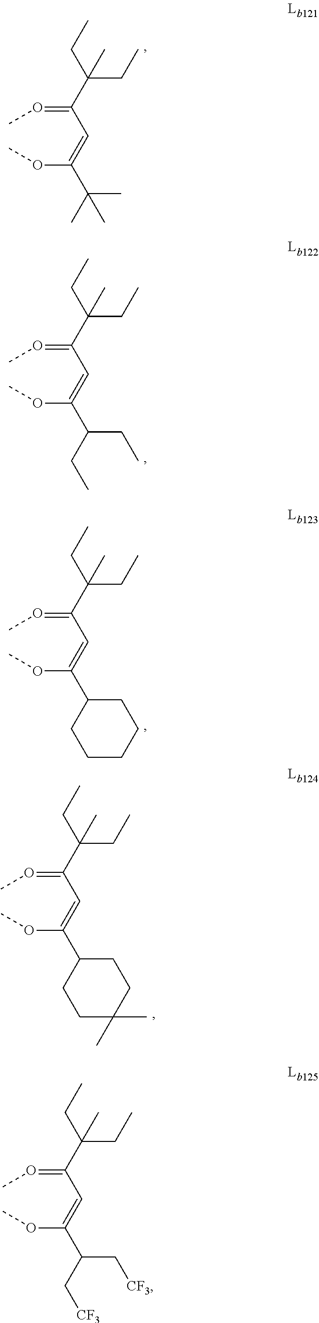 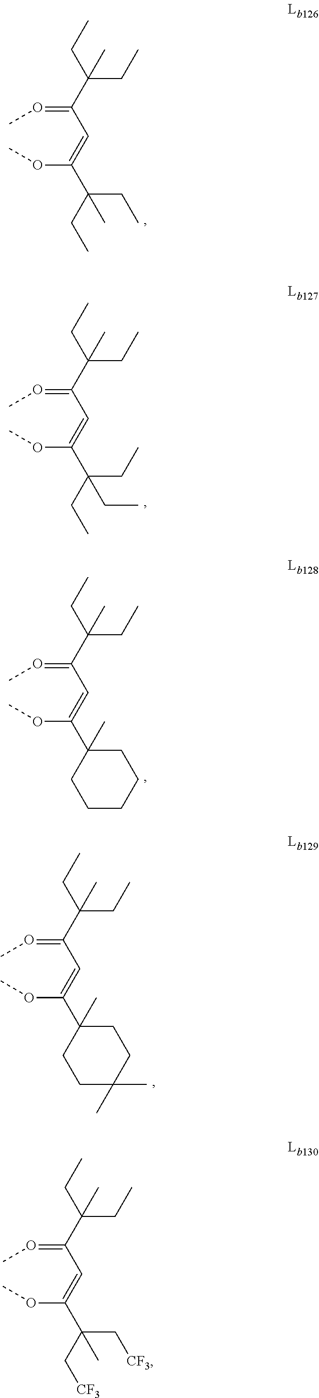 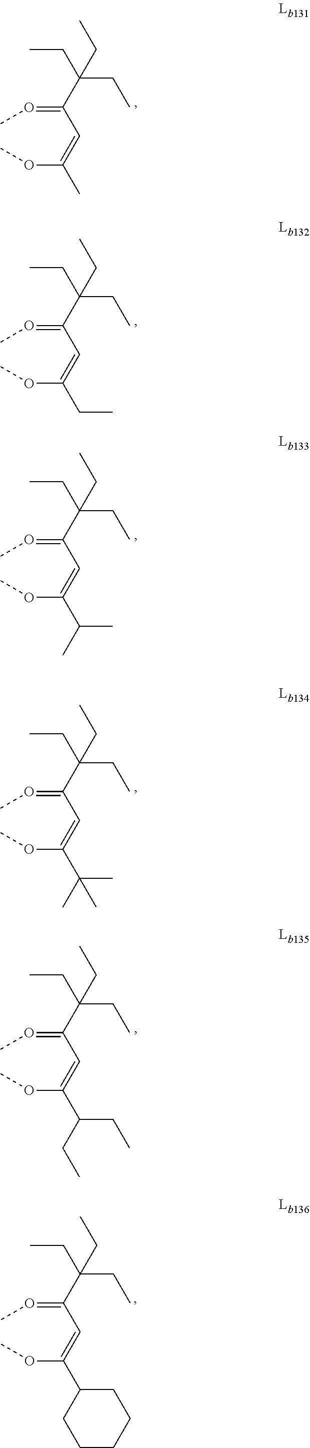 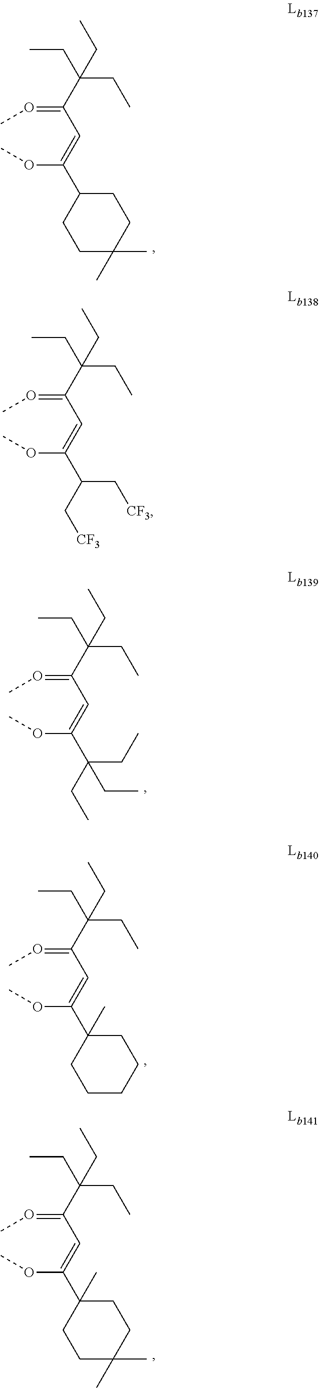 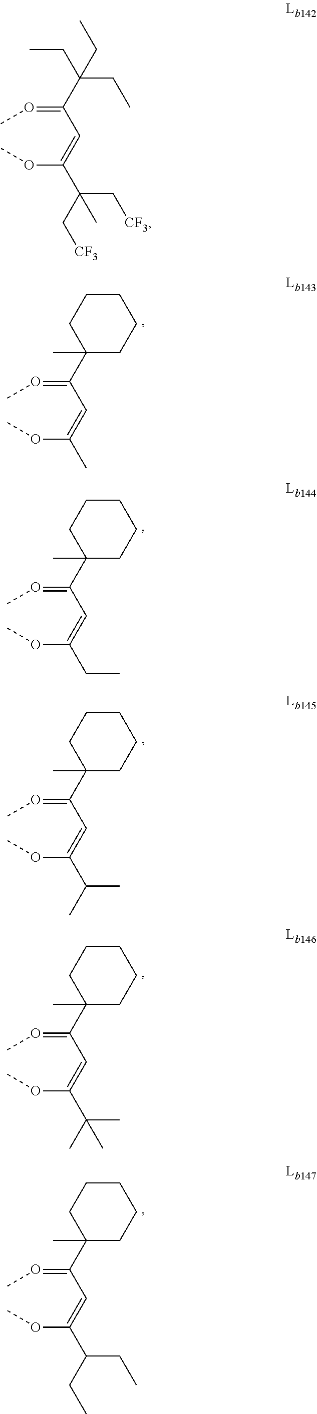 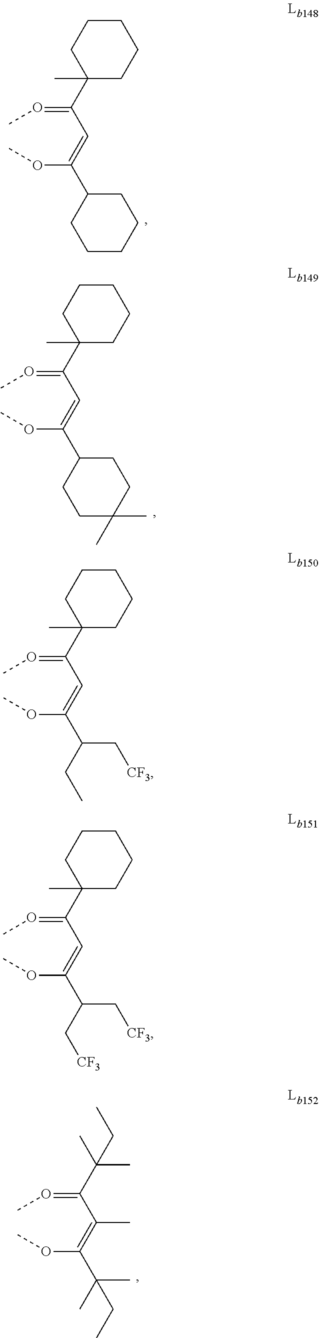 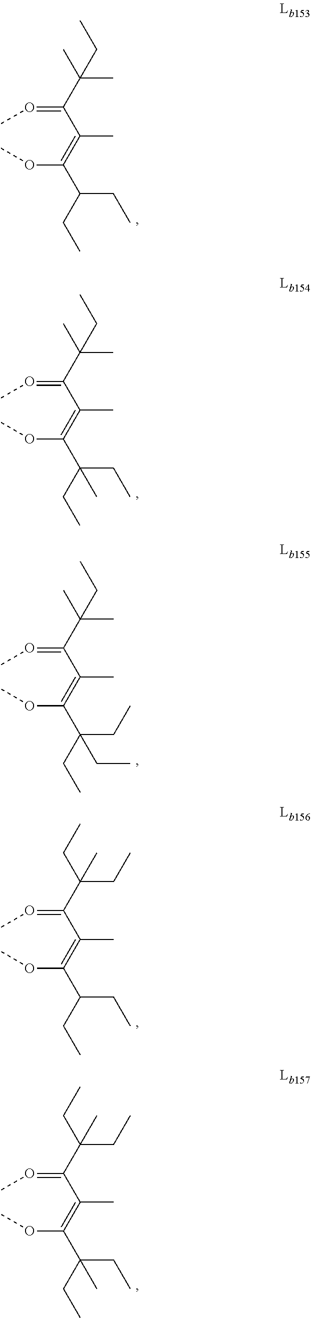 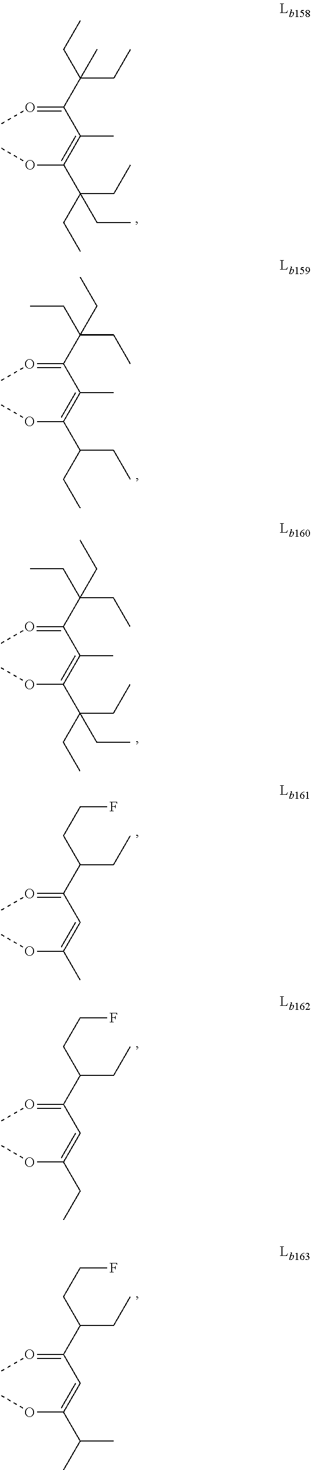 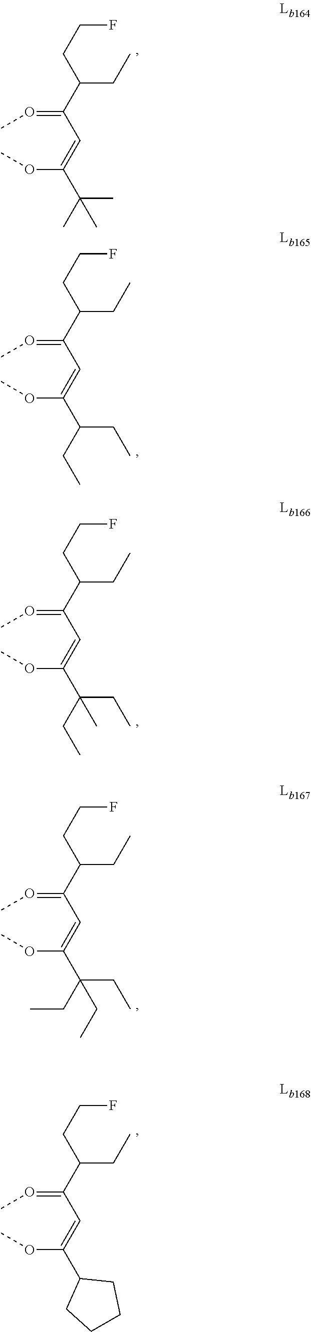 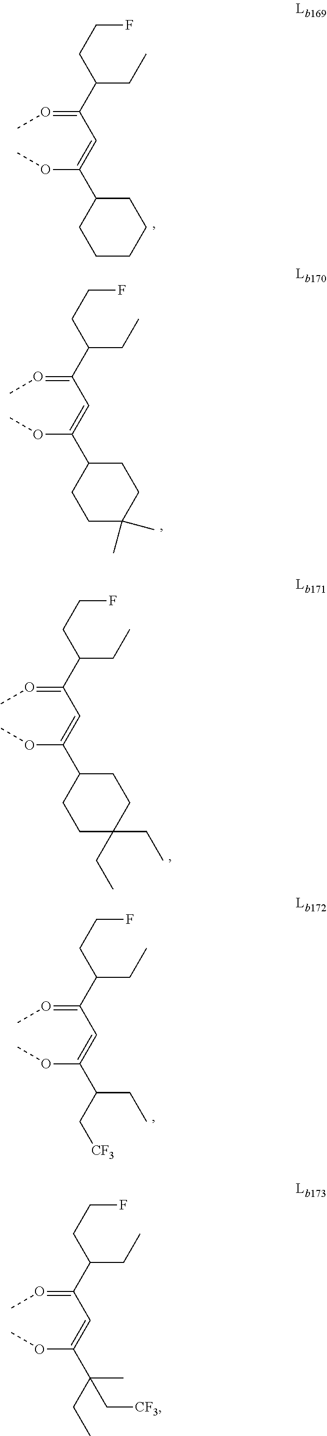 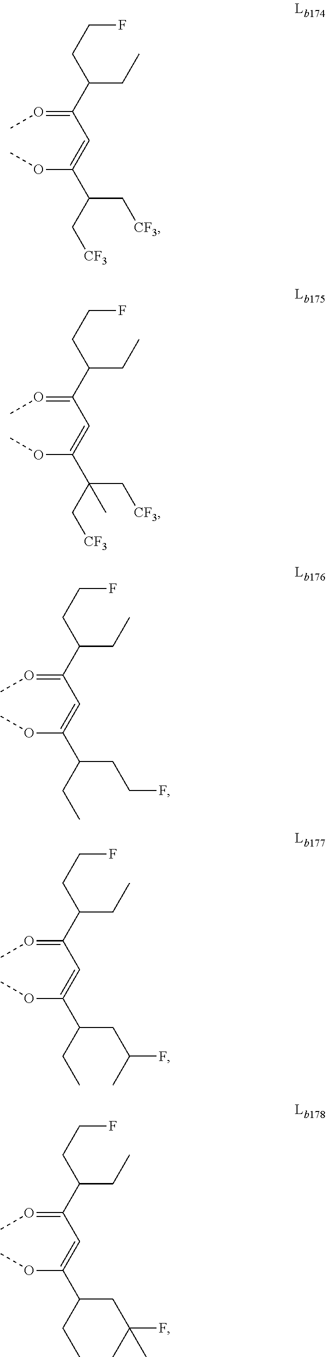 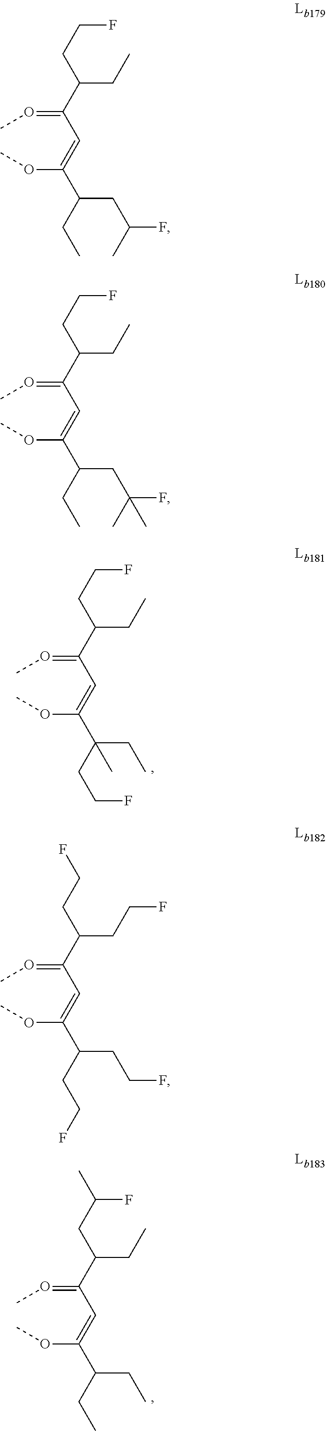 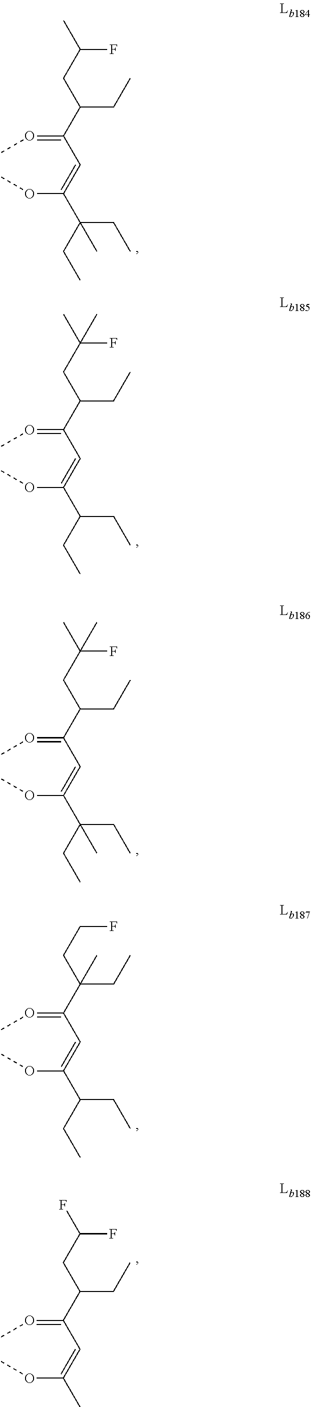 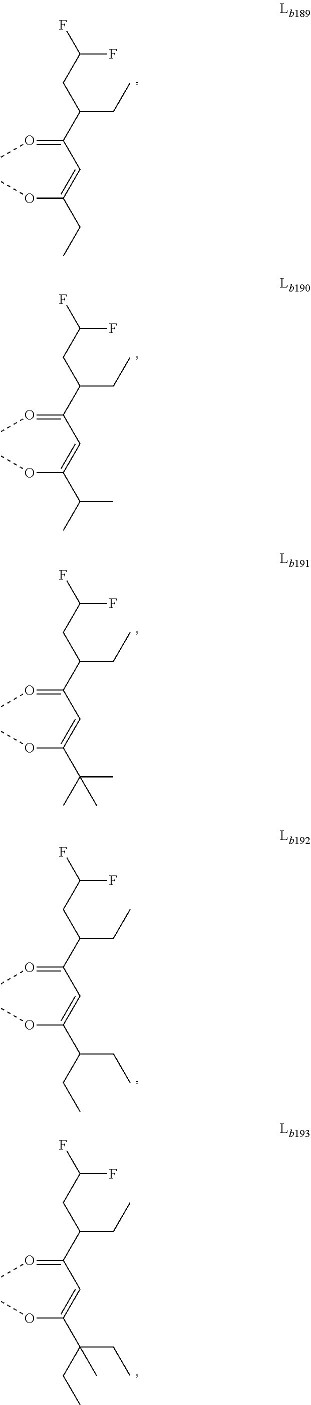 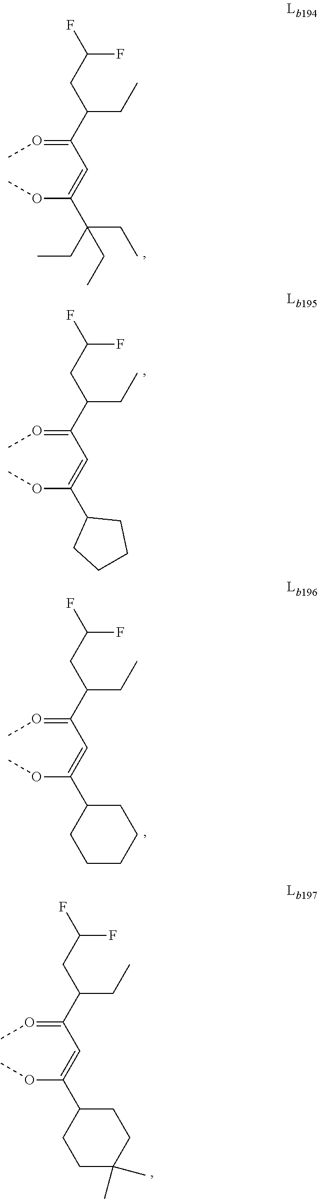 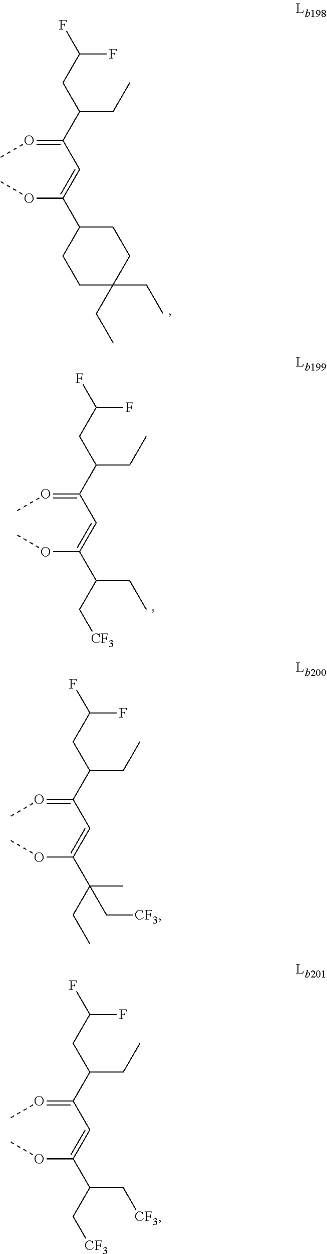 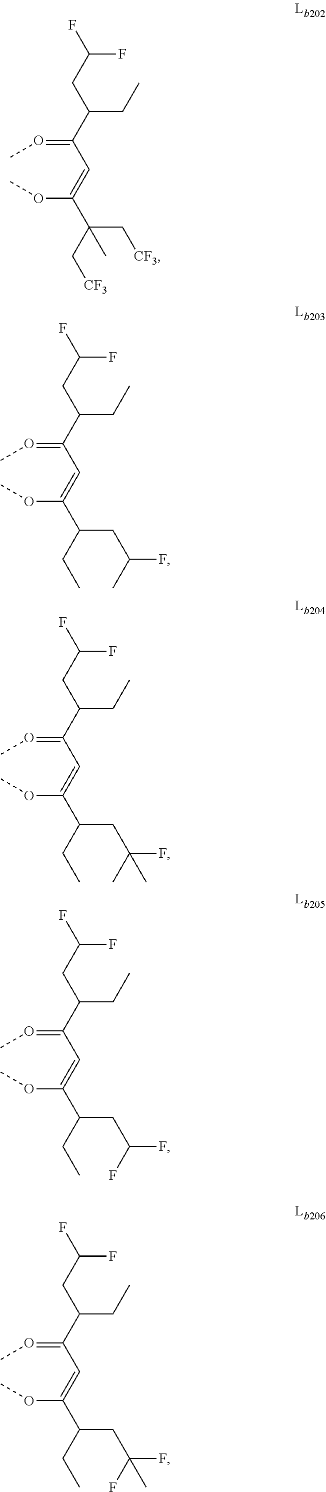 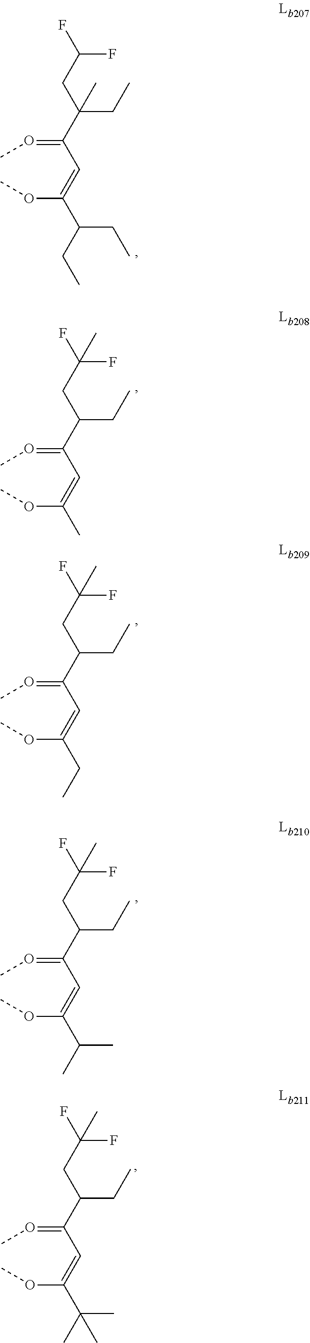 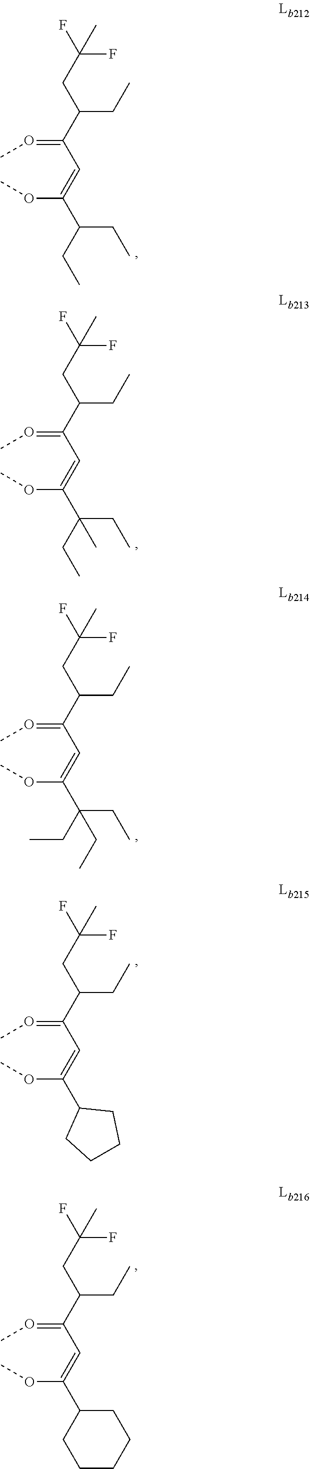 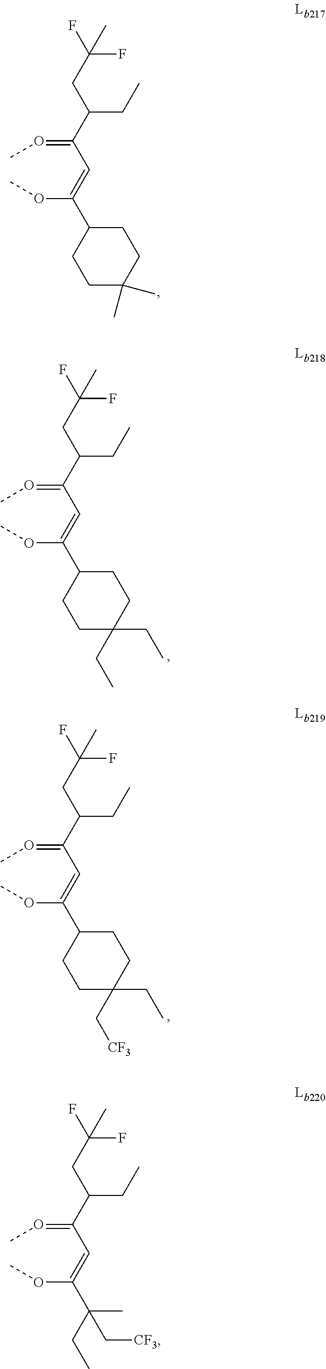 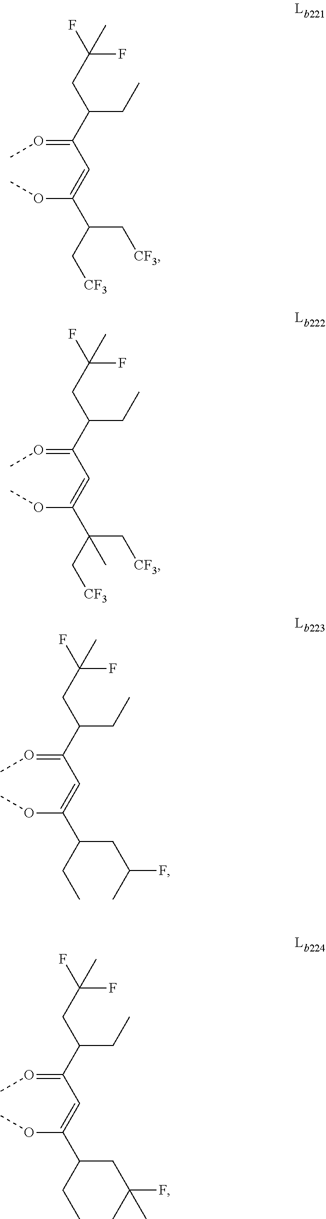 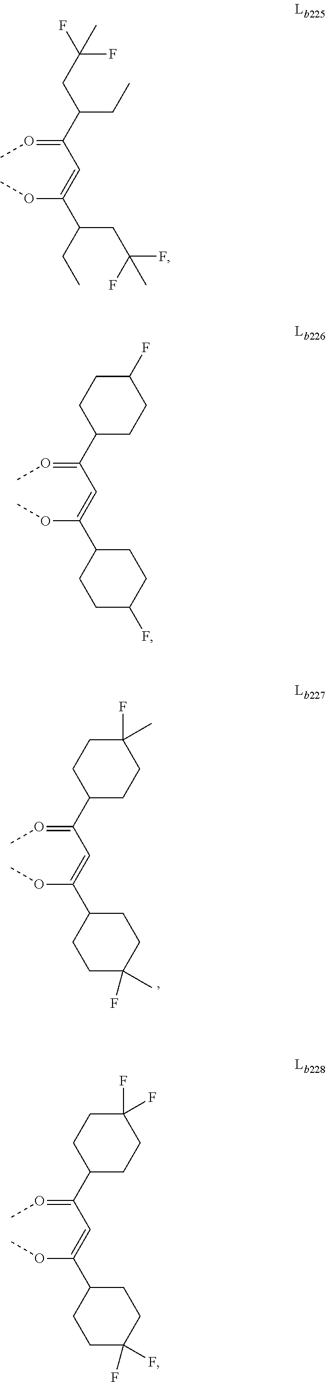 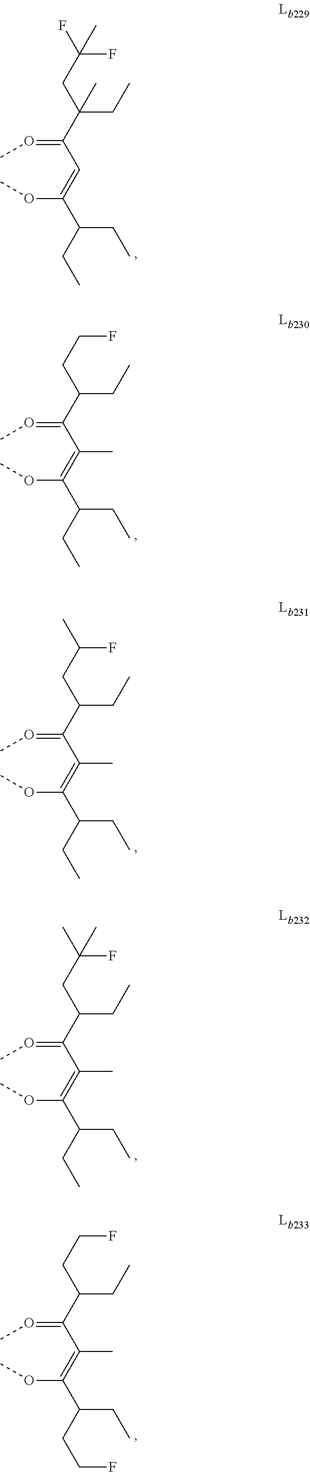 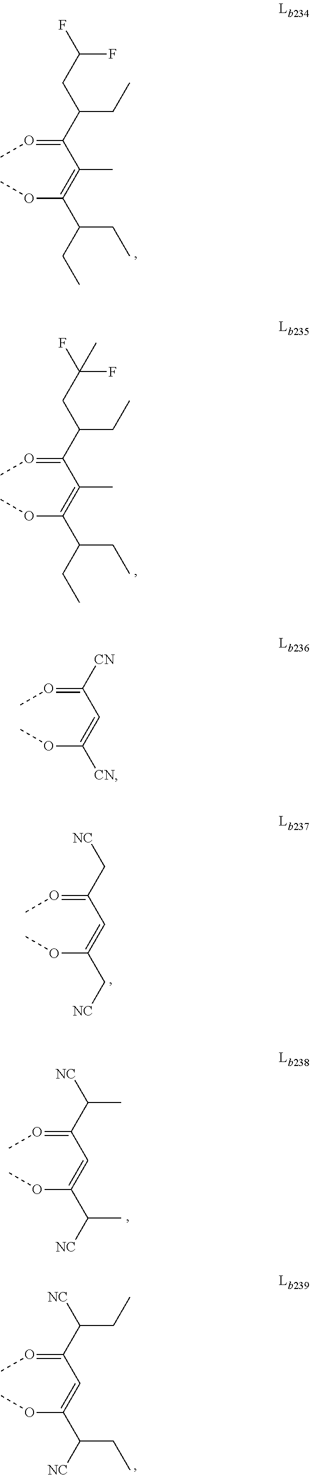 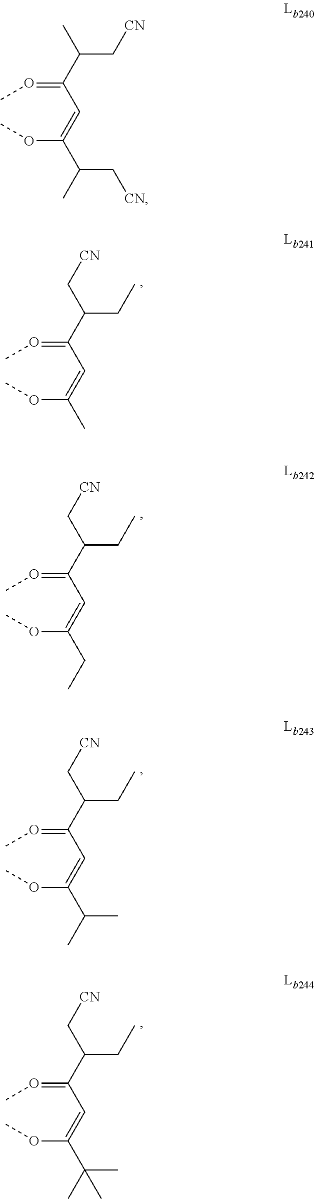 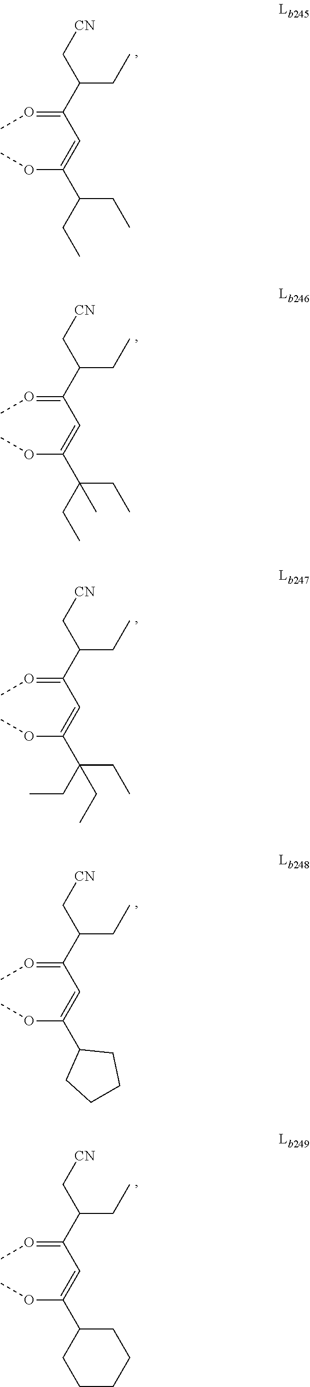 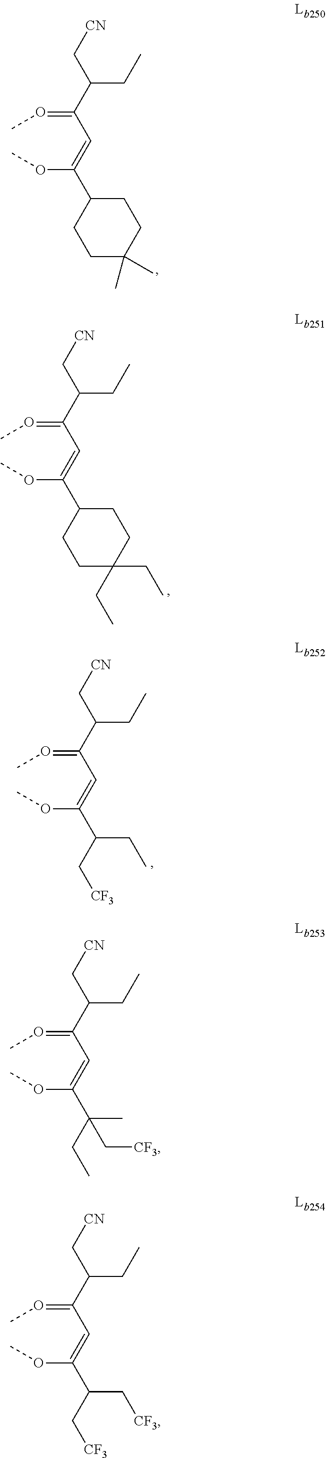 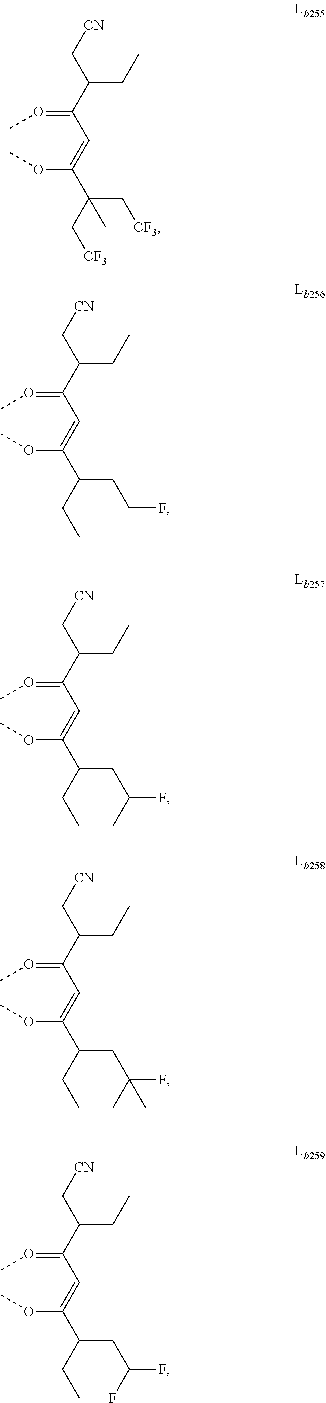 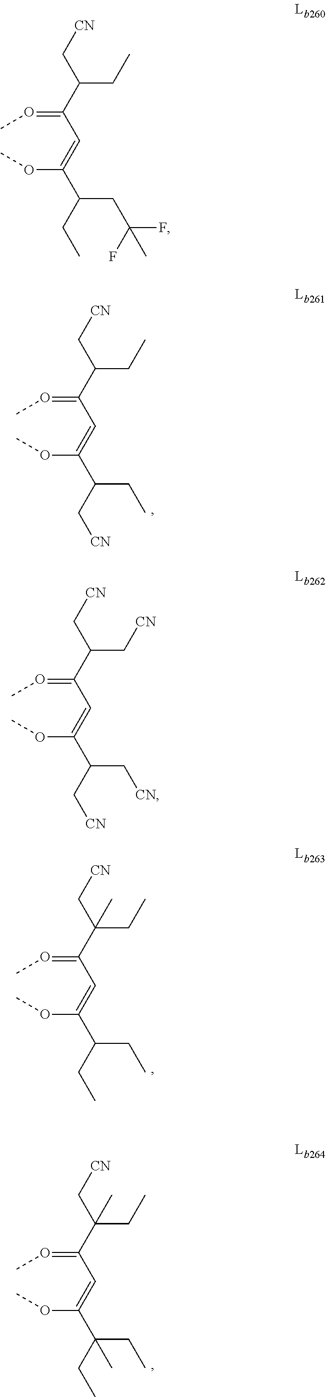 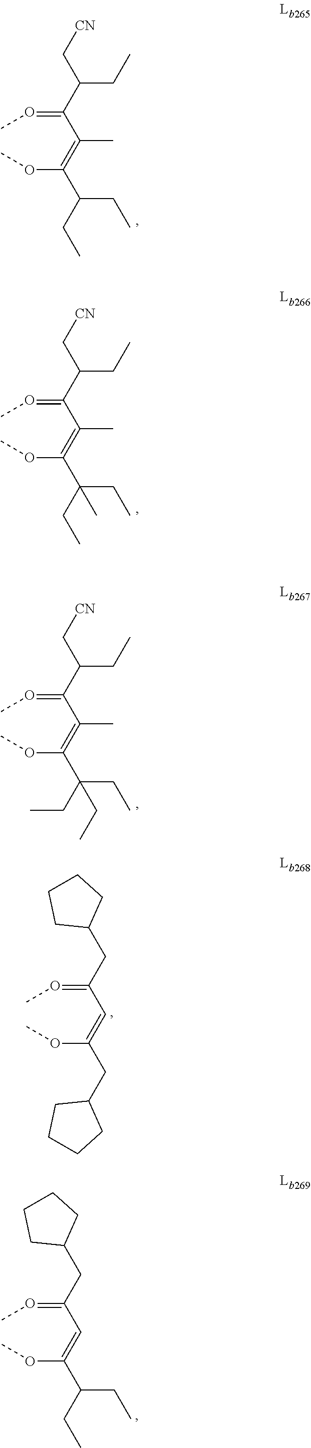 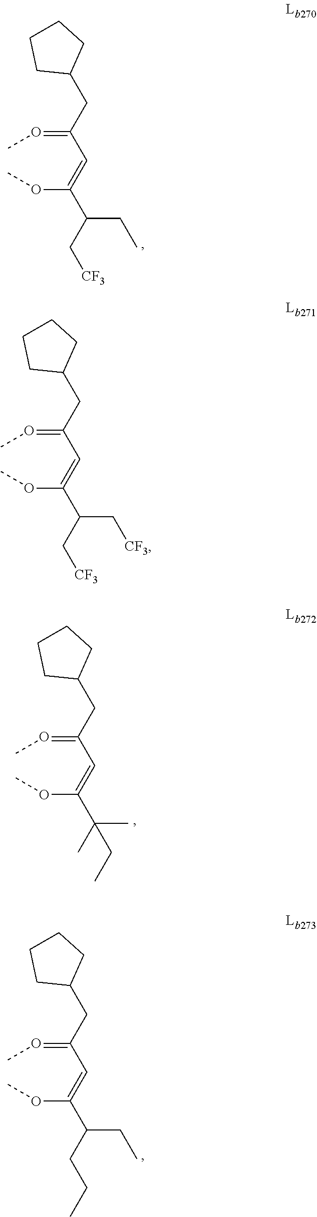 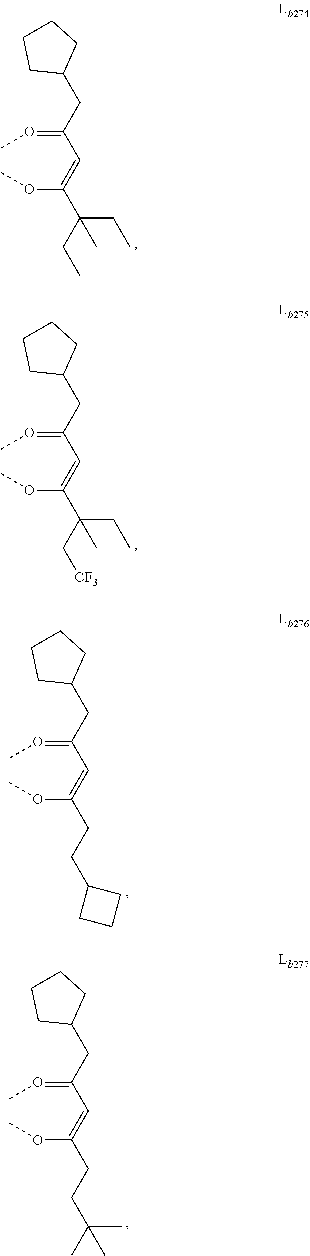 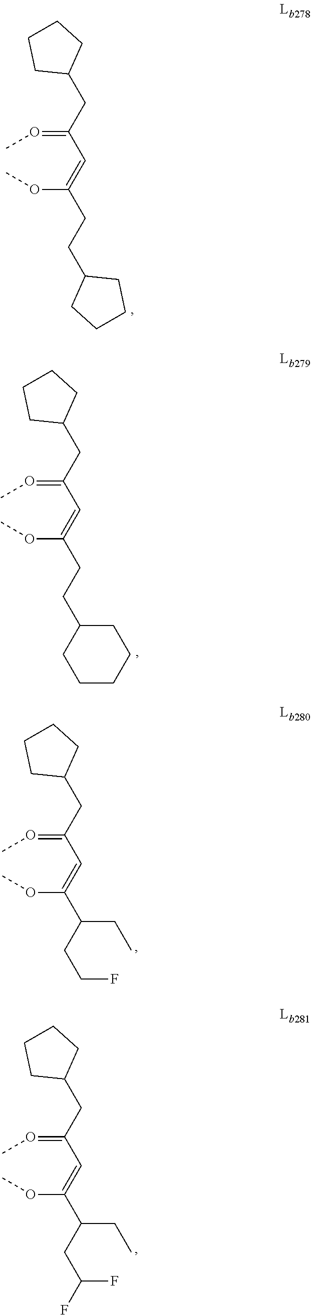 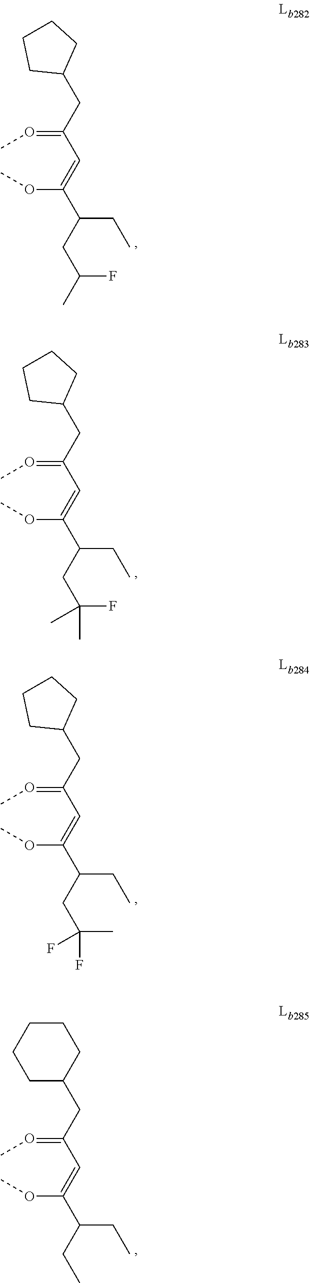 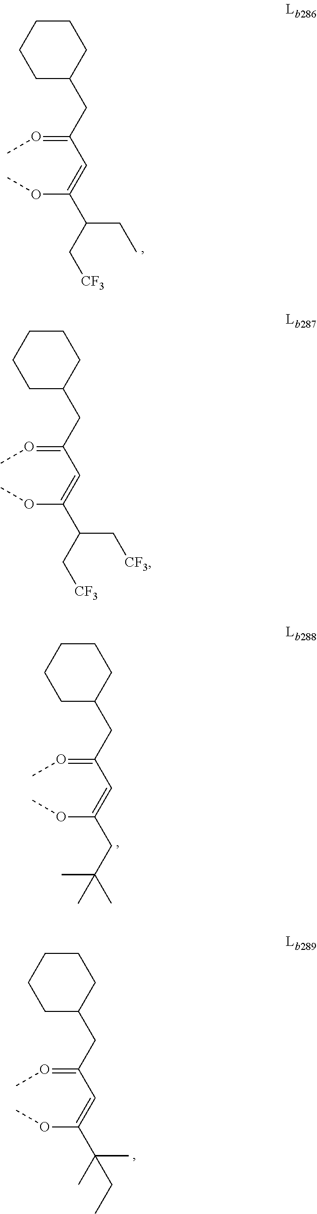 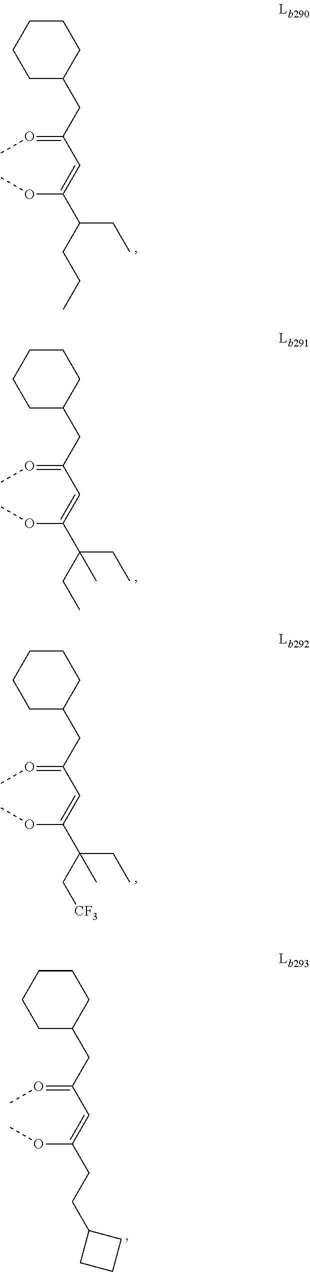 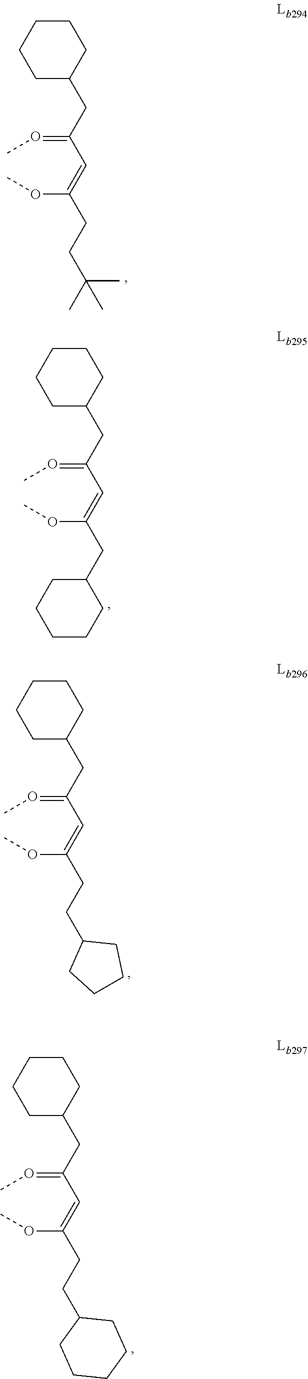 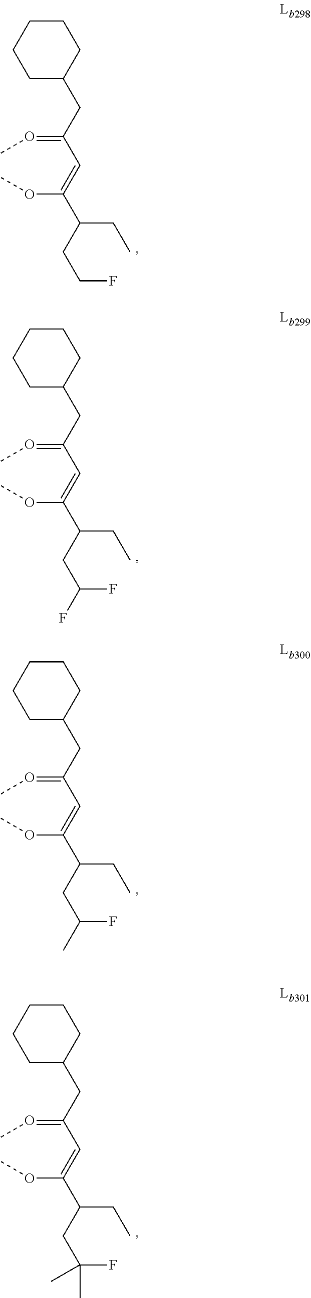 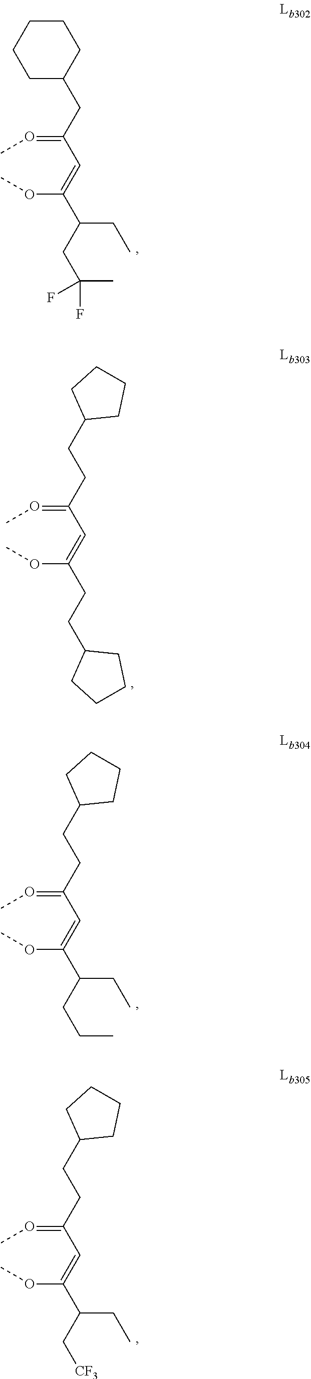 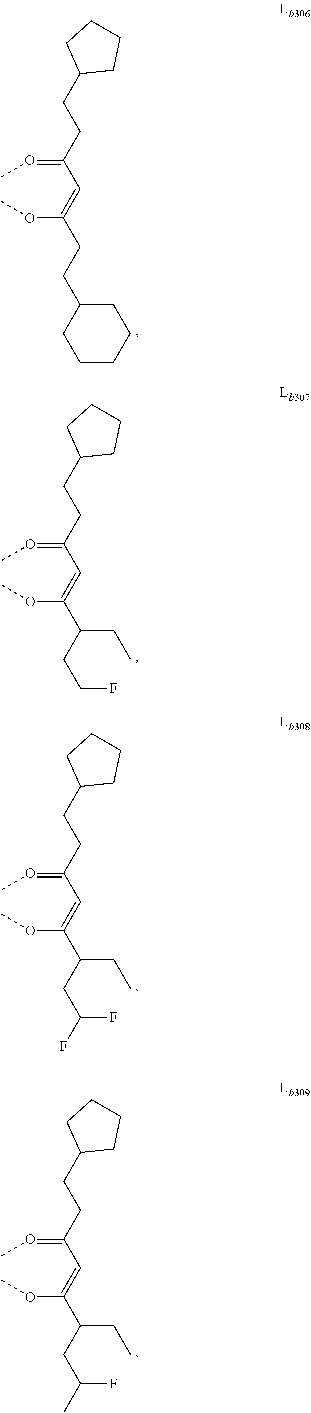 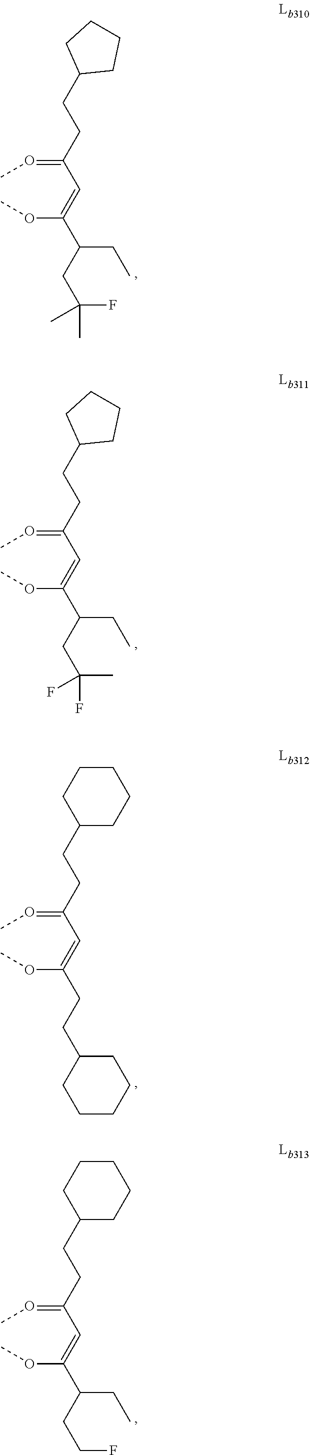 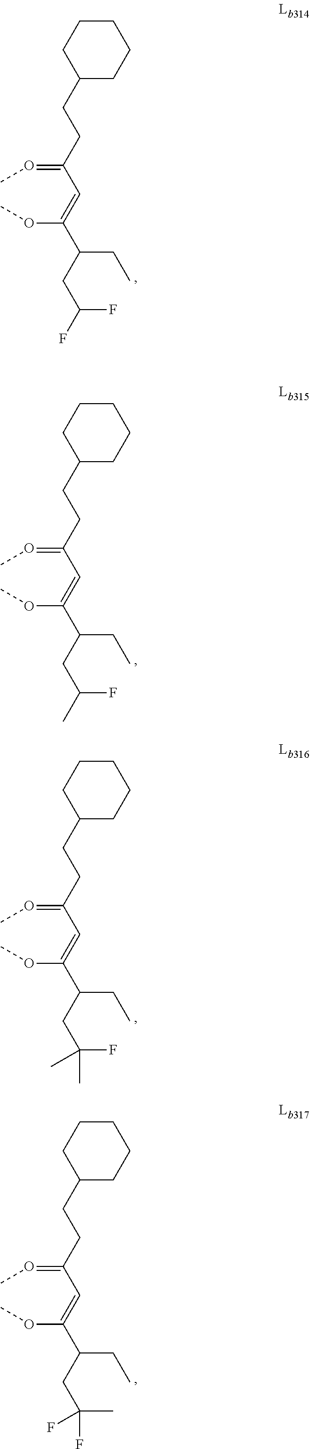 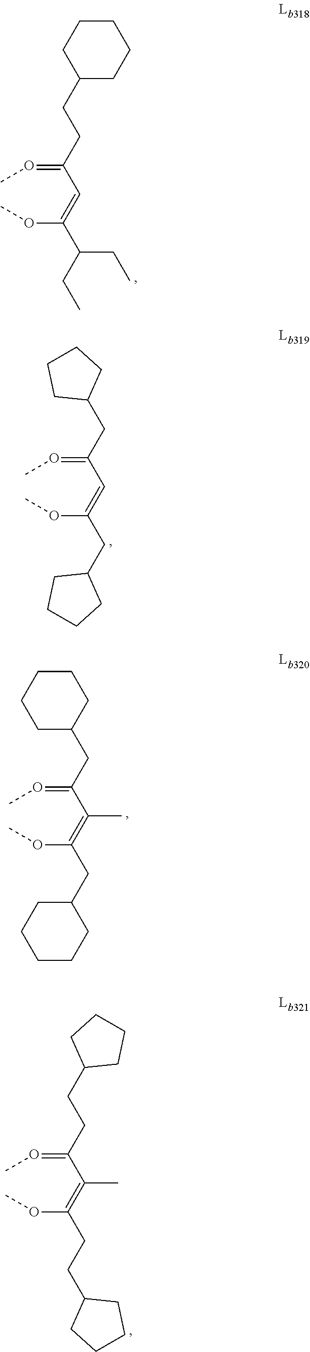 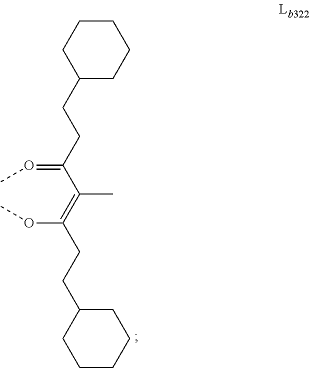
wherein L_{c} is, at each occurrence identically or differently, selected from the group consisting of the following structures:

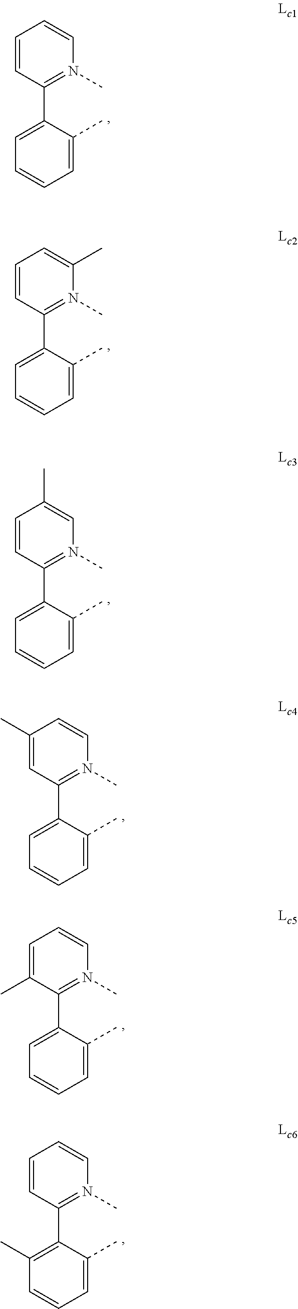

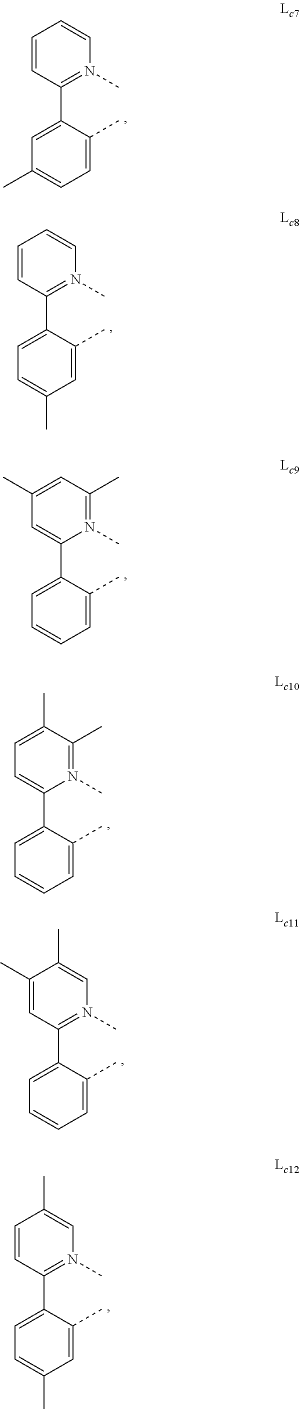

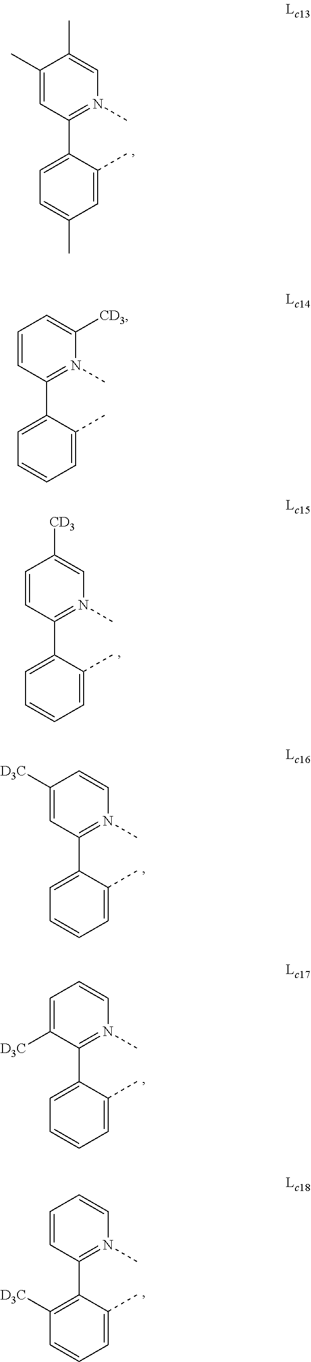

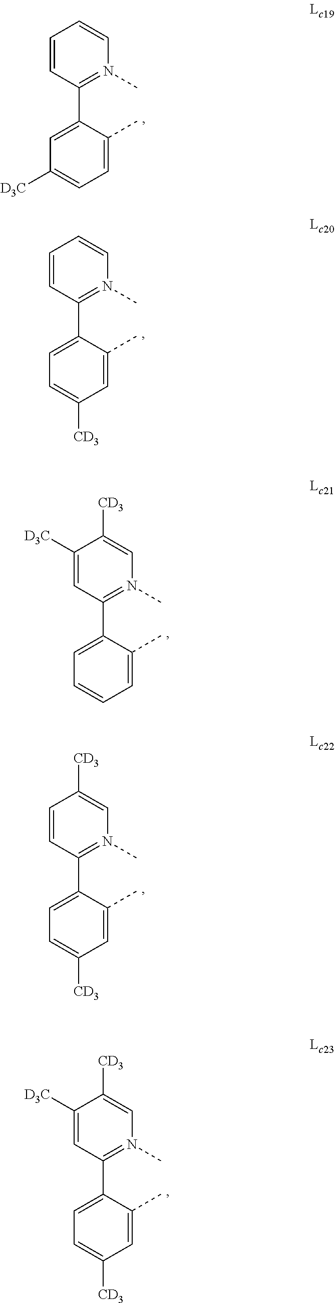

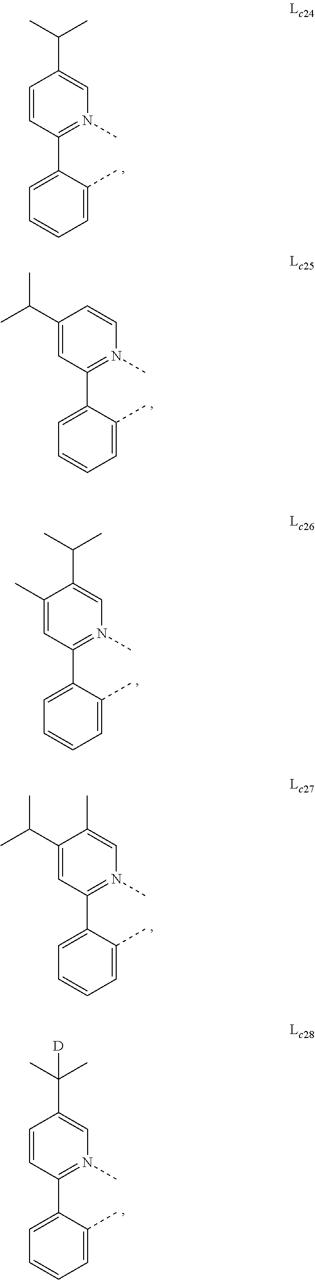

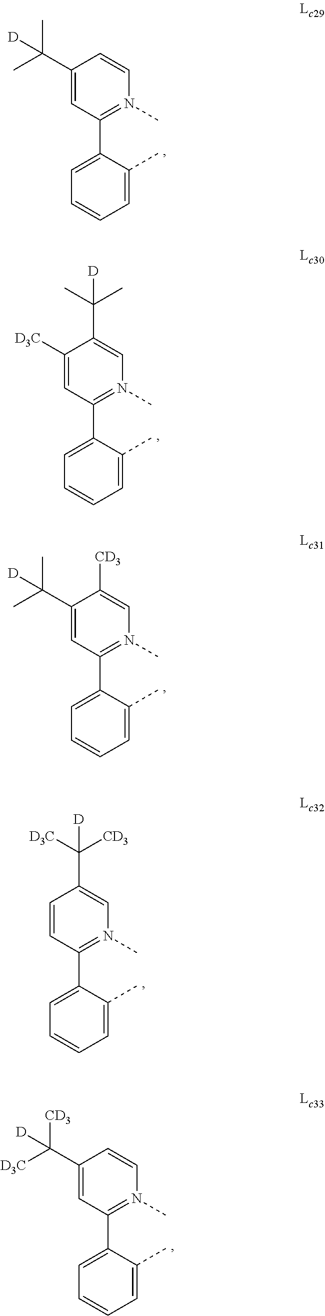

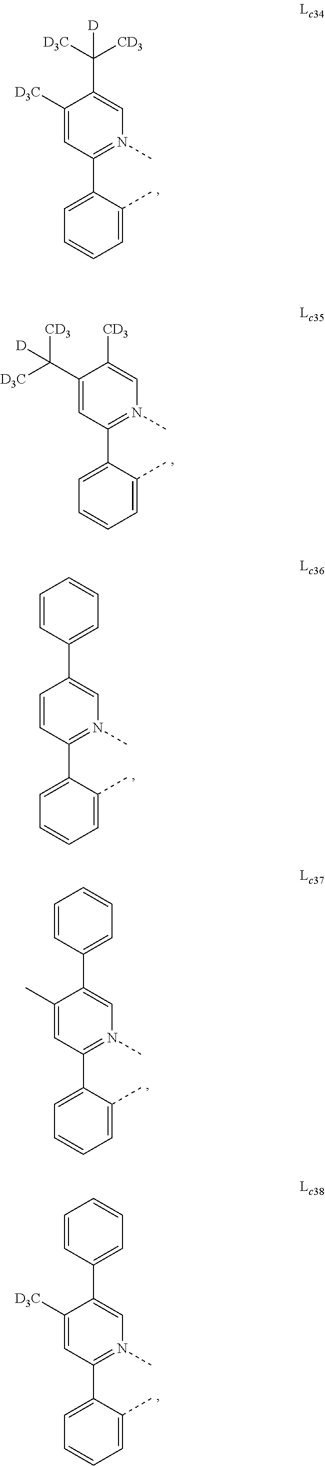

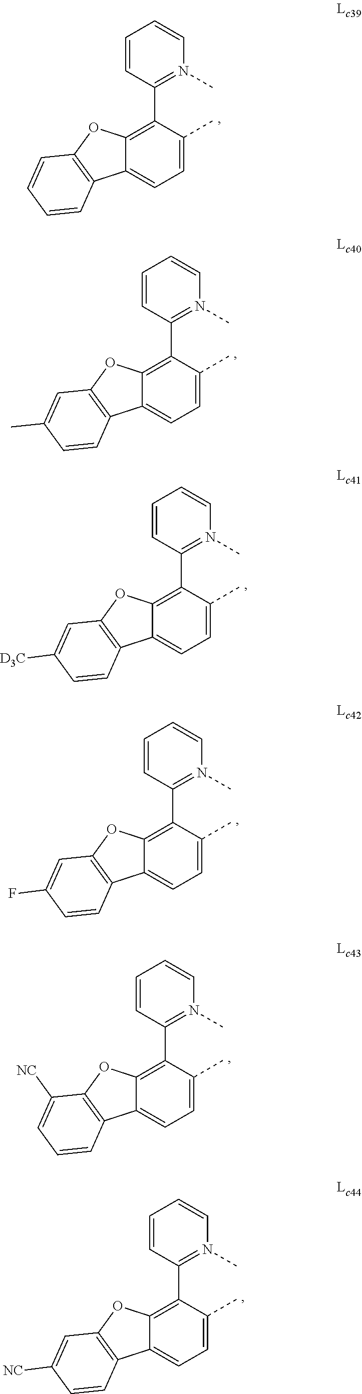

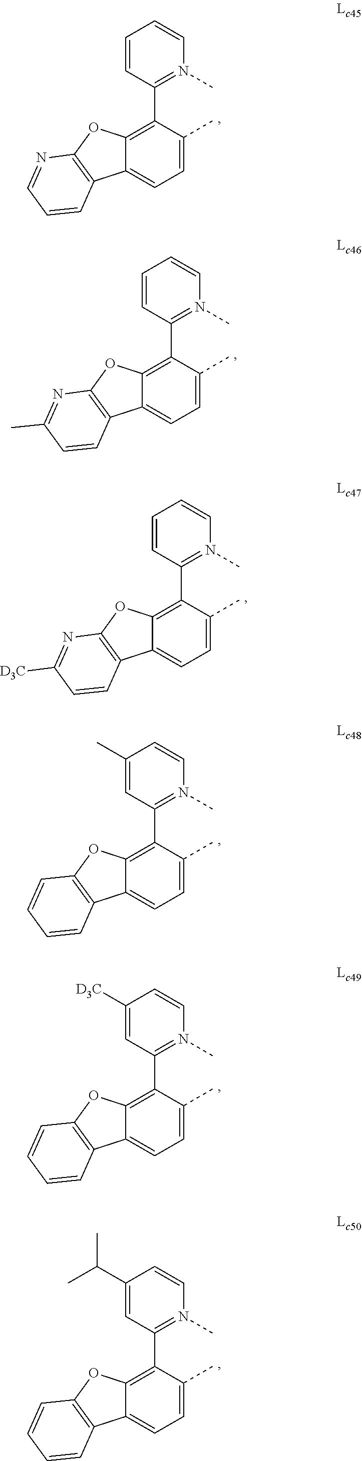

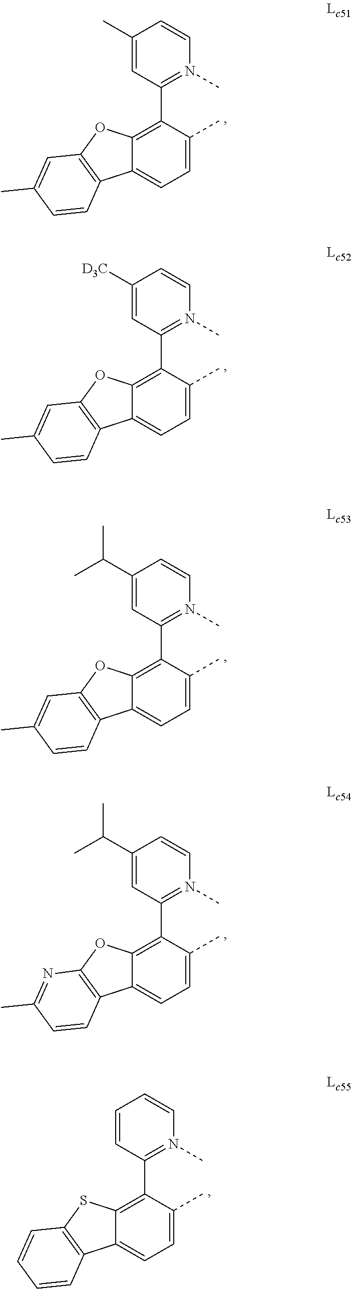

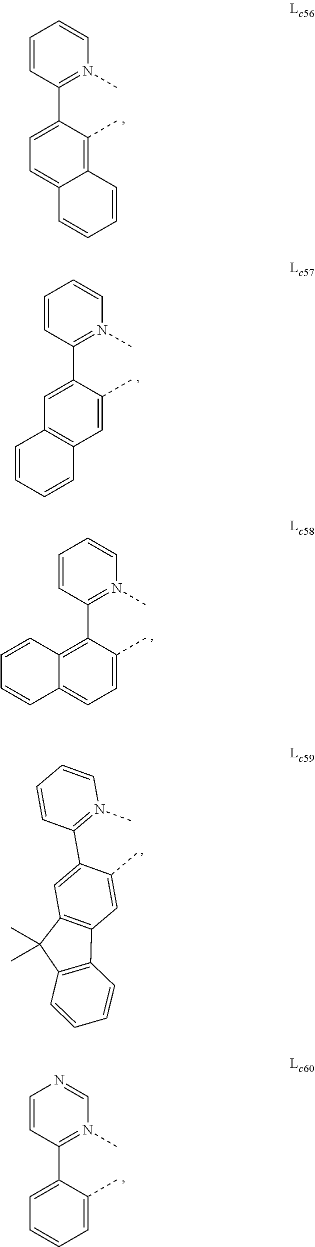

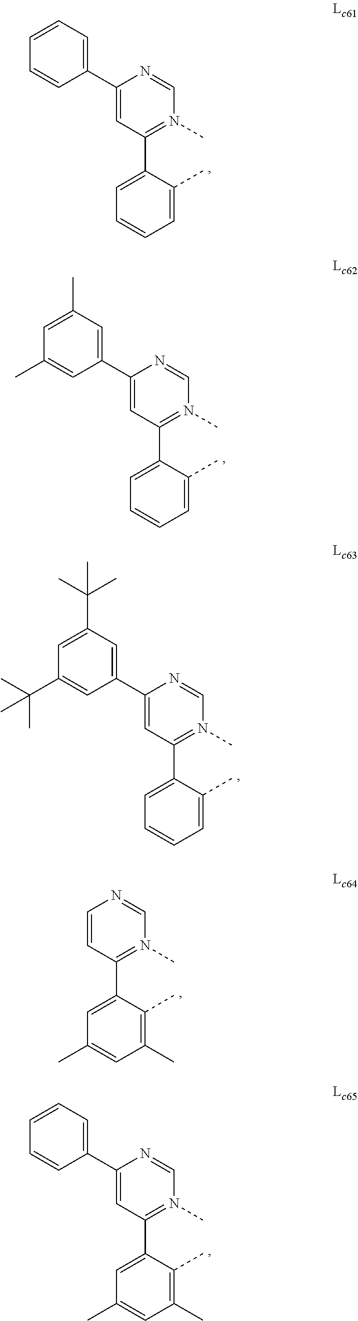

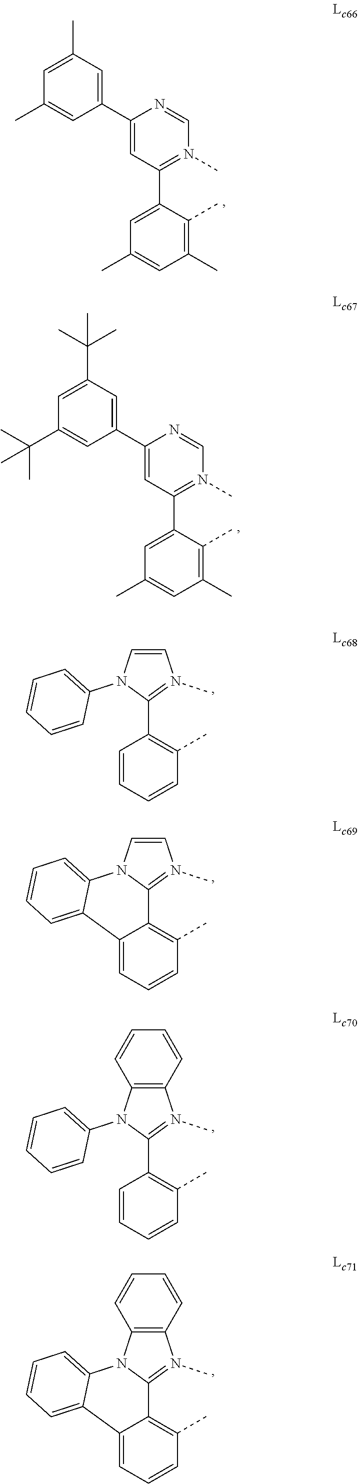

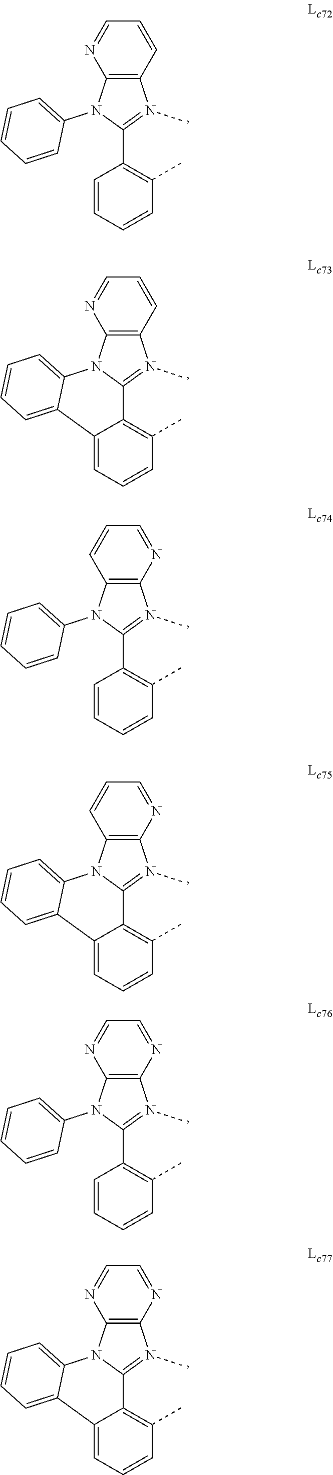

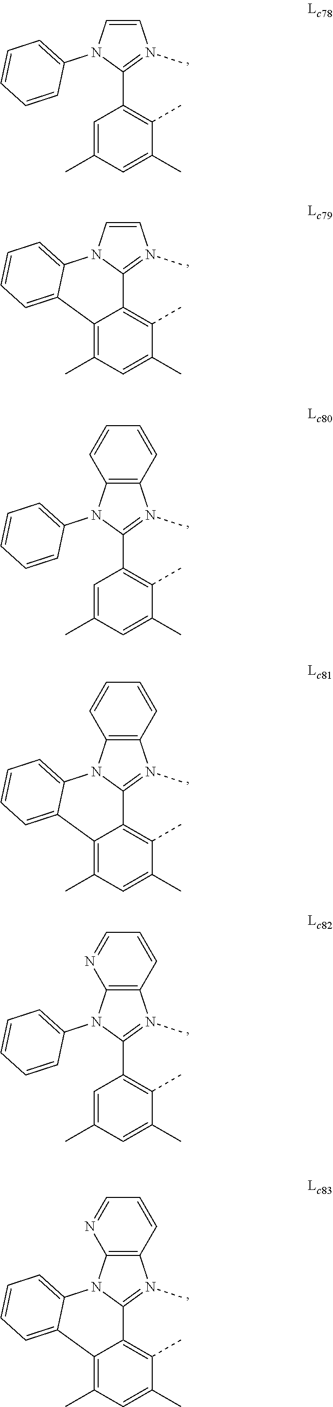

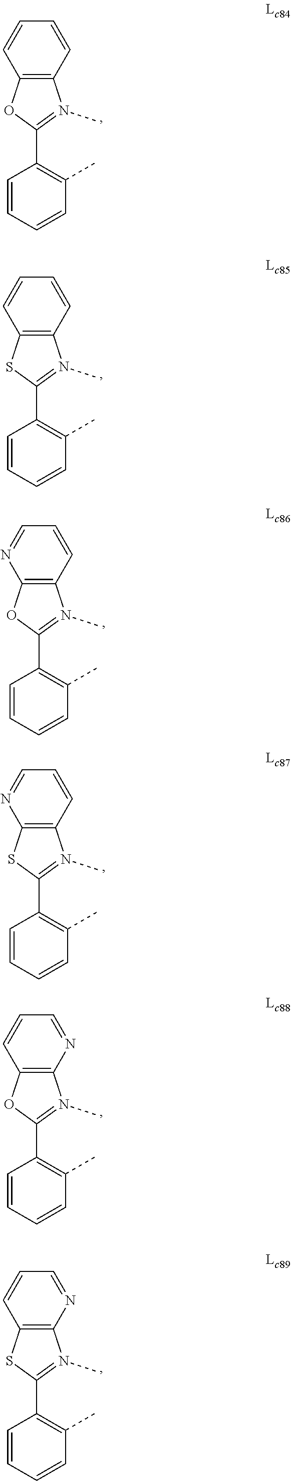

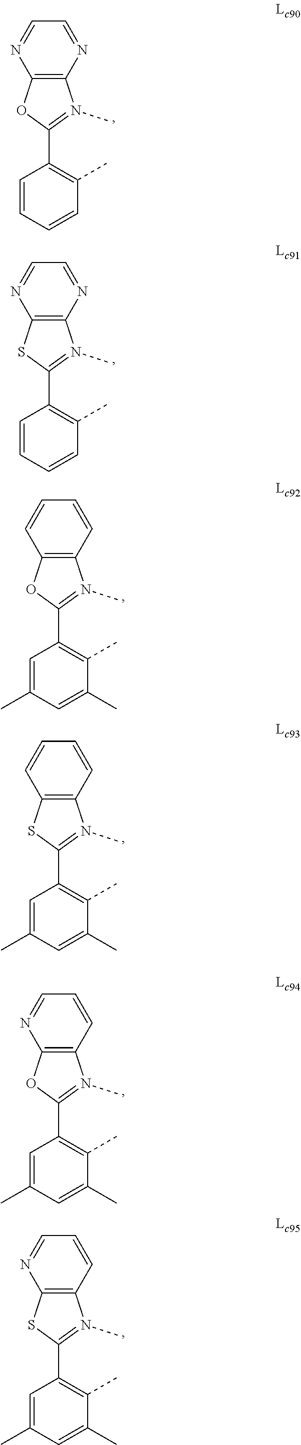

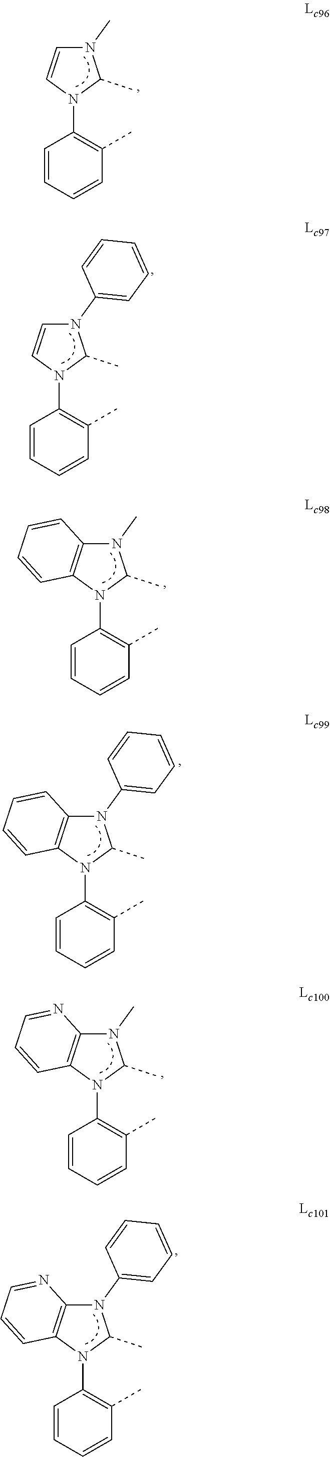

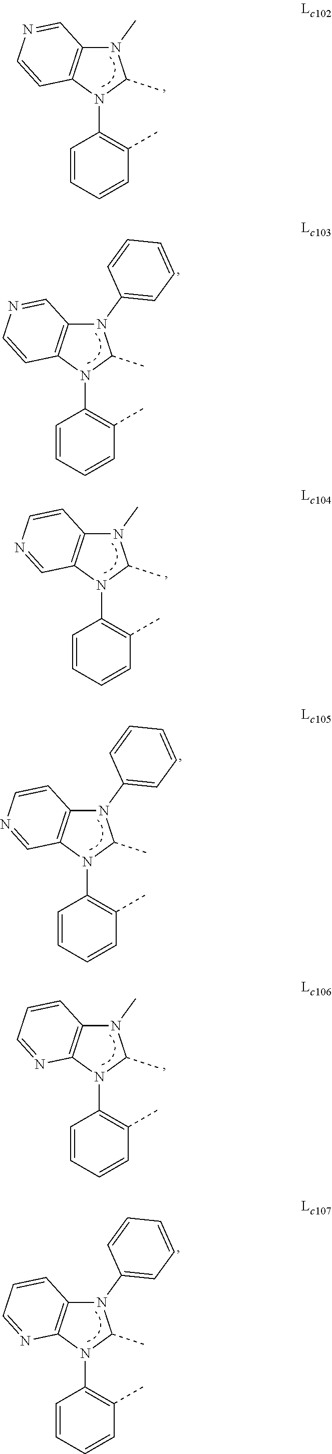

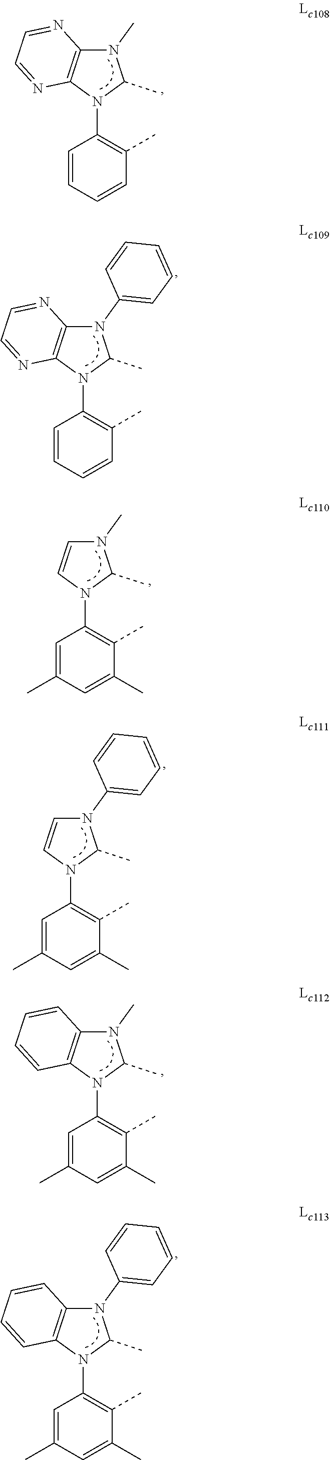

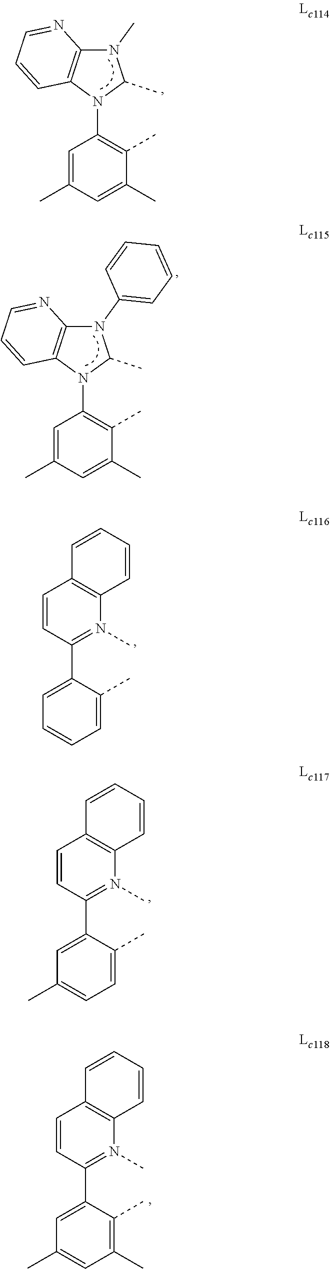

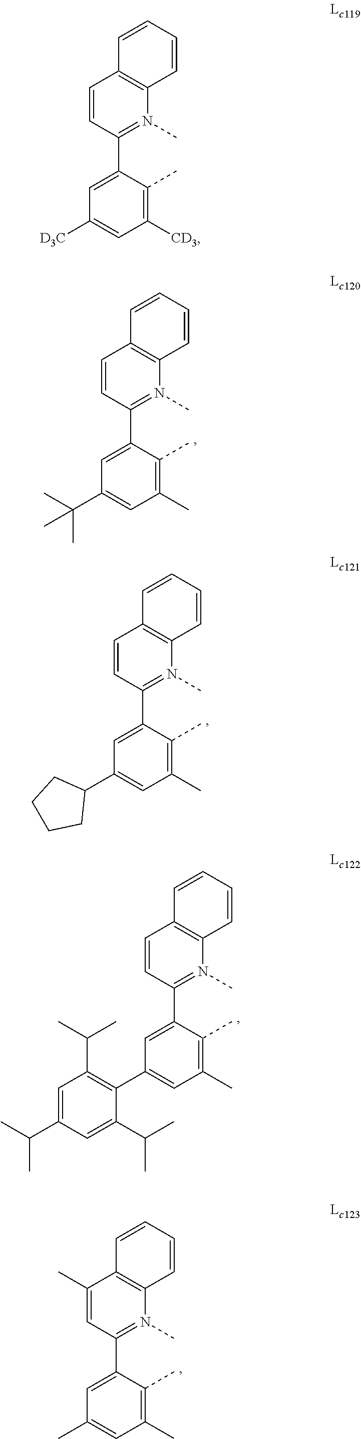

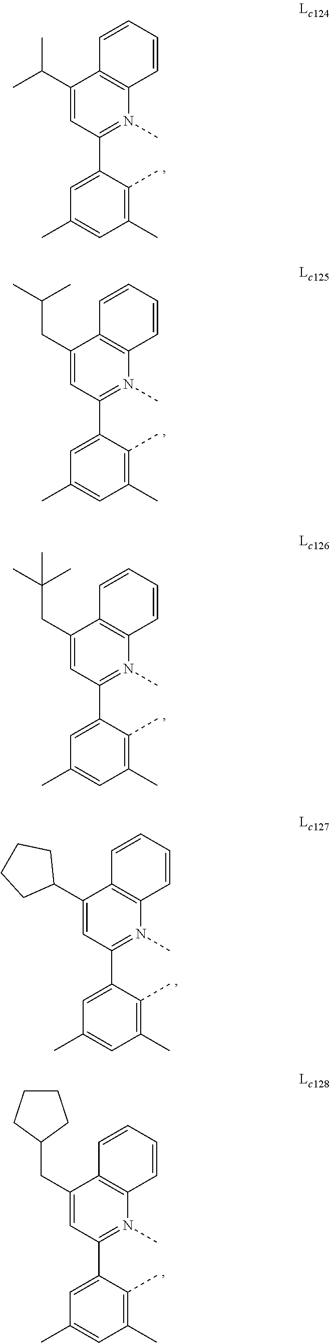

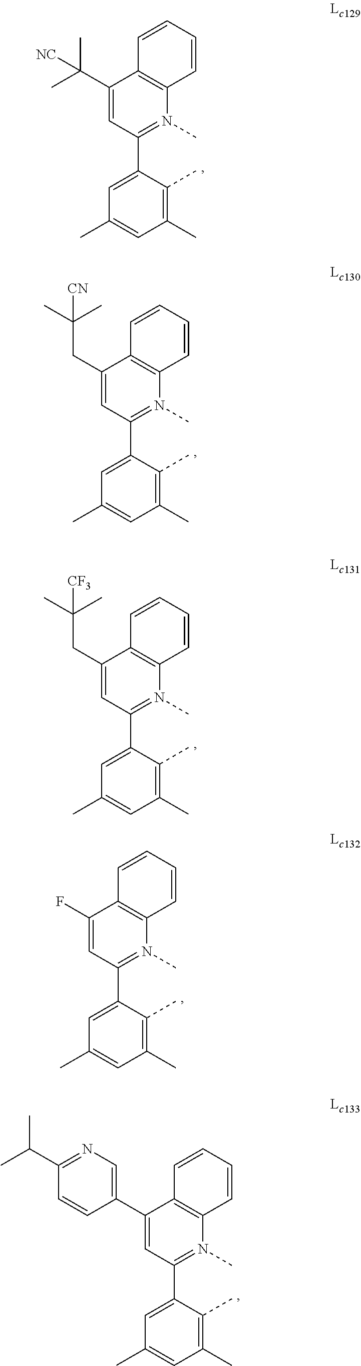

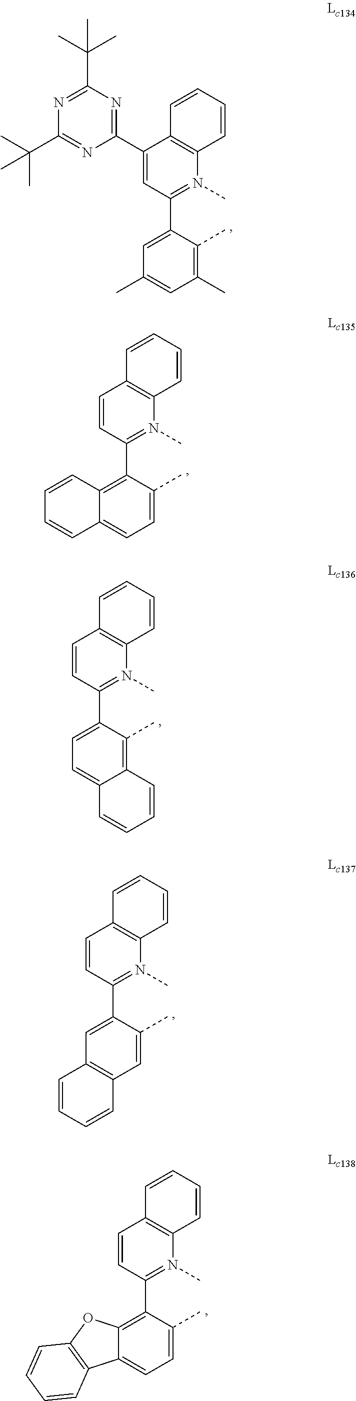

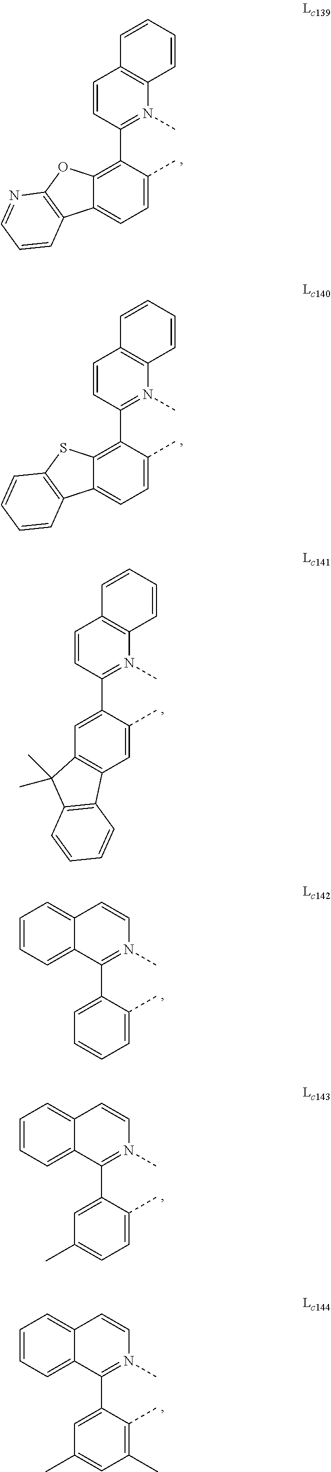

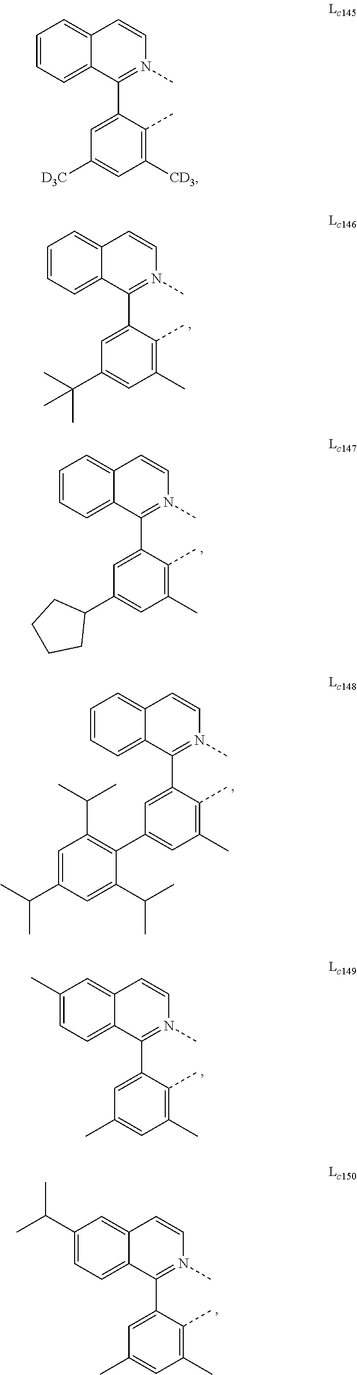

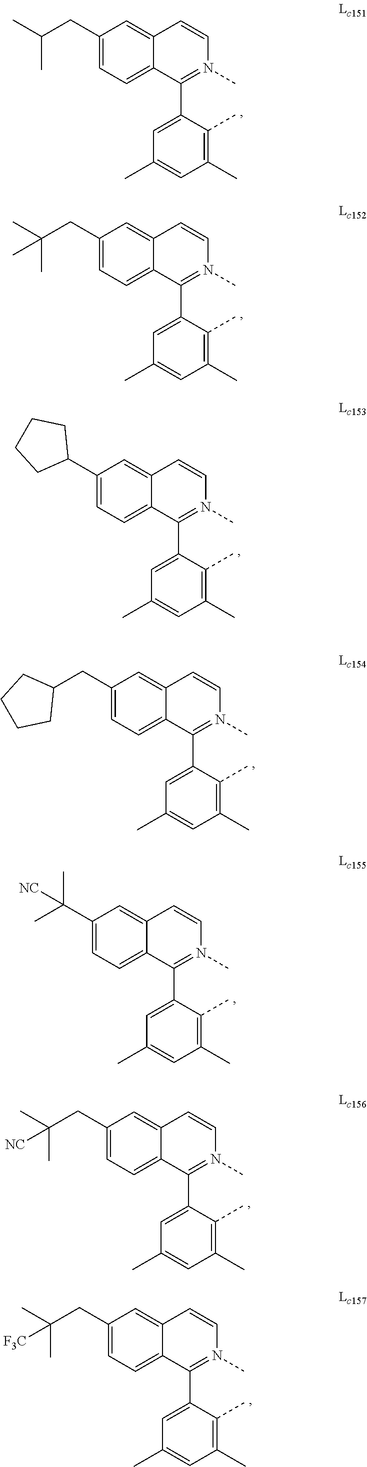

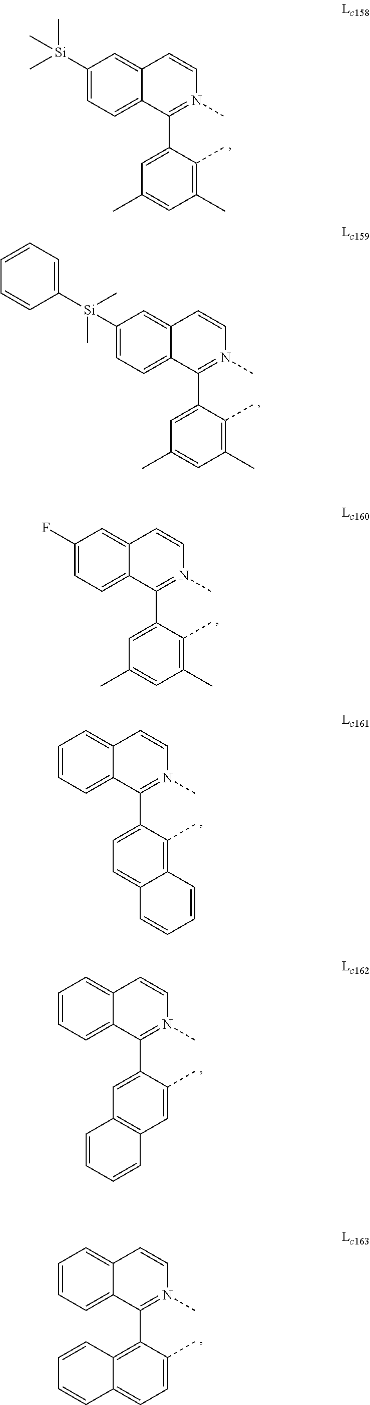

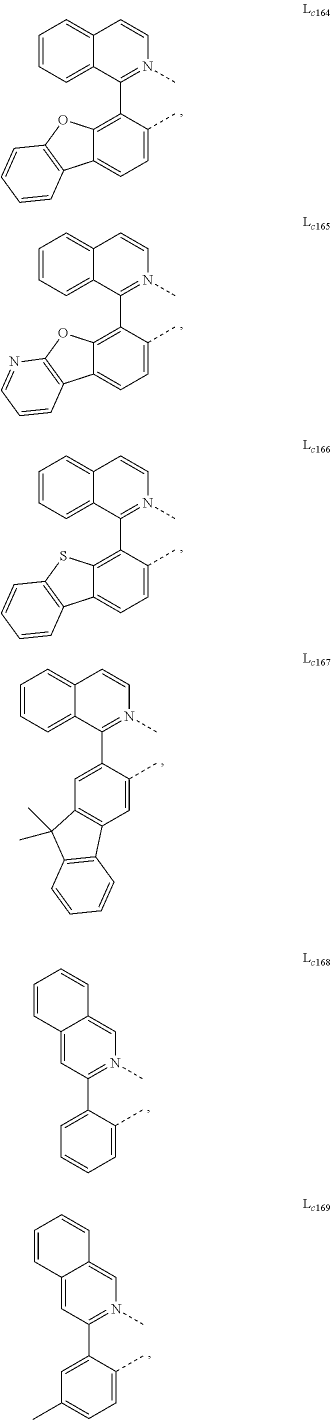

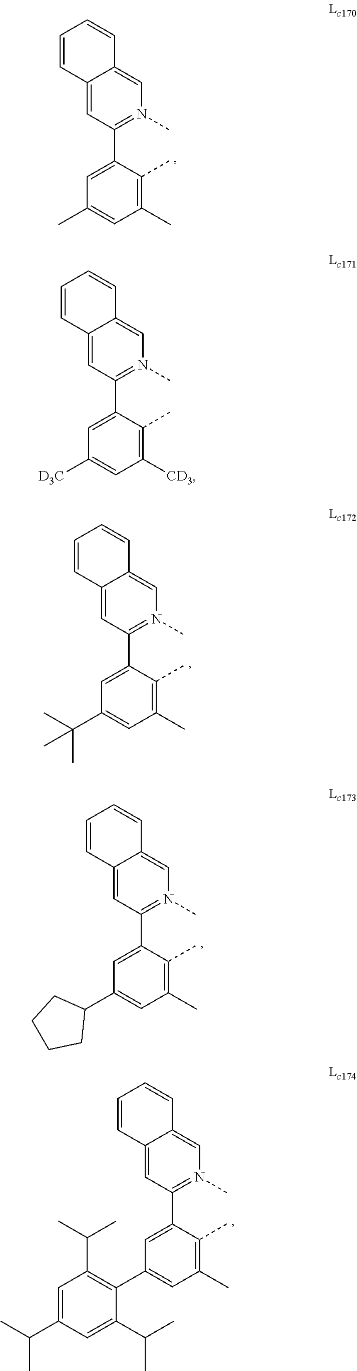

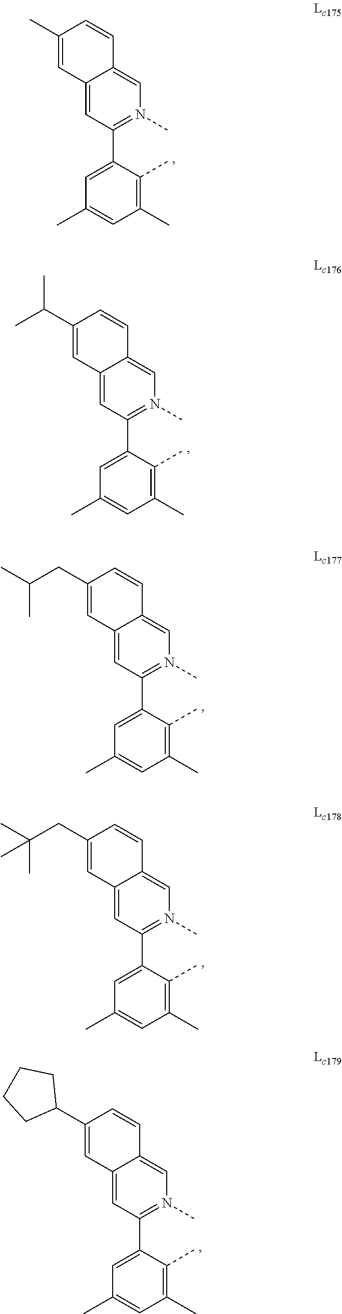

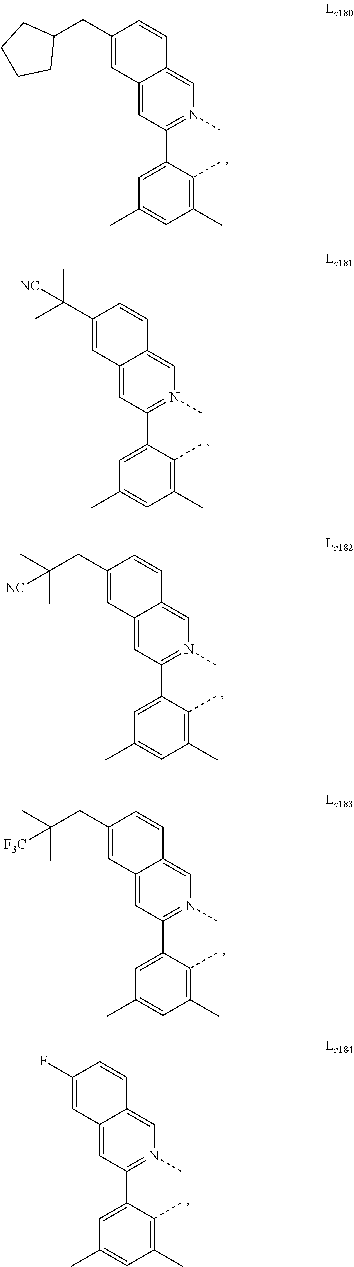

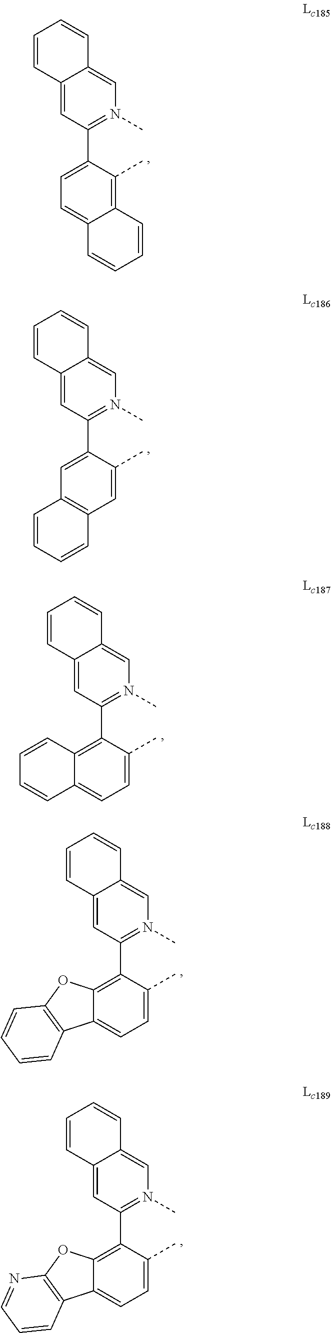

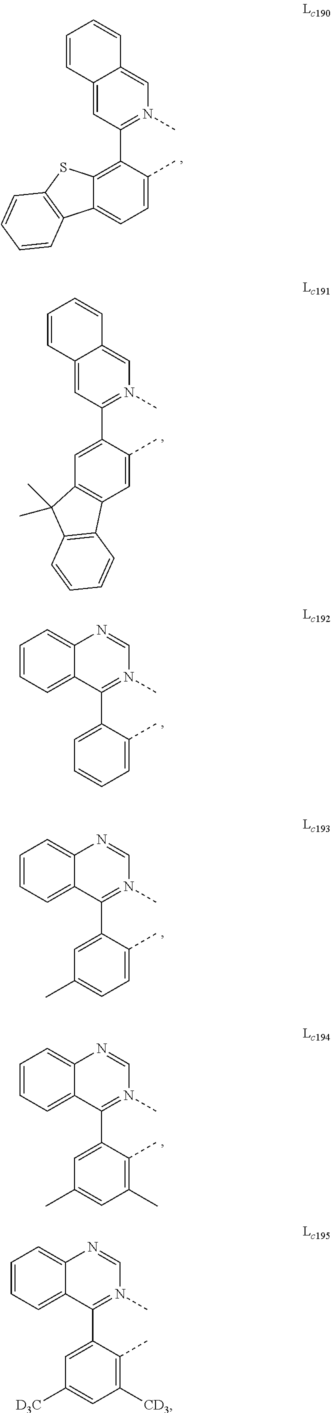

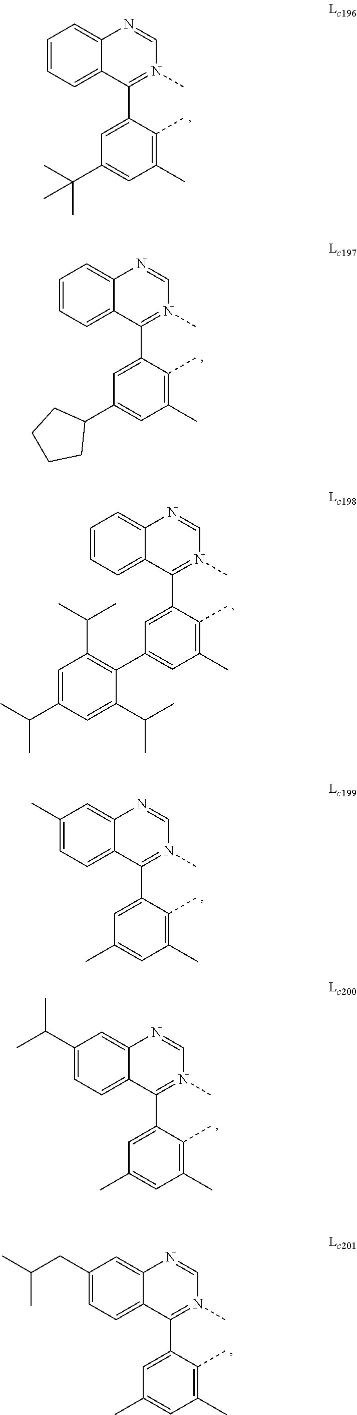

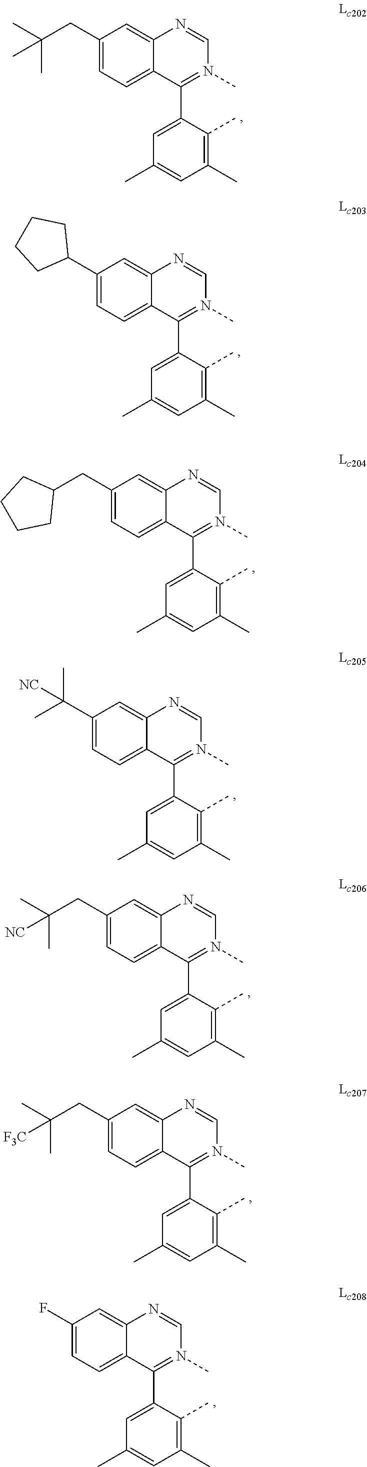

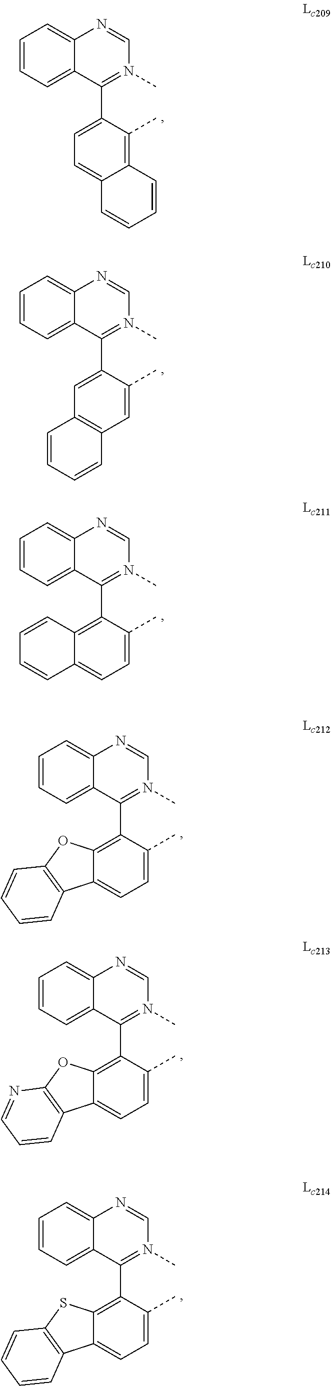

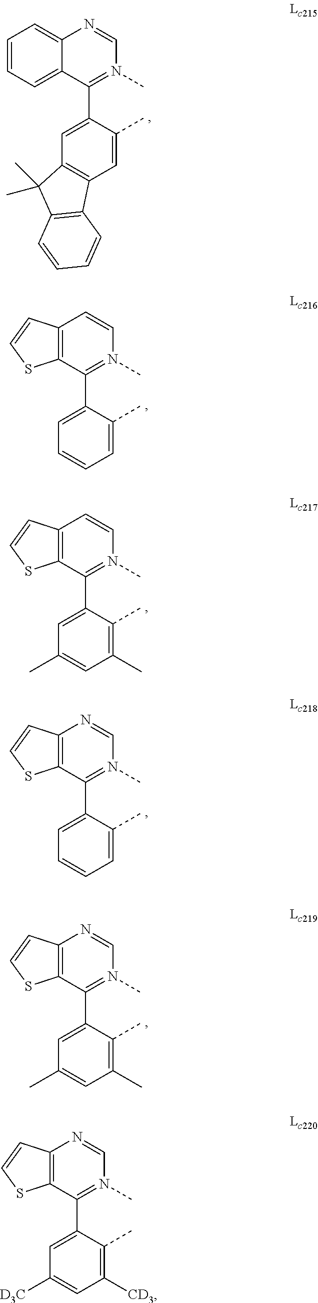

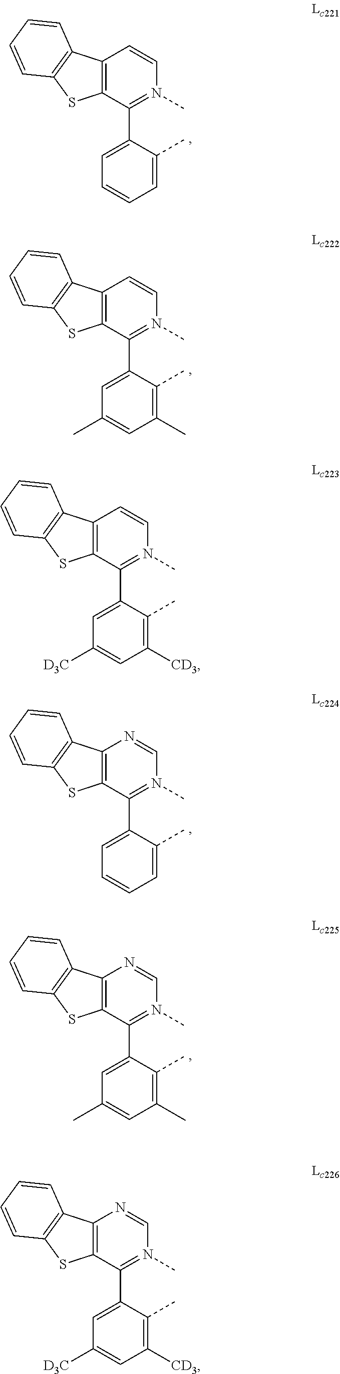

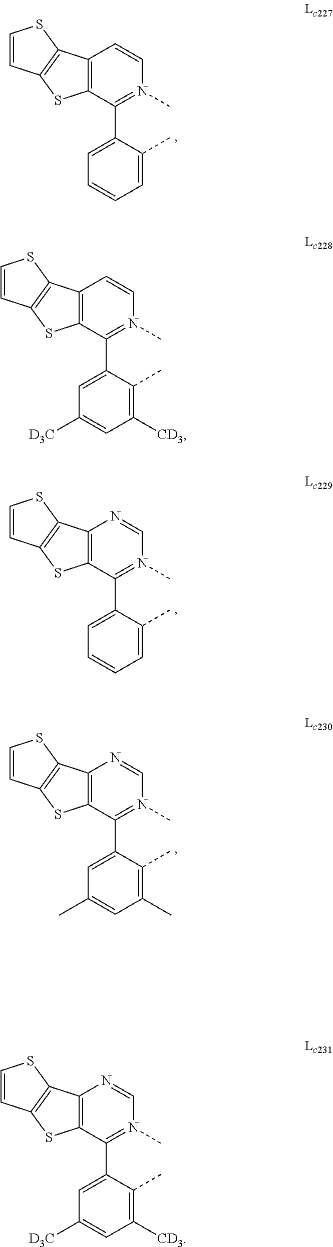

24. The electroluminescent device according to claim 23, wherein the second compound is an Ir complex and has a structure represented by any one of Ir(Lₐ)(L_{b})(L_{c}), Ir(Lₐ)₂(L_{b}), Ir(Lₐ)₂(L_{c}) and Ir(Lₐ)(L_{c})₂; when the second compound has a structure of Ir(Lₐ)(L_{b})(L_{c}), Lₐ is selected from any one of the group consisting of Lₐ₁ to Lₐ₁₆₈, L_{b} is selected from any one of the group consisting of L_{b1} to L_{b322}, and L_{c} is selected from any one of the group consisting of L_{c1} to L_{c231};
when the second compound has a structure of Ir(Lₐ)₂L_{b}, Lₐ is selected from any one or any two of the group consisting of Lₐ₁ to Lₐ₁₆₈, and L_{b} is selected from any one of the group consisting of L_{b1} to L_{b322}; when the second compound has a structure of Ir(Lₐ)₂(L_{c}), Lₐ is selected from any one or any two of the group consisting of Lₐ₁ to Lₐ₁₆₈, and L_{c} is selected from any one of the group consisting of L_{c1} to L_{c231}; when the second compound has a structure of Ir(Lₐ)(L_{c})₂, Lₐ is selected from any one of the group consisting of Lₐ₁ to Lₐ₁₆₈, and L_{c} is selected from any one or any two of the group consisting of L_{c1} to L_{c231};
preferably, the second compound is selected from the group consisting of the following structures:

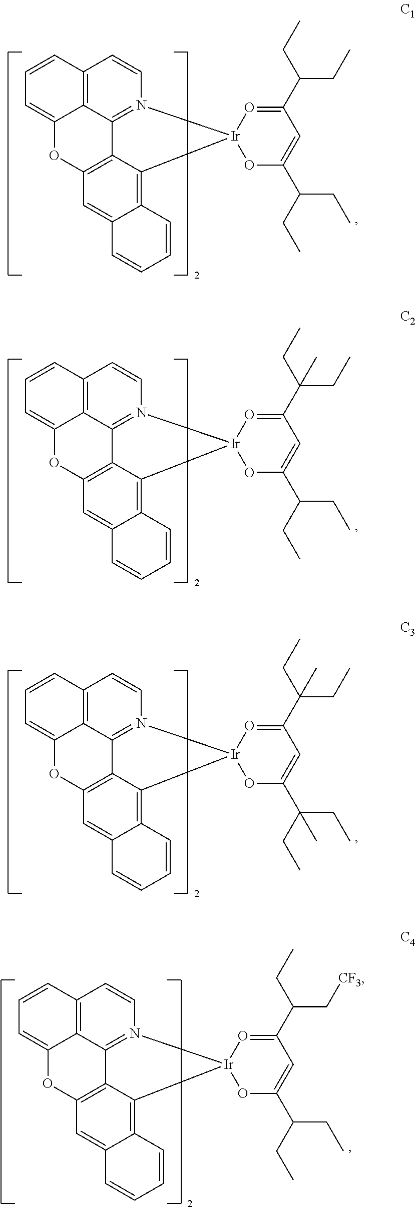

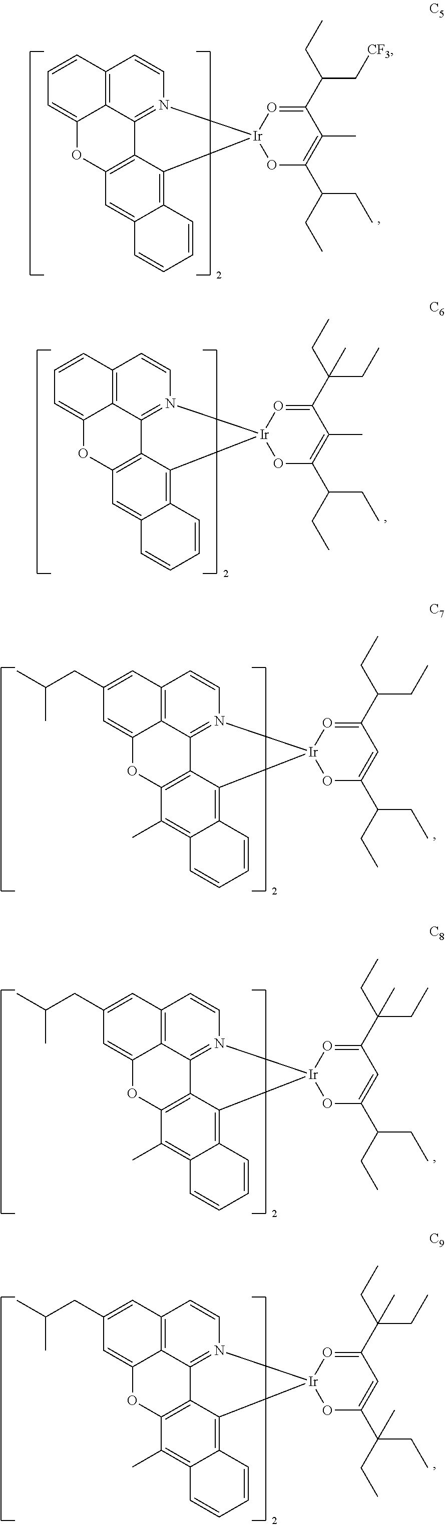

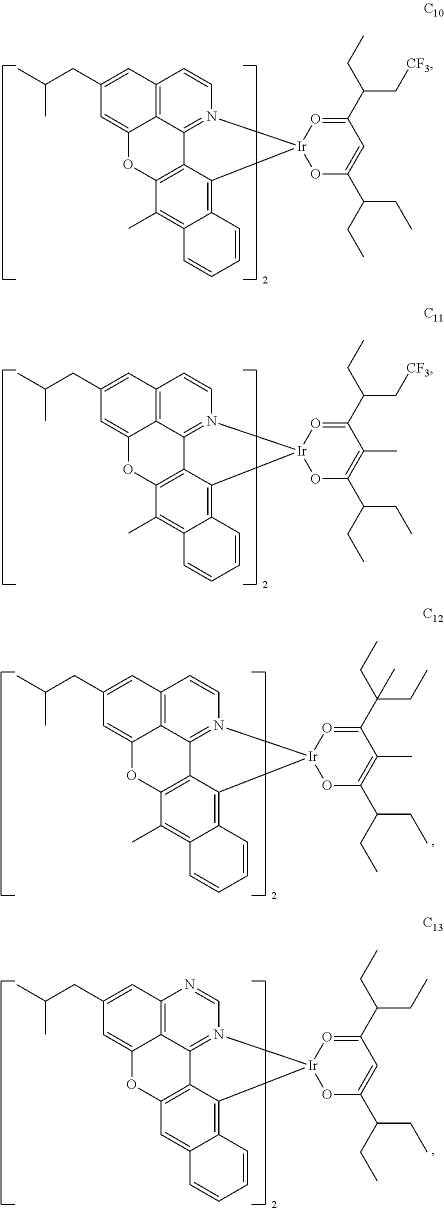

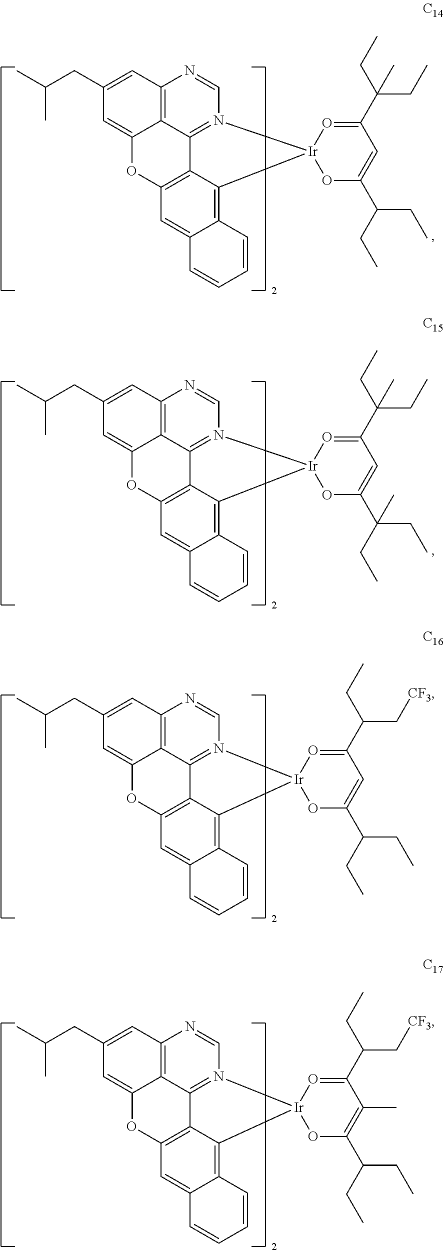

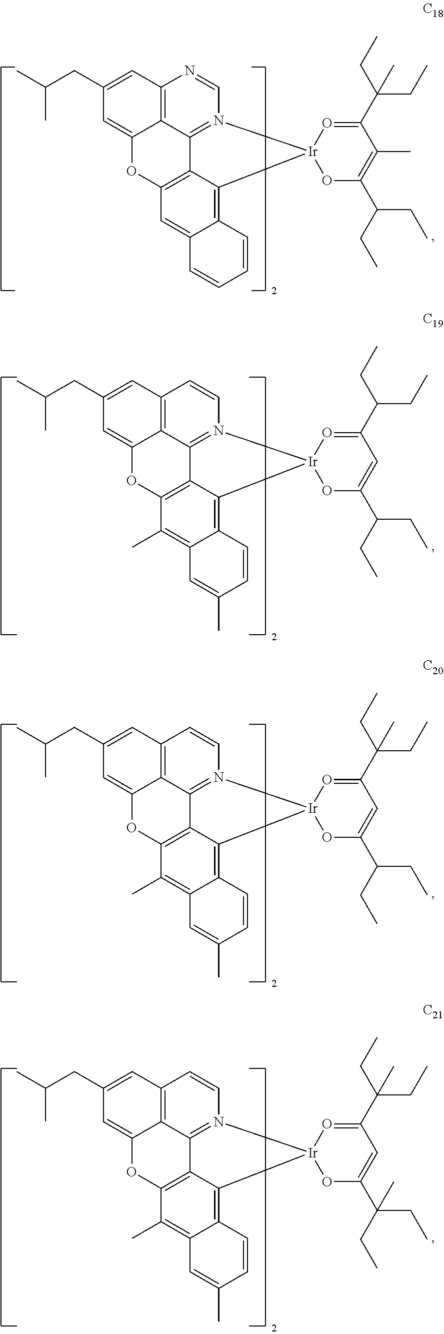

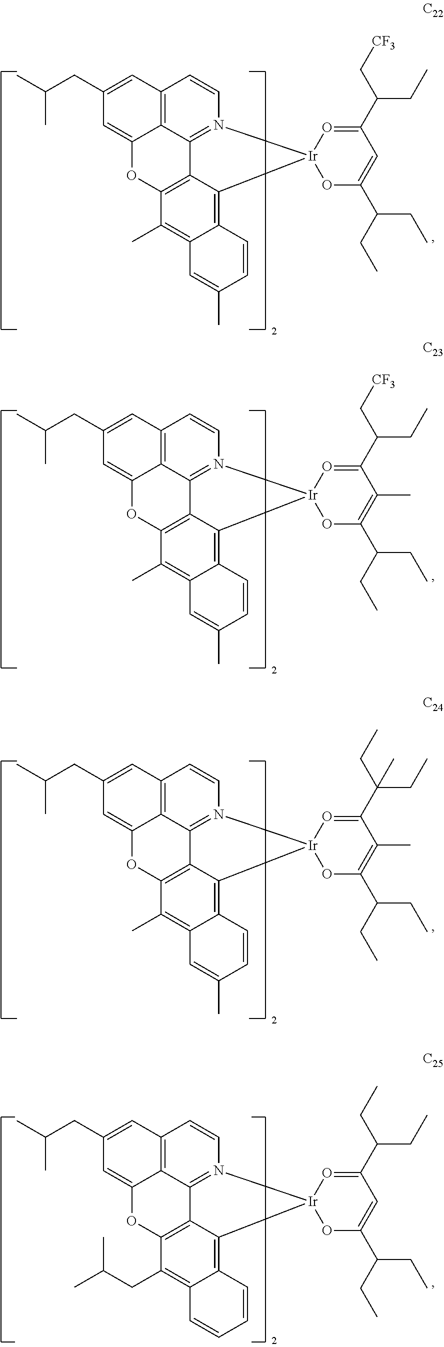

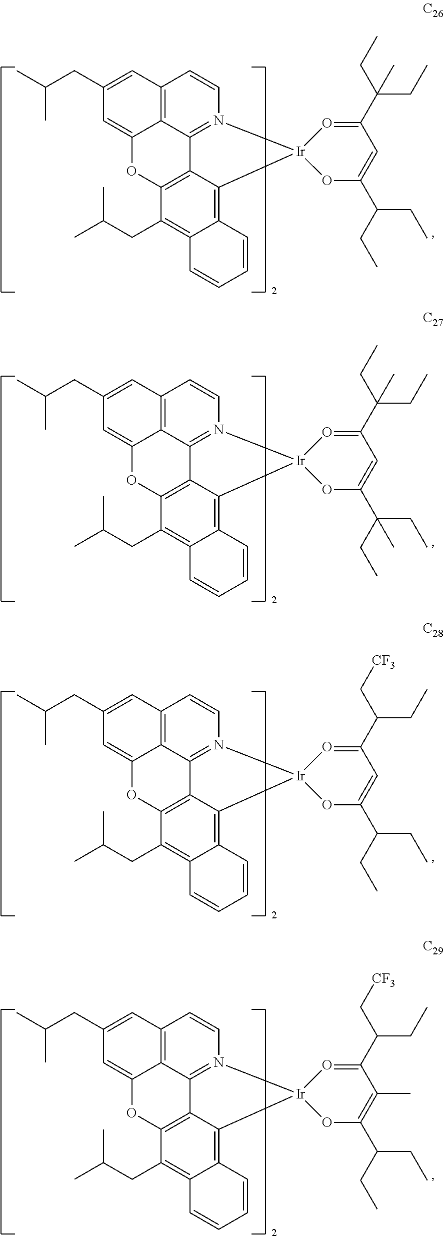

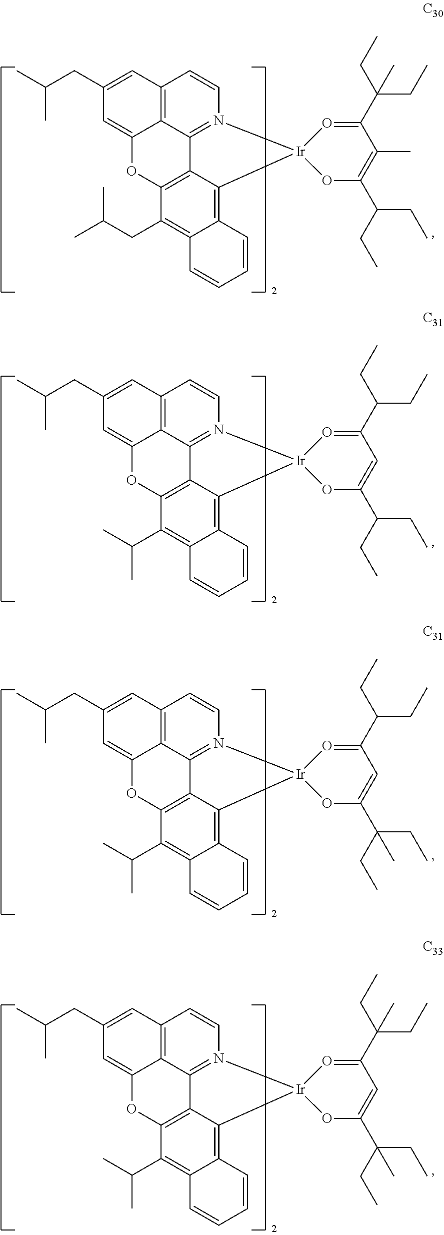

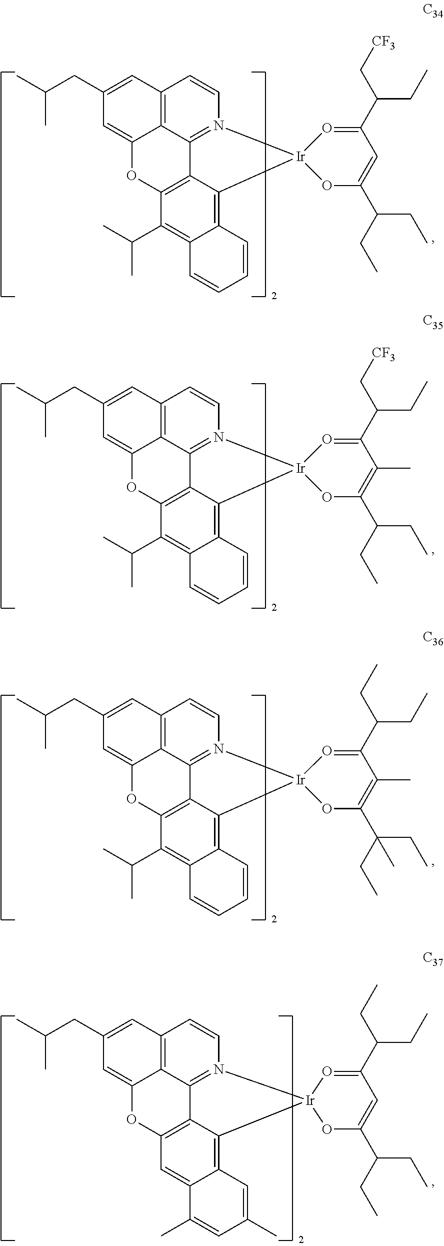

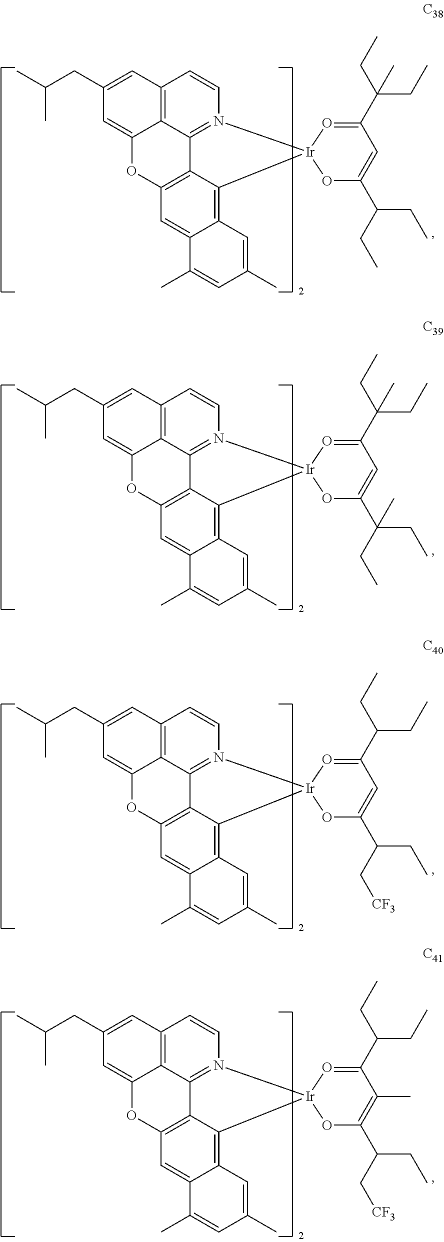

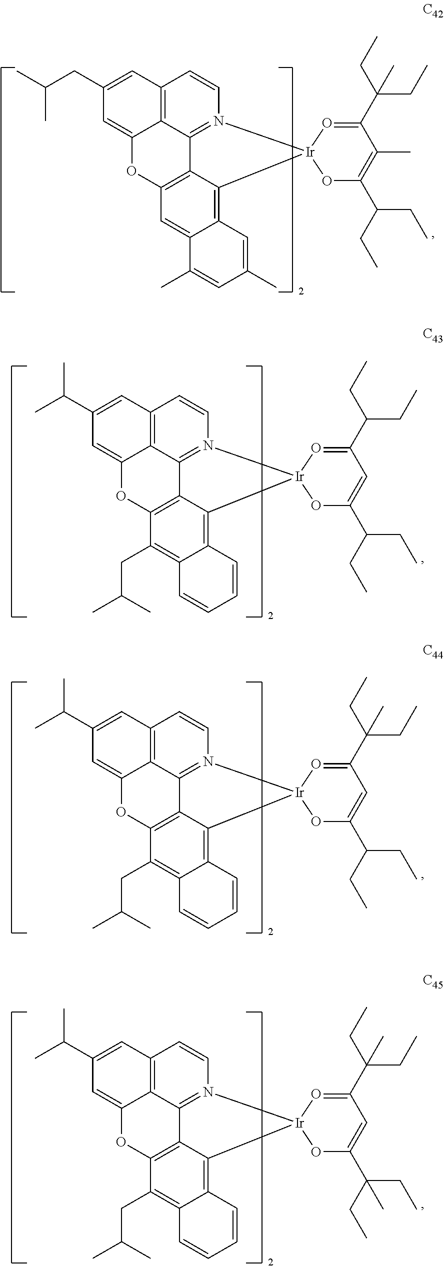

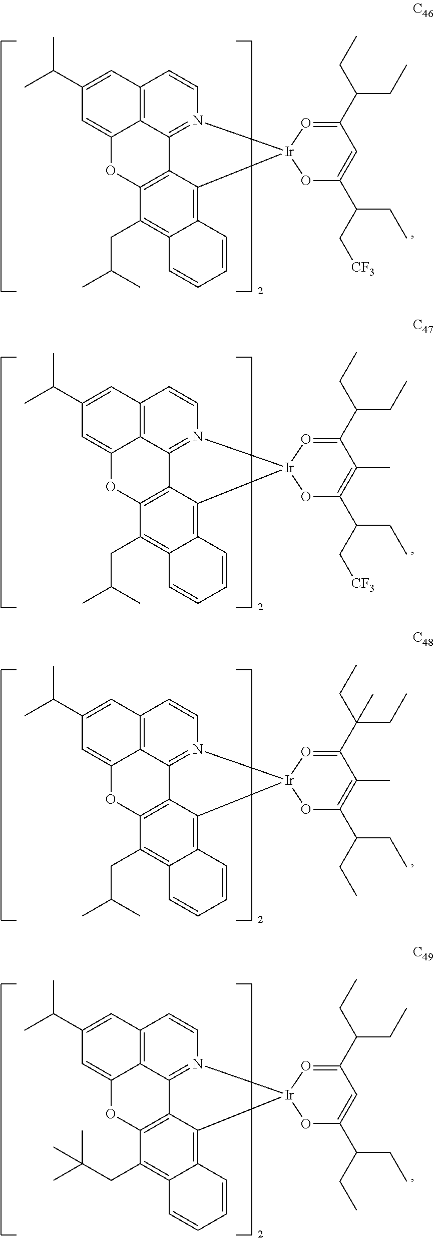

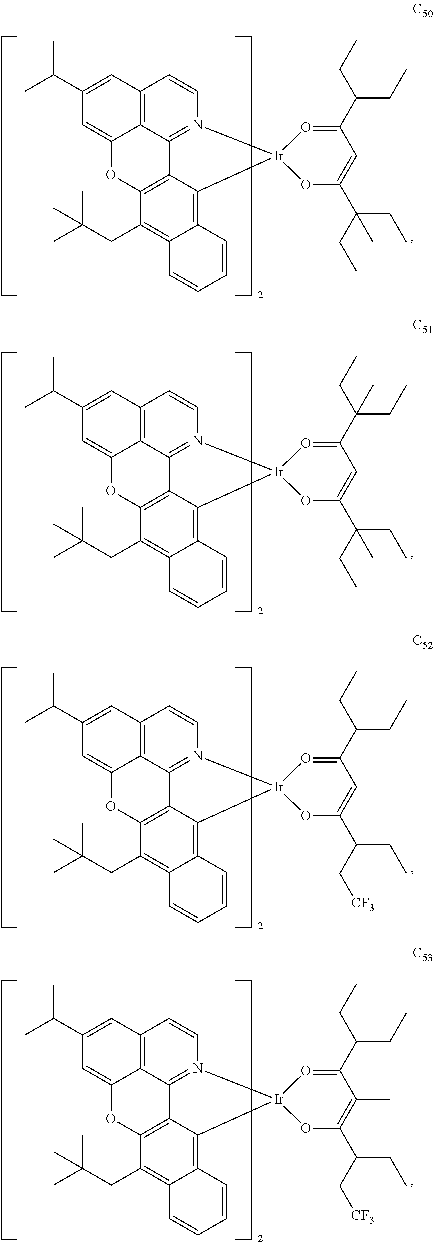

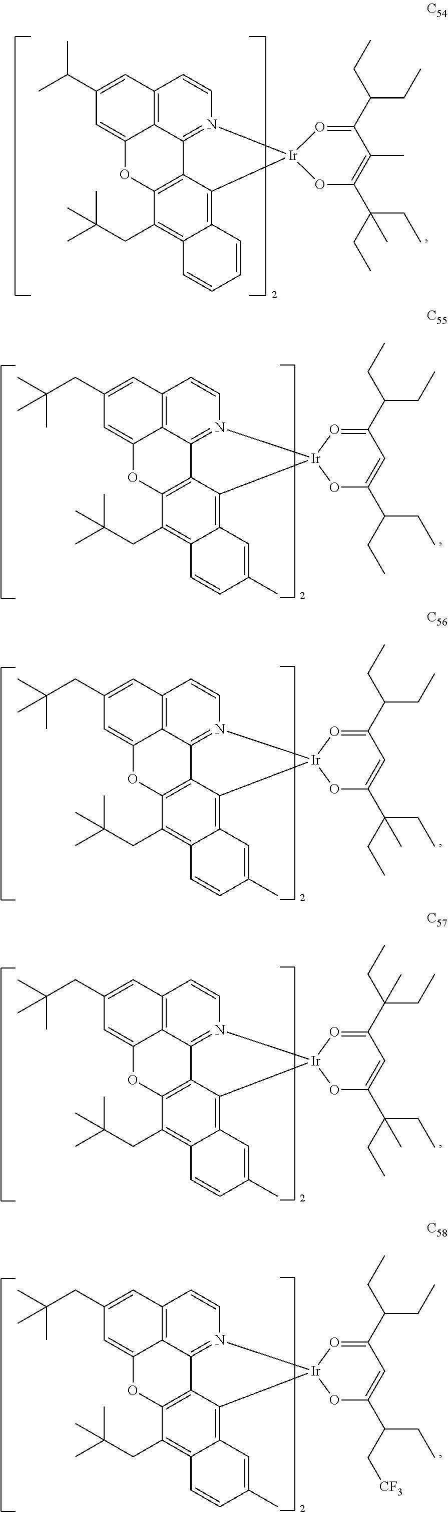

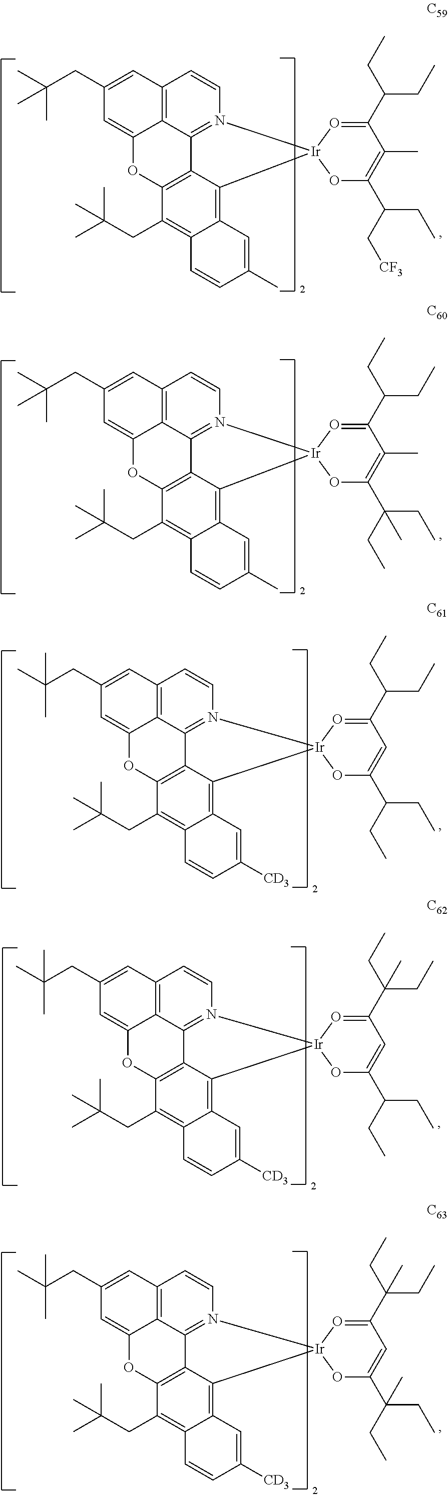

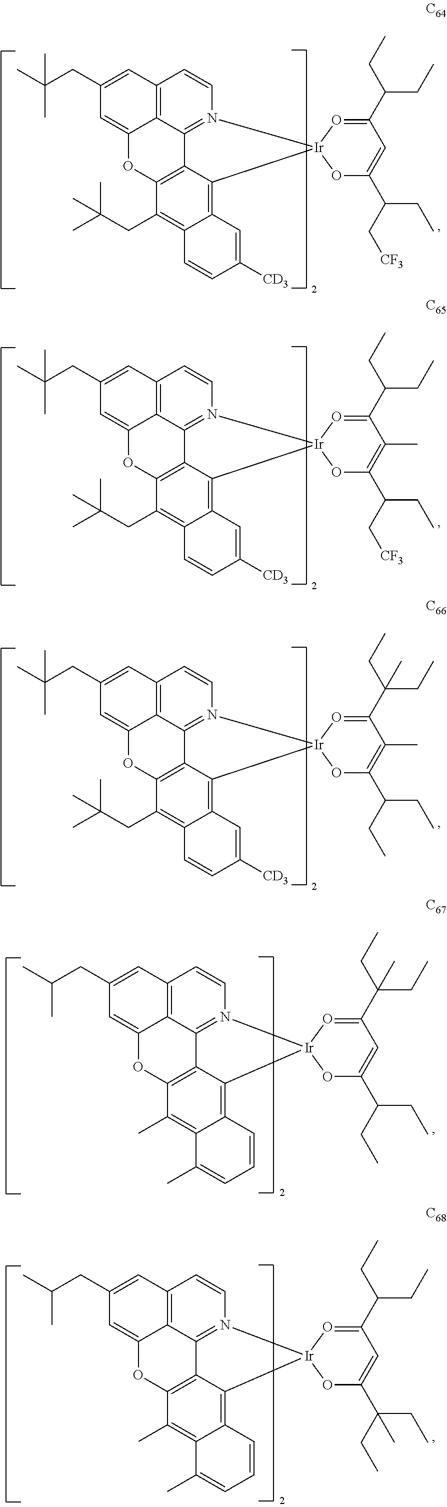

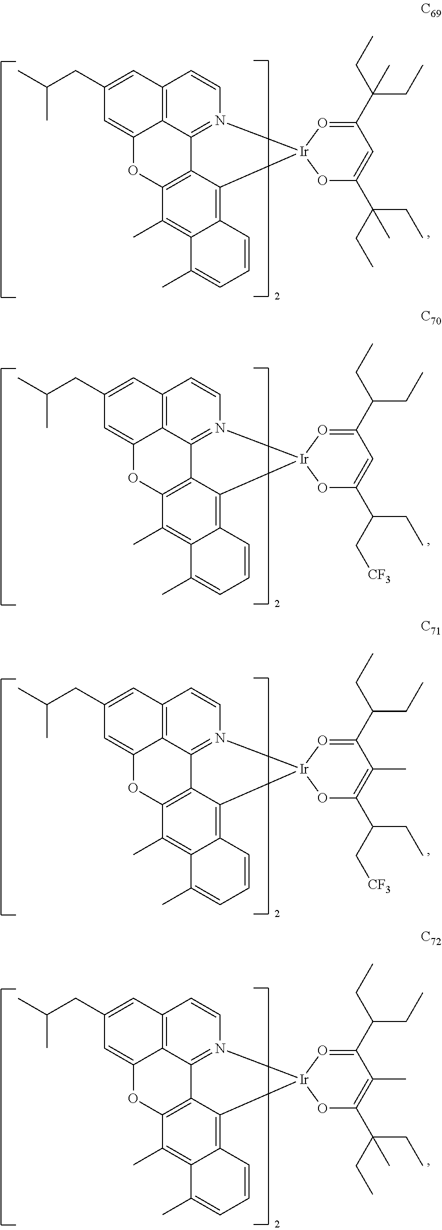

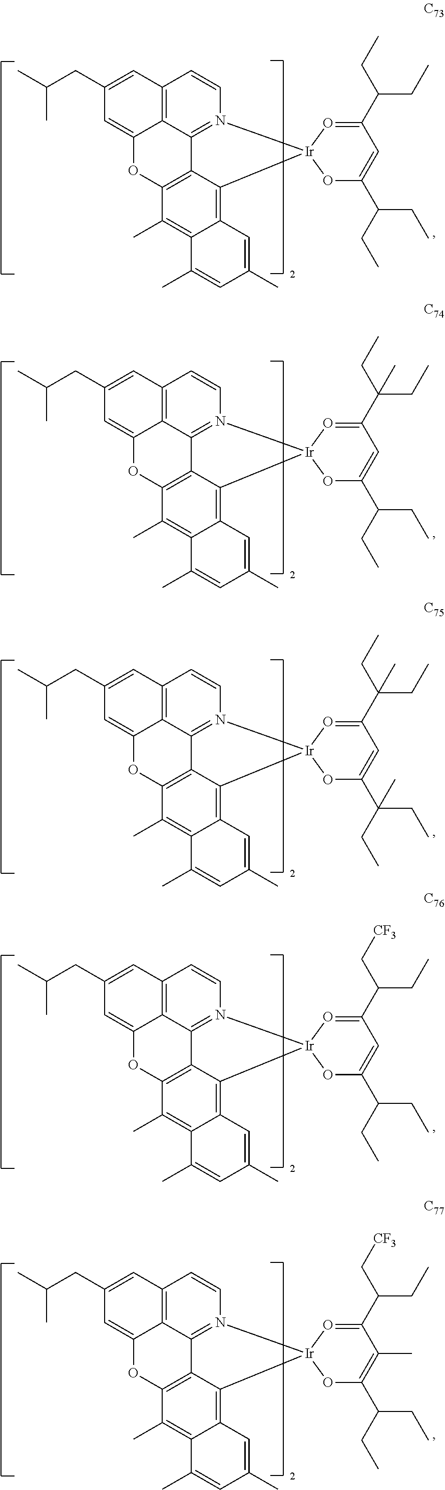

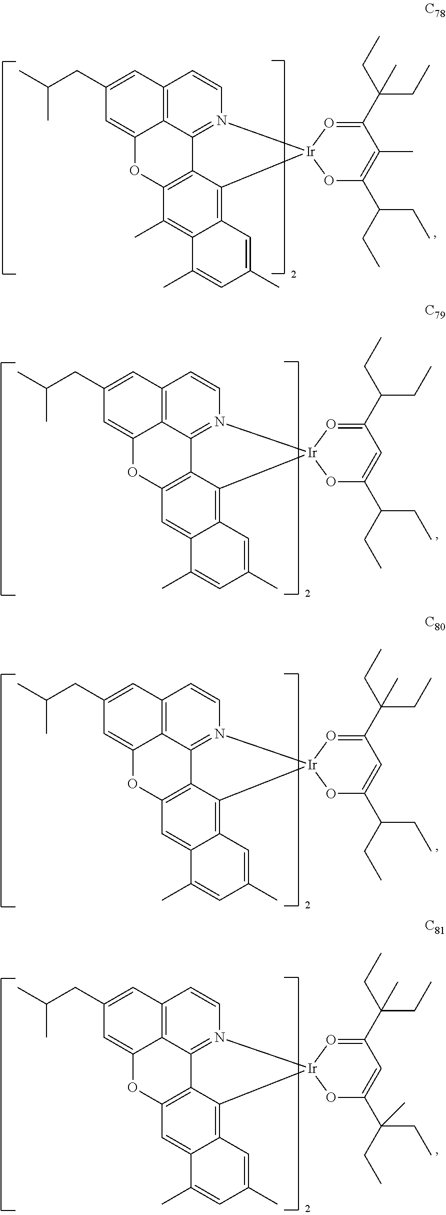

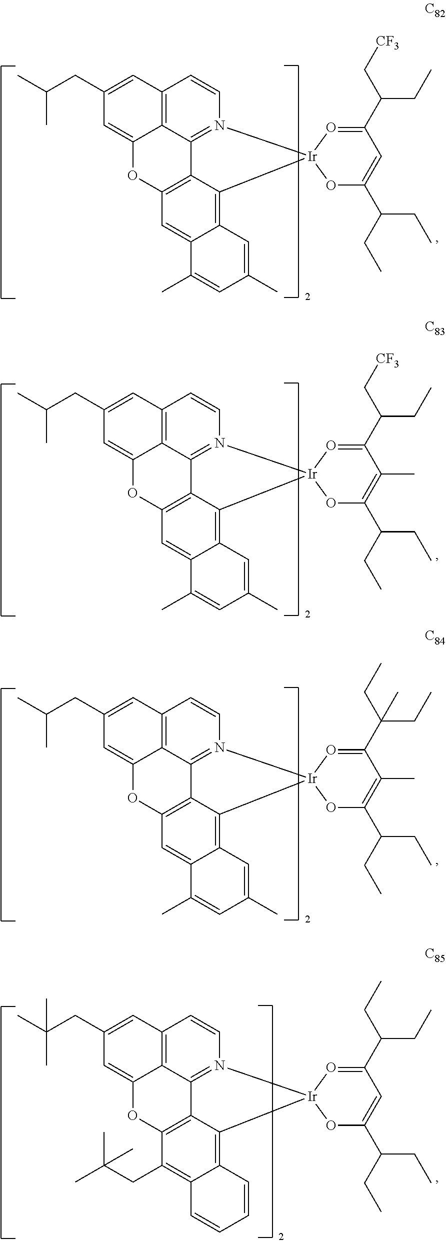

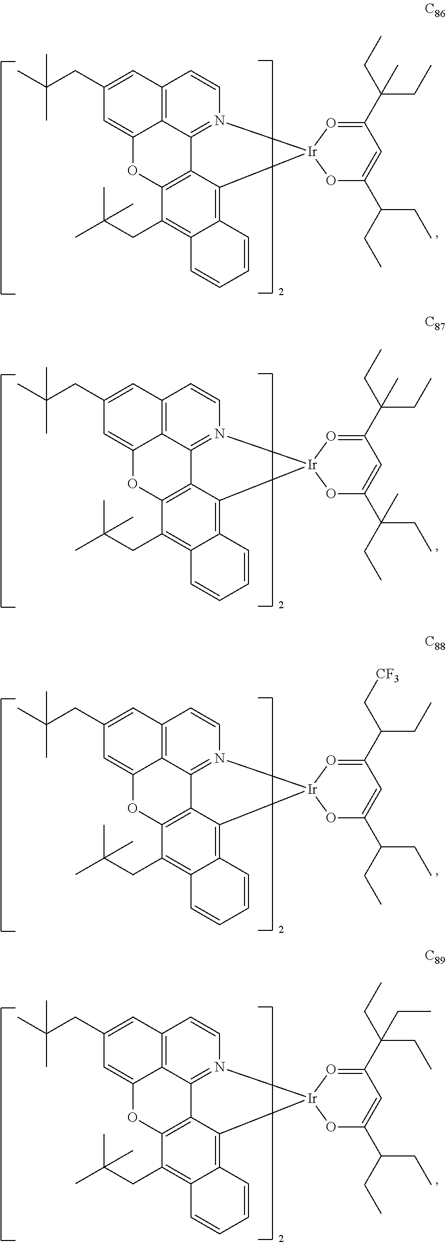

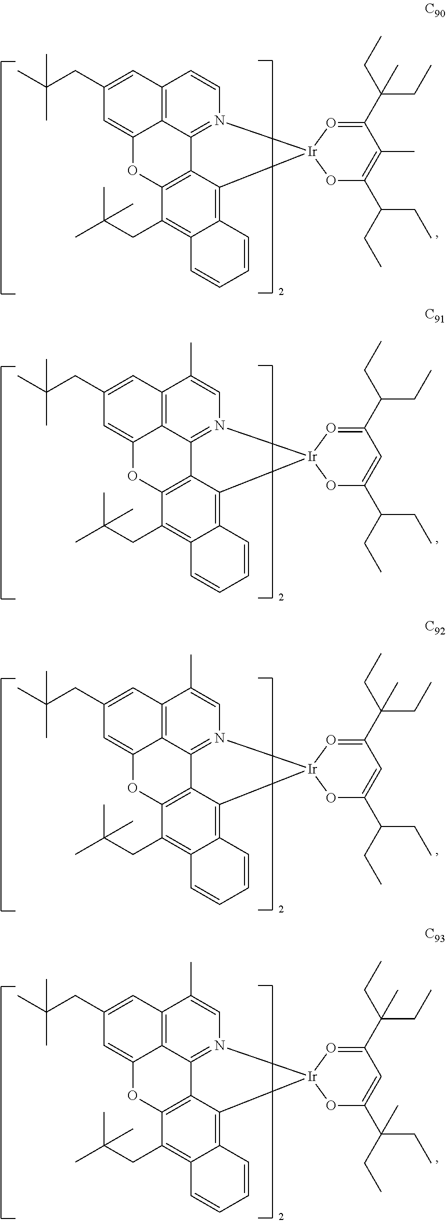

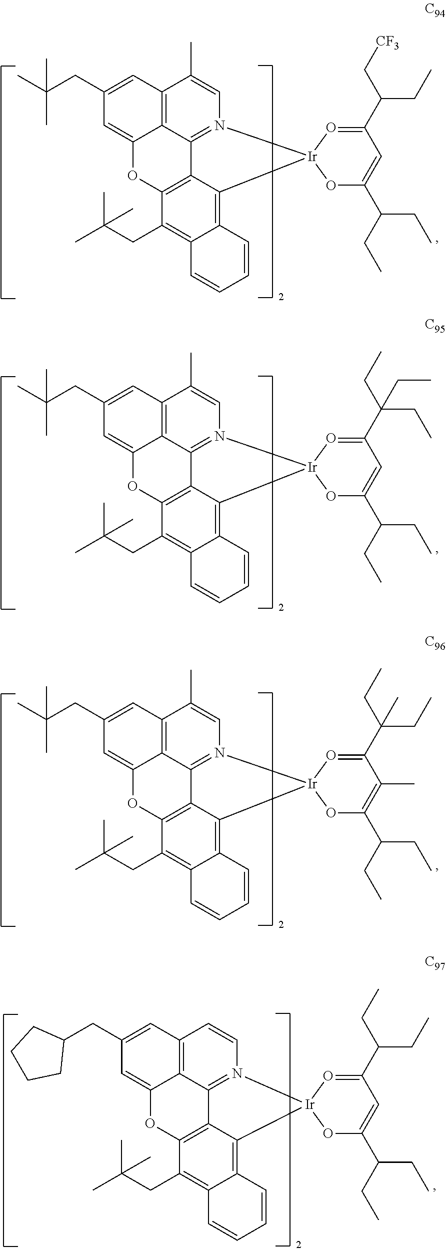

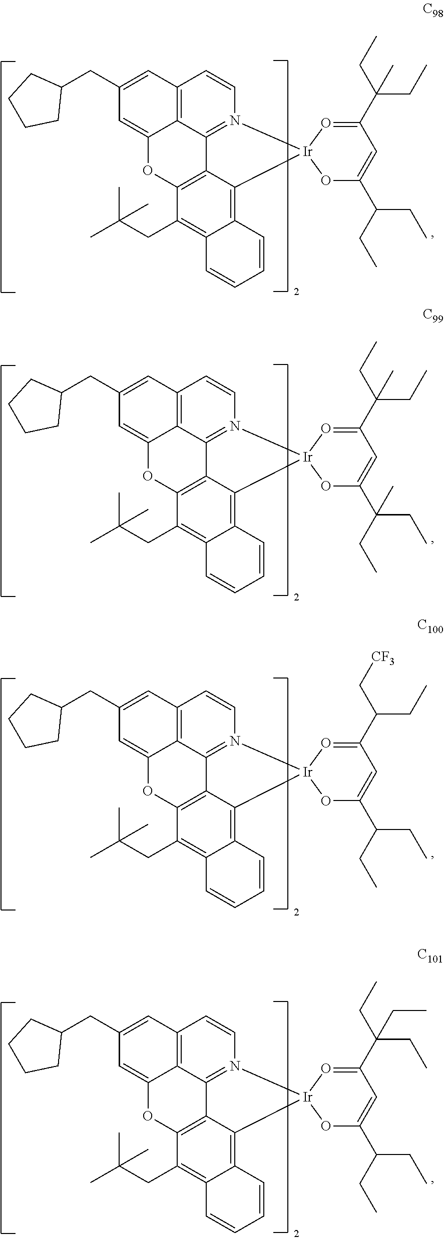

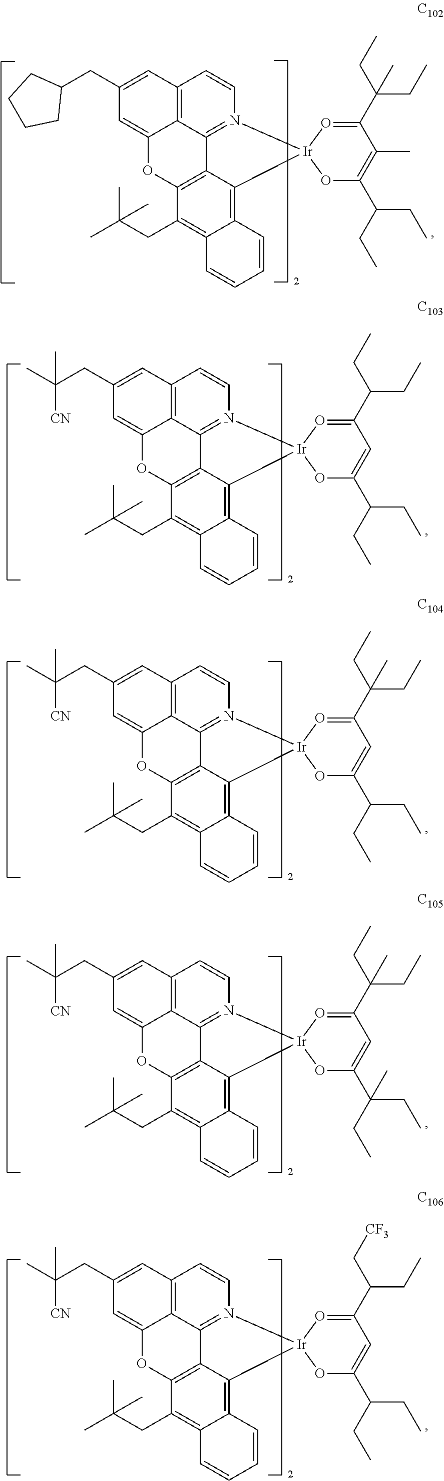

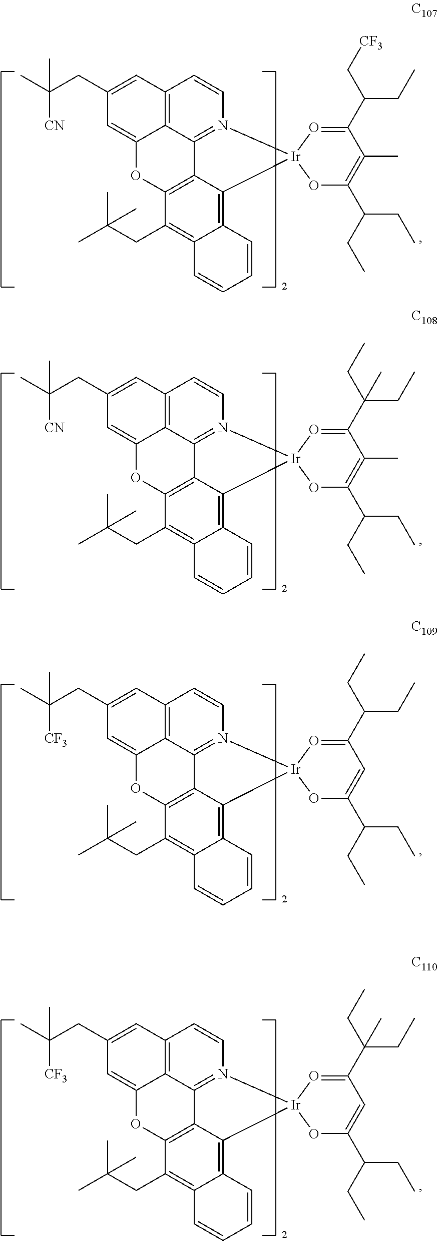

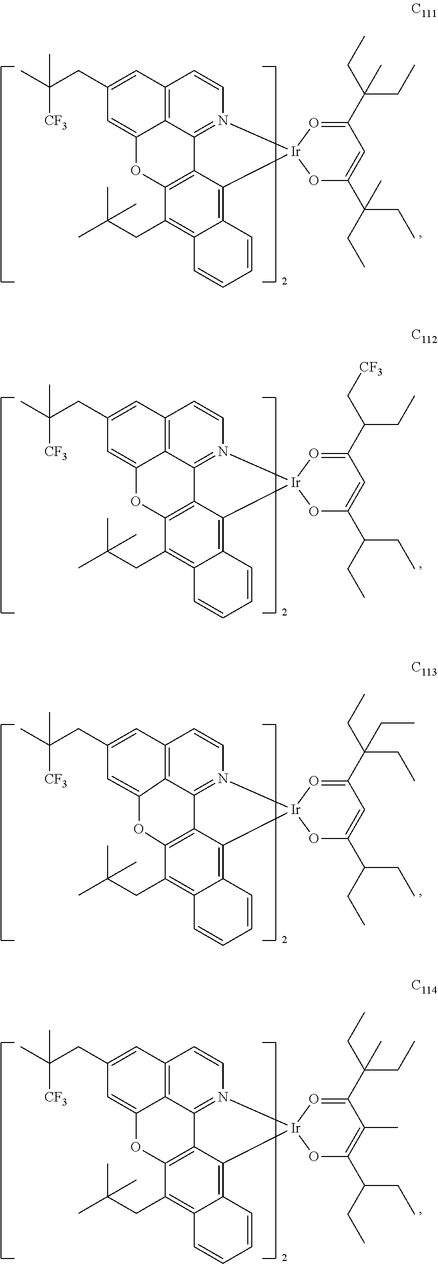

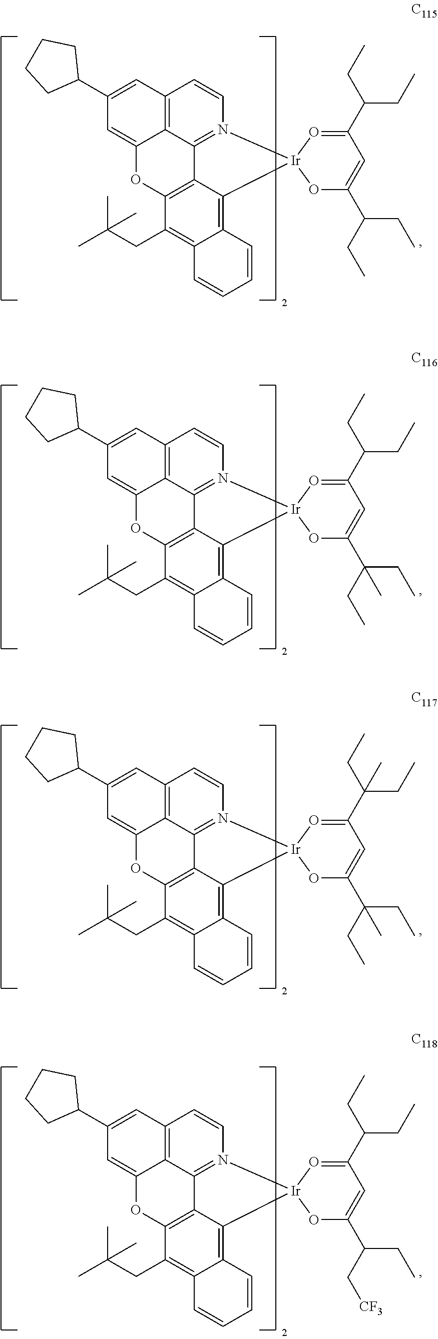

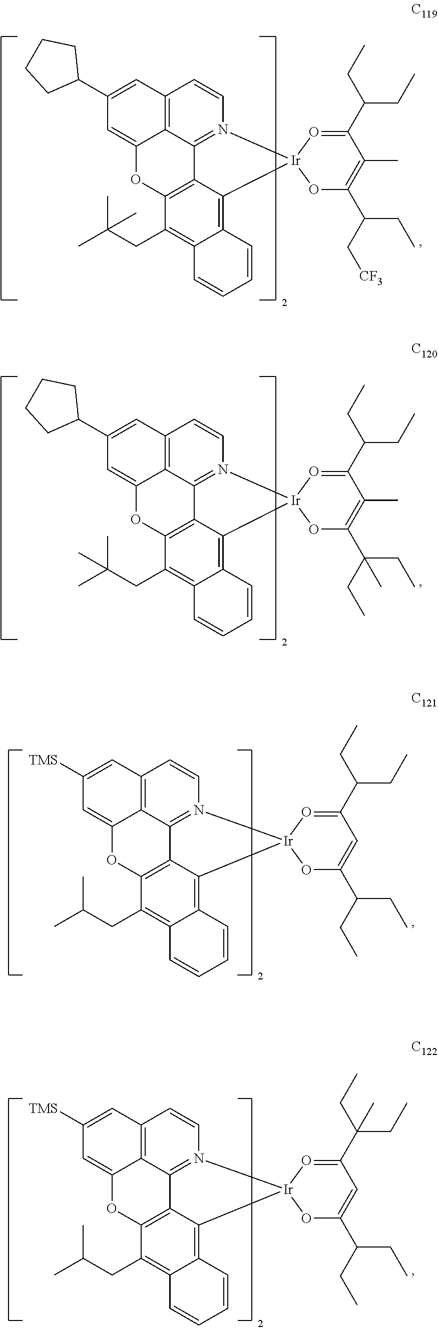

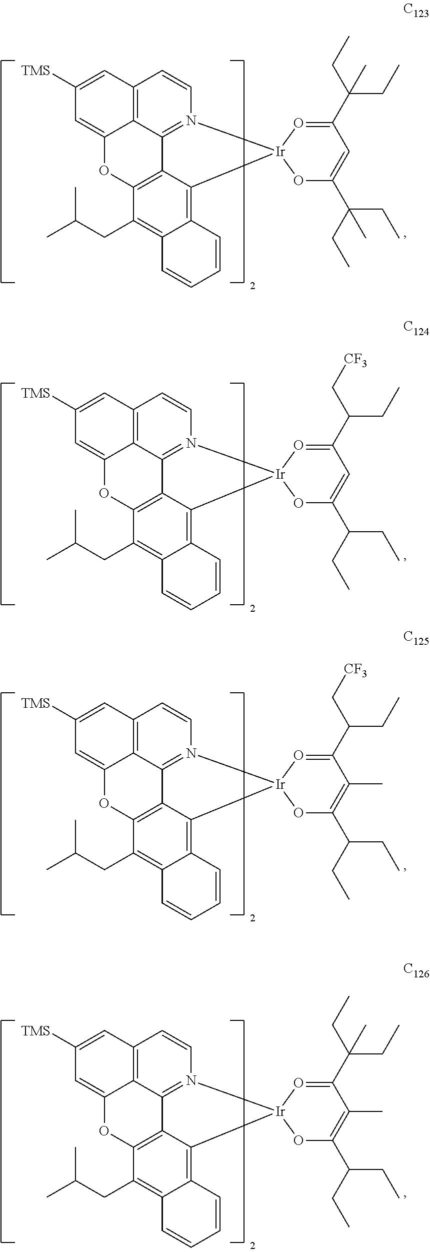

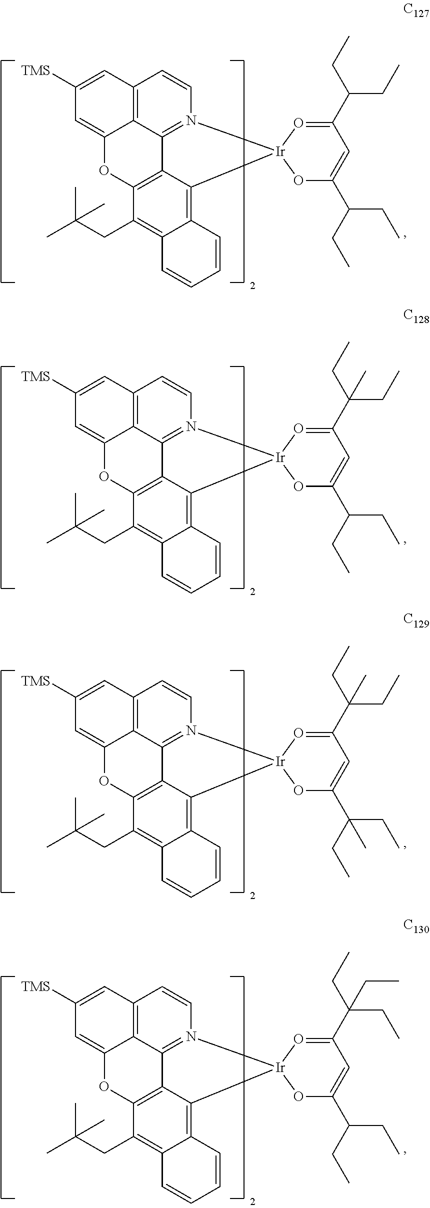

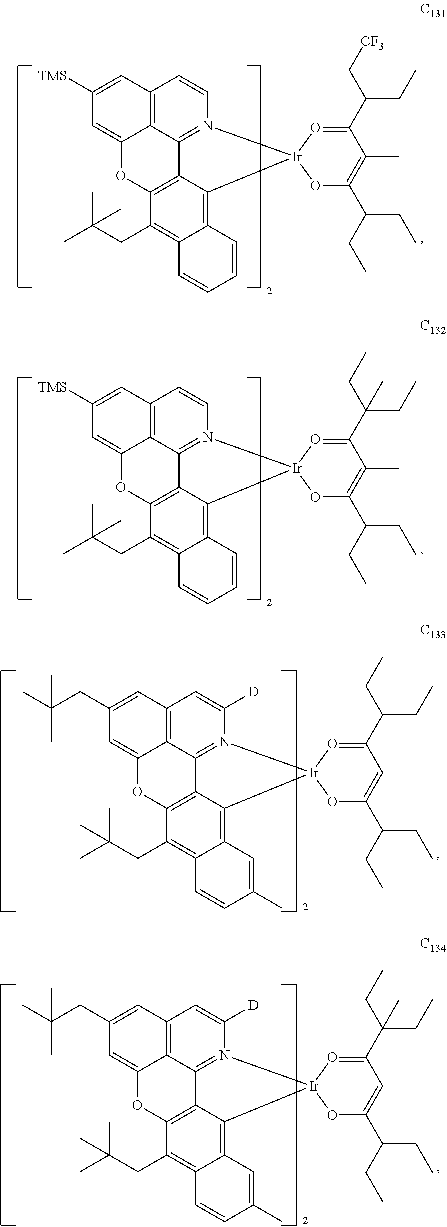

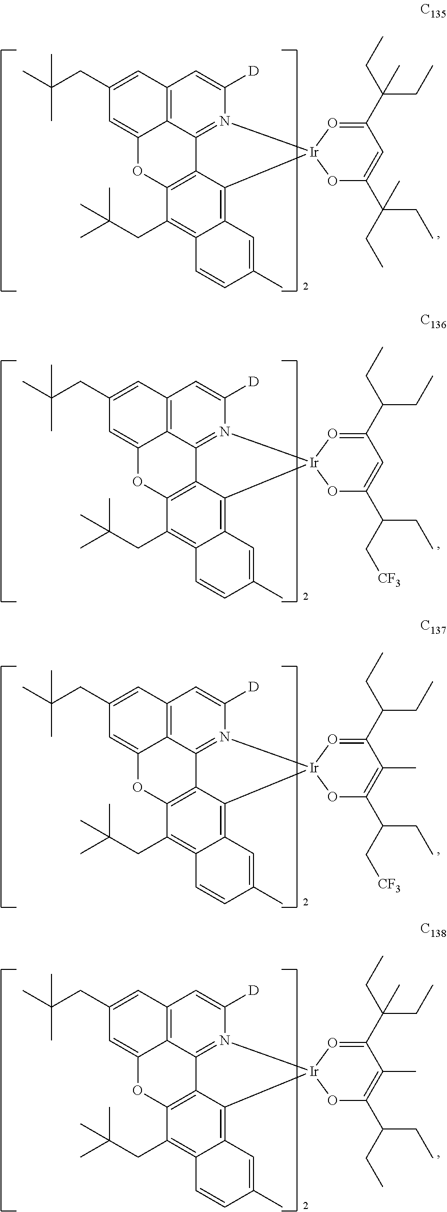

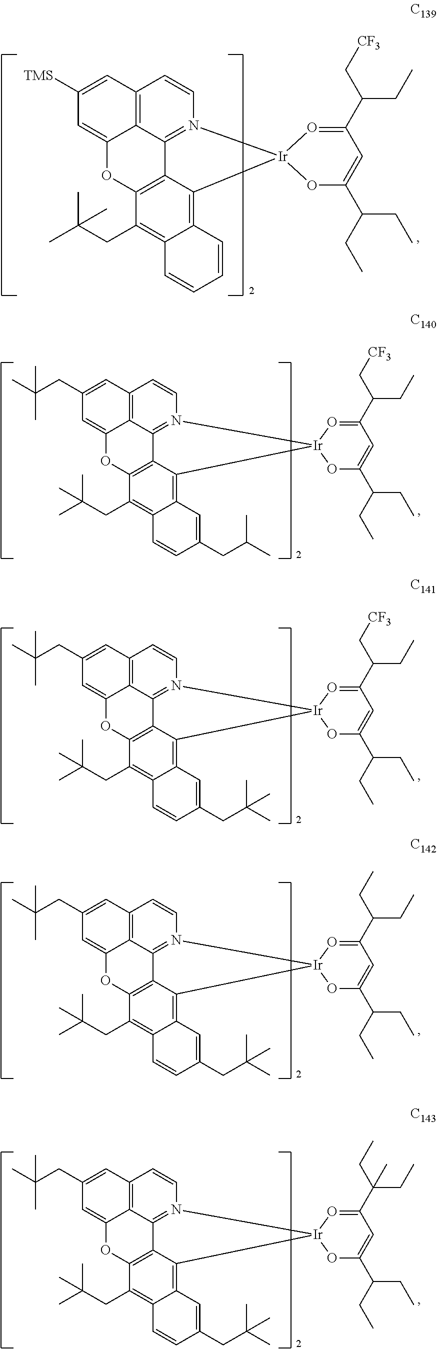

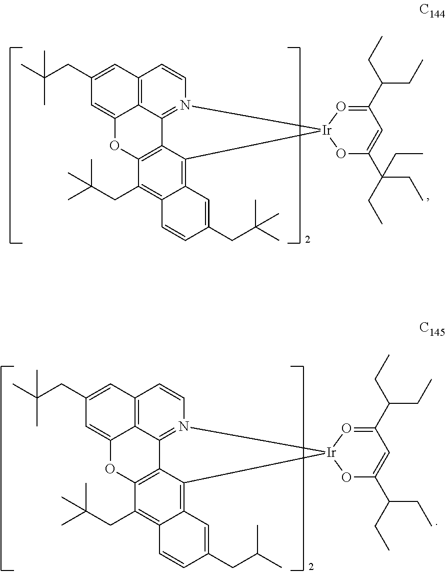

25. The electroluminescent device according to claim 1, wherein the organic layer is a light-emitting layer, the first compound is a host material, and the second compound is a light-emitting material.

26. The electroluminescent device according to claim 25, wherein the device emits red light or white light.

27. A display assembly, comprising the electroluminescent device according to claim 1.

28. A compound combination, comprising at least a first compound and a second compound;
wherein the first compound has a structure of H-L-E, wherein the H has a structure represented by Formula A: 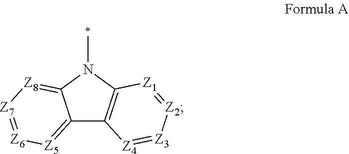
wherein in Formula A,
Z₁ to Z₃ and Z₆ to Z₈ are, at each occurrence identically or differently, selected from CR_{z1} or N, Z₄ and Z₅ are, at each occurrence identically or differently, selected from CR_{z2}, and two substituents R_{z2} in Z₄ and Z₅ are joined to form a ring;
L is selected from a single bond, substituted or unsubstituted arylene having 6 to 30 carbon atoms, substituted or unsubstituted heteroarylene having 3 to 30 carbon atoms or a combination thereof,
E is selected from substituted or unsubstituted aryl having 6 to 30 carbon atoms or substituted or unsubstituted heteroaryl having 3 to 30 carbon atoms;
R_{z1} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof,
R_{z2} is, at each occurrence identically or differently, selected from the group consisting of:
hydrogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, hydroxyl, a sulfanyl group, a sulfinyl group, a sulfonyl group, a phosphino group and combinations thereof, and
adjacent substituents R_{z1}, R_{z2} can be optionally joined to form a ring;
wherein the second compound is a metal complex, wherein the metal is selected from a metal with a relative atomic mass greater than 40, and the metal complex comprises a ligand Lₐ which has a structure represented by Formula C: 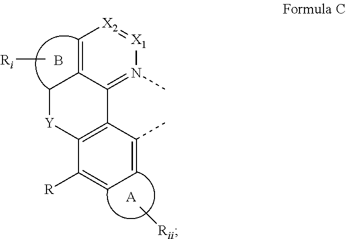
wherein in Formula C, the ring A and the ring B are each independently selected from a five-membered unsaturated carbocyclic ring, an aromatic ring having 6 to 30 carbon atoms or a heteroaromatic ring having 3 to 30 carbon atoms;
Rᵢ represents, at each occurrence identically or differently, mono-substitution, multiple substitutions or non-substitution; and Rᵢᵢ represents, at each occurrence identically or differently, mono-substitution, multiple substitutions or non-substitution;
Y is selected from SiR_{y}R_{y}, GeR_{y}R_{y}, NR_{y}, PR_{y}, O, S or Se;
when two R_{y} are present, the two R_{y} may be identical or different;
X₁ and X₂ are, at each occurrence identically or differently, selected from CRₓ or N;
R, Rᵢ, Rᵢᵢ, Rₓ and R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted amino having 0 to 20 carbon atoms, an acyl group, a carbonyl group, a carboxylic acid group, an ester group, cyano, isocyano, hydroxyl, a sulfanyl group, a sulfinyl group, a sulfonyl group, a phosphino group and combinations thereof, and
adjacent substituents Rᵢ, Rₓ, R_{y}, R and Rᵢᵢ can be optionally joined to form a ring.
